SEQUENCE LISTING (6 sequences)

SEQ ID NO: 1 - DNA, 43 bp, synthetic construct
  source 1..43: mol_type = other DNA
acggaattcgccaccatgaagctcagtaaaaaggaccgaggag

SEQ ID NO: 2 - DNA, 42 bp, synthetic construct
  source 1..42: mol_type = other DNA
cgttgcggccgcatgagcacttttaaagttctgctatgtggc

SEQ ID NO: 3 - DNA, 1885 bp, synthetic construct
  source 1..1885: mol_type = other DNA
gttggctctagggctggcaccaggcactggaattaaacattctggactccagaggggcatgaaaactcttcctgaaggagatgcagaggaagattcgaactggaggaaaaccctaaaataaacaataacaacaaaagttcaaaacctgaaaagtgaaccatgaagctcagtaaaaaggaccgaggagaagatgaagaaagtgattcagcgaaaaagaaattggactggtcctgctcgctcctcgtggcctccctcgcgggcgccttcggctcctccttcctctacggctacaacctgtcggtggtgaatgcccccaccccgtacatcaaggccttttacaatgagtcatgggaaagaaggcatggacgtccaatagacccagacactctgactttgctctggtctgtgactgtgtccatattcgccatcggtggacttgtggggacgttaattgtgaagatgattggaaaggttcttgggaggaagcacactttgctggccaataatgggtttgcaatttctgctgcattgctgatggcctgctcgctccaggcaggagcctttgaaatgctcatcgtgggacgcttcatcatgggcatagatggaggcgtcgccctcagtgtgctccccatgtaccttagtgagatctcacccaaggagatccgtggctctctggggcaggtgactgccatctttatctgcattggcgtgttcactgggcagcttctgggcctgcccgagctgctgggaaaggagagtacctggccatacctgtttggagtgattgtggtccctgccgttgtccagctgctgagccttccctttctcccggacagcccacgctacctgctcttggagaagcacaacgaggcaagagctgtgaaagccttccaaacgttcttgggtaaagcagacgtttcccaagaggtagaggaggtcctggctgagagccgcgtgcagaggagcatccgcctggtgtccgtgctggagctgctgagagctccctacgtccgctggcaggtggtcaccgtgattgtcaccatggcctgctaccagctctgtggcctcaatgcaatttggttctataccaacagcatctttggaaaagctgggatccctccggcaaagatcccatacgtcaccttgagtacagggggcatcgagactttggctgccgtcttctctggtttggtcattgagcacctgggacggagacccctcctcattggtggctttgggctcatgggcctcttctttgggaccctcaccatcacgctgaccctgcaggaccacgccccctgggtcccctacctgagtatcgtgggcattctggccatcatcgcctctttctgcagtgggccaggtggcatcccgttcatcttgactggtgagttcttccagcaatctcagcggccggctgccttcatcattgcaggcaccgtcaactggctctccaactttgctgttgggctcctcttcccattcattcagaaaagtctggacacctactgtttcctagtctttgctacaatttgtatcacaggtgctatctacctgtattttgtgctgcctgagaccaaaaacagaacctatgcagaaatcagccaggcattttccaaaaggaacaaagcatacccaccagaagagaaaatcgactcagctgtcactgatggtaagataaatggaaggccttaacaagtttcctcctccacgttggacaattatgtcaaaaacaggattgtctacatggatgatctcacttttcaggaaacttaaaatttacccattattgggaagcttaaatgaattgaagctatgcaagtcttttatattattaaatatttaaaagtaaacctgtactaatctaaaaaaaaaaaaaaaaaaa

SEQ ID NO: 4 - protein, 540 aa, synthetic construct
MARKQNRNSKELGLVPLTDDTSHAGPPGPGRALLECDHLRSGVPGGRRRKDWSCSLLVASLAGAFGSSFLYGYNLSVVNAPTPYIKAFYNESWERRHGRPIDPDTLTLLWSVTVSIFAIGGLVGTLIVKMIGKVLGRKHTLLANNGFAISAALLMACSLQAGAFEMLIVGRFIMGIDGGVALSVLPMYLSEISPKEIRGSLGQVTAIFICIGVFTGQLLGLPELLGKESTWPYLFGVIVVPAVVQLLSLPFLPDSPRYLLLEKHNEARAVKAFQTFLGKADVSQEVEEVLAESRVQRSIRLVSVLELLRAPYVRWQVVTVIVTMACYQLCGLNAIWFYTNSIFGKAGIPPAKIPYVTLSTGGIETLAAVFSGLVIEHLGRRPLLIGGFGLMGLFFGTLTITLTLQDHAPWVPYLSIVGILAIIASFCSGPGGIPFILTGEFFQQSQRPAAFIIAGTVNWLSNFAVGLLFPFIQKSLDTYCFLVFATICITGAIYLYFVLPETKNRTYAEISQAFSKRNKAYPPEEKIDSAVTDGKINGRP

SEQ ID NO: 5 - DNA, 2642 bp, synthetic construct
  source 1..2642: mol_type = other DNA
gccccgagtctgtgaagcctagccgctgggctggagaagccactgtgggcaccaccgtgggggaaacaggcccgttgccctggcctctttgccctgggccagcctttgtgaagtgggcccctcttctgggccccttgagtaggttccatggcattttctgaactcctggacctcgtgggtggcctgggcaggttccaggttctccagacgatggctctgatggtctccatcatgtggctgtgtacccagagcatgctggagaacttctcggccgccgtgcccagccaccgctgctgggcacccctcctggacaacagcacggctcaggccagcatcctagggagcttgagtcctgaggccctcctggctatttccatcccgccgggccccaaccagaggccccatcagtgccgccgcttccgccagccacagtggcagctcttggaccccaatgccacggccaccagctggagcgaggccgacacggagccgtgtgtggatggctgggtctatgaccgcagcatcttcacctccacaatcgtggccaagtggaacctcgtgtgtgactctcacgctctgaagcccatggcccagtccatctacctggctgggattctggtgggagctgctgcgtgcggccctgcctcagacaggtttgggcgcaggctggtgctaacctggagctaccttcagatggctgtgatgggtacggcagctgccttcgcccctgccttccccgtgtactgcctgttccgcttcctgttggcctttgccgtggcaggcgtcatgatgaacacgggcactctcctgatggagtggacggcggcacgggcccgacccttggtgatgaccttgaactctctgggcttcagcttcggccatggcctgacagctgcagtggcctacggtgtgcgggactggacactgctgcagctggtggtctcggtccccttcttcctctgctttttgtactcctggtggctggcagagtcggcacgatggctcctcaccacaggcaggctggattggggcctgcaggagctgtggagggtggctgccatcaacggaaagggggcagtgcaggacaccctgacccctgaggtcttgctttcagccatgcgggaggagctgagcatgggccagcctcctgccagcctgggcaccctgctccgcatgcccggactgcgcttccggacctgtatctccacgttgtgctggttcgcctttggcttcaccttcttcggcctggccctggacctgcaggccctgggcagcaacatcttcctgctccaaatgttcattggtgtcgtggacatcccagccaagatgggcgccctgctgctgctgagccacctgggccgccgccccacgctggccgcatccctgttgctggcggggctctgcattctggccaacacgctggtgccccacgaaatgggggctctgcgctcagccttggccgtgctggggctgggcggggtgggggctgccttcacctgcatcaccatctacagcagcgagctcttccccactgtgctcaggatgacggcagtgggcttgggccagatggcagcccgtggaggagccatcctggggcctctggtccggctgctgggtgtccatggcccctggctgcccttgctggtgtatgggacggtgccagtgctgagtggcctggccgcactgcttctgcccgagacccagagcttgccgctgcccgacaccatccaagatgtgcagaaccaggcagtaaagaaggcaacacatggcacgctggggaactctgtcctaaaatccacacagttttagcctcctgaggaacctgcgatgggacggtcagaggaagagacttcttctgttctctggagaaggcaggaggaaagcaaagacctccatttccagaggcccagaggctgccctctgaggtccccactctcccccagggctgcccctccaggtgagccctgcccctctcacagtccaaggggcccccttcaatactgaaggggaaaaggacagtttgattggcaggaggtgacccagtgcaccatcaccctgccctgccctcgtggcttcggagagcagaggggtcaggcccaggggaacgagctggccttgccaaccctctgcttgactccgcactgccacttgtccccccacacccgtccacctgcccagagctcagagctaaccaccatccatggtcaagacctctcctagctccacacaagcagtagagtctcagctccacagctttacccagaagccctgtaagcctggcccctggcccctccccatgtccctccaggcctcagccacctgcccgccacatcctctgcctgctgtccccttcccaccctcatccctgaccgactccacttaacccccaaacccagccccccttccaggggtccagggccagcctgagatgcccgtgaaactcctacccacagttacagccacaagcctgcctcctcccaccctgccagcctatgagttcccagagggttggggcagtcccatgaccccatgtcccagctccccacacagcgctgggccagagaggcattggtgcgagggattgaataaagaaacaaatgaaaaaaaaaaaaaaaaaaaaaaaaaaaaaaaaaaaaaaaaaa

SEQ ID NO: 6 - protein, 553 aa, synthetic construct
MAFSELLDLVGGLGRFQVLQTMALMVSIMWLCTQSMLENFSAAVPSHRCWAPLLDNSTAQASILGSLSPEALLAISIPPGPNQRPHQCRRFRQPQWQLLDPNATATSWSEADTEPCVDGWVYDRSIFTSTIVAKWNLVCDSHALKPMAQSIYLAGILVGAAACGPASDRFGRRLVLTWSYLQMAVMGTAAAFAPAFPVYCLFRFLLAFAVAGVMMNTGTLLMEWTAARARPLVMTLNSLGFSFGHGLTAAVAYGVRDWTLLQLVVSVPFFLCFLYSWWLAESARWLLTTGRLDWGLQELWRVAAINGKGAVQDTLTPEVLLSAMREELSMGQPPASLGTLLRMPGLRFRTCISTLCWFAFGFTFFGLALDLQALGSNIFLLQMFIGVVDIPAKMGALLLLSHLGRRPTLAASLLLAGLCILANTLVPHEMGALRSALAVLGLGGVGAAFTCITIYSSELFPTVLRMTAVGLGQMAARGGAILGPLVRLLGVHGPWLPLLVYGTVPVLSGLAALLLPETQSLPLPDTIQDVQNQAVKKATHGTLGNSVLKSTQF

(19) United States
(12) Patent Application Publication
(10) Pub. No.: US 2025/0179032 A1
(43) Pub. Date: Jun. 5, 2025

(54) HETEROCYCLIC INHIBITORS OF GLUT9 FOR TREATMENT OF DISEASE

(71) Applicant: HORIZON THERAPEUTICS IRELAND DAC, DUBLIN 2 (IE)

(72) Inventors: BRAD RICHARD HENKE, BRADENTON, FL (US); DENIS BILLEN, BARRINGTON, IL (US); HARRY B. CHOBANIAN, WESTFIELD, NJ (US); MARTA E WENZLER (PEISKER), CHICAGO, IL (US); EMILE JOHANN VELTHUISEN, RALEIGH, NC (US); NICHOLAS DAVID ADAMS, ROYERSFORD, PA (US); ASHLEY FENWICK, NAPERVILLE, IL (US)

(73) Assignee: HORIZON THERAPEUTICS IRELAND DAC, DUBLIN 2 (IE)

(21) Appl. No.: 18/725,750

(22) PCT Filed: Jan. 6, 2023
(86) PCT No.: PCT/IB2023/000011

§ 371 Date: Oct. 28, 2024

Related U.S. Application Data
(60) Provisional application No. 63/297,511, filed on Jan. 7, 2022.

(51) Int. Cl. C07D 241/20; A61K 31/443; A61K 31/4436; A61K 31/4439; A61K 31/444; A61K 31/454; A61K 31/497; A61K 31/5025; A61K 31/506; A61K 31/5365; A61K 31/538; A61K 31/5383; A61K 31/55; A61K 45/06; C07D 401/04; C07D 401/14; C07D 403/04; C07D 405/04; C07D 405/14; C07D 409/04; C07D 409/14; C07D 413/04; C07D 413/14; C07D 417/04; C07D 417/14; C07D 419/14; C07D 471/04; C07D 471/08; C07D 487/04; C07D 491/048; C07D 491/107; C07D 495/04

(52) U.S. Cl. CPC C07D 241/20; A61K 31/443; A61K 31/4436; A61K 31/4439; A61K 31/444; A61K 31/454; A61K 31/497; A61K 31/5025; A61K 31/506; A61K 31/5365; A61K 31/538; A61K 31/5383; A61K 31/55; A61K 45/06; C07D 401/04; C07D 401/14; C07D 403/04; C07D 405/04; C07D 405/14; C07D 409/04; C07D 409/14; C07D 413/04; C07D 413/14; C07D 417/04; C07D 417/14; C07D 419/14; C07D 471/04; C07D 471/08; C07D 487/04; C07D 491/048; C07D 491/107; C07D 495/04

(57) ABSTRACT

The present invention relates to heterocyclic compounds and methods which may be useful as inhibitors of GLUT9 for the treatment or prevention of hyperuricemia and gout.

### CROSS-REFERENCE TO RELATED APPLICATIONS

This application claims benefit of U.S. Provisional Patent Application No. 63/297,511, filed on Jan. 7, 2022, which is incorporated herein by reference in its entirety.

### REFERENCE TO A SEQUENCE LISTING

This application contains a Sequence Listing which has been submitted electronically in XML format and is hereby incorporated by reference in its entirety. Said XML copy, created on Dec. 27, 2022, is named 58651-602-601_SL.xml and is 11,787 bytes in size.

### BACKGROUND OF THE INVENTION

Disclosed herein are new heterocyclic compounds and compositions and their application as pharmaceuticals for the treatment of disease. Methods of inhibition of glucose transporter member 9 (GLUT9) activity in a human or animal subject are also provided for the treatment diseases such as hyperuricemia and gout.

Uric acid is an end metabolite in the human purine catabolic pathway. Under physiological pH conditions, uric acid exists largely as urate, the ion form of uric acid. The amount of urate in the body depends on the balance between the amount of purines obtained by food intake, the amount of urate synthesized within the body, and the amount of urate that is excreted in urine or through the gastrointestinal tract. Hyperuricemia is a condition characterized by abnormally high levels of uric acid in the blood. In humans, the upper end of the normal range for uric acid is about 360 μmol/L (about 6 mg/dL) for women and about 400 μmol/L (about 6.8 mg/dL) for men. When the concentration of uric acid is above the biochemical limit of solubility, e.g., a serum uric acid level of about 6.8 mg/dL, monosodium urate crystals can precipitate in tissues. Hyperuricemia can lead to the accumulation of monosodium urate crystals and cause symptoms of gout, such as acute inflammation of joints (gout flare), formation of gout tophi, gouty arthritis, and uric acid nephropathy (including uric acid renal stones). Gout is the most prevalent cause of arthritis in developed countries. Control of chronic gout requires maintaining serum uric acid (SUA) below 6 mg/dL.

High serum urate is associated with elevated body mass index, hypercholesterolemia, hypertriglyceridemia, increased fasting plasma glucose, and insulin resistance. In addition, growing evidence has illuminated the dose effect of urate on the risk of metabolic syndrome, and an elevated urate level may have adverse cardiovascular effects. Further, chronic kidney disease, hypertension, diabetes, cardiac disease (such as cardiovascular disease, cardiac failure, atrial fibrillation), arteriosclerotic disease, non-alcoholic fatty liver disease (NAFLD) and non-alcoholic steatohepatitis (NASH), and psoriasis have also been linked to high serum urate levels.

Therefore, treatment of hyperuricemia is necessary. At present, excessive production of uric acid and insufficient excretion are the main causes of hyperuricemia. Over 90% of hyperuricemia is caused by insufficient uric acid excretion. Excretion of uric acid is dependent on urate transporters, such as urate transporter 1 (SLC22A12/URAT1) and glucose transporter 9 (SLC2A9/GLUT9). Therefore, URAT1 and GLUT9 have become important targets for the development of uric acid-lowering drugs.

Urate-lowering therapies include uricases, which degrade uric acid; xanthine oxidase inhibitors, which block uric acid production; and uricosuric drugs, which enhance uric acid excretion. Xanthine oxidase inhibitors such as allopurinol and febuxostat are widely used and orally administered. Uricases such as pegloticase (KRYSTEXXA®), which is a pegylated recombinant mammalian uricase, are administered by infusion and used to treat gout that is refractory to conventional therapies, e.g., oral allopurinol. Most mammals, including humans, do not have functional uricase. Uricase degrades uric acid into allantoin, a more soluble molecule that is easily eliminated through the kidneys. Despite significant advances in research, however, gout remains poorly managed, which can cause the condition to become chronic. Xanthine oxidase inhibitor therapies can cause hypersensitivity syndrome, and uricase therapies can cause immunogenicity, for example, which poses challenges to gout management.

Additional small molecule uricosuric drugs used in the clinic mainly include benzbromarone, probenecid and lesinurad. Benzbromarone and probenecid can inhibit URAT1 and GLUT9 and thus promote uric acid excretion. However, due to the fulminant hepatitis caused by benzbromarone, it has been withdrawn from the market in most countries. Because of low selectivity, probenecid can lead to drug-drug interactions, and therefore, its clinical use is limited. Lesinurad is a more selective URAT1 inhibitor developed by AstraZeneca and was approved by the FDA in October 2015 for the treatment of hyperuricemia, but it should be used in combination with a xanthine oxidase inhibitor (allopurinol/febuxostat). RDEA3170 is derived from lesinurad, which is currently in a phase II clinical trials. In addition to the abovementioned drugs, losartan, fenofibrate, atorvastatin and others also have a uric acid-lowering effect, but their mechanisms have not been fully elucidated. Therefore, it is of great significance to find new drugs to treat hyperuricemia Novel compounds and pharmaceutical compositions, certain of which have been found to inhibit GLUT9 have been discovered, together with methods of synthesizing and using the compounds including methods for the treatment of GLUT9-mediated diseases in a patient by administering the compounds.

### BRIEF SUMMARY OF THE INVENTION

Compounds disclosed herein may possess useful GLUT9 inhibiting activity and may be used in the treatment or prophylaxis of a disease or condition in which GLUT9 plays an active role.

In one aspect, described herein is a compound of Formula (I′):

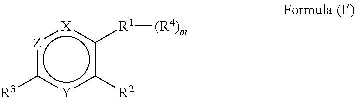

or a pharmaceutically acceptable salt thereof, wherein:
m is an integer from 0 to 4;
X is selected from CH and N;
Y is selected from CH, S, O, and N;
Z is selected from a bond and CR⁷;
R¹ is selected from aryl and heteroaryl;
R² is a mono- or bi-cyclic heterocycloalkyl or a mono- or bi-cyclic heteroaryl, either of which may be optionally substituted with one or more R⁶.
R³ is selected from:
   alkyl optionally substituted with SO₂-alkyl,
   alkoxyalkyl,
   alkoxyalkoxyalkyl,
   haloalkyl,
   haloalkoxyalkyl,
   cycloalkylalkyl, and
   cycloalkoxyalkyl;
each R⁴ is independently selected from:
   alkoxy optionally substituted with R⁵,
   alkylamino optionally substituted with R⁵,
   dialkylamino optionally substituted with R⁵,
   SO₂R^{8a},
   alkylthio,
   haloalkoxy,
   cycloalkoxy,
   cycloalkylalkoxy,
   halo,
   alkyl, and
   haloalkyl;
or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl;
each R⁵ is independently selected from hydroxyl, alkoxy, amino, alkylamino, dialkylamino, haloalkoxy, and alkoxyalkoxy;
each R⁶ is independently selected from hydroxyl, hydroxyalkoxy, carboxyl, methylcarboxyl, carboxylalkyl, carboxylalkoxy, halo, alkyl, alkoxy, SO₂R^{8b}, C(═O)NHSO₂R^{8b}, heterocycloalkyl, cyano, and tetrazolyl;
R⁷ is selected from hydrogen and halo; and
R⁸ and R^{8b} are each independently selected from alkyl, amino, alkylamino, dialkylamino;
wherein X and Y are not both CH.

Also described herein are pharmaceutical compositions comprising a compound described herein, or a pharmaceutically acceptable salt thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by inhalation; and/or (e) administered by nasal administration; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the embodiments disclosed herein, the mammal is a human.

In some embodiments, compounds provided herein are orally administered to a human.

Also described herein, in some embodiments, is a compound described herein, or a pharmaceutically acceptable salt thereof, for use as a medicament.

Also described herein, in some embodiments, is a compound described herein, or a pharmaceutically acceptable salt thereof, for use in the treatment of a condition selected from hyperuricemia and gout.

Also described herein, in some embodiments, is a compound described herein, or a pharmaceutically acceptable salt thereof, for use in the manufacture of a medicament for the prevention or treatment of a disease or condition ameliorated by the inhibition of GLUT9.

Also described herein, in some embodiments, is a method of inhibition of GLUT9 comprising contacting GLUT9 with a compound described herein, or a pharmaceutically acceptable salt thereof.

Also described herein, in some embodiments, is a method of treatment of a GLUT9-mediated disease comprising the administration of a therapeutically effective amount of a compound described herein, or a pharmaceutically acceptable salt thereof, to a patient in need thereof. In some embodiments, said disease is selected from hyperuricemia, gout, and uncontrolled gout including comorbidities and associated diseases.

Also described herein, in some embodiments, is a method of treatment of a GLUT9-mediated disease comprising the administration of: a therapeutically effective amount of a compound described herein, or a pharmaceutically acceptable salt thereof; and another therapeutic agent. In some embodiments, said other agent is selected from inhibitors of uric acid synthesis, uricosurics, and uric acid catabolizing agents. In some such embodiments, said uricosuric is selected from probenecid, lesinurad, benzbromarone, and sulfinpyrazone. In some such embodiments, said inhibitor of uric acid synthesis is selected from allopurinol and febuxostat. In some such embodiments, said uric acid catabolizing agent is pegloticase. In some other embodiments, said other agent is colchichine.

Also described herein, in some embodiments, is a method for lowering blood uric acid levels in a patient comprising the administration of a therapeutically effective amount of a compound described herein, or a pharmaceutically acceptable salt thereof, to a patient.

Articles of manufacture, which include packaging material, a compound described herein, or a pharmaceutically acceptable salt thereof, within the packaging material, and a label that indicates that the compound or composition, or pharmaceutically acceptable salt, tautomers, pharmaceutically acceptable N-oxide, pharmaceutically active metabolite, pharmaceutically acceptable prodrug, or pharmaceutically acceptable solvate thereof, is used for inhibiting GLUT9, or for the treatment, prevention or amelioration of one or more symptoms of a disease or condition that would benefit from inhibiting GLUT9, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1 shows the nucleic acid sequence of human GLUT9B/GLUT9 variant 2/Q96S37-2/Glut9 Isoform2 (SEQ ID NO: 3).

FIG. 2 shows the amino acid sequence of human GLUT9B/GLUT9 variant 2/Q96S37-2/Glut9 Isoform2 (SEQ ID NO: 4).

FIG. 3 shows the nucleic acid sequence of human URAT1 (SEQ ID NO: 5).

FIG. 4 shows the amino acid sequence of human URAT1 (SEQ ID NO: 6).

FIG. 5 shows the pIRESpuro vector from Takara Bio.

FIG. 6 shows the cloning strategy for GLUT9B and URAT1 to generate bicistronic vector for stable cell line generation.

FIG. 7 shows the conversion of uric acid to allantoin.

FIG. 8 shows the Aspergillus oryzae uricase vector from GenScript.

### DETAILED DESCRIPTION OF THE INVENTION

Compounds disclosed herein may possess useful GLUT9 inhibiting activity and may be used in the treatment or prophylaxis of a disease or condition in which GLUT9 plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting GLUT9. Other embodiments provide methods for treating a GLUT9-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of GLUT9.

### Compounds of the Disclosure

Disclosed herein are compounds that are useful for inhibiting GLUT9 activity. In some embodiments, compounds described herein are useful in the treatment or prophylaxis of a disease or condition in which GLUT9 plays an active role.

In one aspect, described herein is a compound of Formula (I′):

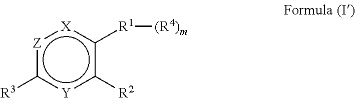

or a pharmaceutically acceptable salt thereof, wherein:
m is an integer from 0 to 4;
X is selected from CH and N;
Y is selected from CH, S, O, and N;
Z is selected from a bond and CR⁷;
R¹ is selected from aryl and heteroaryl;
R² is a mono- or bi-cyclic heterocycloalkyl or a mono- or bi-cyclic heteroaryl, either of which may be optionally substituted with one or more R⁶.
R³ is selected from:
   alkyl optionally substituted with SO₂-alkyl,
   alkoxyalkyl,
   alkoxyalkoxyalkyl,
   haloalkyl,
   haloalkoxyalkyl,
   cycloalkylalkyl, and
   cycloalkoxyalkyl;
each R⁴ is independently selected from:
   alkoxy optionally substituted with R⁵,
   alkylamino optionally substituted with R⁵,
   dialkylamino optionally substituted with R⁵,
   SO₂R^{8a},
   alkylthio,
   haloalkoxy,
   cycloalkoxy,
   cycloalkylalkoxy,
   halo,
   alkyl, and
   haloalkyl;
or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl;
each R⁵ is independently selected from hydroxyl, alkoxy, amino, alkylamino, dialkylamino, haloalkoxy, and alkoxyalkoxy;
each R⁶ is independently selected from hydroxyl, hydroxyalkoxy, carboxyl, methylcarboxyl, carboxylalkyl, carboxylalkoxy, halo, alkyl, alkoxy, SO₂R^{8b}, C(═O)NHSO₂R^{8b}, heterocycloalkyl, cyano, and tetrazolyl;
R⁷ is selected from hydrogen and halo; and
R^{8a} and R^{8b} are each independently selected from alkyl, amino, alkylamino, dialkylamino;
wherein X and Y are not both CH.

For any and all of the embodiments, substituents are selected from among a subset of the listed alternatives. For example, in some embodiments, X is CH. In other embodiments, X is N.

In some embodiments, Y is CH or N. In some embodiments, Y is CH. In some embodiments, Y is N. In some embodiments, Y is O. In some embodiments, Y is S.

In some embodiments, Z is a bond. In some embodiments, Z is CR⁷. In some embodiments, R⁷ is hydrogen. In some embodiments, R⁷ is halogen. In some embodiments, R⁷ is F or Cl. In some embodiments, Z is a bond, CH, CF, or CCl. In some embodiments, Z is CH or CCl. In some embodiments, Z is a bond or CH. In some embodiments, Z is CH.

In some embodiments, X is CH; and Y is S, O, or N.

In some embodiments, X is CH; Y is N; and Z is CR⁷. In some embodiments, X is CH; Y is N; and Z is CH.

In some embodiments, X is N; and Y is CH, S, O, or N.

In some embodiments, X is N; Y is CH; and Z is CR⁷. In some embodiments, X is N; Y is CH; and Z is CH.

In some embodiments, X is N; Y is N; and Z is CR⁷. In some embodiments, X is N; Y is N; and Z is CH.

In some embodiments, X is selected from CH and N; Y is selected from CH and N; and Z is CR⁷. In some embodiments, X is CH; Y is N; and Z is CR⁷; or X is N; Y is CH; and Z is CR⁷; or X is N; Y is N; and Z is CR⁷. In some embodiments, X is CH; Y is N; and Z is CR⁷. In some embodiments, X is N; Y is CH; and Z is CR⁷. In some embodiments, X is N; Y is N; and Z is CR⁷. In some such embodiments, R⁷ is hydrogen, F, or Cl. In some such embodiments, R⁷ is hydrogen. In some such embodiments, R⁷ is F. In some such embodiments, R⁷ is Cl.

In some embodiments, X is selected from CH and N; Y is selected from CH and N; and Z is CH. In some embodiments, X is CH; Y is N; and Z is CH; or X is N; Y is CH; and Z is CH; or X is N; Y is N; and Z is CH. In some embodiments, X is CH; Y is N; and Z is CH. In some embodiments, X is N; Y is CH; and Z is CH. In some embodiments, X is N; Y is N; and Z is CH.

In some embodiments, the compound is a compound of Formula (I′-A), or a pharmaceutically acceptable salt thereof:

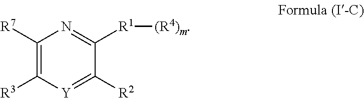

In some embodiments, the compound is a compound of Formula (I′-B) or Formula (I′-C), or a pharmaceutically acceptable salt thereof:

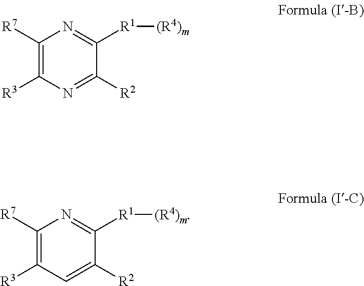

In some embodiments, R¹ is aryl or heteroaryl. In some embodiments, R¹ is phenyl, naphthyl, monocyclic heteroaryl, or bicyclic heteroaryl. In some embodiments, R¹ is phenyl, naphthyl, monocyclic heteroaryl, or bicyclic heteroaryl, wherein the heteroaryl contains 0-4 N atoms, 0-1 O atoms, and 0-1 S atoms in the ring, or wherein the heteroaryl contains 1-4 N atoms, 0-1 O atoms, and 0-1 S atoms in the ring. In some embodiments, R¹ is phenyl, monocyclic heteroaryl, or bicyclic heteroaryl. In some embodiments, R¹ is phenyl, 5-membered monocyclic heteroaryl, 6-membered monocyclic heteroaryl, or bicyclic heteroaryl.

In some embodiments, R¹ is aryl. In some embodiments, R¹ is phenyl or naphthyl. In some embodiments, R¹ is phenyl.

In some embodiments, R¹ is heteroaryl. In some embodiments, R¹ is monocyclic heteroaryl, or bicyclic heteroaryl. In some embodiments, R¹ is monocyclic heteroaryl, or bicyclic heteroaryl, wherein the heteroaryl contains 0-4 N atoms, 0-1 O atoms, and 0-1 S atoms in the ring, or wherein the heteroaryl contains 1-4 N atoms, 0-1 O atoms, and 0-1 S atoms in the ring.

In some embodiments, R¹ is monocyclic heteroaryl. In some embodiments, R¹ is 5-membered monocyclic heteroaryl or 6-membered monocyclic heteroaryl. In some embodiments, R¹ is 5-membered monocyclic heteroaryl or 6-membered monocyclic heteroaryl, wherein the heteroaryl contains 0-4 N atoms, 0-1 O atoms, and 0-1 S atoms in the ring, or wherein the heteroaryl contains 1-4 N atoms, 0-1 O atoms, and 0-1 S atoms in the ring.

In some embodiments, R¹ is 5-membered monocyclic heteroaryl. In some embodiments, R¹ is 5-membered monocyclic heteroaryl, wherein the heteroaryl contains 0-4 N atoms, 0-1 O atoms, and 0-1 S atoms in the ring, or wherein the heteroaryl contains 1-4 N atoms, 0-1 O atoms, and 0-1 S atoms in the ring. In some embodiments, R¹ is pyrrolyl, imidazolyl, pyrazolyl, triazolyl, furyl, thienyl, oxazolyl, isoxazolyl, oxadiazolyl, thiazolyl, thiadiazolyl, isothiazolyl, or tetrazolyl.

In some embodiments, R¹ is 6-membered monocyclic heteroaryl. In some embodiments, R¹ is 6-membered monocyclic heteroaryl, wherein the heteroaryl contains 1-4 N atoms. In some embodiments, R¹ is pyridyl, pyrimidinyl, pyrazinyl, pyridazinyl, or triazinyl.

In some embodiments, R¹ is bicyclic heteroaryl. In some embodiments, R¹ is a C₂-C₉bicyclic heteroaryl. In some embodiments, R¹ is bicyclic heteroaryl containing 0-4 N atoms, 0-1 O atoms, and 0-1 S atoms in the ring system, or containing 1-4 N atoms, 0-1 0 atoms, and 0-1 S atoms in the ring system. In some embodiments, R¹ is bicyclic heteroaryl containing 1 O atom and 0 or 1 N atoms in the ring system. In some embodiments, R¹ is indolyl, isoindolyl, indolizinyl, benzimidazolyl, quinolyl, isoquinolyl, quinoxalinyl, quinazolinyl, indazolyl, benzotriazolyl, benzoxazolyl, benzoxadiazolyl, benzothiazolyl, benzothiadiazolyl, benzofuranyl, benzothienyl, chromonyl, coumarinyl, tetrazolopyridazinyl, furopyridinyl, or pyrrolopyridinyl. In some embodiments, R¹ is benzofuranyl or furopyridinyl. In some embodiments, R¹ is benzofuranyl.

In some embodiments, R¹ is phenyl; 1H-indol-5-yl; 1H-indol-6-yl; thiophen-2-yl; thiazol-5-yl; 3-oxo-2,3-dihydrobenzofuran-5-yl; 2,3-dihydrobenzofuran-5-yl; furo[2,3-b]pyridin-5-yl; 2,3-dihydrofuro[2,3-b]pyridin-5-yl; pyridin-3-yl; pyridin-4-yl; benzo[d]oxazol-6-yl; 2,3-dihydrobenzo[b][1,4]dioxin-6-yl; chroman-6-yl; 3,4-dihydro-2H-benzo[b][1,4]oxazin-6-yl; 3-oxo-3,4-dihydro-2H-benzo[b][1,4]oxazin-6-yl; furo[2,3-b]pyridin-5-yl; 2,3-dihydrobenzofuran-5-yl; 2H-indazol-6-yl; 1H-pyrrolo[2,3-b]pyridin-4-yl; [1,2,4]triazolo[1,5-a]pyridin-6-yl; imidazo[1,2-a]pyrimidin-6-yl; 2H-indazol-6-yl; 2H-indazol-5-yl; quinolin-3-yl; quinolin-4-yl; 1H-pyrazol-5-yl; thieno[3,2-b]thiophen-2-yl; benzo[c][1,2,5]thiadiazol-5-yl; imidazo[1,2-a]pyridin-6-yl; 4,5,6,7-tetrahydropyrazolo[1,5-a]pyridin-3-yl; imidazo[1,5-a]pyridin-6-yl; imidazo[1,2-a]pyridin-7-yl; 6,7-dihydro-5H-pyrazolo[5,1-b][1,3]oxazin-3-yl; thieno[2,3-b]pyridin-5-yl; imidazo[1,2-b]pyridazin-6-yl; [1,2,4]triazolo[1,5-a]pyridin-7-yl; 2H-indazol-7-yl; 1H-pyrrolo[2,3-b]pyridin-4-yl; 2H-indazol-4-yl; 2,3-dihydro-1H-pyrido[2,3-b][1,4]oxazin-7-yl; 1H-pyrazolo[3,4-b]pyridin-5-yl; 2H-pyrazolo[3,4-b]pyridin-5-yl; 3H-imidazo[4,5-b]pyridin-6-yl; imidazo[1,2-b]pyridazin-6-yl; quinolin-4-yl; benzo[b]thiophen-5-yl; chroman-6-yl; 1-oxo-1,3-dihydroisobenzofuran-5-yl; 1,1-dioxido-2,3-dihydrobenzo[b]thiophen-5-yl; 1,1-dioxidothiochroman-6-yl; benzofuran-5-yl; or benzo[d][1,3]dioxol-5-yl.

In some embodiments, R¹ is phenyl; 3-oxo-2,3-dihydrobenzofuran-5-yl; 2,3-dihydrobenzofuran-5-yl; 2,3-dihydrofuro[2,3-b]pyridin-5-yl; 2,3-dihydrobenzo[b][1,4]dioxin-6-yl; chroman-6-yl; 3,4-dihydro-2H-benzo[b][1,4]oxazin-6-yl; 3-oxo-3,4-dihydro-2H-benzo[b][1,4]oxazin-6-yl; furo[2,3-b]pyridin-5-yl; 2,3-dihydrobenzofuran-5-yl; benzo[b]thiophen-5-yl; chroman-6-yl; 1-oxo-1,3-dihydroisobenzofuran-5-yl; 1,1-dioxido-2,3-dihydrobenzo[b]thiophen-5-yl; 1,1-dioxidothiochroman-6-yl; benzofuran-5-yl; or benzo[d][1,3]dioxol-5-yl.

In some embodiments, m is an integer from 0 to 4. In some embodiments, m is 0 to 3. In some embodiments, m is 0 to 2. In some embodiments, m is 0 to 1. In some embodiments, m is 0, 1, 2, 3, or 4. In some embodiments, m is 1 to 4. In some embodiments, m is 0. In some embodiments, m is 1. In some embodiments, m is 2. In some embodiments, m is 3. In some embodiments, m is 4.

In some embodiments, each R⁴ is independently selected from: alkoxy optionally substituted with R⁵, alkylamino optionally substituted with R⁵, dialkylamino optionally substituted with R⁵, SO₂R^{8a}, alkylthio, haloalkoxy, cycloalkoxy, cycloalkylalkoxy, halo, alkyl, and haloalkyl; or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl.

In some embodiments, each R⁴ is independently selected from alkoxy optionally substituted with R⁵, SO₂R^{8a}, haloalkoxy, cycloalkoxy, cycloalkylalkoxy, halo, alkyl, and haloalkyl; or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl.

In some embodiments, each R⁴ is independently selected from: C₁-C₆alkoxy optionally substituted with R⁵, C₁-C₆alkylamino optionally substituted with R⁵, di-(C₁-C₆alkyl)-amino optionally substituted with R⁵, SO₂R^{8a}, C₁-C₆alkylthio, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl; or two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₆alkoxy)-C₁-C₆alkyl.

In some embodiments, each R⁴ is independently selected from C₁-C₆alkoxy optionally substituted with R⁵, SO₂R^{8a}, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl; or two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₆alkoxy)-C₁-C₆alkyl.

In some embodiments, each R⁴ is independently selected from alkoxy optionally substituted with R⁵, SO₂R^{8a}, haloalkoxy, cycloalkoxy, cycloalkylalkoxy, halo, alkyl, and haloalkyl. In some embodiments, each R⁴ is independently selected from alkoxy optionally substituted with R⁵, SO₂R^{8a}, haloalkoxy, cycloalkoxy, cycloalkylalkoxy, halo, alkyl, and haloalkyl. In some embodiments, each R⁴ is independently selected from alkoxy, haloalkoxy, and halo.

In some embodiments, each R⁴ is independently selected from: C₁-C₆alkoxy optionally substituted with R⁵, C₁-C₆alkylamino optionally substituted with R⁵, di-(C₁-C₆alkyl)-amino optionally substituted with R⁵, SO₂R^{8a}, C₁-C₆alkylthio, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl. In some embodiments, each R⁴ is independently selected from C₁-C₆alkoxy optionally substituted with R⁵, SO₂R^{8a}, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl. In some embodiments, each R⁴ is independently selected from C₁-C₆alkoxy, C₁-C₆haloalkoxy, and halo. In some embodiments, each R⁴ is independently selected from C₁-C₆alkoxy, C₁-C₆haloalkoxy, F, and Cl.

In some embodiments, each R⁴ is independently selected from: C₁-C₆alkoxy optionally substituted with R⁵, C₁-C₄alkylamino optionally substituted with R⁵, di-(C₁-C₄alkyl)-amino optionally substituted with R⁵, SO₂R^{8a}, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl. In some embodiments, each R⁴ is independently selected from: C₁-C₆alkoxy optionally substituted with R⁵, C₁-C₄alkylamino optionally substituted with R⁵, di-(C₁-C₄alkyl)-amino optionally substituted with R⁵, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl. In some embodiments, each R⁴ is independently selected from: C₁-C₆alkoxy, C₁-C₄haloalkoxy, halo, C₁-C₄alkyl, and C₁-C₄haloalkyl. In some embodiments, each R⁴ is independently selected from: C₁-C₆alkoxy, C₁-C₄ₕaloalkoxy, and halo.

In some embodiments, when one R⁴ is SO₂R^{8a}, R^{8a} is selected from C₁-C₆alkyl, amino, C₁-C₄alkylamino, di-(C₁-C₄alkyl)-amino. In some embodiments, when one R⁴ is SO₂R^{8a}, R^{8a} is selected from C₁-C₄alkyl, amino, C₁-C₄alkylamino, di-(C₁-C₄alkyl)-amino. In some embodiments, when one R⁴ is SO₂R^{8a}, R^{8a} is selected from —CH₃, —NH₂, —NH(CH₃), and —N(CH₃)₂.

In some embodiments, or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl. In some embodiments, two R⁴ substituents on adjacent carbon atoms of R¹ are taken together to form a fused heterocycloalkyl or a fused heteroaryl.

In some embodiments, two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₆alkoxy)-C₁-C₆alkyl. In some embodiments, two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₄alkoxy)-C₁-C₆alkyl. In some embodiments, two R⁴ substituents on adjacent carbon atoms of R¹ are taken together to form a fused 5-membered heterocycloalkyl comprising 1 or 2 O atoms or a fused 5-membered heteroaryl comprising 1 O atom and 0 or 1 N atoms, either of which may be optionally substituted with one or more C₁-C₄ₐlkyl, halo, C₁-C₄alkoxy or (C₁-C₄alkoxy)-C₁-C₄alkyl. In some embodiments, two R⁴ substituents on adjacent carbon atoms of R¹ are taken together to form a fused dioxolane or a fused furan ring, either of which may be optionally substituted with one or more C₁-C₄ₐlkyl, halo, C₁-C₄alkoxy or (C₁-C₄alkoxy)-C₁-C₄alkyl. In some embodiments, two R⁴ substituents on adjacent carbon atoms of R¹ are taken together to form a fused 5-membered heterocycloalkyl comprising 1 or 2 O atoms or a fused 5-membered heteroaryl comprising 1 O atom and 0 or 1 N atoms. In some embodiments, two R⁴ substituents on adjacent carbon atoms of R¹ are taken together to form a fused dioxolane or a fused furan ring

In some embodiments, R¹ is phenyl, and two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₆alkoxy)-C₁-C₆alkyl. In some embodiments, R¹ is phenyl, and two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₄alkoxy)-C₁-C₆alkyl. In some embodiments, R¹ is phenyl, and two R⁴ substituents on adjacent carbon atoms of R¹ are taken together to form a fused 5-membered heterocycloalkyl comprising 1 or 2 O atoms or a fused 5-membered heteroaryl comprising 1 O atom and 0 or 1 N atoms, either of which may be optionally substituted with one or more C₁-C₄ₐlkyl, halo, C₁-C₄alkoxy or (C₁-C₄alkoxy)-C₁-C₄alkyl. In some embodiments, R¹ is phenyl, and two R⁴ substituents on adjacent carbon atoms of R¹ are taken together to form a fused dioxolane or a fused furan ring, either of which may be optionally substituted with one or more C₁-C₄alkyl, halo, C₁-C₄alkoxy or (C₁-C₄alkoxy)-C₁-C₄alkyl. In some embodiments, R¹ is phenyl, and two R⁴ substituents on adjacent carbon atoms of R¹ are taken together to form a fused 5-membered heterocycloalkyl comprising 1 or 2 O atoms or a fused 5-membered heteroaryl comprising 1 O atom and 0 or 1 N atoms. In some embodiments, R¹ is phenyl, and two R⁴ substituents on adjacent carbon atoms of R¹ are taken together to form a fused dioxolane or a fused furan ring.

In some embodiments, each R^{s} is independently selected from hydroxyl, alkoxy, amino, alkylamino, dialkylamino, haloalkoxy, and alkoxyalkoxy. In some embodiments, each R⁵ is independently selected from hydroxyl, alkoxy, amino, alkylamino, dialkylamino, and haloalkoxy. In some embodiments, each R⁵ is selected from alkoxy, amino, alkylamino, and dialkylamino.

In some embodiments, each R⁵ is independently selected from hydroxyl, C₁-C₆alkoxy, amino, C₁-C₆alkylamino, di-C₁-C₆alkylamino, C₁-C₆haloalkoxy, and (C₁-C₆alkoxy)-C₁-C₆alkoxy. In some embodiments, each R⁵ is independently selected from hydroxyl, C₁-C₆alkoxy, amino, C₁-C₄ₐlkylamino, di-(C₁-C₄alkyl)-amino, C₁-C₆haloalkoxy, and (C₁-C₄alkoxy)-C₁-C₆alkoxy. In some embodiments, each R⁵ is independently selected from hydroxyl, C₁-C₆alkoxy, amino, C₁-C₄alkylamino, di-(C₁-C₄ₐlkyl)-amino, and C₁-C₆haloalkoxy.

In some embodiments, each R⁴ is independently selected from alkoxy optionally substituted with R⁵, SO₂R^{8a}, haloalkoxy, cycloalkoxy, cycloalkylalkoxy, halo, alkyl, and haloalkyl; or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl; each R⁵ is independently selected from hydroxyl, alkoxy, amino, alkylamino, dialkylamino, and haloalkoxy; and m is an integer from 0 to 3.

In some embodiments, each R⁴ is independently selected from alkoxy, haloalkoxy, and halo; or two R⁴ substituents on adjacent carbon atoms of R¹ are taken together to form a fused heterocycloalkyl or a fused heteroaryl; and m is an integer from 0 to 3.

In some embodiments, each R⁴ is independently selected from alkoxy optionally substituted with R⁵, SO₂R^{8a}, haloalkoxy, cycloalkoxy, cycloalkylalkoxy, halo, alkyl, and haloalkyl; or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl; each R⁵ is independently selected from hydroxyl, alkoxy, amino, alkylamino, dialkylamino, and haloalkoxy; and m is an integer from 0 to 3.

In some embodiments, each R⁴ is independently selected from alkoxy, haloalkoxy, and halo; or two R⁴ substituents on adjacent carbon atoms of R¹ are taken together to form a fused heterocycloalkyl or a fused heteroaryl; and m is an integer from 0 to 3.

In some embodiments, each R⁴ is independently selected from: alkoxy optionally substituted with R⁵, SO₂R^{8a}, haloalkoxy, cycloalkoxy, cycloalkylalkoxy, and halo; or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl.

In some embodiments, each R⁴ is independently selected from alkoxy optionally substituted with R⁵, SO₂R^{8a}, and halo; or two R⁴ substituents on adjacent carbon atoms of R¹ are taken together to form a fused heterocycloalkyl or a fused heteroaryl.

In some embodiments, each R⁴ is independently selected from: C₁-C₆alkoxy optionally substituted with R⁵, C₁-C₆alkylamino optionally substituted with R⁵, di-(C₁-C₆alkyl)-amino optionally substituted with R⁵, SO₂R^{8a}, C₁-C₆alkylthio, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl; or two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₆alkoxy)-C₁-C₆alkyl; and each R⁵ is independently selected from hydroxyl, C₁-C₆alkoxy, amino, C₁-C₆alkylamino, di-C₁-C₆alkylamino, C₁-C₆haloalkoxy, and (C₁-C₆alkoxy)-C₁-C₆alkoxy.

In some embodiments, each R⁴ is independently selected from: C₁-C₆alkoxy optionally substituted with R⁵, C₁-C₄alkylamino optionally substituted with R⁵, di-(C₁-C₄alkyl)-amino optionally substituted with R⁵, SO₂R^{8a}, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl; or two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₄alkoxy)-C₁-C₆alkyl; and each R⁵ is independently selected from hydroxyl, C₁-C₆alkoxy, amino, C₁-C₄ₐlkylamino, di-(C₁-C₄alkyl)-amino, C₁-C₆haloalkoxy, and (C₁-C₄alkoxy)-C₁-C₆alkoxy.

In some embodiments, each R⁴ is independently selected from: alkoxy optionally substituted with R⁵, SO₂R^{8a}, haloalkoxy, cycloalkoxy, cycloalkylalkoxy, and halo; or two R⁴ substituents taken together form a fused heterocycloalkyl or a fused heteroaryl either of which may be optionally substituted with one or more alkyl, halo, oxo, alkyl, or alkoxyalkyl. In some embodiments, each R⁴ is independently selected from alkoxy optionally substituted with R⁵, SO₂R^{8a}, and halo; or two R⁴ substituents taken together form a fused heterocycloalkyl or heteroaryl. In some embodiments, R⁴ is alkoxy.

In some embodiments, R³ is selected from: alkyl optionally substituted with SO₂-alkyl, alkoxyalkyl, alkoxyalkoxyalkyl, haloalkyl, haloalkoxyalkyl, cycloalkylalkyl, and cycloalkoxyalkyl. In some embodiments, R³ is selected from alkyl optionally substituted with SO₂-alkyl, alkoxyalkyl, haloalkyl, haloalkoxyalkyl, cycloalkylalkyl, and cycloalkoxyalkyl. In some embodiments, R³ is selected from alkyl, alkoxyalkyl, haloalkyl, and haloalkoxyalkyl. In some embodiments, R³ is selected from alkyl and alkoxyalkyl. In some embodiments, R³ is alkoxyalkyl. In some embodiments, R³ is alkyl. In some embodiments, R³ is haloalkyl. In some embodiments, R³ is haloalkoxyalkyl. In some embodiments, R³ is selected from: alkyl optionally substituted with SO₂-alkyl, haloalkyl, haloalkoxyalkyl, cycloalkylalkyl, and cycloalkoxyalkyl.

In some embodiments, R³ is selected from: C₁-C₆alkyl optionally substituted with SO₂-(C₁-C₆alkyl), (C₁-C₆alkoxy)-C₁-C₆alkyl, (C₁-C₆alkoxy)-(C₁-C₆alkoxy)-C₁-C₆alkyl, C₁-C₆haloalkyl, (C₁-C₆haloalkoxy)-C₁-C₆alkyl, (C₃-C₆cycloalkyl)-C₁-C₆alkyl, and (C₃-C₆cycloalkoxy)-C₁-C₆alkyl. In some embodiments, R³ is selected from: (C₁-C₄alkoxy)-C₁-C₆alkyl, C₁-C₆haloalkyl, (C₁-C₄haloalkoxy)-C₁-C₆alkyl, (C₃-C₆cycloalkyl)-C₁-C₆alkyl, and (C₃-C₆cycloalkoxy)-C₁-C₆alkyl. In some embodiments, R³ is selected from: (C₁-C₄alkoxy)-C₁-C₆alkyl, C₁-C₆haloalkyl, and (C₁-C₄haloalkoxy)-C₁-C₆alkyl. In some embodiments, R³ is selected from: (C₁-C₄alkoxy)-C₁-C₆alkyl and (C₁-C₄haloalkoxy)-C₁-C₆alkyl. In some embodiments, R³ is (C₁-C₄alkoxy)-C₁-C₆alkyl. In some embodiments, R³ is C₁-C₆alkyl. In some embodiments, R³ is C₁-C₆haloalkyl. In some embodiments, R³ is (C₁-C₄haloalkoxy)-C₁-C₆alkyl. In some embodiments, R³ is —CH₂CH₂CH₃, —CH₂CH₂CH₂CH₃, —CH₂CH₂CH₂CH₂CH₃, —CH₂CH₂CH₂F, —CH₂CH₂CH₂CH₂F, —CH₂CH₂CH₂CH₂CH₂F, —CH₂CH₂CHF₂, —CH₂CH₂CH₂CHF₂, —CH₂CH₂CH₂CH₂CHF₂, —CH₂CH₂CF₃, —CH₂CH₂CH₂CF₃, —CH₂CH₂CH₂CH₂CF₃, —CH₂CH₂CF₂CF₃, —CH₂CH₂CF₂CH₃, —CH₂CH₂CH₂CHF₂, —CH₂CF₂CH₂CH₃, —CH₂OCH₃, —CH₂OCH₂CH₃, —CH₂CH₂OCH₃, —CH₂CH₂OCH₂CH₃, —CH₂CH₂CH₂OCH₃, —CH₂CH₂CH₂OCH₂CH₃, —CH₂CH₂CH₂CH₂OCH₃, —CH₂CH₂CH₂CH₂OCH₂CH₃, —CH₂OCF₃, —CH₂OCF₂CH₃, —CH₂OCF₂CF₃, —CH₂CH₂CH₂OCF₃, —CH₂CH₂CH₂OCF₂CH₃, —CH₂CH₂CH₂OCF₂CF₃, —CH₂CH₂CH₂CH₂OCF₃, —CH₂(cyclopropyl), —CH₂CH₂(cyclopropyl), —CH₂CH₂CH₂(cyclopropyl), or —CH₂CH₂CH₂CH₂(cyclopropyl). In some embodiments, R³ is —CH₂CH₂CH₃, —CH₂CH₂CH₂CH₃, —CH₂CH₂CH₂CH₂CH₃, —CH₂CH₂CH₂F, —CH₂CH₂CH₂CH₂F, —CH₂CH₂CH₂CH₂CH₂F, —CH₂CH₂CHF₂, —CH₂CH₂CH₂CHF₂, —CH₂CH₂CH₂CH₂CHF₂, —CH₂CH₂CF₃, —CH₂CH₂CH₂CF₃, —CH₂CH₂CH₂CH₂CF₃, —CH₂CH₂CF₂CF₃, —CH₂CH₂CF₂CH₃, —CH₂CH₂CH₂CHF₂, —CH₂CF₂CH₂CH₃, —CH₂CH₂CH₂OCH₃, —CH₂CH₂CH₂CH₂OCH₃, —CH₂CH₂CH₂OCF₃, or —CH₂CH₂CH₂CH₂OCF₃.

In some embodiments, R² is a mono- or bi-cyclic heterocycloalkyl or a mono- or bi-cyclic heteroaryl, either of which may be optionally substituted with one or more R⁶. In some embodiments, R² is a mono- or bi-cyclic heterocycloalkyl, either of which may be optionally substituted with one or more R⁶. In some embodiments, R² is a mono- or bi-cyclic heteroaryl, either of which may be optionally substituted with one or more R⁶.

In some embodiments, R² is a mono- or bi-cyclic heterocycloalkyl or a mono- or bi-cyclic heteroaryl, either of which may be optionally substituted with one to three R⁶. In some embodiments, R² is a mono- or bi-cyclic heterocycloalkyl, which may be optionally substituted with one to three R⁶. In some embodiments, R² is a mono- or bi-cyclic heteroaryl, which may be optionally substituted with one to three R⁶.

In some embodiments, R² is a mono- or bi-cyclic heterocycloalkyl or a mono- or bi-cyclic heteroaryl, either of which may be optionally substituted with one or two R⁶. In some embodiments, R² is a mono- or bi-cyclic heterocycloalkyl, which may be optionally substituted with one or two R⁶. In some embodiments, R² is a mono- or bi-cyclic heteroaryl, which may be optionally substituted with one or two R⁶.

In some embodiments, R² is a mono- or bi-cyclic heterocycloalkyl or a mono- or bi-cyclic heteroaryl, either of which is substituted with one R⁶. In some embodiments, R² is a mono- or bi-cyclic heterocycloalkyl, which is substituted with one R⁶. In some embodiments, R² is a mono- or bi-cyclic heteroaryl, which is substituted with one R⁶.

In some embodiments, R² is a mono- or bi-cyclic heterocycloalkyl, which is substituted with one R⁶. In some embodiments, R² is a mono-cyclic heterocycloalkyl, fused bi-cyclic heterocycloalkyl, bridged bi-cyclic heterocycloalkyl, or spiro bi-cyclic heterocycloalkyl, any of which is substituted with one R⁶.

In some embodiments, R² is a mono-cyclic heterocycloalkyl, which is substituted with one R⁶. In some embodiments, R² is a mono-cyclic heterocycloalkyl, which is substituted with one R⁶, wherein the heterocycloalkyl is a 3, 4, 5, or 6-membered ring and contains 0-2 N atoms, 0-2 O atoms and 0-1 S atoms in the ring. In some embodiments, R² is a mono-cyclic heterocycloalkyl, which is substituted with one R⁶, wherein the heterocycloalkyl is a 3, 4, 5, or 6-membered ring and contains 1-2 N atoms, 0-2 O atoms and 0-1 S atoms in the ring. In some embodiments, R² is an aziridinyl, azetidinyl, pyrrolidinyl, piperidinyl, piperazinyl, morpholinyl, or thiomorpholinyl, any of which is substituted with one R⁶. In some embodiments, R² is an aziridinyl, azetidinyl, pyrrolidinyl, or piperidinyl, any of which is substituted with one R⁶. In some embodiments, R² is a pyrrolidinyl or piperidinyl, either of which is substituted with one R⁶. In some embodiments, R² is a pyrrolidinyl which is substituted with one R⁶. In some embodiments, R² is a piperidinyl which is substituted with one R⁶.

In some embodiments, R² is a fused bi-cyclic heterocycloalkyl, bridged bi-cyclic heterocycloalkyl, or spiro bi-cyclic heterocycloalkyl, any of which is substituted with one R⁶.

In some embodiments, each R⁶ is independently selected from hydroxyl, hydroxyalkoxy, carboxyl, methylcarboxyl, carboxylalkyl, carboxylalkoxy, halo, alkyl, alkoxy, SO₂R^{8b}, C(═O)NHSO₂R^{8b}, heterocycloalkyl, cyano, and tetrazolyl. In some embodiments, each R⁶ is independently selected from hydroxyl, carboxyl, carboxylalkyl, carboxylalkoxy, SO₂R^{8b}, C(═O)NHSO₂R^{8b}, heterocycloalkyl, and tetrazolyl. In some embodiments, R⁶ is selected from carboxyl, carboxylalkyl, C(═O)NHSO₂R^{8b}, heterocycloalkyl, and tetrazolyl. In some embodiments, R⁶ is selected from carboxyl, carboxylalkyl, and heterocycloalkyl.

In some embodiments, each R⁶ is independently selected from hydroxyl, hydroxyalkoxy, carboxyl, methylcarboxyl, carboxylalkyl, carboxylalkoxy, halo, alkyl, alkoxy, SO₂R^{8b}, C(═O)NHSO₂R^{8b}, heterocycloalkyl, cyano, and tetrazolyl. In some embodiments, each R⁶ is independently selected from carboxyl, carboxylalkyl, halo, alkyl, SO₂R^{8b}, C(═O)NHSO₂R^{8b}, and tetrazolyl. In some embodiments, each R⁶ is independently selected from carboxyl, carboxylalkyl, halo, alkyl, SO₂R^{8b}, C(═O)NHSO₂R^{8b}, and tetrazolyl. In some embodiments, R⁶ is carboxyl.

In some embodiments, each R⁶ is independently selected from —C(═O)OH, —CH₂C(═O)OH, —C(═O)O—C₁-C₆alkyl, —C(═O)NHSO₂R^{8b}, and tetrazolyl. In some embodiments, R⁶ is selected from —C(═O)OH, —CH₂C(═O)OH, —C(═O)O—C₁-C₄alkyl, —C(═O)NHSO₂R^{8b}, and tetrazolyl. In some embodiments, R⁶ is —C(═O)OH.

In some embodiments, when one R⁶ is —C(═O)NHSO₂R^{8b}, R^{8b} is selected from C₁-C₆alkyl, amino, C₁-C₄alkylamino, di-(C₁-C₄alkyl)-amino. In some embodiments, when one R⁶ is —C(═O)NHSO₂R^{8b}, R^{8b} is selected from C₁-C₄alkyl, amino, C₁-C₄alkylamino, di-(C₁-C₄alkyl)-amino. In some embodiments, when one R⁶ is —C(═O)NHSO₂R^{8b}, R^{8b} is selected from —CH₃, —NH₂, —NH(CH₃), and —N(CH₃)₂.

In some embodiments, R² is a mono- or bi-cyclic heterocycloalkyl, which is substituted with one or more R⁶; each R⁶ is independently selected from hydroxyl, carboxyl, carboxylalkyl, carboxylalkoxy, SO₂R^{8b}, C(═O)NHSO₂R^{8b}, heterocycloalkyl, and tetrazolyl; and R^{8b} is selected from alkyl, amino, alkylamino, dialkylamino.

In some embodiments, R² is a mono-cyclic heterocycloalkyl, which is substituted with one R⁶; R⁶ is selected from carboxyl, carboxylalkyl, C(═O)NHSO₂R^{1b}, heterocycloalkyl, and tetrazolyl; and R^{8b} is selected from alkyl, amino, alkylamino, dialkylamino.

In some embodiments, R² is a piperidinyl, which is substituted with one R⁶; and R⁶ is selected from carboxyl, carboxylalkyl, and heterocycloalkyl.

In some embodiments, the compound is a compound of Formula (IV):

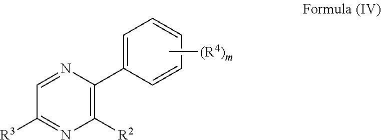

or a pharmaceutically acceptable salt thereof, wherein:
m is an integer from 0 to 3;
R² is a mono- or bi-cyclic heterocycloalkyl, which is substituted with one or more R⁶,
R³ is selected from alkyl optionally substituted with SO₂-alkyl, alkoxyalkyl, alkoxyalkoxyalkyl, haloalkyl, haloalkoxyalkyl, cycloalkylalkyl, and cycloalkoxyalkyl;
each R⁴ is independently selected from alkoxy optionally substituted with R⁵, SO₂R^{8a}, haloalkoxy, cycloalkoxy, cycloalkylalkoxy, halo, alkyl, and haloalkyl; and
or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl;
each R⁵ is independently selected from hydroxyl, alkoxy, amino, alkylamino, dialkylamino, and haloalkoxy;
each R⁶ is independently selected from hydroxyl, carboxyl, carboxylalkyl, carboxylalkoxy, SO₂R^{8b}, C(═O)NHSO₂R^{8b}, heterocycloalkyl, and tetrazolyl; and
R^{8b} is selected from alkyl, amino, alkylamino, dialkylamino.

In some embodiments, R² is a mono-cyclic heterocycloalkyl, which is substituted with one R⁶.

In some embodiments, the compound is a compound of Formula (V), or a pharmaceutically acceptable salt thereof:

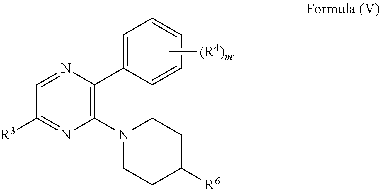

In some embodiments,
R³ is selected from alkyl, alkoxyalkyl, haloalkyl, and haloalkoxyalkyl;
each R⁴ is independently selected from alkoxy, haloalkoxy, and halo;
or two R⁴ substituents on adjacent carbon atoms of R¹ are taken together to form a fused heterocycloalkyl or a fused heteroaryl;
R⁶ is selected from carboxyl, carboxylalkyl, C(═O)NHSO₂R^{8b}, heterocycloalkyl, and tetrazolyl; and
R^{8b} is selected from alkyl, amino, alkylamino, dialkylamino.

In some embodiments, R³ is selected from alkyl and alkoxyalkyl;
each R⁴ is independently selected from alkoxy, haloalkoxy, and halo;
or two R⁴ substituents on adjacent carbon atoms of R¹ are taken together form a fused heterocycloalkyl or a fused heteroaryl; and
R⁶ is selected from carboxyl, carboxylalkyl, and heterocycloalkyl.

In some embodiments, the compound is a compound of Formula (VI):

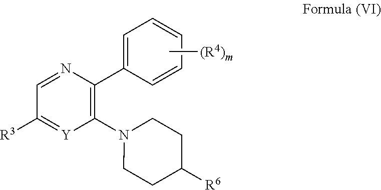

or a pharmaceutically acceptable salt thereof, wherein:
m is an integer from 0 to 4;
Y is N or CH;
R³ is selected from:
   C₁-C₆alkyl optionally substituted with SO₂-(C₁-C₆alkyl),
   (C₁-C₆alkoxy)-C₁-C₆alkyl,
   (C₁-C₆alkoxy)-(C₁-C₆alkoxy)-C₁-C₆alkyl,
   C₁-C₆haloalkyl,
   (C₁-C₆haloalkoxy)-C₁-C₆alkyl,
   (C₃-C₆cycloalkyl)-C₁-C₆alkyl, and
   (C₃-C₆cycloalkoxy)-C₁-C₆alkyl;
each R⁴ is independently selected from:
   C₁-C₆alkoxy optionally substituted with R⁵,
   C₁-C₆alkylamino optionally substituted with R⁵,
   di-(C₁-C₆alkyl)-amino optionally substituted with R⁵,
   SO₂R^{8a},
   C₁-C₆alkylthio,
   C₁-C₆haloalkoxy,
   C₃-C₆cycloalkoxy,
   (C₃-C₆cycloalkyl)-C₁-C₆alkoxy,
   halo,
   C₁-C₆alkyl, and
   C₁-C₆haloalkyl;
or two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₆alkoxy)-C₁-C₆alkyl;
each R⁵ is independently selected from hydroxyl, C₁-C₆alkoxy, amino, C₁-C₆alkylamino, di-C₁-C₆alkylamino, C₁-C₆haloalkoxy, and (C₁-C₆alkoxy)-C₁-C₆alkoxy;
R⁶ is selected from —C(═O)OH, —CH₂C(═O)OH, —C(═O)O—C₁-C₆alkyl, —C(═O)NHSO₂R^{8b}, and tetrazolyl;
R^{8a} and R^{8b} are each independently selected from C₁-C₆alkyl, amino, C₁-C₆alkylamino, di-(C₁-C₆alkyl)-amino.

In some embodiments, R³ is selected from: C₃-C₆alkyl, (C₁-C₄alkoxy)-C₃-C₆alkyl, C₃-C₆haloalkyl, and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is (C₁-C₄alkoxy)-C₃-C₆alkyl. In some embodiments, R³ is (C₁-C₄haloalkoxy)-C₃-C₆alkyl. In some embodiments, Y is N. In some embodiments, Y is CH.

In some embodiments,
Y is N or CH;
R³ is selected from: C₁-C₆alkyl, (C₁-C₄alkoxy)-C₁-C₆alkyl, C₁-C₆haloalkyl, (C₁-C₄haloalkoxy)-C₁-C₆alkyl, (C₃-C₆cycloalkyl)-C₁-C₆alkyl, and (C₃-C₆cycloalkoxy)-C₁-C₆alkyl;
each R⁴ is independently selected from: C₁-C₆alkoxy optionally substituted with R⁵, C₁-C₄ₐlkylamino optionally substituted with R⁵, di-(C₁-C₄ₐlkyl)-amino optionally substituted with R⁵, SO₂R^{8a}, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl;
or two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₄alkoxy)-C₁-C₆alkyl;
each R⁵ is independently selected from hydroxyl, C₁-C₆alkoxy, amino, C₁-C₄alkylamino, di-(C₁-C₄alkyl)-amino, C₁-C₆haloalkoxy, and (C₁-C₄alkoxy)-C₁-C₆alkoxy;
R⁶ is selected from —C(═O)OH, —CH₂C(═O)OH, —C(═O)O—C₁-C₄alkyl, —C(═O)NHSO₂R^{8b}, and tetrazolyl;
R^{8a} and R^{8b} are each independently selected from C₁-C₆alkyl, amino, C₁-C₄alkylamino, di-(C₁-C₄ₐlkyl)-amino.

In some embodiments, R³ is selected from: C₃-C₆alkyl, (C₁-C₄alkoxy)-C₃-C₆alkyl, C₃-C₆haloalkyl, and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is (C₁-C₄alkoxy)-C₃-C₆alkyl. In some embodiments, R³ is (C₁-C₄haloalkoxy)-C₃-C₆alkyl. In some embodiments, Y is N. In some embodiments, Y is CH.

In some embodiments, the compound is a compound of Formula (VI-1):

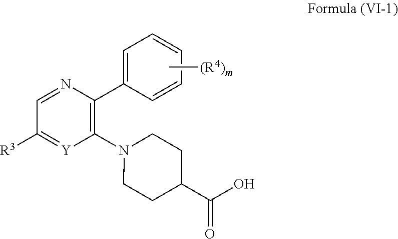

or a pharmaceutically acceptable salt thereof, wherein:
m is an integer from 0 to 4;
Y is N or CH;
R³ is selected from: C₁-C₆alkyl, (C₁-C₄alkoxy)-C₁-C₆alkyl, C₁-C₆haloalkyl, (C₁-C₄haloalkoxy)-C₁-C₆alkyl, (C₃-C₆cycloalkyl)-C₁-C₆alkyl, and (C₃-C₆cycloalkoxy)-C₁-C₆alkyl;
each R⁴ is independently selected from: C₁-C₆alkoxy optionally substituted with R⁵, C₁-C₄ₐlkylamino optionally substituted with R⁵, di-(C₁-C₄alkyl)-amino optionally substituted with R⁵, C₁-C₆haloalkoxy, C₃-C₆cycloalkoxy, (C₃-C₆cycloalkyl)-C₁-C₆alkoxy, halo, C₁-C₆alkyl, and C₁-C₆haloalkyl;
or two R⁴ substituents on adjacent carbon atoms of R¹ are taken together to form a fused 5-membered heterocycloalkyl or a fused 5-membered heteroaryl, either of which may be optionally substituted with one or more C₁-C₆alkyl, halo, oxo, C₁-C₆alkoxy, or (C₁-C₄alkoxy)-C₁-C₆alkyl;
each R⁵ is independently selected from hydroxyl, C₁-C₆alkoxy, amino, C₁-C₄alkylamino, di-(C₁-C₄alkyl)-amino, C₁-C₆haloalkoxy, and (C₁-C₄alkoxy)-C₁-C₆alkoxy.

In some embodiments, R³ is selected from: C₃-C₆alkyl, (C₁-C₄alkoxy)-C₃-C₆alkyl, C₃-C₆haloalkyl, and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is (C₁-C₄alkoxy)-C₃-C₆alkyl. In some embodiments, R³ is (C₁-C₄haloalkoxy)-C₃-C₆alkyl. In some embodiments, Y is N. In some embodiments, Y is CH.

In some embodiments, the compound is a compound of Formula (VI-2):

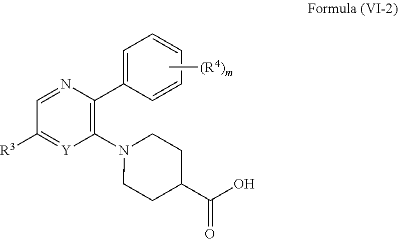

or a pharmaceutically acceptable salt thereof, wherein:
m is an integer from 0 to 4;
Y is N or CH;
R³ is selected from: C₁-C₆alkyl, (C₁-C₄alkoxy)-C₁-C₆alkyl, C₁-C₆haloalkyl, and (C₁-C₄haloalkoxy)-C₁-C₆alkyl;
each R⁴ is independently selected from: C₁-C₆alkoxy, C₁-C₄ₕaloalkoxy, halo, C₁-C₄alkyl, and C₁-C₄haloalkyl;
or two R⁴ substituents on adjacent carbon atoms of R¹ are taken together to form a fused 5-membered heterocycloalkyl comprising 1 or 2 O atoms or a fused 5-membered heteroaryl comprising 1 O atom and 0 or 1 N atoms, either of which may be optionally substituted with one or more C₁-C₄alkyl, halo, C₁-C₄alkoxy or (C₁-C₄alkoxy)-C₁-C₄alkyl.

In some embodiments, R³ is selected from: C₃-C₆alkyl, (C₁-C₄alkoxy)-C₃-C₆alkyl, C₃-C₆haloalkyl, and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is (C₁-C₄alkoxy)-C₃-C₆alkyl. In some embodiments, R³ is (C₁-C₄haloalkoxy)-C₃-C₆alkyl. In some embodiments, Y is N. In some embodiments, Y is CH.

In some embodiments, the compound is a compound of Formula (VI-3):

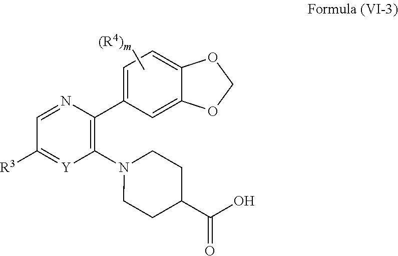

or a pharmaceutically acceptable salt thereof, wherein:
m is an integer from 0 to 2;
Y is N or CH;
R³ is selected from: C₁-C₆alkyl, (C₁-C₄alkoxy)-C₁-C₆alkyl, C₁-C₆haloalkyl, and (C₁-C₄haloalkoxy)-C₁-C₆alkyl; and each R⁴ is independently selected from: C₁-C₆alkoxy, C₁-C₄ₕaloalkoxy, halo, C₁-C₄alkyl, and C₁-C₄haloalkyl.

In some embodiments, m is 0 or 1; and R⁴ is selected from: C₁-C₆alkoxy, C₁-C₄haloalkoxy, and halo. In some embodiments, R³ is selected from: C₃-C₆alkyl, (C₁-C₄alkoxy)-C₃-C₆alkyl, C₃-C₆haloalkyl, and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is (C₁-C₄alkoxy)-C₃-C₆alkyl. In some embodiments, R³ is (C₁-C₄haloalkoxy)-C₃-C₆alkyl. In some embodiments, Y is N. In some embodiments, Y is CH.

In some embodiments, the compound is a compound of Formula (VI-4):

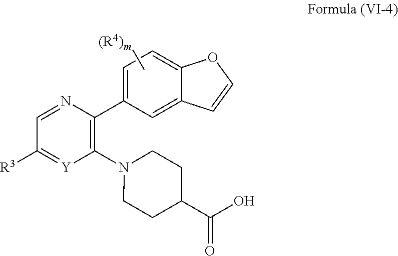

or a pharmaceutically acceptable salt thereof, wherein:
m is an integer from 0 to 2;
Y is N or CH;
R³ is selected from: C₁-C₆alkyl, (C₁-C₄alkoxy)-C₁-C₆alkyl, C₁-C₆haloalkyl, and (C₁-C₄haloalkoxy)-C₁-C₆alkyl; and
each R⁴ is independently selected from: C₁-C₆alkoxy, C₁-C₄haloalkoxy, halo, C₁-C₄alkyl, and C₁-C₄haloalkyl.

In some embodiments, m is 0 or 1; and R⁴ is selected from: C₁-C₆alkoxy, C₁-C₄haloalkoxy, and halo. In some embodiments, R³ is selected from: C₃-C₆alkyl, (C₁-C₄alkoxy)-C₃-C₆alkyl, C₃-C₆haloalkyl, and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is selected from: (C₁-C₄alkoxy)-C₃-C₆alkyl and (C₁-C₄haloalkoxy)-C₃-C₆alkyl. In some embodiments, R³ is (C₁-C₄alkoxy)-C₃-C₆alkyl. In some embodiments, R³ is (C₁-C₄haloalkoxy)-C₃-C₆alkyl. In some embodiments, Y is N. In some embodiments, Y is CH.

In some embodiments, the compound is a compound of Formula (I):

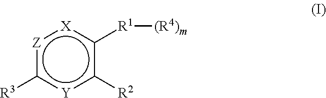

or a pharmaceutically acceptable salt thereof, wherein:
m is an integer from 0 to 4;
X is selected from CH and N;
Y is selected from CH, S, O, and N;
Z is selected from a bond and CR⁷;
R¹ is selected from aryl and heteroaryl,
R² is a mono- or bi-cyclic heterocycloalkyl or a mono- or bi-cyclic heteroaryl, either of which may be optionally substituted with one or more R⁶.
R³ is selected from:
   alkyl optionally substituted with SO₂-alkyl,
   alkoxyalkyl,
   haloalkyl,
   haloalkoxyalkyl,
   cycloalkylalkyl, and
   cycloalkoxyalkyl;
each R⁴ is independently selected from:
   alkoxy optionally substituted with R⁵,
   SO₂R^{8a},
   haloalkoxy,
   cycloalkoxy,
   cycloalkylalkoxy, and
   halo,
or two R⁴ substituents taken together form a fused heterocycloalkyl or a fused heteroaryl either of which may be optionally substituted with one or more alkyl, halo, oxo, alkyl, or alkoxyalkyl;
each R⁵ is independently selected from hydroxyl, alkoxy, amino, alkylamino, dialkylamino, and haloalkoxy;
each R⁶ is independently selected from hydroxyl, hydroxyalkoxy, carboxyl, methylcarboxyl, carboxylalkyl, carboxylalkoxy, halo, alkyl, alkoxy, SO₂R^{8b}, C(═O)NHSO₂R^{8b}, heterocycloalkyl, cyano, and tetrazolyl;
R⁷ is selected from hydrogen and halo; and
R^{8a} and R^{8b} are each independently selected from alkyl, amino, alkylamino, and dialkylamino;
wherein X and Y are not both CH.

In some embodiments, the compound is a compound of Formula (II):

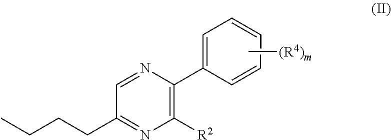

or a pharmaceutically acceptable salt thereof, wherein:
m is an integer from 0 to 3;
R² is a mono- or bi-cyclic heterocycloalkyl, optionally substituted with one or more R⁶.
each R⁴ is independently selected from alkoxy optionally substituted with R⁵,
SO₂R^{8a}, and halo, or two R⁴ substituents taken together form a fused heterocycloalkyl or heteroaryl;
R⁵ is selected from alkoxy, amino, alkylamino, dialkylamino;
each R⁶ is independently selected from carboxyl, carboxylalkyl, halo, alkyl,
SO₂R^{8b}, C(═O)NHSO₂R^{8b}, and tetrazolyl; and
R^{8a} and R^{8b} are each independently selected from alkyl, amino, alkylamino, dialkylamino.

In some embodiments, the compound is a compound of Formula (III):

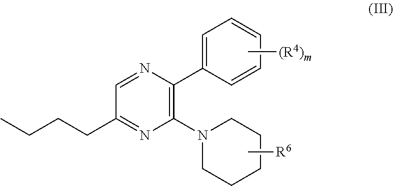

or a pharmaceutically acceptable salt thereof, wherein:
m is an integer from 0 to 3;
each R⁴ is independently selected from alkoxy optionally substituted with R⁵,
SO₂R^{8a}, and halo, or two R⁴ substituents taken together form a fused heterocycloalkyl or heteroaryl;
R⁵ is selected from alkoxy, amino, alkylamino, dialkylamino;
R⁶ is selected from carboxyl, carboxylalkyl, halo, alkyl, SO₂R^{8b},
C(═O)NHSO₂Rg^{b}, and tetrazolyl; and
R^{8a} and R^{8b} are each independently selected from alkyl, amino, alkylamino,
dialkylamino.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

That is, any combination of the groups described above for the various variables is contemplated herein. Throughout the specification, groups and substituents thereof are chosen by one skilled in the field to provide stable moieties and compounds.

Exemplary compounds ofthe invention include the compounds described in the following Table:

TABLE 1

| Cmpd | | |
|---|---|---|
| # | Structure | Name |
| 1 | 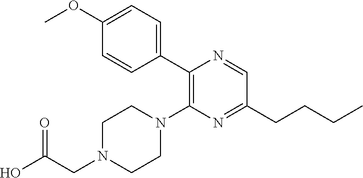 [SMILES: CCCCc1cnc(-c2ccc(OC)cc2)c(N2CCN(CC(=O)O)CC2)n1] | 2-(4-(6-butyl-3-(4- methoxyphenyl)pyrazin-2-yl)piperazin-1- yl)acetic acid |
| 2 | 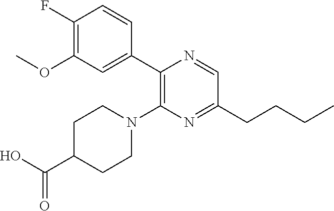 [SMILES: CCCCc1cnc(-c2ccc(F)c(OC)c2)c(N2CCC(C(=O)O)CC2)n1] | 1-(6-butyl-3-(4-fluoro-3- methoxyphenyl)pyrazin-2-yl)piperidine- 4-carboxylic acid |
| 3 | 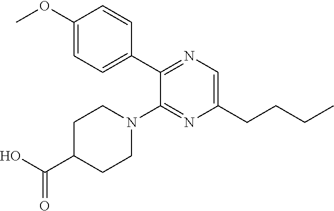 [SMILES: CCCCc1cnc(-c2ccc(OC)cc2)c(N2CCC(C(=O)O)CC2)n1] | 1-(6-butyl-3-(4-methoxyphenyl)pyrazin- 2-yl)piperidine-4-carboxylic acid |
| 4 | 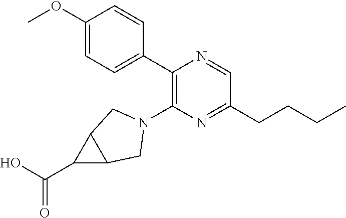 [SMILES: CCCCc1cnc(-c2ccc(OC)cc2)c(N2CC3C(C2)C3C(=O)O)n1] | 3-(6-butyl-3-(4-methoxyphenyl)pyrazin- 2-yl)-3-azabicyclo[3.1.0]hexane-6- carboxylic acid |
| 5 | 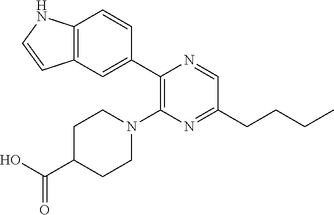 [SMILES: CCCCc1cnc(-c2ccc3[nH]ccc3c2)c(N2CCC(C(=O)O)CC2)n1] | 1-(6-butyl-3-(1H-indol-5-yl)pyrazin-2- yl)piperidine-4-carboxylic acid |
| 6 | 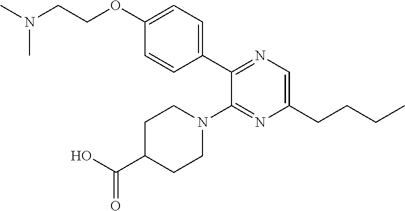 [SMILES: CCCCc1cnc(-c2ccc(OCCN(C)C)cc2)c(N2CCC(C(=O)O)CC2)n1] | 1-(6-butyl-3-(4-(2- (dimethylamino)ethoxy)phenyl)pyrazin- 2-yl)piperidine-4-carboxylic acid |
| 7 | 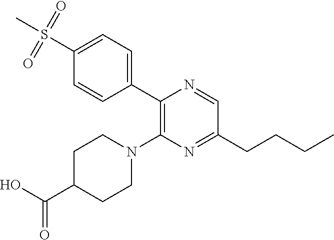 [SMILES: CCCCc1cnc(-c2ccc(S(C)(=O)=O)cc2)c(N2CCC(C(=O)O)CC2)n1] | 1-(6-butyl-3-(4- (methylsulfonyl)phenyl)pyrazin-2- yl)piperidine-4-carboxylic acid |
| 8 | 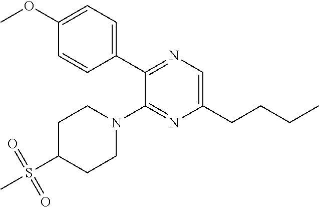 [SMILES: CCCCc1cnc(-c2ccc(OC)cc2)c(N2CCC(S(C)(=O)=O)CC2)n1] | 5-butyl-2-(4-methoxyphenyl)-3-(4- (methylsulfonyl)piperidin-1-yl)pyrazine |
| 9 | 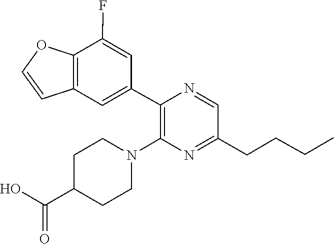 [SMILES: CCCCc1cnc(-c2cc(F)c3occc3c2)c(N2CCC(C(=O)O)CC2)n1] | 1-(6-butyl-3-(7-fluorobenzofuran-5- yl)pyrazin-2-yl)piperidine-4-carboxylic acid |
| 10 | 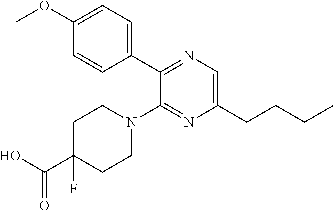 [SMILES: CCCCc1cnc(-c2ccc(OC)cc2)c(N2CCC(F)(C(=O)O)CC2)n1] | 1-(6-butyl-3-(4-methoxyphenyl)pyrazin- 2-yl)-4-fluoropiperidine-4-carboxylic acid |
| 11 | 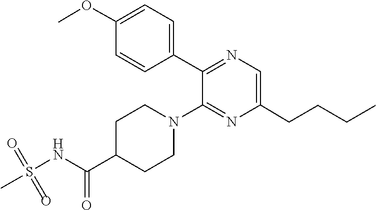 | 1-(6-butyl-3-(4-methoxyphenyl)pyrazin- 2-yl)-N-(methylsulfonyl)piperidine-4- carboxamide |
| 12 | 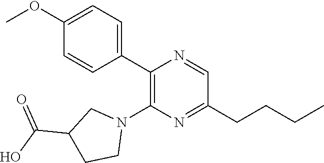 [SMILES: CCCCc1cnc(-c2ccc(OC)cc2)c(N2CCC(C(=O)O)C2)n1] | 1-(6-butyl-3-(4-methoxyphenyl)pyrazin- 2-yl)pyrrolidine-3-carboxylic acid |
| 13 | 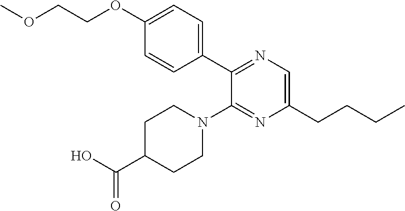 [SMILES: CCCCc1cnc(-c2ccc(OCCOC)cc2)c(N2CCC(C(=O)O)CC2)n1] | 1-(6-butyl-3-(4-(2- methoxyethoxy)phenyl)pyrazin-2- yl)piperidine-4-carboxylic acid |
| 14 | 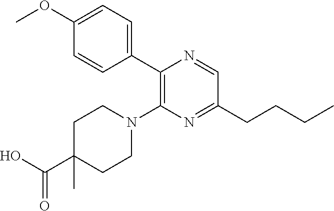 [SMILES: CCCCc1cnc(-c2ccc(OC)cc2)c(N2CCC(C)(C(=O)O)CC2)n1] | 1-(6-butyl-3-(4-methoxyphenyl)pyrazin- 2-yl)-4-methylpiperidine-4-carboxylic acid |
| 15 | 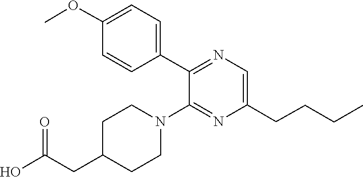 [SMILES: CCCCc1cnc(-c2ccc(OC)cc2)c(N2CCC(CC(=O)O)CC2)n1] | 2-(1-(6-butyl-3-(4- methoxyphenyl)pyrazin-2-yl)piperidin-4- yl)acetic acid |
| 16 | 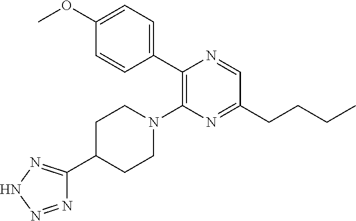 [SMILES: CCCCc1cnc(-c2ccc(OC)cc2)c(N2CCC(c3nn[nH]n3)CC2)n1] | 3-(4-(2H-tetrazol-5-yl)piperidin-1-yl)-5- butyl-2-(4-methoxyphenyl)pyrazine |
| 17 | 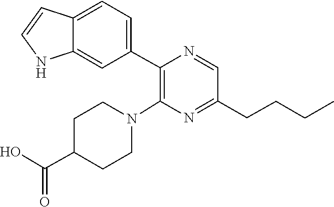 [SMILES: CCCCc1cnc(-c2ccc3cc[nH]c3c2)c(N2CCC(C(=O)O)CC2)n1] | 1-(6-butyl-3-(1H-indol-6-yl)pyrazin-2- yl)piperidine-4-carboxylic acid |
| 18 | 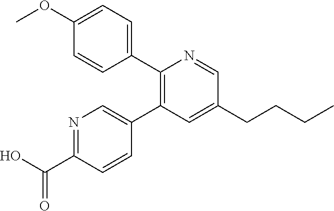 [SMILES: CCCCc1cnc(-c2ccc(OC)cc2)c(-c2ccc(C(=O)O)nc2)c1] | 5′-butyl-2′-(4-methoxyphenyl)-[3,3′- bipyridine]-6-carboxylic acid |
| 19 | 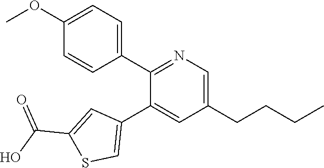 [SMILES: CCCCc1cnc(-c2ccc(OC)cc2)c(-c2csc(C(=O)O)c2)c1] | 4-(5-butyl-2-(4-methoxyphenyl)pyridin- 3-yl)thiophene-2-carboxylic acid |
| 20 | 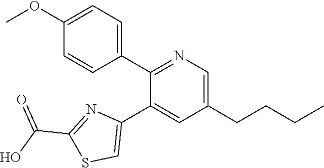 [SMILES: CCCCc1cnc(-c2ccc(OC)cc2)c(-c2csc(C(=O)O)n2)c1] | 4-(5-butyl-2-(4-methoxyphenyl)pyridin- 3-yl)thiazole-2-carboxylic acid |
| 21 | 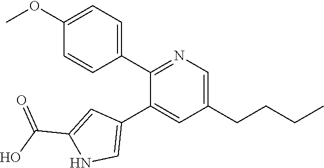 [SMILES: CCCCc1cnc(-c2ccc(OC)cc2)c(-c2c[nH]c(C(=O)O)c2)c1] | 4-(5-butyl-2-(4-methoxyphenyl)pyridin- 3-yl)-1H-pyrrole-2-carboxylic acid |
| 22 | 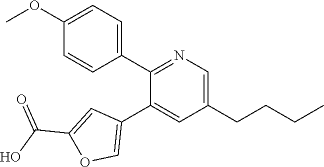 [SMILES: CCCCc1cnc(-c2ccc(OC)cc2)c(-c2coc(C(=O)O)c2)c1] | 4-(5-butyl-2-(4-methoxyphenyl)pyridin- 3-yl)furan-2-carboxylic acid |
| 23 | 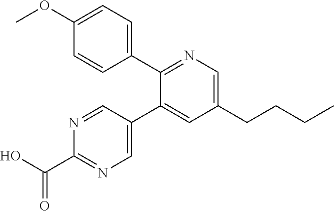 [SMILES: CCCCc1cnc(-c2ccc(OC)cc2)c(-c2cnc(C(=O)O)nc2)c1] | 5-(5-butyl-2-(4-methoxyphenyl)pyridin- 3-yl)pyrimidine-2-carboxylic acid |
| 24 | 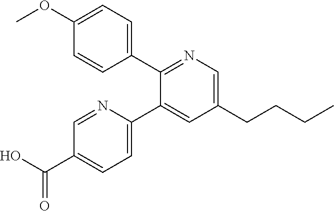 [SMILES: CCCCc1cnc(-c2ccc(OC)cc2)c(-c2ccc(C(=O)O)cn2)c1] | 5′-butyl-2′-(4-methoxyphenyl)-[2,3′- bipyridine]-5-carboxylic acid |
| 25 | 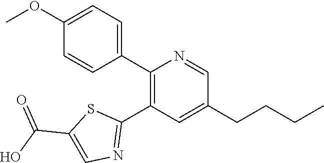 [SMILES: CCCCc1cnc(-c2ccc(OC)cc2)c(-c2ncc(C(=O)O)s2)c1] | 2-(5-butyl-2-(4-methoxyphenyl)pyridin- 3-yl)thiazole-5-carboxylic acid |
| 26 | 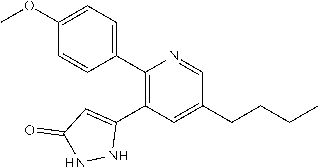 [SMILES: CCCCc1cnc(-c2ccc(OC)cc2)c(-c2cc(=O)[nH][nH]2)c1] | 5-(5-butyl-2-(4-methoxyphenyl)pyridin- 3-yl)-1,2-dihydro-3H-pyrazol-3-one |
| 27 | 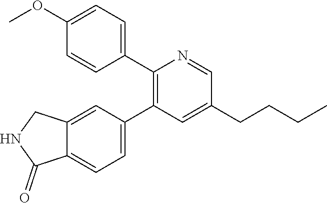 [SMILES: CCCCc1cnc(-c2ccc(OC)cc2)c(-c2ccc3c(c2)CNC3=O)c1] | 5-(5-butyl-2-(4-methoxyphenyl)pyridin- 3-yl)isoindolin-1-one |
| 28 | 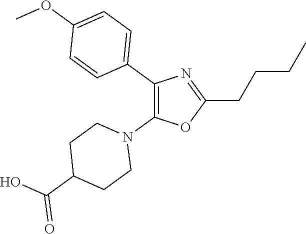 [SMILES: CCCCc1nc(-c2ccc(OC)cc2)c(N2CCC(C(=O)O)CC2)o1] | 1-(2-butyl-4-(4-methoxyphenyl)oxazol-5- yl)piperidine-4-carboxylic acid |
| 29 | 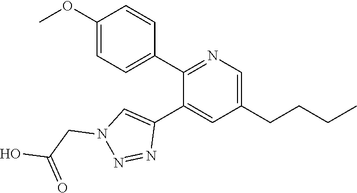 [SMILES: CCCCc1cnc(-c2ccc(OC)cc2)c(-c2cn(CC(=O)O)nn2)c1] | 2-(4-(5-butyl-2-(4- methoxyphenyl)pyridin-3-yl)-1H-1,2,3- triazol-1-yl)acetic acid |
| 30 | 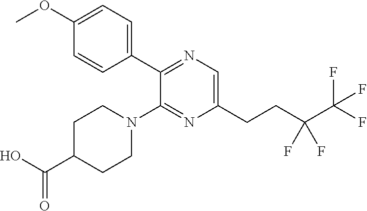 [SMILES: COc1ccc(-c2ncc(CCC(F)(F)C(F)(F)F)nc2N2CCC(C(=O)O)CC2)cc1] | 1-(3-(4-methoxyphenyl)-6-(3,3,4,4,4- pentafluorobutyl)pyrazin-2-yl)piperidine- 4-carboxylic acid |
| 31 | 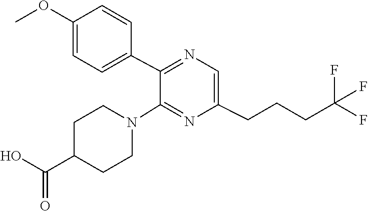 [SMILES: COc1ccc(-c2ncc(CCCC(F)(F)F)nc2N2CCC(C(=O)O)CC2)cc1] | 1-(3-(4-methoxyphenyl)-6-(4,4,4- trifluorobutyl)pyrazin-2-yl)piperidine-4- carboxylic acid |
| 32 | 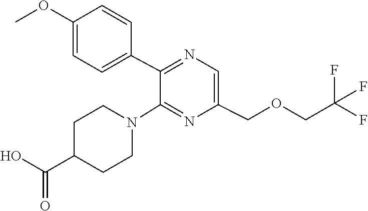 [SMILES: COc1ccc(-c2ncc(COCC(F)(F)F)nc2N2CCC(C(=O)O)CC2)cc1] | 1-(3-(4-methoxyphenyl)-6-((2,2,2- trifluoroethoxy)methyl)pyrazin-2- yl)piperidine-4-carboxylic acid |
| 33 | 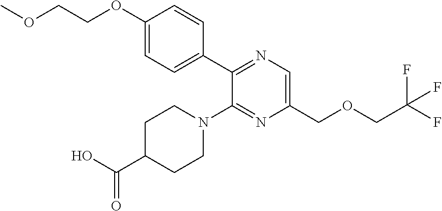 [SMILES: COCCOc1ccc(-c2ncc(COCC(F)(F)F)nc2N2CCC(C(=O)O)CC2)cc1] | 1-(3-(4-(2-methoxyethoxy)phenyl)-6- ((2,2,2-trifluoroethoxy)methyl)pyrazin-2- yl)piperidine-4-carboxylic acid |
| 34 | 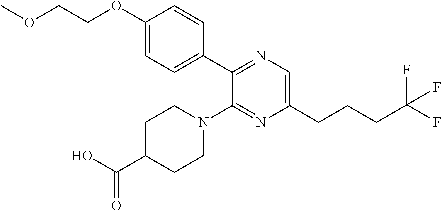 [SMILES: COCCOc1ccc(-c2ncc(CCCC(F)(F)F)nc2N2CCC(C(=O)O)CC2)cc1] | 1-(3-(4-(2-methoxyethoxy)phenyl)-6- (4,4,4-trifluorobutyl)pyrazin-2- yl)piperidine-4-carboxylic acid |
| 35 | 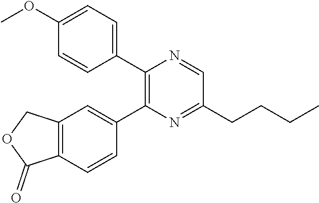 [SMILES: CCCCc1cnc(-c2ccc(OC)cc2)c(-c2ccc3c(c2)COC3=O)n1] | 5-(6-butyl-3-(4-methoxyphenyl)pyrazin- 2-yl)isobenzofuran-1(3H)-one |
| 36 | 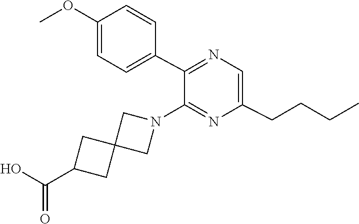 [SMILES: CCCCc1cnc(-c2ccc(OC)cc2)c(N2CC3(CC(C(=O)O)C3)C2)n1] | 2-(6-butyl-3-(4-methoxyphenyl)pyrazin- 2-yl)-2-azaspiro[3.3]heptane-6-carboxylic acid |
| 37 | 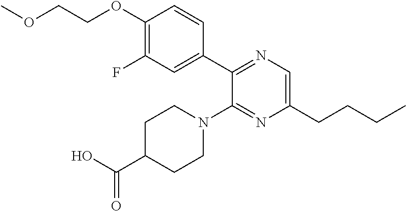 [SMILES: CCCCc1cnc(-c2ccc(OCCOC)c(F)c2)c(N2CCC(C(=O)O)CC2)n1] | 1-(6-butyl-3-(3-fluoro-4-(2- methoxyethoxy)phenyl)pyrazin-2- yl)piperidine-4-carboxylic acid |
| 38 | 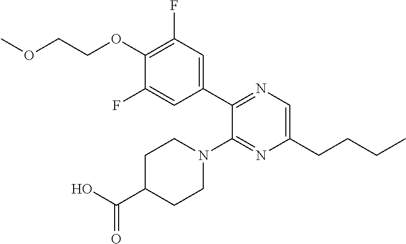 [SMILES: CCCCc1cnc(-c2cc(F)c(OCCOC)c(F)c2)c(N2CCC(C(=O)O)CC2)n1] | 1-(6-butyl-3-(3,5-difluoro-4-(2- methoxyethoxy)phenyl)pyrazin-2- yl)piperidine-4-carboxylic acid |
| 39 | 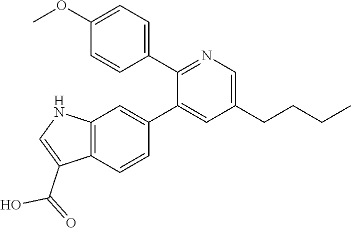 [SMILES: CCCCc1cnc(-c2ccc(OC)cc2)c(-c2ccc3c(C(=O)O)c[nH]c3c2)c1] | 6-(5-butyl-2-(4-methoxyphenyl)pyridin- 3-yl)-1H-indole-3-carboxylic acid |
| 40 | 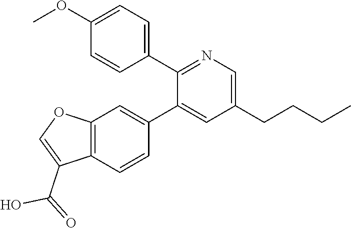 [SMILES: CCCCc1cnc(-c2ccc(OC)cc2)c(-c2ccc3c(C(=O)O)coc3c2)c1] | 6-(5-butyl-2-(4-methoxyphenyl)pyridin- 3-yl)benzofuran-3-carboxylic acid |
| 41 | 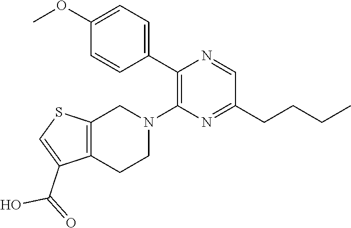 [SMILES: CCCCc1cnc(-c2ccc(OC)cc2)c(N2CCc3c(C(=O)O)csc3C2)n1] | 6-(6-butyl-3-(4-methoxyphenyl)pyrazin- 2-yl)-4,5,6,7-tetrahydrothieno[2,3- c]pyridine-3-carboxylic acid |
| 42 | 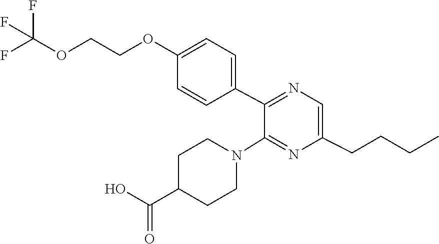 [SMILES: CCCCc1cnc(-c2ccc(OCCOC(F)(F)F)cc2)c(N2CCC(C(=O)O)CC2)n1] | 1-(6-butyl-3-(4-(2- (trifluoromethoxy)ethoxy)phenyl)pyrazin- 2-yl)piperidine-4-carboxylic acid |
| 43 | 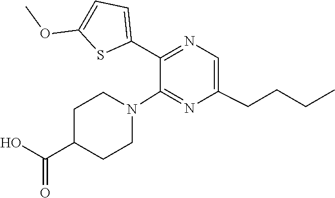 [SMILES: CCCCc1cnc(-c2ccc(OC)s2)c(N2CCC(C(=O)O)CC2)n1] | 1-(6-butyl-3-(5-methoxythiophen-2- yl)pyrazin-2-yl)piperidine-4-carboxylic acid |
| 44 | 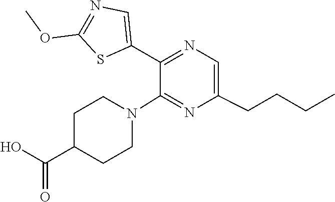 [SMILES: CCCCc1cnc(-c2cnc(OC)s2)c(N2CCC(C(=O)O)CC2)n1] | 1-(6-butyl-3-(2-methoxythiazol-5- yl)pyrazin-2-yl)piperidine-4-carboxylic acid |
| 45 | 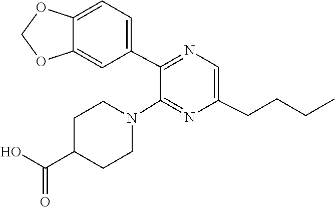 [SMILES: CCCCc1cnc(-c2ccc3c(c2)OCO3)c(N2CCC(C(=O)O)CC2)n1] | 1-(3-benzo[d][1,3]dioxol-5-yl)-6- butylpyrazin-2-yl)piperidine-4-carboxylic acid |
| 46 | 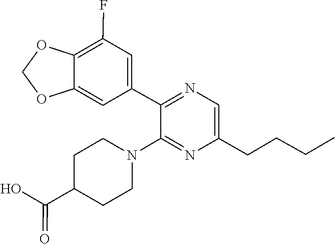 [SMILES: CCCCc1cnc(-c2cc(F)c3c(c2)OCO3)c(N2CCC(C(=O)O)CC2)n1] | 1-(6-butyl-3-(7- fluorobenzo[d][1,3]dioxol-5-yl)pyrazin-2- yl)piperidine-4-carboxylic acid |
| 47 | 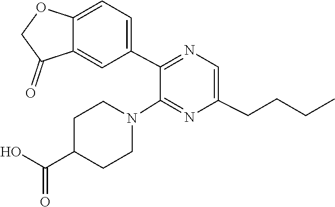 [SMILES: CCCCc1cnc(-c2ccc3c(c2)C(=O)CO3)c(N2CCC(C(=O)O)CC2)n1] | 1-(6-butyl-3-(3-oxo-2,3- dihydrobenzofuran-5-yl)pyrazin-2- yl)piperidine-4-carboxylic acid |
| 48 | 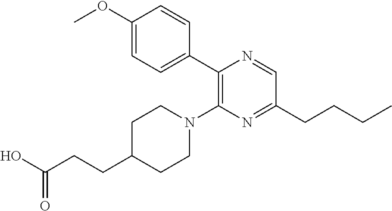 [SMILES: CCCCc1cnc(-c2ccc(OC)cc2)c(N2CCC(CCC(=O)O)CC2)n1] | 3-(1-(6-butyl-3-(4- methoxyphenyl)pyrazin-2-yl)piperidin-4- yl)propanoic acid |
| 49 | 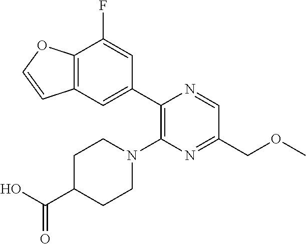 [SMILES: COCc1cnc(-c2cc(F)c3occc3c2)c(N2CCC(C(=O)O)CC2)n1] | 1-(3-(7-fluorobenzofuran-5-yl)-6- (methoxymethyl)pyrazin-2-yl)piperidine- 4-carboxylic acid |
| 50 | 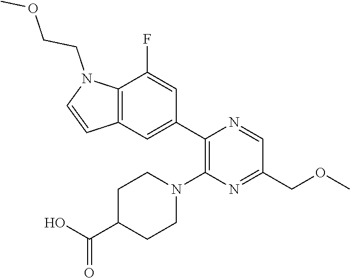 | 1-(3-(7-fluoro-1-(2-methoxyethyl)-1H- indol-5-yl)-6-(methoxymethyl)pyrazin-2- yl)piperidine-4-carboxylic acid |
| 51 | 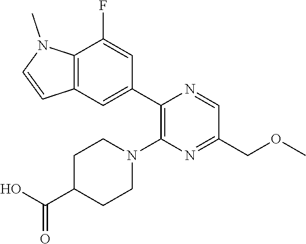 [SMILES: COCc1cnc(-c2cc(F)c3c(ccn3C)c2)c(N2CCC(C(=O)O)CC2)n1] | 1-(3-(7-fluoro-1-methyl-1H-indol-5-yl)-6- (methoxymethyl)pyrazin-2-yl)piperidine- 4-carboxylic acid |
| 52 | 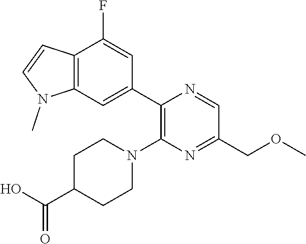 [SMILES: COCc1cnc(-c2cc(F)c3ccn(C)c3c2)c(N2CCC(C(=O)O)CC2)n1] | 1-(3-(4-fluoro-1-methyl-1H-indol-6-yl)-6- (methoxymethyl)pyrazin-2-yl)piperidine- 4-carboxylic acid |
| 53 | 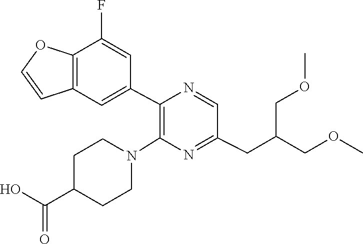 [SMILES: COCC(COC)Cc1cnc(-c2cc(F)c3occc3c2)c(N2CCC(C(=O)O)CC2)n1] | 1-(3-(7-fluorobenzofuran-5-yl)-6-(3- methoxy-2- (methoxymethyl)propyl)pyrazin-2- yl)piperidine-4-carboxylic acid |
| 54 | 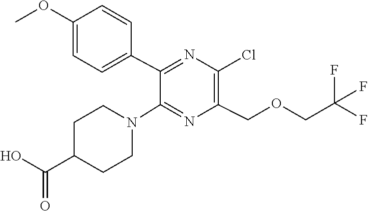 [SMILES: COc1ccc(-c2nc(Cl)c(COCC(F)(F)F)nc2N2CCC(C(=O)O)CC2)cc1] | 1-(5-chloro-3-(4-methoxyphenyl)-6- ((2,2,2-trifluoroethoxy)methyl)pyrazin-2- yl)piperidine-4-carboxylic acid |
| 55 | 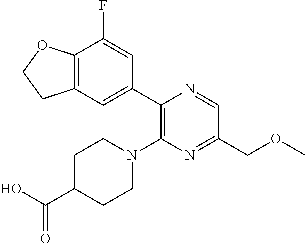 [SMILES: COCc1cnc(-c2cc(F)c3c(c2)CCO3)c(N2CCC(C(=O)O)CC2)n1] | 1-(3-(7-fluoro-2,3-dihydrobenzofuran-5- yl)-6-(methoxymethyl)pyrazin-2- yl)piperidine-4-carboxylic acid |
| 56 | 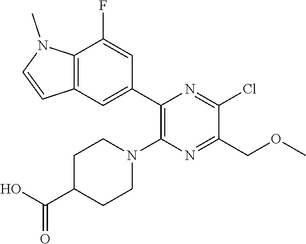 [SMILES: COCc1nc(N2CCC(C(=O)O)CC2)c(-c2cc(F)c3c(ccn3C)c2)nc1Cl] | 1-(5-chloro-3-(7-fluoro-1-methyl-1H- indol-5-yl)-6-(methoxymethyl)pyrazin-2- yl)piperidine-4-carboxylic acid |
| 57 | 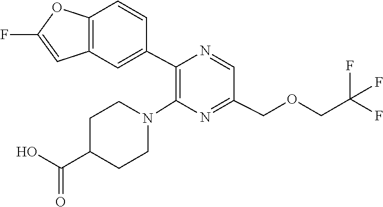 [SMILES: O=C(O)C1CCN(c2nc(COCC(F)(F)F)cnc2-c2ccc3oc(F)cc3c2)CC1] | 1-(3-(2-fluorobenzofuran-5-yl)-6-((2,2,2- trifluoroethoxy)methyl)pyrazin-2- yl)piperidine-4-carboxylic acid |
| 58 | 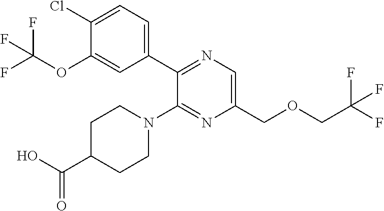 [SMILES: O=C(O)C1CCN(c2nc(COCC(F)(F)F)cnc2-c2ccc(Cl)c(OC(F)(F)F)c2)CC1] | 1-(3-(4-chloro-3- (trifluoromethoxy)phenyl)-6-((2,2,2- trifluoroethoxy)methyl)pyrazin-2- yl)piperidine-4-carboxylic acid |
| 59 | 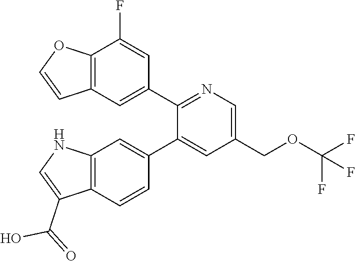 [SMILES: O=C(O)c1c[nH]c2cc(-c3cc(COC(F)(F)F)cnc3-c3cc(F)c4occc4c3)ccc12] | 6-(2-(7-fluorobenzofuran-5-yl)-5- ((trifluoromethoxy)methyl)pyridin-3-yl)- 1H-indole-3-carboxylic acid |
| 60 | 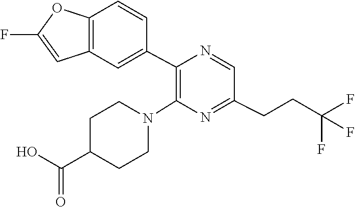 [SMILES: O=C(O)C1CCN(c2nc(CCC(F)(F)F)cnc2-c2ccc3oc(F)cc3c2)CC1] | 1-(3-(2-fluorobenzofuran-5-yl)-6-(3,3,3- trifluoropropyl)pyrazin-2-yl)piperidine-4- carboxylic acid |
| 61 | 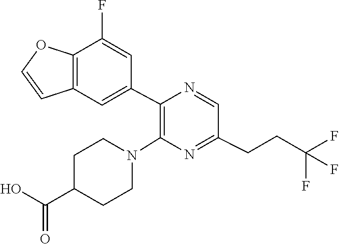 [SMILES: O=C(O)C1CCN(c2nc(CCC(F)(F)F)cnc2-c2cc(F)c3occc3c2)CC1] | 1-(3-(7-fluorobenzofuran-5-yl)-6-(3,3,3- trifluoropropyl)pyrazin-2-yl)piperidine-4- carboxylic acid |
| 62 | 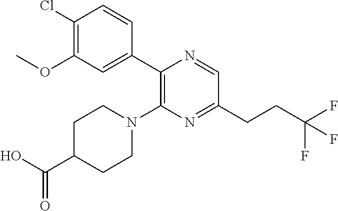 [SMILES: COc1cc(-c2ncc(CCC(F)(F)F)nc2N2CCC(C(=O)O)CC2)ccc1Cl] | 1-(3-(4-chloro-3-methoxyphenyl)-6- (3,3,3-trifluoropropyl)pyrazin-2- yl)piperidine-4-carboxylic acid |
| 63 | 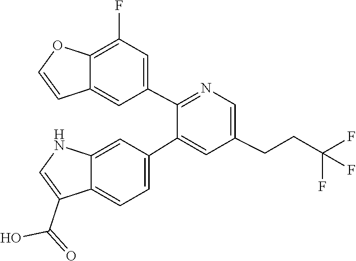 [SMILES: O=C(O)c1c[nH]c2cc(-c3cc(CCC(F)(F)F)cnc3-c3cc(F)c4occc4c3)ccc12] | 6-(2-(7-fluorobenzofuran-5-yl)-5-(3,3,3- trifluoropropyl)pyridin-3-yl)-1H-indole- 3-carboxylic acid |
| 64 | 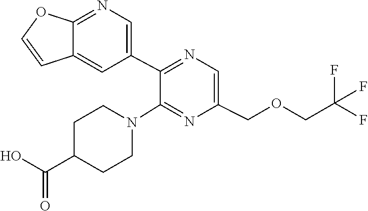 [SMILES: O=C(O)C1CCN(c2nc(COCC(F)(F)F)cnc2-c2cnc3occc3c2)CC1] | 1-(3-(furo[2,3-b]pyridin-5-yl)-6-((2,2,2- trifluoroethoxy)methyl)pyrazin-2- yl)piperidine-4-carboxylic acid |
| 65 | 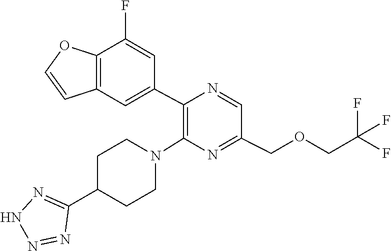 [SMILES: Fc1cc(-c2ncc(COCC(F)(F)F)nc2N2CCC(c3nn[nH]n3)CC2)cc2ccoc12] | 3-(4-(2H-tetrazol-5-yl)piperidin-1-yl)-2- (7-fluorobenzofuran-5-yl)-5-((2,2,2- trifluoroethoxy)methyl)pyrazine |
| 66 | 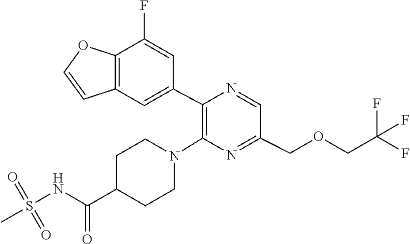 [SMILES: CS(=O)(=O)NC(=O)C1CCN(c2nc(COCC(F)(F)F)cnc2-c2cc(F)c3occc3c2)CC1] | 1-(3-(7-fluorobenzofuran-5-yl)-6-((2,2,2- trifluoroethoxy)methyl)pyrazin-2-yl)-N- (methylsulfonyl)piperidine-4- carboxamide |
| 67 | 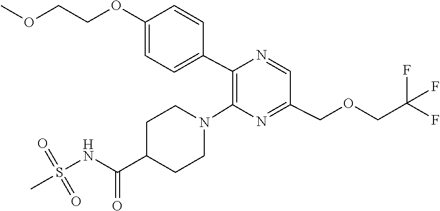 [SMILES: COCCOc1ccc(-c2ncc(COCC(F)(F)F)nc2N2CCC(C(=O)NS(C)(=O)=O)CC2)cc1] | 1-(3-(4-(2-methoxyethoxy)phenyl)-6- ((2,2,2-trifluoroethoxy)methyl)pyrazin-2- yl)-N-(methylsulfonyl)piperidine-4- carboxamide |
| 68 | 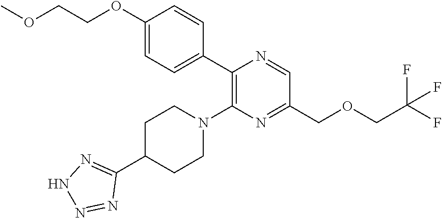 [SMILES: COCCOc1ccc(-c2ncc(COCC(F)(F)F)nc2N2CCC(c3nn[nH]n3)CC2)cc1] | 3-(4-(2H-tetrazol-5-yl)piperidin-1-yl)-2- (4-(2-methoxyethoxy)phenyl)-5-((2,2,2- trifluoroethoxy)methyl)pyrazine |
| 69 | 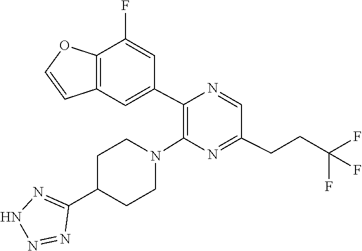 [SMILES: Fc1cc(-c2ncc(CCC(F)(F)F)nc2N2CCC(c3nn[nH]n3)CC2)cc2ccoc12] | 3-(4-(2H-tetrazol-5-yl)piperidin-1-yl)-2- (7-fluorobenzofuran-5-yl)-5-(3,3,3- trifluoropropyl)pyrazine |
| 70 | 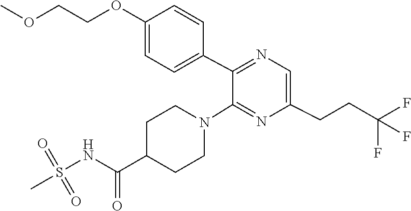 [SMILES: COCCOc1ccc(-c2ncc(CCC(F)(F)F)nc2N2CCC(C(=O)NS(C)(=O)=O)CC2)cc1] | 1-(3-(4-(2-methoxyethoxy)phenyl)-6- (3,3,3-trifluoropropyl)pyrazin-2-yl)-N- (methylsulfonyl)piperidine-4- carboxamide |
| 71 | 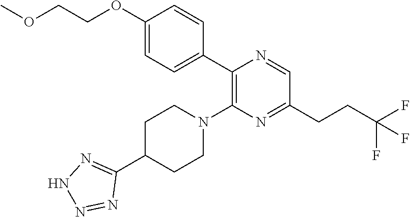 [SMILES: COCCOc1ccc(-c2ncc(CCC(F)(F)F)nc2N2CCC(c3nn[nH]n3)CC2)cc1] | 3-(4-(2H-tetrazol-5-yl)piperidin-1-yl)-2- (4-(2-methoxyethoxy)phenyl)-5-(3,3,3- trifluoropropyl)pyrazine |
| 72 | 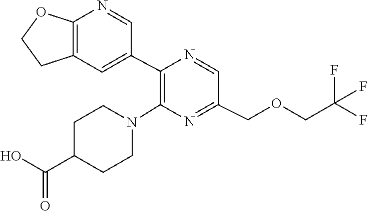 [SMILES: O=C(O)C1CCN(c2nc(COCC(F)(F)F)cnc2-c2cnc3c(c2)CCO3)CC1] | 1-(3-(2,3-dihydrofuro[2,3-b]pyridin-5-yl)- 6-((2,2,2-trifluoroethoxy)methyl)pyrazin- 2-yl)piperidine-4-carboxylic acid |
| 73 | 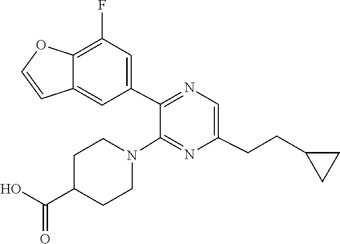 [SMILES: O=C(O)C1CCN(c2nc(CCC3CC3)cnc2-c2cc(F)c3occc3c2)CC1] | 1-(6-(2-cyclopropylethyl)-3-(7- fluorobenzofuran-5-yl)pyrazin-2- yl)piperidine-4-carboxylic acid |
| 74 | 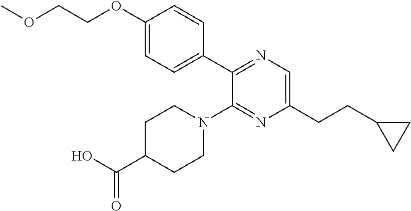 [SMILES: COCCOc1ccc(-c2ncc(CCC3CC3)nc2N2CCC(C(=O)O)CC2)cc1] | 1-(6-(2-cyclopropylethyl)-3-(4-(2- methoxyethoxy)phenyl)pyrazin-2- yl)piperidine-4-carboxylic acid |
| 75 | 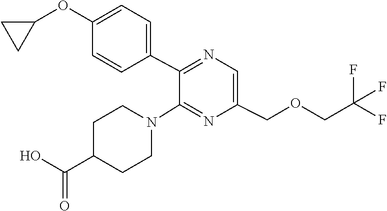 [SMILES: O=C(O)C1CCN(c2nc(COCC(F)(F)F)cnc2-c2ccc(OC3CC3)cc2)CC1] | 1-(3-(4-cyclopropoxyphenyl)-6-((2,2,2- trifluoroethoxy)methyl)pyrazin-2- yl)piperidine-4-carboxylic acid |
| 76 | 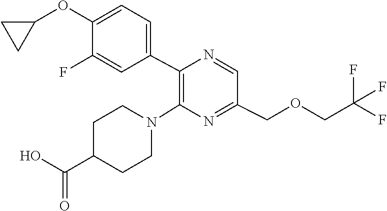 [SMILES: O=C(O)C1CCN(c2nc(COCC(F)(F)F)cnc2-c2ccc(OC3CC3)c(F)c2)CC1] | 1-(3-(4-cyclopropoxy-3-fluorophenyl)-6- ((2,2,2-trifluoroethoxy)methyl)pyrazin-2- yl)piperidine-4-carboxylic acid |
| 77 | 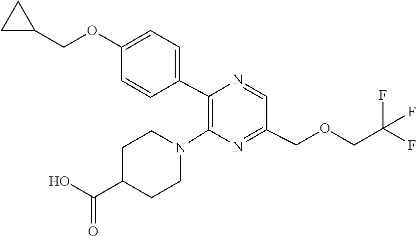 [SMILES: O=C(O)C1CCN(c2nc(COCC(F)(F)F)cnc2-c2ccc(OCC3CC3)cc2)CC1] | 1-(3-(4-(cyclopropylmethoxy)phenyl)-6- ((2,2,2-trifluoroethoxy)methyl)pyrazin-2- yl)piperidine-4-carboxylic acid |
| 78 | 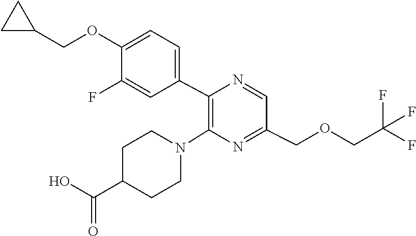 [SMILES: O=C(O)C1CCN(c2nc(COCC(F)(F)F)cnc2-c2ccc(OCC3CC3)c(F)c2)CC1] | 1-(3-(4-(cyclopropylmethoxy)-3- fluorophenyl)-6-((2,2,2- trifluoroethoxy)methyl)pyrazin-2- yl)piperidine-4-carboxylic acid |
| 79 | 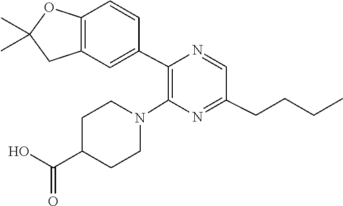 [SMILES: CCCCc1cnc(-c2ccc3c(c2)CC(C)(C)O3)c(N2CCC(C(=O)O)CC2)n1] | 1-(6-butyl-3-(2,2-dimethyl-2,3- dihydrobenzofuran-5-yl)pyrazin-2- yl)piperidine-4-carboxylic acid |
| 80 | 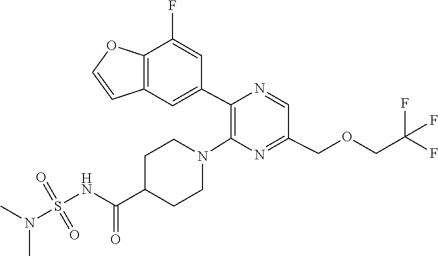 [SMILES: CN(C)S(=O)(=O)NC(=O)C1CCN(c2nc(COCC(F)(F)F)cnc2-c2cc(F)c3occc3c2)CC1] | N-(N,N-dimethylsulfamoyl)-1-(3-(7- fluorobenzofuran-5-yl)-6-((2,2,2- trifluoroethoxy)methyl)pyrazin-2- yl)piperidine-4-carboxamide |
| 81 | 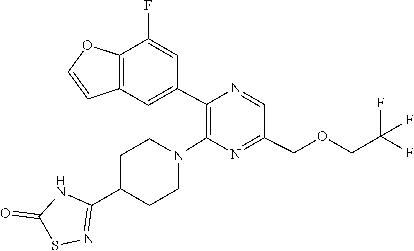 [SMILES: O=c1[nH]c(C2CCN(c3nc(COCC(F)(F)F)cnc3-c3cc(F)c4occc4c3)CC2)ns1] | 3-(1-(3-(7-fluorobenzofuran-5-yl)-6- ((2,2,2-trifluoroethoxy)methyl)pyrazin-2- yl)piperidin-4-yl)-1,2,4-thiadiazol-5(4H)-one |
| 82 | 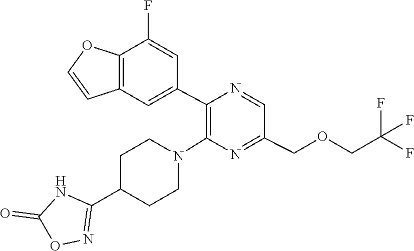 [SMILES: O=c1[nH]c(C2CCN(c3nc(COCC(F)(F)F)cnc3-c3cc(F)c4occc4c3)CC2)no1] | 3-(1-(3-(7-fluorobenzofuran-5-yl)-6- ((2,2,2-trifluoroethoxy)methyl)pyrazin-2- yl)piperidin-4-yl)-1,2,4-oxadiazol-5(4H)-one |
| 83 | 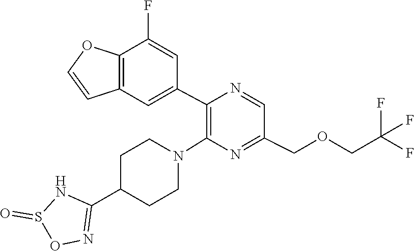 [SMILES: O=S1NC(C2CCN(c3nc(COCC(F)(F)F)cnc3-c3cc(F)c4occc4c3)CC2)=NO1] | 4-(1-(3-(7-fluorobenzofuran-5-yl)-6- ((2,2,2-trifluoroethoxy)methyl)pyrazin-2- yl)piperidin-4-yl)-3H-1,2,3,5- oxathiadiazole 2-oxide |
| 84 | 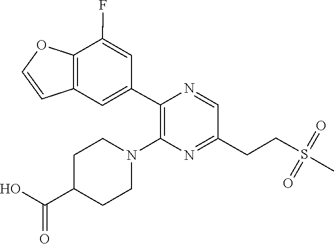 [SMILES: CS(=O)(=O)CCc1cnc(-c2cc(F)c3occc3c2)c(N2CCC(C(=O)O)CC2)n1] | 1-(3-(7-fluorobenzofuran-5-yl)-6-(2- (methylsulfonyl)ethyl)pyrazin-2- yl)piperidine-4-carboxylic acid |
| 85 | 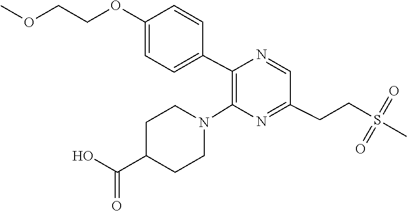 | 1-(3-(4-(2-methoxyethoxy)phenyl)-6-(2- (methylsulfonyl)ethyl)pyrazin-2- yl)piperidine-4-carboxylic acid |
| 86 | 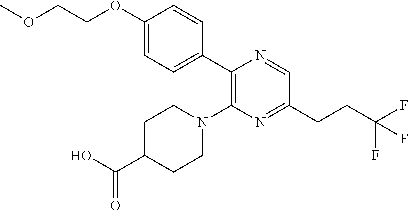 [SMILES: COCCOc1ccc(-c2ncc(CCC(F)(F)F)nc2N2CCC(C(=O)O)CC2)cc1] | 1-(3-(4-(2-methoxyethoxy)phenyl)-6- (3,3,3-trifluoropropyl)pyrazin-2- yl)piperidine-4-carboxylic acid |
| 87 | 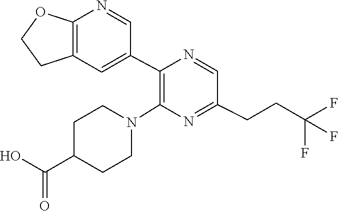 [SMILES: O=C(O)C1CCN(c2nc(CCC(F)(F)F)cnc2-c2cnc3c(c2)CCO3)CC1] | 1-(3-(2,3-dihydrofuro[2,3-b]pyridin-5-yl)- 6-(3,3,3-trifluoropropyl)pyrazin-2- yl)piperidine-4-carboxylic acid |
| 88 | 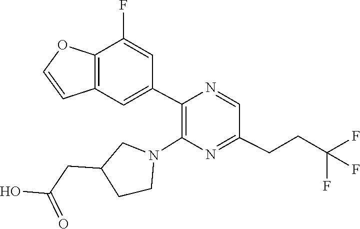 [SMILES: O=C(O)CC1CCN(c2nc(CCC(F)(F)F)cnc2-c2cc(F)c3occc3c2)C1] | 2-(1-(3-(7-fluorobenzofuran-5-yl)-6- (3,3,3-trifluoropropyl)pyrazin-2- yl)pyrrolidin-3-yl)acetic acid |
| 89 | 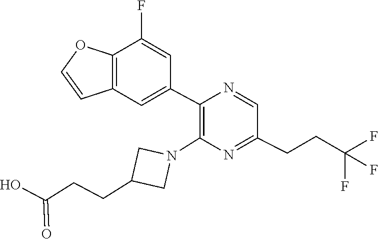 [SMILES: O=C(O)CCC1CN(c2nc(CCC(F)(F)F)cnc2-c2cc(F)c3occc3c2)C1] | 3-(1-(3-(7-fluorobenzofuran-5-yl)-6- (3,3,3-trifluoropropyl)pyrazin-2- yl)azetidin-3-yl)propanoic acid |
| 90 | 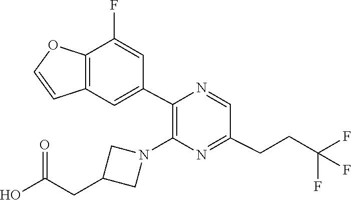 [SMILES: O=C(O)CC1CN(c2nc(CCC(F)(F)F)cnc2-c2cc(F)c3occc3c2)C1] | 2-(1-(3-(7-fluorobenzofuran-5-yl)-6- (3,3,3-trifluoropropyl)pyrazin-2- yl)azetidin-3-yl)acetic acid |
| 91 | 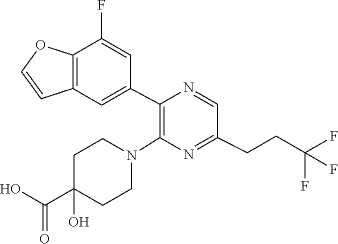 [SMILES: O=C(O)C1(O)CCN(c2nc(CCC(F)(F)F)cnc2-c2cc(F)c3occc3c2)CC1] | 1-(3-(7-fluorobenzofuran-5-yl)-6-(3,3,3- trifluoropropyl)pyrazin-2-yl)-4- hydroxypiperidine-4-carboxylic acid |
| 92 | 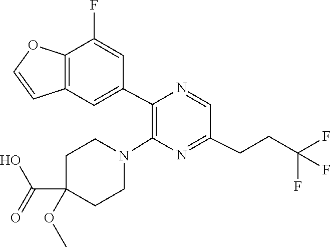 [SMILES: COC1(C(=O)O)CCN(c2nc(CCC(F)(F)F)cnc2-c2cc(F)c3occc3c2)CC1] | 1-(3-(7-fluorobenzofuran-5-yl)-6-(3,3,3- trifluoropropyl)pyrazin-2-yl)-4- methoxypiperidine-4-carboxylic acid |
| 93 | 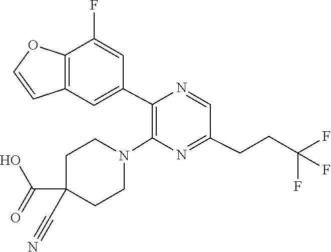 [SMILES: N#CC1(C(=O)O)CCN(c2nc(CCC(F)(F)F)cnc2-c2cc(F)c3occc3c2)CC1] | 4-cyano-1-(3-(7-fluorobenzofuran-5-yl)- 6-(3,3,3-trifluoropropyl)pyrazin-2- yl)piperidine-4-carboxylic acid |
| 94 | 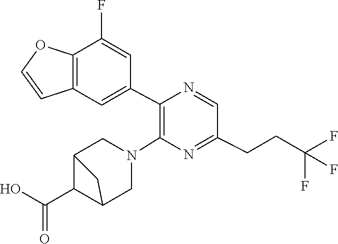 [SMILES: O=C(O)C1C2CC1CN(c1nc(CCC(F)(F)F)cnc1-c1cc(F)c3occc3c1)C2] | 3-(3-(7-fluorobenzofuran-5-yl)-6-(3,3,3- trifluoropropyl)pyrazin-2-yl)-3- azabicyclo[3.1.1]heptane-6-carboxylic acid |
| 95 | 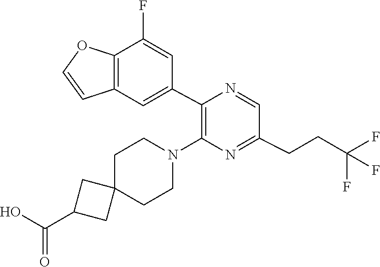 [SMILES: O=C(O)C1CC2(CCN(c3nc(CCC(F)(F)F)cnc3-c3cc(F)c4occc4c3)CC2)C1] | 7-(3-(7-fluorobenzofuran-5-yl)-6-(3,3,3- trifluoropropyl)pyrazin-2-yl)-7- azaspiro[3.5]nonane-2-carboxylic acid |
| 96 | 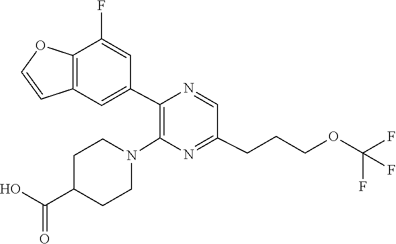 [SMILES: O=C(O)C1CCN(c2nc(CCCOC(F)(F)F)cnc2-c2cc(F)c3occc3c2)CC1] | 1-(3-(7-fluorobenzofuran-5-yl)-6-(3- (trifluoromethoxy)propyl)pyrazin-2- yl)piperidine-4-carboxylic acid |
| 97 | 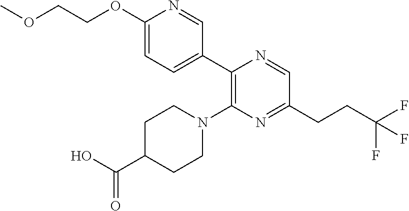 [SMILES: COCCOc1ccc(-c2ncc(CCC(F)(F)F)nc2N2CCC(C(=O)O)CC2)cn1] | 1-(3-(6-(2-methoxyethoxy)pyridin-3-yl)- 6-(3,3,3-trifluoropropyl)pyrazin-2- yl)piperidine-4-carboxylic acid |
| 98 | 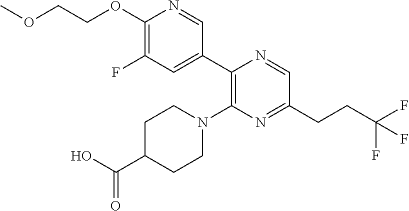 [SMILES: COCCOc1ncc(-c2ncc(CCC(F)(F)F)nc2N2CCC(C(=O)O)CC2)cc1F] | 1-(3-(5-fluoro-6-(2- methoxyethoxy)pyridin-3-yl)-6-(3,3,3- trifluoropropyl)pyrazin-2-yl)piperidine-4- carboxylic acid |
| 99 | 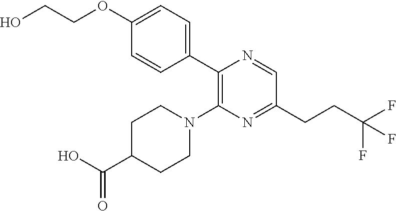 [SMILES: O=C(O)C1CCN(c2nc(CCC(F)(F)F)cnc2-c2ccc(OCCO)cc2)CC1] | 1-(3-(4-(2-hydroxyethoxy)phenyl)-6- (3,3,3-trifluoropropyl)pyrazin-2- yl)piperidine-4-carboxylic acid |
| 100 | 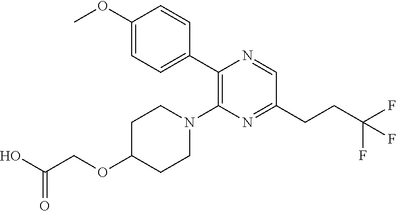 [SMILES: COc1ccc(-c2ncc(CCC(F)(F)F)nc2N2CCC(OCC(=O)O)CC2)cc1] | 2-((1-(3-(4-methoxyphenyl)-6-(3,3,3- trifluoropropyl)pyrazin-2-yl)piperidin-4- yl)oxy)acetic acid |
| 101 | 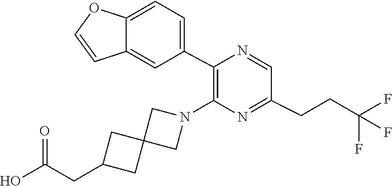 [SMILES: O=C(O)CC1CC2(C1)CN(c1nc(CCC(F)(F)F)cnc1-c1ccc3occc3c1)C2] | 2-(2-(3-(benzofuran-5-yl)-6-(3,3,3- trifluoropropyl)pyrazin-2-yl)-2- azaspiro[3.3]heptan-6-yl)acetic acid |
| 102 | 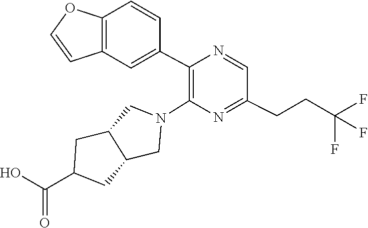 | (3aR,6aS)-2-(3-(benzofuran-5-yl)-6- (3,3,3-trifluoropropyl)pyrazin-2- yl)octahydrocyclopenta[c]pyrrole-5- carboxylic acid |
| 103 | 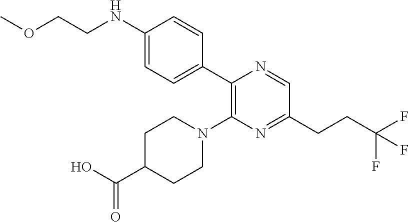 [SMILES: COCCNc1ccc(-c2ncc(CCC(F)(F)F)nc2N2CCC(C(=O)O)CC2)cc1] | 1-(3-(4-((2-methoxyethyl)amino)phenyl)- 6-(3,3,3-trifluoropropyl)pyrazin-2- yl)piperidine-4-carboxylic acid |
| 104 | 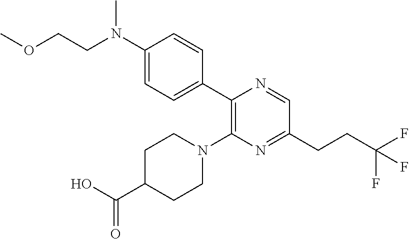 [SMILES: COCCN(C)c1ccc(-c2ncc(CCC(F)(F)F)nc2N2CCC(C(=O)O)CC2)cc1] | 1-(3-(4-((2- methoxyethyl)(methyl)amino)phenyl)-6- (3,3,3-trifluoropropyl)pyrazin-2- yl)piperidine-4-carboxylic acid |
| 105 | 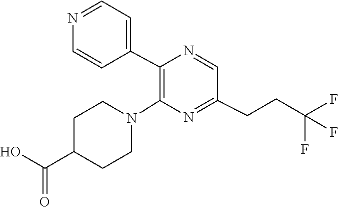 [SMILES: O=C(O)C1CCN(c2nc(CCC(F)(F)F)cnc2-c2ccncc2)CC1] | 1-(3-(pyridin-4-yl)-6-(3,3,3- trifluoropropyl)pyrazin-2-yl)piperidine-4- carboxylic acid |
| 106 | 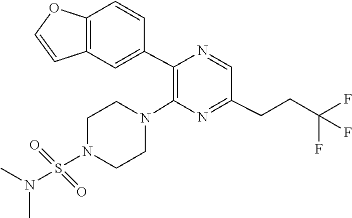 [SMILES: CN(C)S(=O)(=O)N1CCN(c2nc(CCC(F)(F)F)cnc2-c2ccc3occc3c2)CC1] | 4-(3-(benzofuran-5-yl)-6-(3,3,3- trifluoropropyl)pyrazin-2-yl)-N,N- dimethylpiperazine-1-sulfonamide |
| 107 | 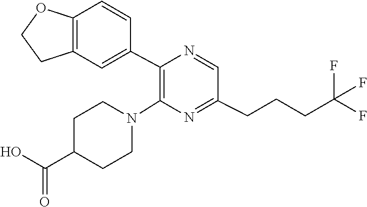 [SMILES: O=C(O)C1CCN(c2nc(CCCC(F)(F)F)cnc2-c2ccc3c(c2)CCO3)CC1] | 1-(3-(2,3-dihydrobenzofuran-5-yl)-6- (4,4,4-trifluorobutyl)pyrazin-2- yl)piperidine-4-carboxylic acid |
| 108 | 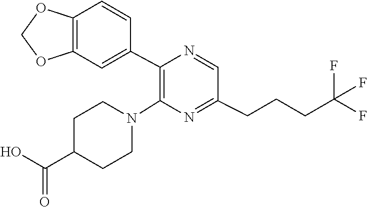 [SMILES: O=C(O)C1CCN(c2nc(CCCC(F)(F)F)cnc2-c2ccc3c(c2)OCO3)CC1] | 1-(3-(benzo[d][1,3]dioxol-5-yl)-6-(4,4,4- trifluorobutyl)pyrazin-2-yl)piperidine-4- carboxylic acid |
| 109 | 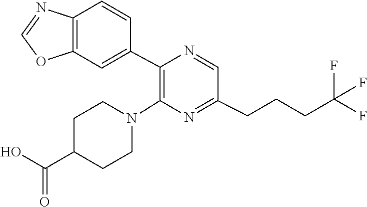 [SMILES: O=C(O)C1CCN(c2nc(CCCC(F)(F)F)cnc2-c2ccc3ncoc3c2)CC1] | 1-(3-(benzo[d]oxazol-6-yl)-6-(4,4,4- trifluorobutyl)pyrazin-2-yl)piperidine-4- carboxylic acid |
| 110 | 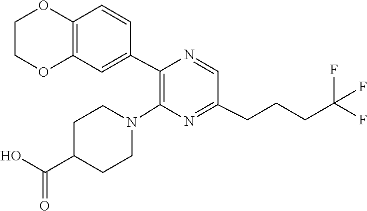 [SMILES: O=C(O)C1CCN(c2nc(CCCC(F)(F)F)cnc2-c2ccc3c(c2)OCCO3)CC1] | 1-(3-(2,3-dihydrobenzo[b][1,4]dioxin-6- yl)-6-(4,4,4-trifluorobutyl)pyrazin-2- yl)piperidine-4-carboxylic acid |
| 111 | 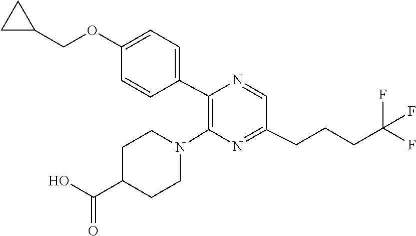 [SMILES: O=C(O)C1CCN(c2nc(CCCC(F)(F)F)cnc2-c2ccc(OCC3CC3)cc2)CC1] | 1-(3-(4-(cyclopropylmethoxy)phenyl)-6- (4,4,4-trifluorobutyl)pyrazin-2- yl)piperidine-4-carboxylic acid |
| 112 | 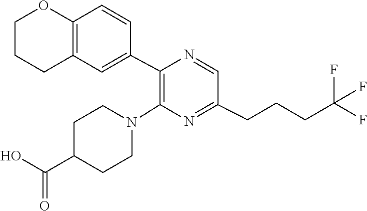 [SMILES: O=C(O)C1CCN(c2nc(CCCC(F)(F)F)cnc2-c2ccc3c(c2)CCCO3)CC1] | 1-(3-(chroman-6-yl)-6-(4,4,4- trifluorobutyl)pyrazin-2-yl)piperidine-4- carboxylic acid |
| 113 | 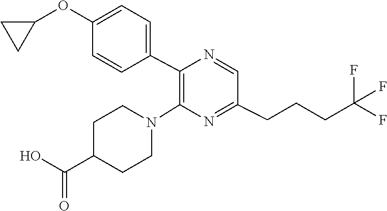 [SMILES: O=C(O)C1CCN(c2nc(CCCC(F)(F)F)cnc2-c2ccc(OC3CC3)cc2)CC1] | 1-(3-(4-cyclopropoxyphenyl)-6-(4,4,4- trifluorobutyl)pyrazin-2-yl)piperidine-4- carboxylic acid |
| 114 | 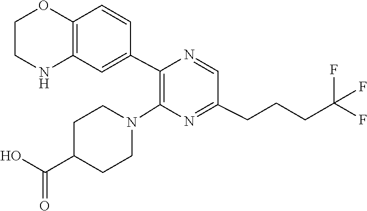 [SMILES: O=C(O)C1CCN(c2nc(CCCC(F)(F)F)cnc2-c2ccc3c(c2)NCCO3)CC1] | 1-(3-(3,4-dihydro-2H- benzo[b][1,4]oxazin-6-y)-6-(4,4,4- trifluorobutyl)pyrazin-2-yl)piperidine-4- carboxylic acid |
| 115 | 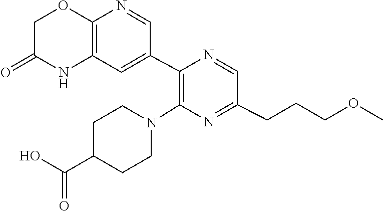 [SMILES: COCCCc1cnc(-c2cnc3c(c2)NC(=O)CO3)c(N2CCC(C(=O)O)CC2)n1] | 1-(3-(3-oxo-3,4-dihydro-2H- benzo[b][1,4]oxazin-6-yl)-6-(4,4,4- trifluorobutyl)pyrazin-2-yl)piperidine-4- carboxylic acid |
| 116 | 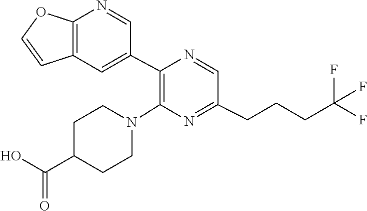 [SMILES: O=C(O)C1CCN(c2nc(CCCC(F)(F)F)cnc2-c2cnc3occc3c2)CC1] | 1-(3-(furo[2,3-b]pyridin-5-yl)-6-(4,4,4- trifluorobutyl)pyrazin-2-yl)piperidine-4- carboxylic acid |
| 117 | 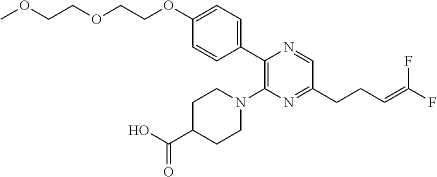 [SMILES: COCCOCCOc1ccc(-c2ncc(CCC=C(F)F)nc2N2CCC(C(=O)O)CC2)cc1] | 1-(3-(4-(2-(2- methoxyethoxy)ethoxy)phenyl)-6-(4,4,4- trifluorobutyl)pyrazin-2-yl)piperidine-4- carboxylic acid |
| 118 | 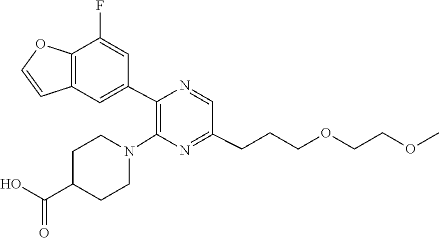 [SMILES: COCCOCCCc1cnc(-c2cc(F)c3occc3c2)c(N2CCC(C(=O)O)CC2)n1] | 1-(3-(7-fluorobenzofuran-5-yl)-6-(3-(2- methoxyethoxy)propyl)pyrazin-2- yl)piperidine-4-carboxylic acid |
| 119 | 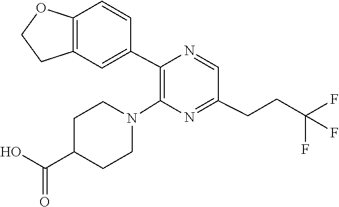 [SMILES: O=C(O)C1CCN(c2nc(CCC(F)(F)F)cnc2-c2ccc3c(c2)CCO3)CC1] | 1-(3-(2,3-dihydrobenzofuran-5-yl)-6- (3,3,3-trifluoropropyl)pyrazin-2- yl)piperidine-4-carboxylic acid |
| 120 | 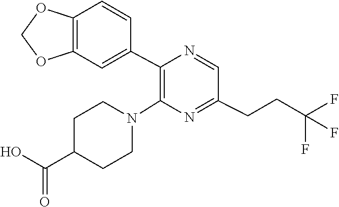 [SMILES: O=C(O)C1CCN(c2nc(CCC(F)(F)F)cnc2-c2ccc3c(c2)OCO3)CC1] | 1-(3-(benzo[d][1,3]dioxol-5-yl)-6-(3,3,3- trifluoropropyl)pyrazin-2-yl)piperidine-4- carboxylic acid |
| 121 | 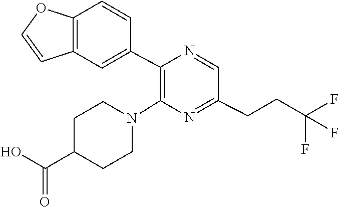 [SMILES: O=C(O)C1CCN(c2nc(CCC(F)(F)F)cnc2-c2ccc3occc3c2)CC1] | 1-(3-(benzofuran-5-yl)-6-(3,3,3- trifluoropropyl)pyrazin-2-yl)piperidin-4- carboxylic acid |
| 122 | 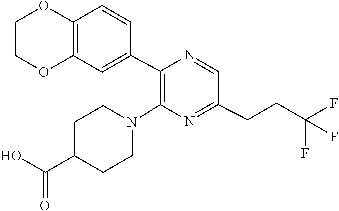 [SMILES: O=C(O)C1CCN(c2nc(CCC(F)(F)F)cnc2-c2ccc3c(c2)OCCO3)CC1] | 1-(3-(2,3-dihyrobenzo[b][1,4]dioxin-6- yl)-6-(3,3,3-trifluoropropyl)pyrazin-2- yl)piperidine-4-carboxylic acid |
| 123 | 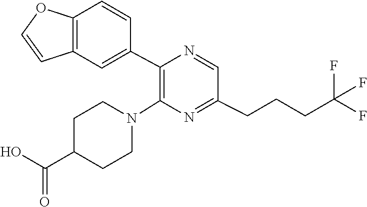 [SMILES: O=C(O)C1CCN(c2nc(CCCC(F)(F)F)cnc2-c2ccc3occc3c2)CC1] | 1-(3-(benzofuran-5-yl)-6-(4,4,4- trifluorobutyl)pyrazin-2-yl)piperidine-4- carboxylic acid |
| 124 | 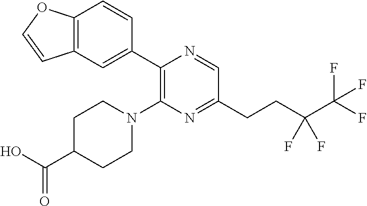 [SMILES: O=C(O)C1CCN(c2nc(CCC(F)(F)C(F)(F)F)cnc2-c2ccc3occc3c2)CC1] | 1-(3-(benzofuran-5-yl)-6-(3,3,4,4,4- pentafluorobutyl)pyrazin-2-yl)piperidine- 4-carboxylic acid |
| 125 | 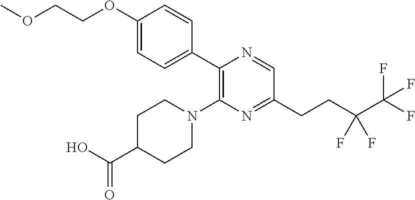 [SMILES: COCCOc1ccc(-c2ncc(CCC(F)(F)C(F)(F)F)nc2N2CCC(C(=O)O)CC2)cc1] | 1-(3-(4-(2-methoxyethoxy)phenyl)-6- (3,3,4,4,4-pentafluorobutyl)pyrazin-2- yl)piperidine-4-carboxylic acid |
| 126 | 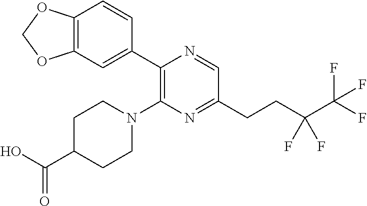 [SMILES: O=C(O)C1CCN(c2nc(CCC(F)(F)C(F)(F)F)cnc2-c2ccc3c(c2)OCO3)CC1] | 1-(3-(benzo[d][1,3]dioxol-5-yl)-6- (3,3,4,4,4-pentafluorobutyl)pyrazin-2- yl)piperidine-4-carboxylic acid |
| 127 | 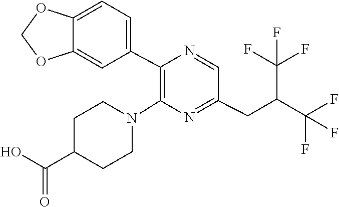 [SMILES: O=C(O)C1CCN(c2nc(CC(C(F)(F)F)C(F)(F)F)cnc2-c2ccc3c(c2)OCO3)CC1] | 1-(3-(benzo[d][1,3]dioxol-5-yl)-6-(3,3,3- trifluoro-2- (trifluoromethyl)propyl)pyrazin-2- yl)piperidine-4-carboxylic acid |
| 128 | 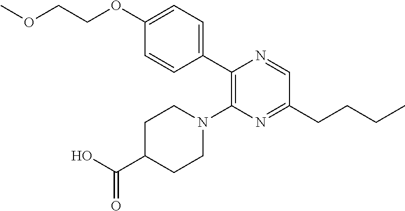 [SMILES: CCCCc1cnc(-c2ccc(OCCOC)cc2)c(N2CCC(C(=O)O)CC2)n1] | 1-(3-(4-(2-methoxyethoxy)phenyl)-6- pentylpyrazin-2-yl)piperidine-4- carboxylic acid |
| 129 | 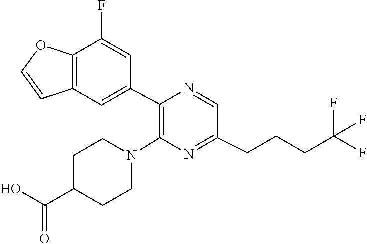 [SMILES: O=C(O)C1CCN(c2nc(CCCC(F)(F)F)cnc2-c2cc(F)c3occc3c2)CC1] | 1-(3-(7-fluorobenzofuran-5-yl)-6-(4,4,4- trifluorobutyl)pyrazin-2-yl)piperidine-4- carboxylic acid |
| 130 | 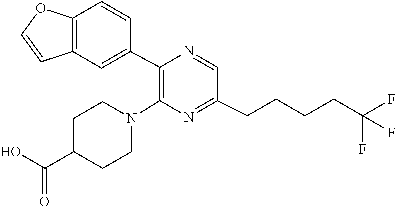 [SMILES: O=C(O)C1CCN(c2nc(CCCCC(F)(F)F)cnc2-c2ccc3occc3c2)CC1] | 1-(3-(benzofuran-5-yl)-6-(5,5,5- trifluoropentyl)pyrazin-2-yl)piperidine-4- carboxylic acid |
| 131 | 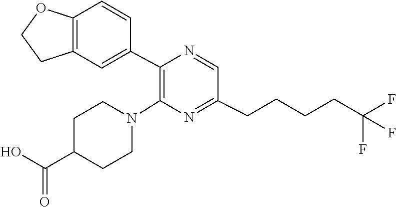 [SMILES: O=C(O)C1CCN(c2nc(CCCCC(F)(F)F)cnc2-c2ccc3c(c2)CCO3)CC1] | 1-(3-(2,3-dihydrobenzofuran-5-yl)-6- (5,5,5-trifluoropentyl)pyrazin-2- yl)piperidine-4-carboxylic acid |
| 132 | 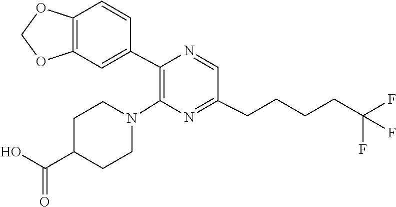 [SMILES: O=C(O)C1CCN(c2nc(CCCCC(F)(F)F)cnc2-c2ccc3c(c2)OCO3)CC1] | 1-(3-(benzo[d][1,3]dioxol-5-yl)-6-(5,5,5- trifluoropentyl)pyrazin-2-yl)piperidine-4- carboxylic acid |
| 133 | 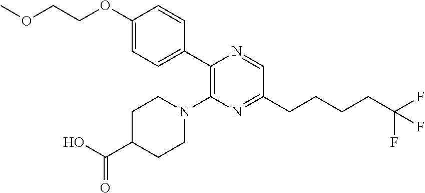 [SMILES: COCCOc1ccc(-c2ncc(CCCCC(F)(F)F)nc2N2CCC(C(=O)O)CC2)cc1] | 1-(3-(4-(2-methoxyethoxy)phenyl)-6- (5,5,5-trifluoropentyl)pyrazin-2- yl)piperidine-4-carboxylic acid |
| 134 | 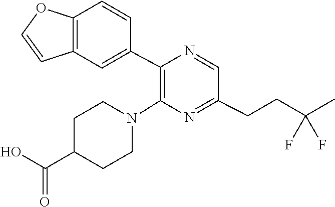 [SMILES: CC(F)(F)CCc1cnc(-c2ccc3occc3c2)c(N2CCC(C(=O)O)CC2)n1] | 1-(3-(benzofuran-5-yl)-6-(3,3- difluorobutyl)pyrazin-2-yl)piperidine-4- carboxylic acid |
| 135 | 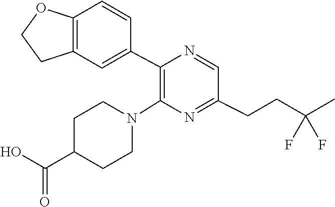 [SMILES: CC(F)(F)CCc1cnc(-c2ccc3c(c2)CCO3)c(N2CCC(C(=O)O)CC2)n1] | 1-(6-(3,3-difluorobutyl)-3-(2,3- dihydrobenzofuran-5-yl)pyrazin-2- yl)piperidine-4-carboxylic acid |
| 136 | 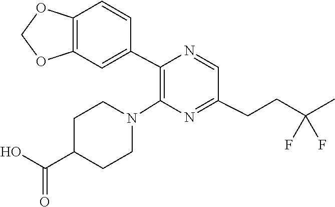 [SMILES: CC(F)(F)CCc1cnc(-c2ccc3c(c2)OCO3)c(N2CCC(C(=O)O)CC2)n1] | 1-(3-benzo[d][1,3]dioxol-5-yl)-6-(3,3- difluorobutyl)pyrazin-2-yl)piperidine-4- carboxylic acid |
| 137 | 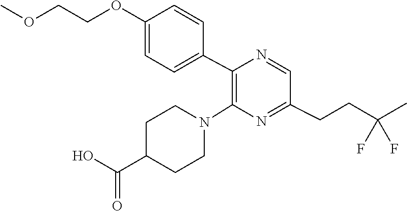 [SMILES: COCCOc1ccc(-c2ncc(CCC(C)(F)F)nc2N2CCC(C(=O)O)CC2)cc1] | 1-(6-(3,3-difluorobutyl)-3-(4-(2- methoxyethoxy)phenyl)pyrazin-2- yl)piperidine-4-carboxylic acid |
| 138 | 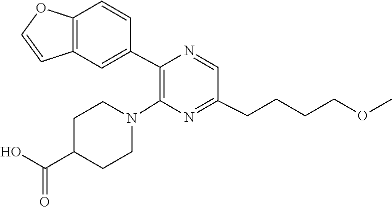 [SMILES: COCCCCc1cnc(-c2ccc3occc3c2)c(N2CCC(C(=O)O)CC2)n1] | 1-(3-(benzofuran-5-yl)-6-(4- methoxybutyl)pyrazin-2-yl)piperidine-4- carboxylic acid |
| 139 | 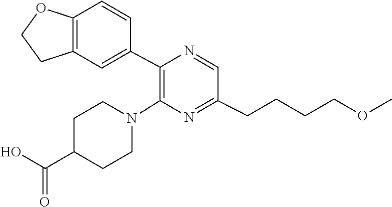 [SMILES: COCCCCc1cnc(-c2ccc3c(c2)CCO3)c(N2CCC(C(=O)O)CC2)n1] | 1-(3-(2,3-dihydrobenzofuran-5-yl)-6-(4- methoxybutyl)pyrazin-2-yl)piperidine-4- carboxylic acid |
| 140 | 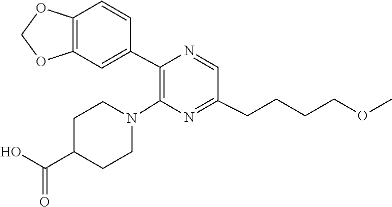 [SMILES: COCCCCc1cnc(-c2ccc3c(c2)OCO3)c(N2CCC(C(=O)O)CC2)n1] | 1-(3-(benzo[d][1,3]dioxol-5-yl)-6-(4- methoxybutyl)pyrazin-2-yl)piperidine-4- carboxylic acid |
| 141 | 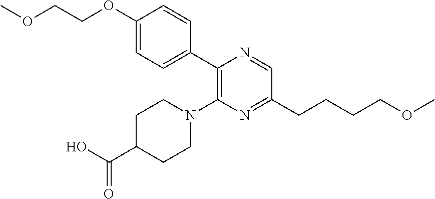 | 1-(6-(4-methoxybutyl)-3-(4-(2- methoxyethoxy)phenyl)pyrazin-2- yl)piperidine-4-carboxylic acid |
| 142 | 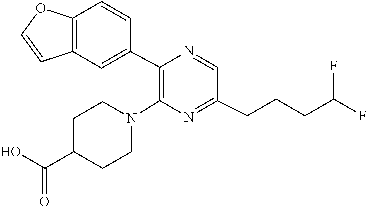 [SMILES: O=C(O)C1CCN(c2nc(CCCC(F)F)cnc2-c2ccc3occc3c2)CC1] | 1-(3-(benzofuran-5-yl)-6-(4,4- difluorobutyl)pyrazin-2-yl)piperidine-4- carboxylic acid |
| 143 | 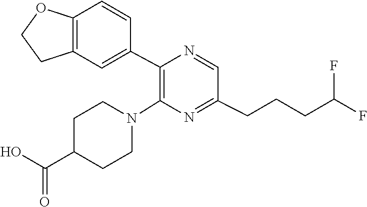 [SMILES: O=C(O)C1CCN(c2nc(CCCC(F)F)cnc2-c2ccc3c(c2)CCO3)CC1] | 1-(6-(4,4-difluorobutyl)-3-(2,3- dihydrobenzofuran-5-yl)pyrazin-2- yl)piperidine-4-carboxylic acid |
| 144 | 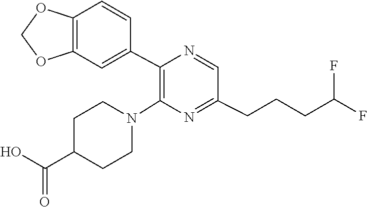 [SMILES: O=C(O)C1CCN(c2nc(CCCC(F)F)cnc2-c2ccc3c(c2)OCO3)CC1] | 1-(3-(benzo[d][1,3]dioxol-5-yl)-6-(4,4- difluorobutyl)pyrazin-2-yl)piperidine-4- carboxylic acid |
| 145 | 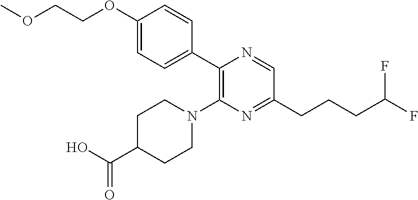 [SMILES: COCCOc1ccc(-c2ncc(CCCC(F)F)nc2N2CCC(C(=O)O)CC2)cc1] | 1-(6-(4,4-difluorobutyl)-3-(4-(2- methoxyethoxy)phenyl)pyrazin-2- yl)piperidine-4-carboxylic acid |
| 146 | 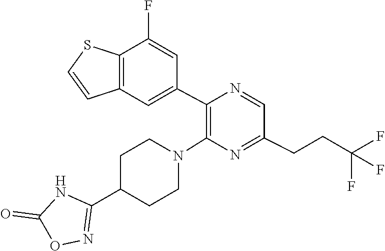 [SMILES: O=c1[nH]c(C2CCN(c3nc(CCC(F)(F)F)cnc3-c3cc(F)c4sccc4c3)CC2)no1] | 3-(1-(3-(7-fluorobenzofuran-5-yl)-6- (3,3,3-trifluoropropyl)pyrazin-2- yl)piperidin-4-yl)-1,2,4-oxadiazol-5(4H)-one |
| 147 | 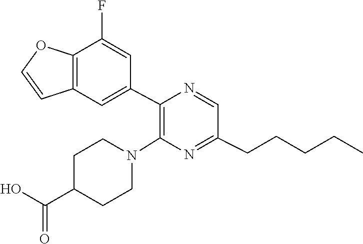 [SMILES: CCCCCc1cnc(-c2cc(F)c3occc3c2)c(N2CCC(C(=O)O)CC2)n1] | 1-(3-(7-fluorobenzofuran-5-yl)-6- pentylpyrazin-2-yl)piperidine-4- carboxylic acid |
| 148 | 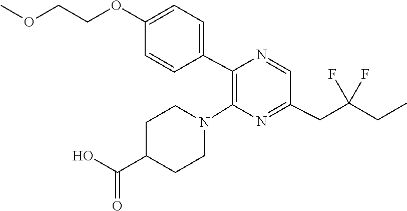 [SMILES: CCC(F)(F)Cc1cnc(-c2ccc(OCCOC)cc2)c(N2CCC(C(=O)O)CC2)n1] | 1-(6-(2,2-difluorobutyl)-3-(4-(2- methoxyethoxy)phenyl)pyrazin-2- yl)piperidine-4-carboxylic acid |
| 149 | 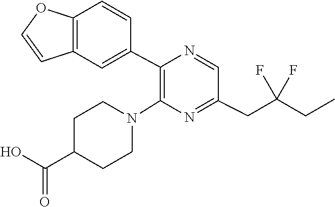 [SMILES: CCC(F)(F)Cc1cnc(-c2ccc3occc3c2)c(N2CCC(C(=O)O)CC2)n1] | 1-(3-(benzofuran-5-yl)-6-(2,2- difluorobutyl)pyrazin-2-yl)piperidine-4- carboxylic acid |
| 150 | 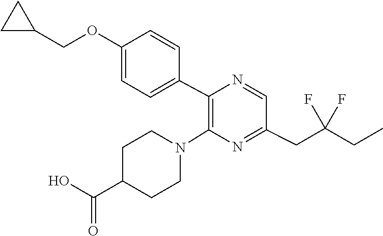 [SMILES: CCC(F)(F)Cc1cnc(-c2ccc(OCC3CC3)cc2)c(N2CCC(C(=O)O)CC2)n1] | 1-(3-(4-(cyclopropylmethoxy)phenyl)-6- (2,2-difluorobutyl)pyrazin-2- yl)piperidine-4-carboxylic acid |
| 151 | 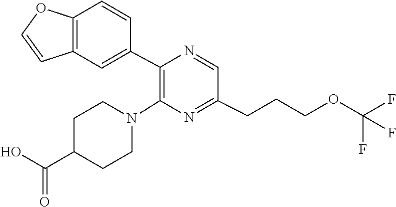 [SMILES: O=C(O)C1CCN(c2nc(CCCOC(F)(F)F)cnc2-c2ccc3occc3c2)CC1] | 1-(3-(benzofuran-5-yl)-6-(3- (trifluoromethoxy)propyl)pyrazin-2- yl)piperidine-4-carboxylic acid |
| 152 | 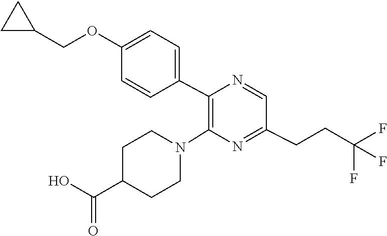 [SMILES: O=C(O)C1CCN(c2nc(CCC(F)(F)F)cnc2-c2ccc(OCC3CC3)cc2)CC1] | 1-(3-(4-(cyclopropylmethoxy)phenyl)-6- (3,3,3-trifluoropropyl)pyrazin-2- yl)piperidine-4-carboxylic acid |
| 153 | 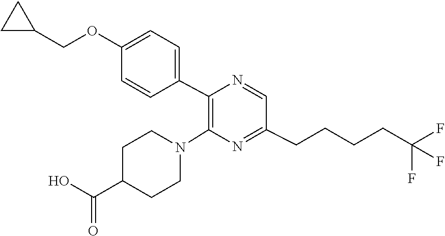 [SMILES: O=C(O)C1CCN(c2nc(CCCCC(F)(F)F)cnc2-c2ccc(OCC3CC3)cc2)CC1] | 1-(3-(4-(cyclopropylmethoxy)phenyl)-6- (5,5,5-trifluoropentyl)pyrazin-2- yl)piperidine-4-carboxylic acid |
| 154 | 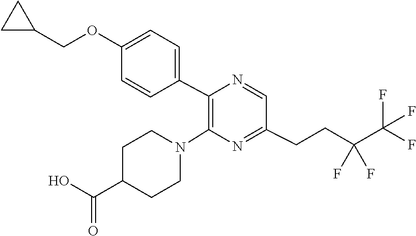 [SMILES: O=C(O)C1CCN(c2nc(CCC(F)(F)C(F)(F)F)cnc2-c2ccc(OCC3CC3)cc2)CC1] | 1-(3-(4-(cyclopropylmethoxy)phenyl)-6- (3,3,4,4,4-pentafluorobutyl)pyrazin-2- yl)piperidine-4-carboxylic acid |
| 155 | 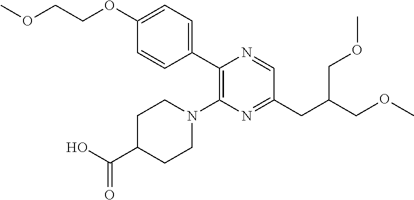 | 1-(6-(3-methoxy-2- (methoxymethyl)propyl)-3-(4-(2- methoxyethoxy)phenyl)pyrazin-2- yl)piperidine-4-carboxylic acid |
| 156 | 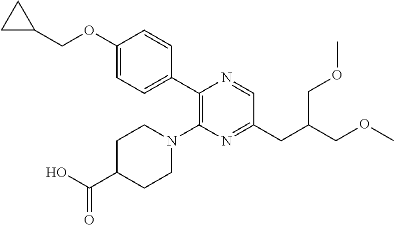 [SMILES: COCC(COC)Cc1cnc(-c2ccc(OCC3CC3)cc2)c(N2CCC(C(=O)O)CC2)n1] | 1-(3-(4-(cyclopropylmethoxy)phenyl)-6- (3-methoxy-2- (methoxymethyl)propyl)pyrazin-2- yl)piperidine-4-carboxylic acid |
| 157 | 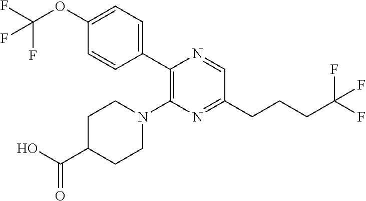 [SMILES: O=C(O)C1CCN(c2nc(CCCC(F)(F)F)cnc2-c2ccc(OC(F)(F)F)cc2)CC1] | 1-(6-(4,4,4-trifluorobutyl)-3-(4- (trifluoromethoxy)phenyl)pyrazin-2- yl)piperidine-4-carboxylic acid |
| 158 | 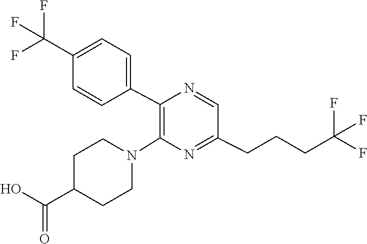 [SMILES: O=C(O)C1CCN(c2nc(CCCC(F)(F)F)cnc2-c2ccc(C(F)(F)F)cc2)CC1] | 1-(6-(4,4,4-trifluorobutyl)-3-(4- (trifluoromethyl)phenyl)pyrazin-2- yl)piperidine-4-carboxylic acid |
| 159 | 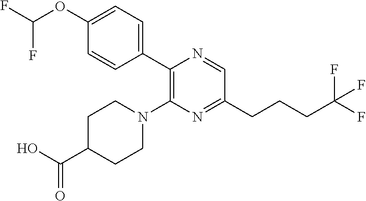 [SMILES: O=C(O)C1CCN(c2nc(CCCC(F)(F)F)cnc2-c2ccc(OC(F)F)cc2)CC1] | 1-(3-(4-(difluoromethoxy)phenyl)-6- (4,4,4-trifluorobutyl)pyrazin-2- yl)piperidine-4-carboxylic acid |
| 160 | 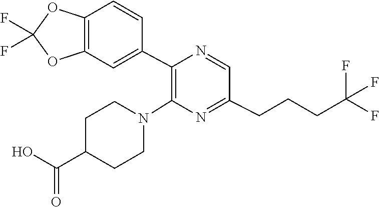 [SMILES: O=C(O)C1CCN(c2nc(CCCC(F)(F)F)cnc2-c2ccc3c(c2)OC(F)(F)O3)CC1] | 1-(3-(2,2-difluorobenzo[d][1,3]dioxol-5- yl)-6-(4,4,4-trifluorobutyl)pyrazin-2- yl)piperidine-4-carboxylic acid |
| 161 | 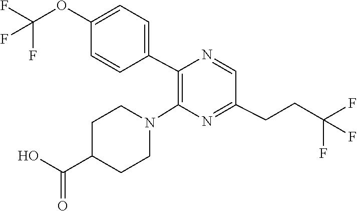 [SMILES: O=C(O)C1CCN(c2nc(CCC(F)(F)F)cnc2-c2ccc(OC(F)(F)F)cc2)CC1] | 1-(3-(4-(trifluoromethoxy)phenyl)-6- (3,3,3-trifluoropropyl)pyrazin-2- yl)piperidine-4-carboxylic acid |
| 162 | 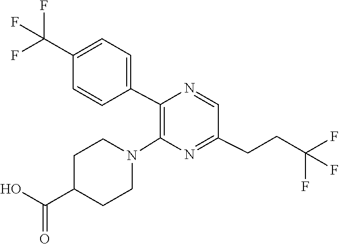 [SMILES: O=C(O)C1CCN(c2nc(CCC(F)(F)F)cnc2-c2ccc(C(F)(F)F)cc2)CC1] | 1-(3-(4-(trifluoromethyl)phenyl)-6-(3,3,3- trifluoropropyl)pyrazin-2-yl)piperidine-4- carboxylic acid |
| 163 | 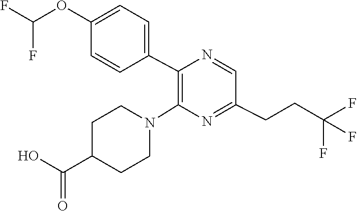 [SMILES: O=C(O)C1CCN(c2nc(CCC(F)(F)F)cnc2-c2ccc(OC(F)F)cc2)CC1] | 1-(3-(4-(difluoromethoxy)phenyl)-6- (3,3,3-trifluoropropyl)pyrazin-2- yl)piperidine-4-carboxylic acid |
| 164 | 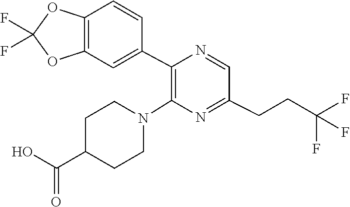 [SMILES: O=C(O)C1CCN(c2nc(CCC(F)(F)F)cnc2-c2ccc3c(c2)OC(F)(F)O3)CC1] | 1-(3-(2,2-difluorobenzo[d][1,3]dioxol-5- yl)-6-(3,3,3-trifluoropropyl)pyrazin-2- yl)piperidine-4-carboxylic acid |
| 165 | 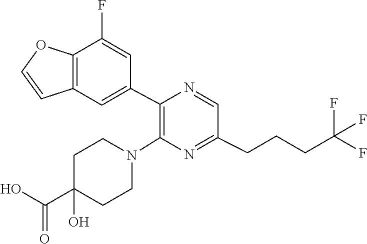 [SMILES: O=C(O)C1(O)CCN(c2nc(CCCC(F)(F)F)cnc2-c2cc(F)c3occc3c2)CC1] | 1-(3-(7-fluorobenzofuran-5-yl)-6-(4,4,4- trifluorobutyl)pyrazin-2-yl)-4- hydroxypiperidine-4-carboxylic acid |
| 166 | 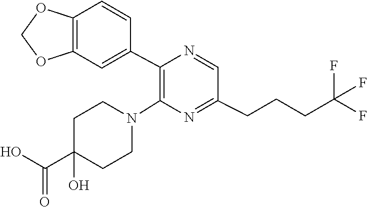 [SMILES: O=C(O)C1(O)CCN(c2nc(CCCC(F)(F)F)cnc2-c2ccc3c(c2)OCO3)CC1] | 1-(3-(benzo[d][1,3]dioxol-5-yl)-6-(4,4,4- trifluorobutyl)pyrazin-2-yl)-4- hydroxypiperidine-4-carboxylic acid |
| 167 | 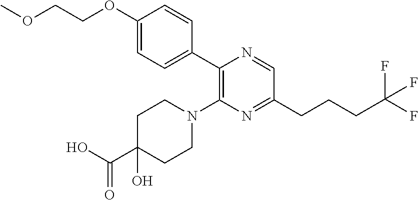 [SMILES: COCCOc1ccc(-c2ncc(CCCC(F)(F)F)nc2N2CCC(O)(C(=O)O)CC2)cc1] | 4-hydroxy-1-(3-(4-(2- methoxyethoxy)phenyl)-6-(4,4,4- trifluorobutyl)pyrazin-2-yl)piperidine-4- carboxylic acid |
| 168 | 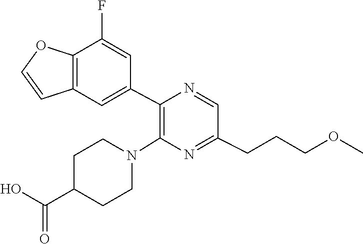 [SMILES: COCCCc1cnc(-c2cc(F)c3occc3c2)c(N2CCC(C(=O)O)CC2)n1] | 1-(2-(7-fluorobenzofuran-5-yl)-5-(3- methoxypropyl)pyridin-3-yl)piperidine-4- carboxylic acid |
| 169 | 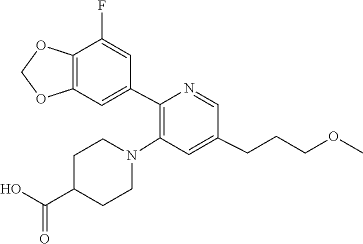 [SMILES: COCCCc1cnc(-c2cc(F)c3c(c2)OCO3)c(N2CCC(C(=O)O)CC2)c1] | 1-(2-(7-fluorobenzo[d][1,3]dioxol-5-yl)- 5-(3-methoxypropyl)pyridin-3- yl)piperidine-4-carboxylic acid |
| 170 | 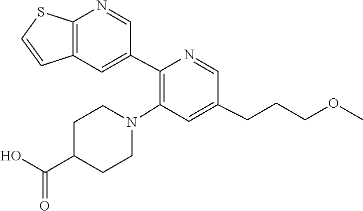 [SMILES: COCCCc1cnc(-c2cnc3sccc3c2)c(N2CCC(C(=O)O)CC2)c1] | 1-(2-(furo[2,3-b]pyridin-5-yl)-5-(3- methoxypropyl)pyridin-3-yl)piperidine-4- carboxylic acid |
| 171 | 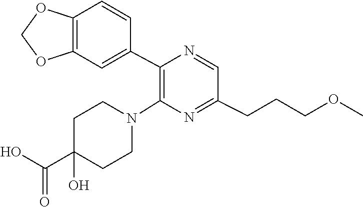 [SMILES: COCCCc1cnc(-c2ccc3c(c2)OCO3)c(N2CCC(O)(C(=O)O)CC2)n1] | 1-(3-(benzo[d][1,3]dioxol-5-yl)-6-(3- methoxypropyl)pyrazin-2-yl)-4- hydroxypiperidine-4-carboxylic acid |
| 172 | 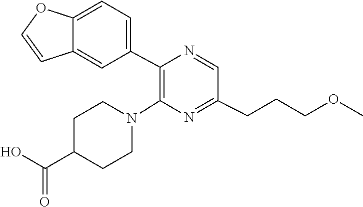 [SMILES: COCCCc1cnc(-c2ccc3occc3c2)c(N2CCC(C(=O)O)CC2)n1] | 1-(3-(benzofuran-5-yl)-6-(3- methoxypropyl)pyrazin-2-yl)piperidine-4- carboxylic acid |
| 173 | 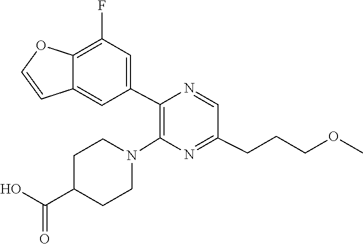 [SMILES: COCCCc1cnc(-c2cc(F)c3occc3c2)c(N2CCC(C(=O)O)CC2)n1] | 1-(3-(7-fluorobenzofuran-5-yl)-6-(3- methoxypropyl)pyrazin-2-yl)piperidine-4- carboxylic acid |
| 174 | 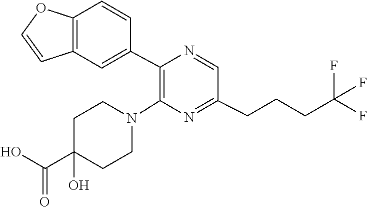 [SMILES: O=C(O)C1(O)CCN(c2nc(CCCC(F)(F)F)cnc2-c2ccc3occc3c2)CC1] | 1-(3-(benzofuran-5-yl)-6-(4,4,4- trifluorobutyl)pyrazin-2-yl)-4- hydroxypiperidine-4-carboxylic acid |
| 175 | 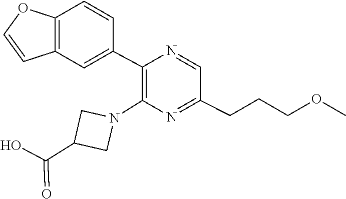 [SMILES: COCCCc1cnc(-c2ccc3occc3c2)c(N2CC(C(=O)O)C2)n1] | 1-(3-(benzofuran-5-yl)-6-(3- methoxypropyl)pyrazin-2-yl)azetidine-3- carboxylic acid |
| 176 | 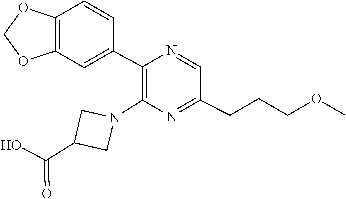 [SMILES: COCCCc1cnc(-c2ccc3c(c2)OCO3)c(N2CC(C(=O)O)C2)n1] | 1-(3-(benzo[d][1,3]dioxol-5-yl)-6-(3- methoxypropyl)pyrazin-2-yl)azetidine-3- carboxylic acid |
| 177 | 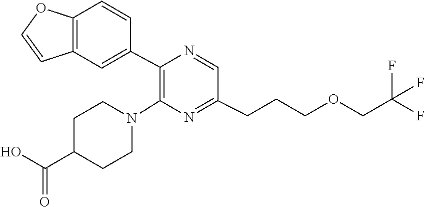 [SMILES: O=C(O)C1CCN(c2nc(CCCOCC(F)(F)F)cnc2-c2ccc3occc3c2)CC1] | 1-(3-(benzofuran-5-yl)-6-(3-(2,2,2- trifluoroethoxy)propyl)pyrazin-2- yl)piperidine-4-carboxylic acid |
| 178 | 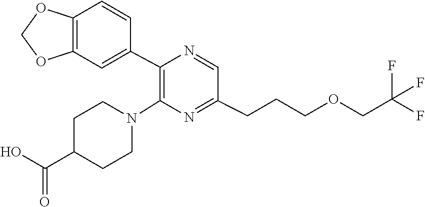 [SMILES: O=C(O)C1CCN(c2nc(CCCOCC(F)(F)F)cnc2-c2ccc3c(c2)OCO3)CC1] | 1-(3-(benzo[d][1,3]dioxol-5-yl)-6-(3- (2,2,2-trifluoroethoxy)propyl)pyrazin-2- yl)piperidine-4-carboxylic acid |
| 179 | 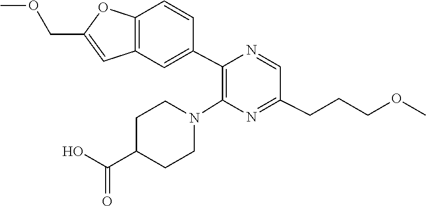 | 1-(3-(2-(methoxymethyl)benzofuran-5- yl)-6-(3-methoxypropyl)pyrazin-2- yl)piperidine-4-carboxylic acid |
| 180 | 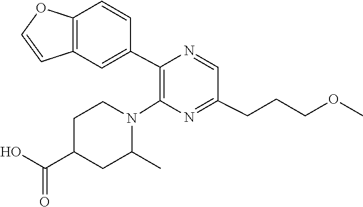 [SMILES: COCCCc1cnc(-c2ccc3occc3c2)c(N2CCC(C(=O)O)CC2C)n1] | 1-(3-(benzofuran-5-yl)-6-(3- methoxypropyl)pyrazin-2-yl)-2- methylpiperidine-4-carboxylic acid |
| 181 | 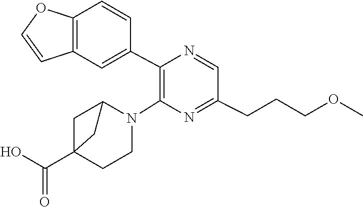 [SMILES: COCCCc1cnc(-c2ccc3occc3c2)c(N2CCC3(C(=O)O)CC2C3)n1] | 2-(3-(benzofuran-5-yl)-6-(3- methoxypropyl)pyrazin-2-yl)-2- azabicyclo[3.1.1]heptane-5-carboxylic acid |
| 182 | 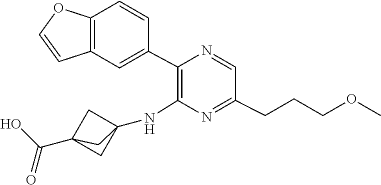 [SMILES: COCCCc1cnc(-c2ccc3occc3c2)c(NC23CC(C(=O)O)(C2)C3)n1] | 3-((3-(benzofuran-5-yl)-6-(3- methoxypropyl)pyrazin-2- yl)amino)bicyclo[1.1.1]pentane-1- carboxylic acid |
| 183 | 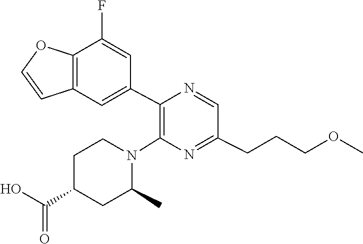 [SMILES: COCCCc1cnc(-c2cc(F)c3occc3c2)c(N2CC[C@@H](C(=O)O)C[C@@H]2C)n1] | (2S,4R)-1-(3-(7-fluorobenzofuran-5-yl)- 6-(3-methoxypropyl)pyrazin-2-yl)-2- methylpiperidine-4-carboxylic acid |
| | (absolute stereochemistry not determined) | |
| 184 | 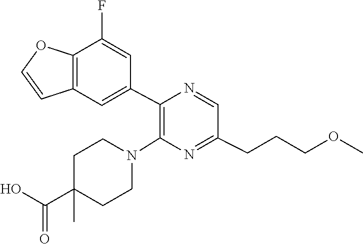 [SMILES: COCCCc1cnc(-c2cc(F)c3occc3c2)c(N2CCC(C)(C(=O)O)CC2)n1] | 1-(3-(7-fluorobenzofuran-5-yl)-6-(3- methoxypropyl)pyrazin-2-yl)-4- methylpiperidine-4-carboxylic acid |
| 185 | 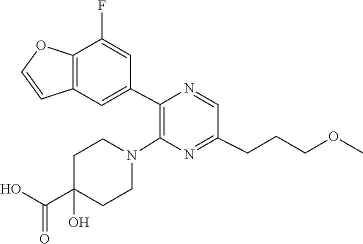 [SMILES: COCCCc1cnc(-c2cc(F)c3occc3c2)c(N2CCC(O)(C(=O)O)CC2)n1] | 1-(3-(7-fluorobenzofuran-5-yl)-6-(3- methoxypropyl)pyrazin-2-yl)-4- hydroxypiperidine-4-carboxylic acid |
| 186 | 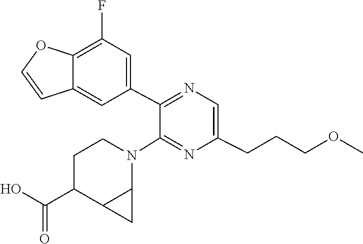 [SMILES: COCCCc1cnc(-c2cc(F)c3occc3c2)c(N2CCC(C(=O)O)C3CC32)n1] | 2-(3-(7-fluorobenzofuran-5-yl)-6-(3- methoxypropyl)pyrazin-2-yl)-2- azabicyclo[4.1.0]heptane-5-carboxylic acid |
| 187 | 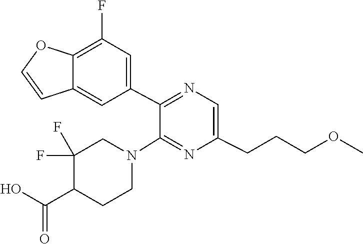 [SMILES: COCCCc1cnc(-c2cc(F)c3occc3c2)c(N2CCC(C(=O)O)C(F)(F)C2)n1] | 3,3-difluoro-1-(3-(7-fluorobenzofuran-5- yl)-6-(3-methoxypropyl)pyrazin-2- yl)piperidine-4-carboxylic acid |
| 188 | 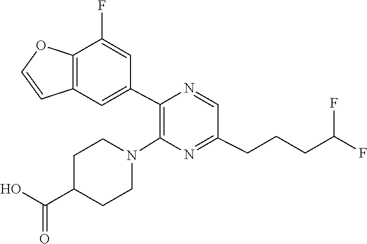 [SMILES: O=C(O)C1CCN(c2nc(CCCC(F)F)cnc2-c2cc(F)c3occc3c2)CC1] | 1-(6-(4,4-difluorobutyl)-3-(7- fluorobenzofuran-5-yl)pyrazin-2- yl)piperidine-4-carboxylic acid |
| 189 | 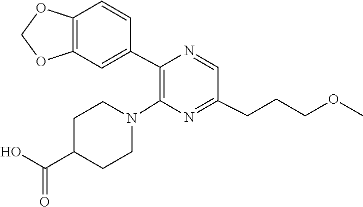 [SMILES: COCCCc1cnc(-c2ccc3c(c2)OCO3)c(N2CCC(C(=O)O)CC2)n1] | 1-(3-(benzo[d][1,3]dioxol-5-yl)-6-(3- methoxypropyl)pyrazin-2-yl)piperidine-4- carboxylic acid |
| 190 | 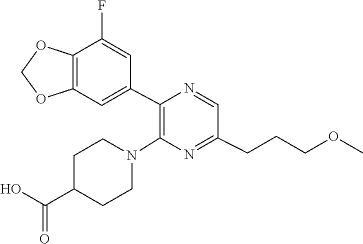 [SMILES: COCCCc1cnc(-c2cc(F)c3c(c2)OCO3)c(N2CCC(C(=O)O)CC2)n1] | 1-(3-(7-fluorobenzo[d][1,3]dioxol-5-yl)- 6-(3-methoxypropyl)pyrazin-2- yl)piperidine-4-carboxylic acid |
| 191 | 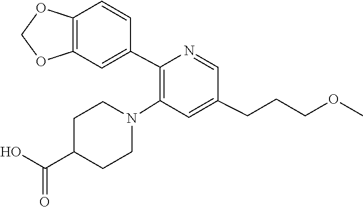 [SMILES: COCCCc1cnc(-c2ccc3c(c2)OCO3)c(N2CCC(C(=O)O)CC2)c1] | 1-(2-(benzo[d][1,3]dioxol-5-yl)-5-(3- methoxypropyl)pyridin-3-yl)piperidine-4- carboxylic acid |
| 192 | 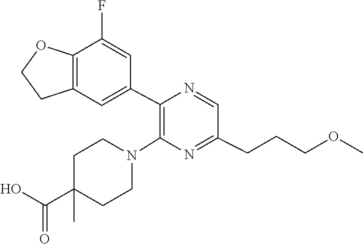 [SMILES: COCCCc1cnc(-c2cc(F)c3c(c2)CCO3)c(N2CCC(C)(C(=O)O)CC2)n1] | 1-(3-(7-fluoro-2,3-dihydrobenzofuran-5- yl)-6-(3-methoxypropyl)pyrazin-2-yl)-4- methylpiperidine-4-carboxylic acid |
| 193 | 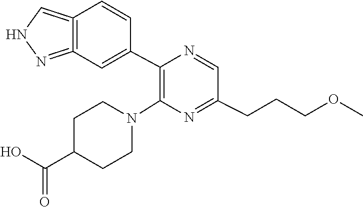 | 1-(3-(2H-indazol-6-yl)-6-(3- methoxypropyl)pyrazin-2-yl)piperidine-4- carboxylic acid |
| 194 | 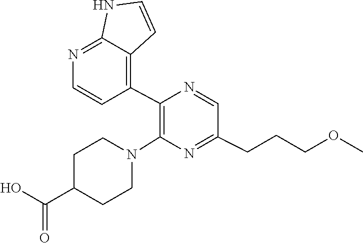 [SMILES: COCCCc1cnc(-c2ccnc3[nH]ccc23)c(N2CCC(C(=O)O)CC2)n1] | 1-(6-(3-methoxypropyl)-3-(1H- pyrrolo[2,3-b]pyridin-4-yl)pyrazin-2- yl)piperidine-4-carboxylic acid |
| 195 | 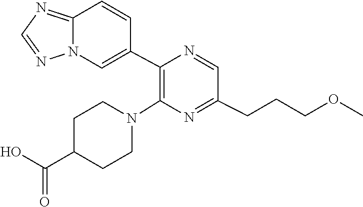 [SMILES: COCCCc1cnc(-c2ccc3ncnn3c2)c(N2CCC(C(=O)O)CC2)n1] | 1-(3-([1,2,4]triazolo[1,5-a]pyridin-6-yl)- 6-(3-methoxypropyl)pyrazin-2- yl)piperidine-4-carboxylic acid |
| 196 | 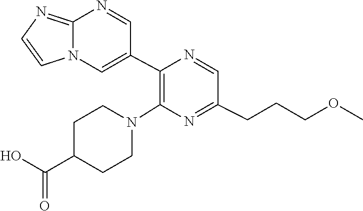 [SMILES: COCCCc1cnc(-c2cnc3nccn3c2)c(N2CCC(C(=O)O)CC2)n1] | 1-(3-(imidazo[1,2-a]pyrimidin-6-yl)-6-(3- methoxypropyl)pyrazin-2-yl)piperidine-4- carboxylic acid |
| 197 | 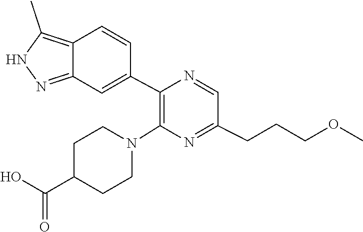 [SMILES: COCCCc1cnc(-c2ccc3c(C)[nH]nc3c2)c(N2CCC(C(=O)O)CC2)n1] | 1-(6-(3-methoxypropyl)-3-(3-methyl-2H- indazol-6-yl)pyrazin-2-yl)piperidine-4- carboxylic acid |
| 198 | 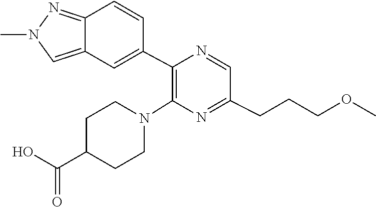 | 1-(6-(3-methoxypropyl)-3-(2-methyl-2H- indazol-5-yl)pyrazin-2-yl)piperidine-4- carboxylic acid |
| 199 | 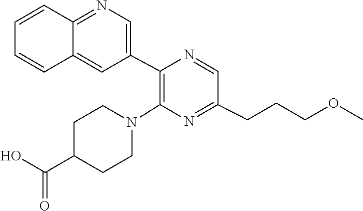 [SMILES: COCCCc1cnc(-c2cnc3ccccc3c2)c(N2CCC(C(=O)O)CC2)n1] | 1-(6-(3-methoxypropyl)-3-(quinolin-3- yl)pyrazin-2-yl)piperidine-4- carboxylic acid |
| 200 | 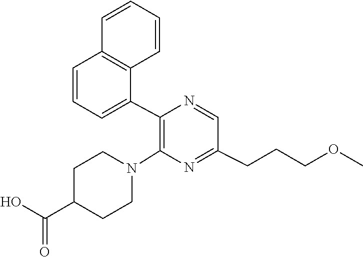 [SMILES: COCCCc1cnc(-c2cccc3ccccc23)c(N2CCC(C(=O)O)CC2)n1] | 1-(6-(3-methoxypropyl)-3-(quinolin-4- yl)pyrazin-2-yl)piperidine-4- carboxylic acid |
| 201 | 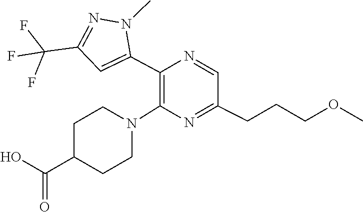 [SMILES: COCCCc1cnc(-c2cc(C(F)(F)F)nn2C)c(N2CCC(C(=O)O)CC2)n1] | 1-(6-(3-methoxypropyl)-3-(1-methyl-3- (trifluoromethyl)-1H-pyrazol-5- yl)pyrazin-2-yl)piperidine-4-carboxylic acid |
| 202 | 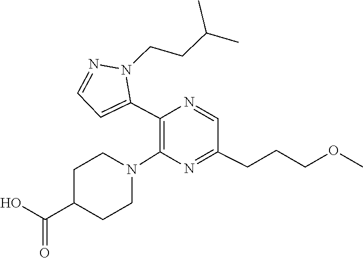 [SMILES: COCCCc1cnc(-c2ccnn2CCC(C)C)c(N2CCC(C(=O)O)CC2)n1] | 1-(3-(1-isopentyl-1H-pyrazol-5-yl)-6-(3- methoxypropyl)pyrazin-2-yl)piperidine-4- carboxylic acid |
| 203 | 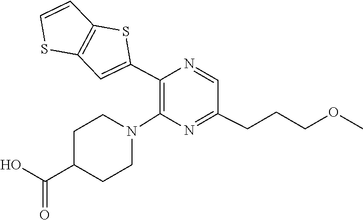 [SMILES: COCCCc1cnc(-c2cc3sccc3s2)c(N2CCC(C(=O)O)CC2)n1] | 1-(6-(3-methoxypropyl)-3-(thieno[3,2- b]thiophen-2-yl)pyrazin-2-yl)piperidine- 4-carboxylic acid |
| 204 | 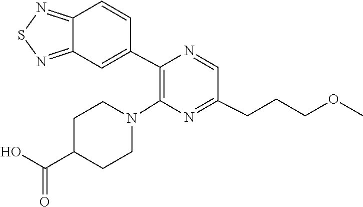 [SMILES: COCCCc1cnc(-c2ccc3nsnc3c2)c(N2CCC(C(=O)O)CC2)n1] | 1-(3-(benzo[c][1,2,5]thiadiazol-5-yl)-6- (3-methoxypropyl)pyrazin-2- yl)piperidine-4-carboxylic acid |
| 205 | 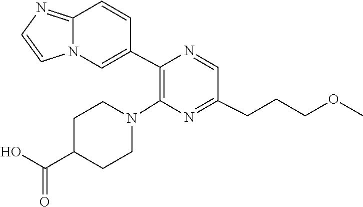 [SMILES: COCCCc1cnc(-c2ccc3nccn3c2)c(N2CCC(C(=O)O)CC2)n1] | 1-(3-(imidazo[1,2-a]pyridin-6-yl)-6-(3- methoxypropyl)pyrazin-2-yl)piperidine-4- carboxylic acid |
| 206 | 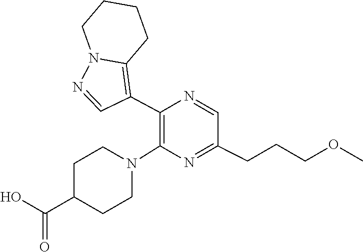 [SMILES: COCCCc1cnc(-c2cnn3c2CCCC3)c(N2CCC(C(=O)O)CC2)n1] | 1-(6-(3-methoxypropyl)-3-(4,5,6,7- tetrahydropyrazolo[1,5-a]pyridin-3- yl)pyrazin-2-yl)piperidine-4-carboxylic acid |
| 207 | 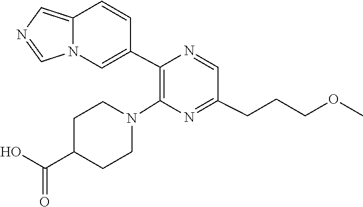 [SMILES: COCCCc1cnc(-c2ccc3cncn3c2)c(N2CCC(C(=O)O)CC2)n1] | 1-(3-(imidazo[1,5-a]pyridin-6-yl)-6-(3- methoxypropyl)pyrazin-2-yl)piperidine-4- carboxylic acid |
| 208 | 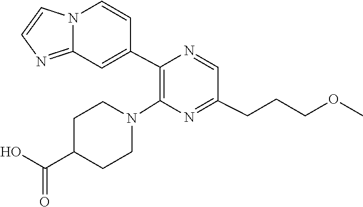 [SMILES: COCCCc1cnc(-c2ccn3ccnc3c2)c(N2CCC(C(=O)O)CC2)n1] | 1-(3-(imidazo[1,2-a]pyridin-7-yl)-6-(3- methoxypropyl)pyrazin-2-yl)piperidine-4- carboxylic acid |
| 209 | 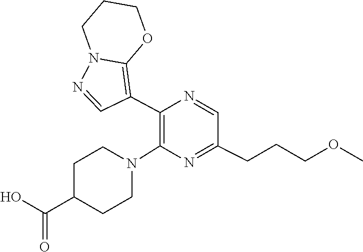 [SMILES: COCCCc1cnc(-c2cnn3c2OCCC3)c(N2CCC(C(=O)O)CC2)n1] | 1-(3-(6,7-dihydro-5H-pyrazolo[5,1- b][1,3]oxazin-3-yl)-6-(3- methoxypropyl)pyrazin-2-yl)piperidine-4- carboxylic acid |
| 210 | 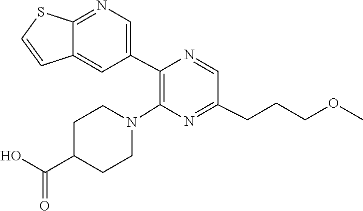 [SMILES: COCCCc1cnc(-c2cnc3sccc3c2)c(N2CCC(C(=O)O)CC2)n1] | 1-(6-(3-methoxypropyl)-3-(thieno[2,3- b]pyridin-5-yl)pyrazin-2-yl)piperidine-4- carboxylic acid |
| 211 | 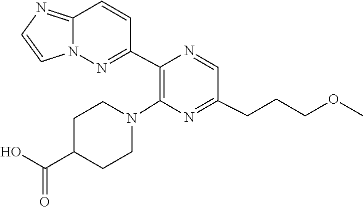 [SMILES: COCCCc1cnc(-c2ccc3nccn3n2)c(N2CCC(C(=O)O)CC2)n1] | 1-(3-(imidazo[1,2-b]pyridazin-6-yl)-6-(3- methoxypropyl)pyrazin-2-yl)piperidine-4- carboxylic acid |
| 212 | 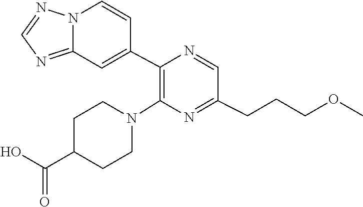 [SMILES: COCCCc1cnc(-c2ccn3ncnc3c2)c(N2CCC(C(=O)O)CC2)n1] | 1-(3-([1,2,4]triazolo[1,5-a]pyridin-7-yl)- 6-(3-methoxypropyl)pyrazin-2- yl)piperidine-4-carboxylic acid |
| 213 | 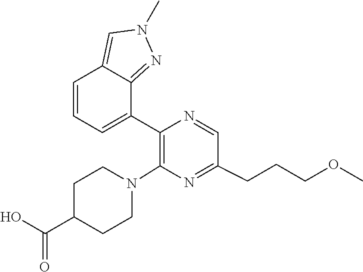 [SMILES: COCCCc1cnc(-c2cccc3cn(C)nc23)c(N2CCC(C(=O)O)CC2)n1] | 1-(6-(3-methoxypropyl)-3-(2-methyl-2H- indazol-7-yl)pyrazin-2-yl)piperidine-4- carboxylic acid |
| 214 | 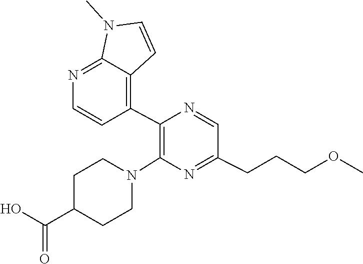 [SMILES: COCCCc1cnc(-c2ccnc3c2ccn3C)c(N2CCC(C(=O)O)CC2)n1] | 1-(6-(3-methoxypropyl)-3-(1-methyl-1H- pyrrolo[2,3-b]pyridin-4-yl)pyrazin-2- yl)piperidine-4-carboxylic acid |
| 215 | 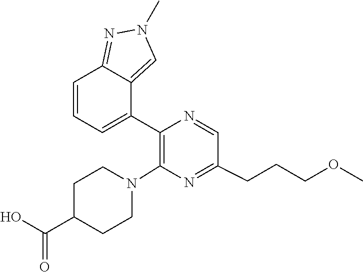 [SMILES: COCCCc1cnc(-c2cccc3nn(C)cc23)c(N2CCC(C(=O)O)CC2)n1] | 1-(6-(3-methoxypropyl)-3-(2-methyl-2H- indazol-4-yl)pyrazin-2-yl)piperidine-4- carboxylic acid |
| 216 | 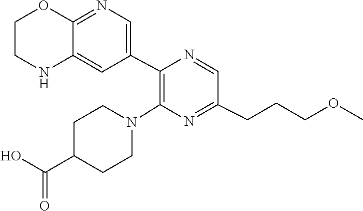 [SMILES: COCCCc1cnc(-c2cnc3c(c2)NCCO3)c(N2CCC(C(=O)O)CC2)n1] | 1-(3-(2,3-dihydro-1H-pyrido[2,3- b][1,4]oxazin-7-yl)-6-(3- methoxypropyl)pyrazin-2-yl)piperidine-4- carboxylic acid |
| 217 | 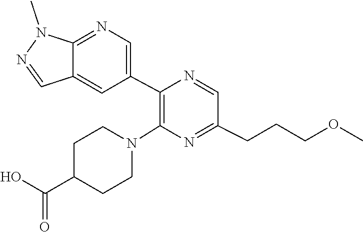 [SMILES: COCCCc1cnc(-c2cnc3c(cnn3C)c2)c(N2CCC(C(=O)O)CC2)n1] | 1-(6-(3-methoxypropyl)-3-(1-methyl-1H- pyrazolo[3,4-b]pyridin-5-yl)pyrazin-2- yl)piperidine-4-carboxylic acid |
| 218 | 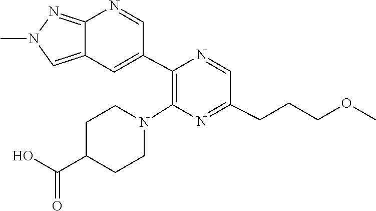 | 1-(6-(3-methoxypropyl)-3-(2-methyl-2H- pyrazolo[3,4-b]pyridin-5-yl)pyrazin-2- yl)piperidine-4-carboxylic acid |
| 219 | 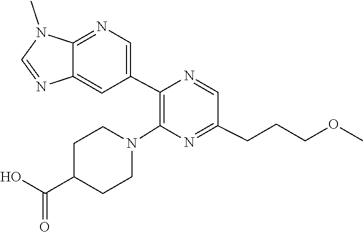 [SMILES: COCCCc1cnc(-c2cnc3c(c2)ncn3C)c(N2CCC(C(=O)O)CC2)n1] | 1-(6-(3-methoxypropyl)-3-(3-methyl-3H- imidazo[4,5-b]pyridin-6-yl)pyrazin-2- yl)piperidine-4-carboxylic acid |
| 220 | 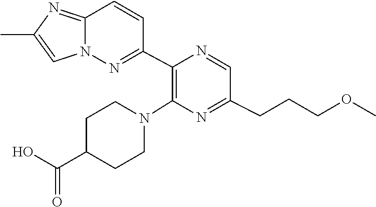 | 1-(3-(2,8-dimethylimidazo[1,2- b]pyridazin-6-yl)-6-(3- methoxypropyl)pyrazin-2-yl)piperidine-4- carboxylic acid |
| 221 | 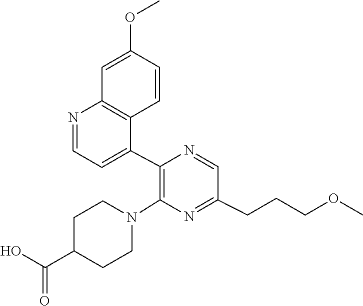 [SMILES: COCCCc1cnc(-c2ccnc3cc(OC)ccc23)c(N2CCC(C(=O)O)CC2)n1] | 1-(6-(3-methoxypropyl)-3-(7- methoxyquinolin-4-yl)pyrazin-2- yl)piperidine-4-carboxylic acid |
| 222 | 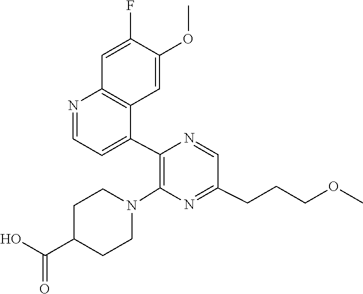 [SMILES: COCCCc1cnc(-c2ccnc3cc(F)c(OC)cc23)c(N2CCC(C(=O)O)CC2)n1] | 1-(3-(7-fluoro-6-methoxyquinolin-4-yl)- 6-(3-methoxypropyl)pyrazin-2- yl)piperidine-4-carboxylic acid |
| 223 | 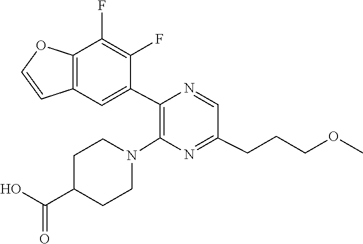 [SMILES: COCCCc1cnc(-c2cc3ccoc3c(F)c2F)c(N2CCC(C(=O)O)CC2)n1] | 1-(3-(6,7-difluorobenzofuran-5-yl)-6-(3- methoxypropyl)pyrazin-2-yl)piperidine-4- carboxylic acid |
| 224 | 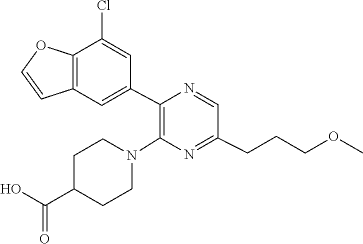 [SMILES: COCCCc1cnc(-c2cc(Cl)c3occc3c2)c(N2CCC(C(=O)O)CC2)n1] | 1-(3-(7-chlorobenzofuran-5-yl)-6-(3- methoxypropyl)pyrazin-2-yl)piperidine-4- carboxylic acid |
| 225 | 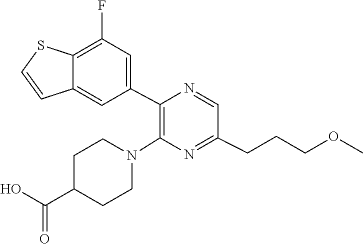 [SMILES: COCCCc1cnc(-c2cc(F)c3sccc3c2)c(N2CCC(C(=O)O)CC2)n1] | 1-(3-(7-fluorobenzo[b]thiophen-5-yl)-6- (3-methoxypropyl)pyrazin-2- yl)piperidine-4-carboxylic acid |
| 226 | 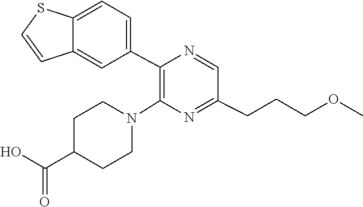 [SMILES: COCCCc1cnc(-c2ccc3sccc3c2)c(N2CCC(C(=O)O)CC2)n1] | 1-(3-(benzo[b]thiophen-5-yl)-6-(3- methoxypropyl)pyrazin-2-yl)piperidine-4- carboxylic acid |
| 227 | 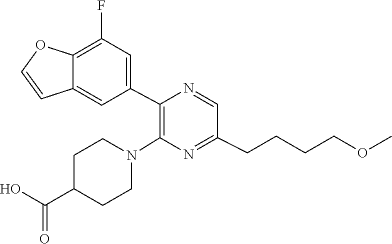 [SMILES: COCCCCc1cnc(-c2cc(F)c3occc3c2)c(N2CCC(C(=O)O)CC2)n1] | 1-(3-(7-fluorobenzofuran-5-yl)-6-(4- methoxybutyl)pyrazin-2-yl)piperidine-4- carboxylic acid |
| 228 | 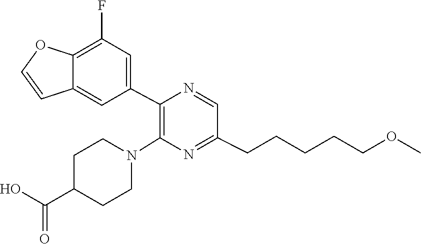 [SMILES: COCCCCCc1cnc(-c2cc(F)c3occc3c2)c(N2CCC(C(=O)O)CC2)n1] | 1-(3-(7-fluorobenzofuran-5-yl)-6-(5- methoxypentyl)pyrazin-2-yl)piperidine-4- carboxylic acid |
| 229 | 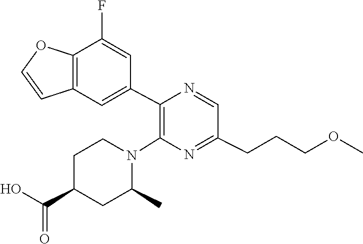 [SMILES: COCCCc1cnc(-c2cc(F)c3occc3c2)c(N2CC[C@H](C(=O)O)C[C@@H]2C)n1] | (2S,4S)-1-(3-(7-fluorobenzofuran-5-yl)-6- (3-methoxypropyl)pyrazin-2-yl)-2- methylpiperidine-4-carboxylic acid |
| | (absolute stereochemistry not determined) | |
| 230 | 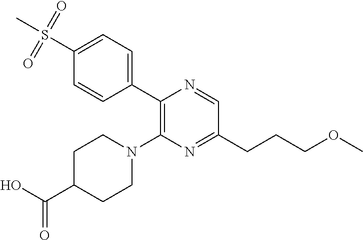 [SMILES: COCCCc1cnc(-c2ccc(S(C)(=O)=O)cc2)c(N2CCC(C(=O)O)CC2)n1] | 1-(6-(3-methoxypropyl)-3-(4- (methylsulfonyl)phenyl)pyrazin-2- yl)piperidine-4-carboxylic acid |
| 231 | 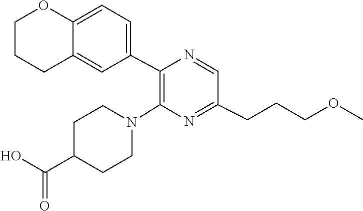 [SMILES: COCCCc1cnc(-c2ccc3c(c2)CCCO3)c(N2CCC(C(=O)O)CC2)n1] | 1-(3-(chroman-6-yl)-6-(3- methoxypropyl)pyrazin-2-yl)piperidine-4- carboxylic acid |
| 232 | 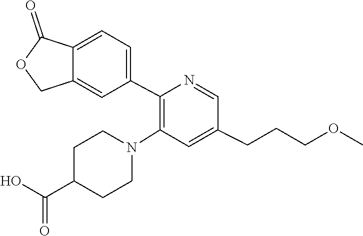 [SMILES: COCCCc1cnc(-c2ccc3c(c2)COC3=O)c(N2CCC(C(=O)O)CC2)c1] | 1-(5-(3-methoxypropyl)-2-(1-oxo-1,3- dihydroisobenzofuran-5-yl)pyridin-3- yl)piperidine-4-carboxylic acid |
| 233 | 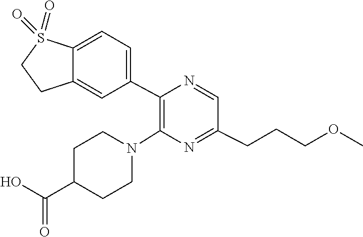 [SMILES: COCCCc1cnc(-c2ccc3c(c2)CCS3(=O)=O)c(N2CCC(C(=O)O)CC2)n1] | 1-(3-(1,1-dioxido-2,3- dihydrobenzo[b]thiophen-5-yl)-6-(3- methoxypropyl)pyrazin-2-yl)piperidine-4- carboxylic acid |
| 234 | 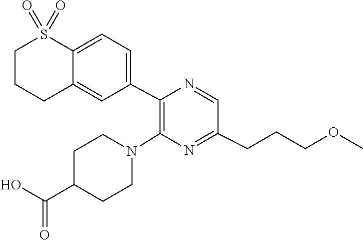 [SMILES: COCCCc1cnc(-c2ccc3c(c2)CCCS3(=O)=O)c(N2CCC(C(=O)O)CC2)n1] | 1-(3-(1,1-dioxidothiochroman-6-yl)-6-(3- methoxypropyl)pyrazin-2-yl)piperidine-4- carboxylic acid |
| 235 | 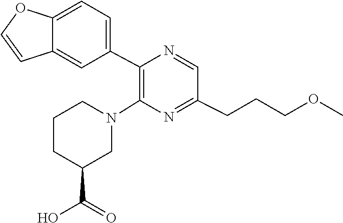 [SMILES: COCCCc1cnc(-c2ccc3occc3c2)c(N2CCC[C@H](C(=O)O)C2)n1] | (S)-1-(3-(benzofuran-5-yl)-6-(3- methoxypropyl)pyrazin-2-yl)piperidine-3- carboxylic acid |
| | (absolute stereochemistry not determined) | |
| 236 | 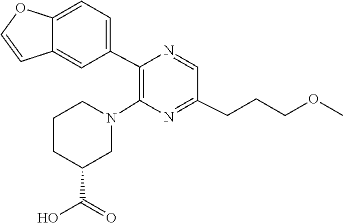 [SMILES: COCCCc1cnc(-c2ccc3occc3c2)c(N2CCC[C@@H](C(=O)O)C2)n1] | (R)-1-(3-(benzofuran-5-yl)-6-(3- methoxypropyl)pyrazin-2-yl)piperidine-3- carboxylic acid |
| | (absolute stereochemistry not determined) | |
| 237 | 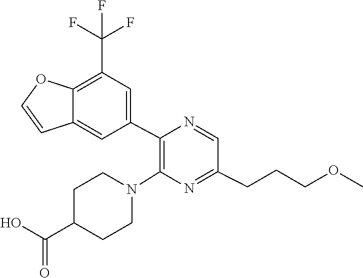 [SMILES: COCCCc1cnc(-c2cc(C(F)(F)F)c3occc3c2)c(N2CCC(C(=O)O)CC2)n1] | 1-(6-(3-methoxypropyl)-3-(7- (trifluoromethyl)benzofuran-5-yl)pyrazin- 2-yl)piperidine-4-carboxylic acid |
| 238 | 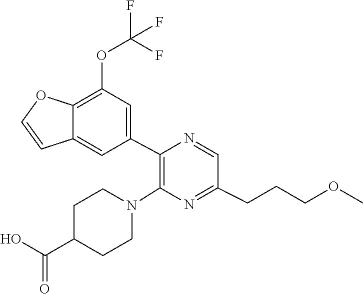 [SMILES: COCCCc1cnc(-c2cc(OC(F)(F)F)c3occc3c2)c(N2CCC(C(=O)O)CC2)n1] | 1-(6-(3-methoxypropyl)-3-(7- (trifluoromethoxy)benzofuran-5- yl)pyrazin-2-yl)piperidine-4-carboxylic acid |
| 239 | 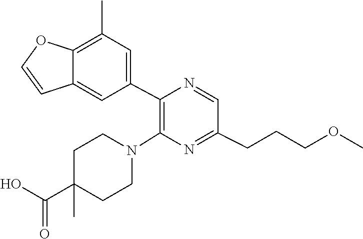 [SMILES: COCCCc1cnc(-c2cc(C)c3occc3c2)c(N2CCC(C)(C(=O)O)CC2)n1] | 1-(6-(3-methoxypropyl)-3-(7- methylbenzofuran-5-yl)pyrazin-2- yl)piperidine-4-carboxylic acid |
| 240 | 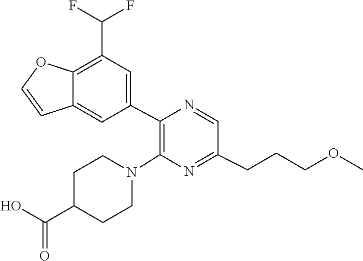 [SMILES: COCCCc1cnc(-c2cc(C(F)F)c3occc3c2)c(N2CCC(C(=O)O)CC2)n1] | 1-(3-(7-(difluoromethyl)benzofuran-5-yl)- 6-(3-methoxypropyl)pyrazin-2- yl)piperidine-4-carboxylic acid |
| 241 | 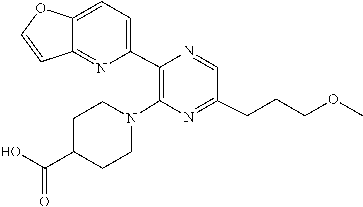 [SMILES: COCCCc1cnc(-c2ccc3occc3n2)c(N2CCC(C(=O)O)CC2)n1] | 1-(3-(furo[3,2-b]pyridin-5-yl)-6-(3- methoxypropyl)pyrazin-2-yl)piperidine-4- carboxylic acid |
| 242 | 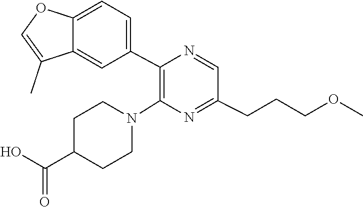 [SMILES: COCCCc1cnc(-c2ccc3occ(C)c3c2)c(N2CCC(C(=O)O)CC2)n1] | 1-(6-(3-methoxypropyl)-3-(3- methylbenzofuran-5-yl)pyrazin-2- yl)piperidine-4-carboxylic acid |
| 243 | 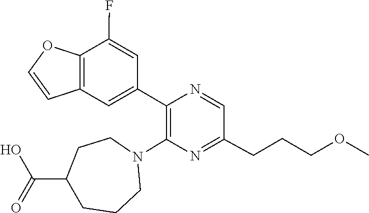 [SMILES: COCCCc1cnc(-c2cc(F)c3occc3c2)c(N2CCCC(C(=O)O)CC2)n1] | 1-(3-(7-fluorobenzofuran-5-yl)-6-(3- methoxypropyl)pyrazin-2-yl)azepane-4- carboxylic acid |
| 244 | 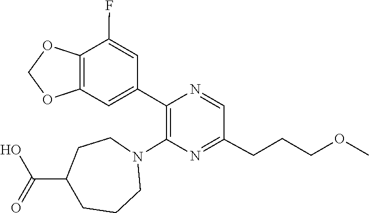 [SMILES: COCCCc1cnc(-c2cc(F)c3c(c2)OCO3)c(N2CCCC(C(=O)O)CC2)n1] | 1-(3-(7-fluorobenzo[d][1,3]dioxol-5-yl)- 6-(3-methoxypropyl)pyrazin-2- yl)azepane-4-carboxylic acid |
| 245 | 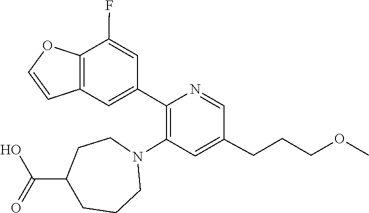 [SMILES: COCCCc1cnc(-c2cc(F)c3occc3c2)c(N2CCCC(C(=O)O)CC2)c1] | 1-(2-(7-fluorobenzofuran-5-yl)-5-(3- methoxypropyl)pyridin-3-yl)azepane-4- carboxylic acid |
| 246 | 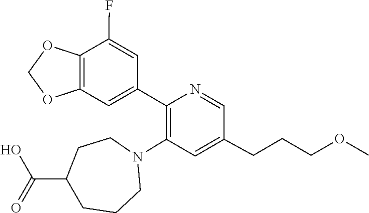 [SMILES: COCCCc1cnc(-c2cc(F)c3c(c2)OCO3)c(N2CCCC(C(=O)O)CC2)c1] | 1-(2-(7-fluorobenzo[d][1,3]dioxol-5-yl)- 5-(3-methoxypropyl)pyridin-3- yl)azepane-4-carboxylic acid |
| 247 | 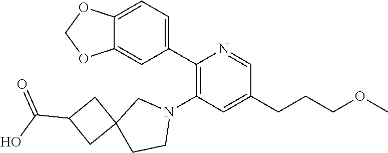 [SMILES: COCCCc1cnc(-c2ccc3c(c2)OCO3)c(N2CCC3(CC(C(=O)O)C3)C2)c1] | 6-(2-(benzo[d][1,3]dioxol-5-yl)-5-(3- methoxypropyl)pyridin-3-yl)-6- azaspiro[3.4]octane-2-carboxylic acid |
| 248 | 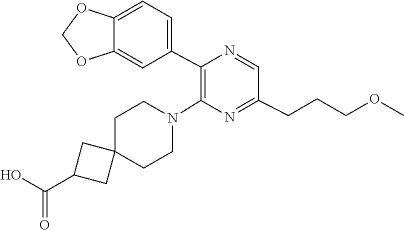 [SMILES: COCCCc1cnc(-c2ccc3c(c2)OCO3)c(N2CCC3(CC2)CC(C(=O)O)C3)n1] | 7-(2-(benzo[d][1,3]dioxol-5-yl)-5-(3- methoxypropyl)pyridin-3-yl)-7- azaspiro[3.5]nonane-2-carboxylic acid |
| 249 | 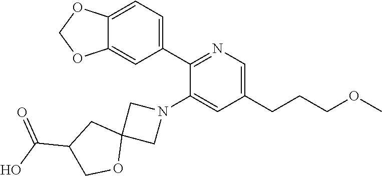 [SMILES: COCCCc1cnc(-c2ccc3c(c2)OCO3)c(N2CC3(CC(C(=O)O)CO3)C2)c1] | 2-(2-(benzo[d][1,3]dioxol-5-yl)-5-(3- methoxypropyl)pyridin-3-yl)-5-oxa-2- azaspiro[3.4]octane-7-carboxylic acid |
| 250 | 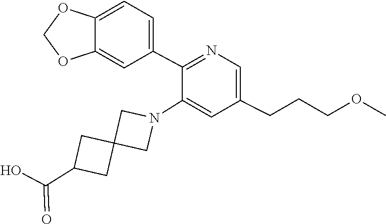 [SMILES: COCCCc1cnc(-c2ccc3c(c2)OCO3)c(N2CC3(CC(C(=O)O)C3)C2)c1] | 2-(2-(benzo[d][1,3]dioxol-5-yl)-5-(3- methoxypropyl)pyridin-3-yl)-2- azaspiro[3.3]heptane-6-carboxylic acid |
| 251 | 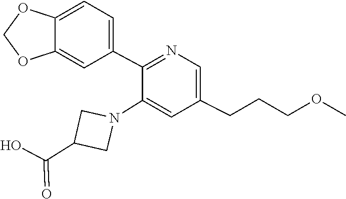 [SMILES: COCCCc1cnc(-c2ccc3c(c2)OCO3)c(N2CC(C(=O)O)C2)c1] | 1-(2-(benzo[d][1,3]dioxol-5-yl)-5-(3- methoxypropyl)pyridin-3-yl)azetidine-3- carboxylic acid |
| 252 | 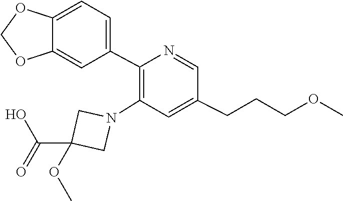 [SMILES: COCCCc1cnc(-c2ccc3c(c2)OCO3)c(N2CC(OC)(C(=O)O)C2)c1] | 1-(2-(benzo[d][1,3]dioxol-5-yl)-5-(3- methoxypropyl)pyridin-3-yl)-3- methoxyazetidine-3-carboxylic acid |
| 253 | 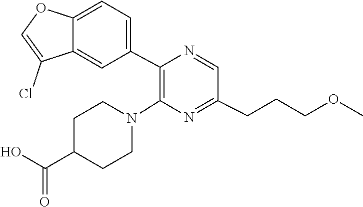 [SMILES: COCCCc1cnc(-c2ccc3occ(Cl)c3c2)c(N2CCC(C(=O)O)CC2)n1] | 1-(3-(3-chlorobenzofuran-5-yl)-6-(3- methoxypropyl)pyrazin-2-yl)piperidine-4- carboxylic acid |
| 254 | 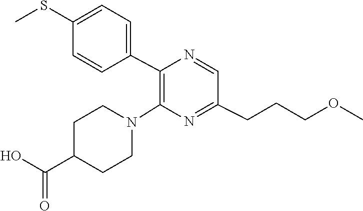 | 1-(6-(3-methoxypropyl)-3-(4- (methylthio)phenyl)pyrazin-2- yl)piperidine-4-carboxylic acid |
| 255 | 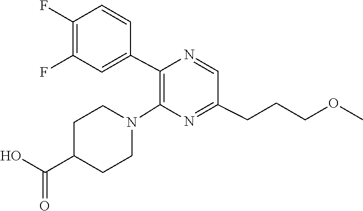 [SMILES: COCCCc1cnc(-c2ccc(F)c(F)c2)c(N2CCC(C(=O)O)CC2)n1] | 1-(3-(3,4-difluorophenyl)-6-(3- methoxypropyl)pyrazin-2-yl)piperidine-4- carboxylic acid |

In some embodiments, the compound is a pharmaceutically acceptable salt of a compound described in Table 1.

Also provided herein is a compound selected from the Examples disclosed herein.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(═O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(˜CH═CH—), (˜C:: C-)]. Examples of suitable alkenyl radicals include ethenyl, propenyl, 2-methylpropenyl, 1,4-butadienyl and the like. Unless otherwise specified, the term “alkenyl” may include “alkenylene” groups. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkylidene,” as used herein, alone or in combination, refers to an alkenyl group in which one carbon atom of the carbon-carbon double bond belongs to the moiety to which the alkenyl group is attached.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like. In some embodiments, an alkoxy group refers to a (alkyl)-O— group, where alkyl is as defined herein. In some embodiments, the alkoxy group is a C₁-C₆alkoxy, which refers to a (C₁-C₆alkyl)-O— group.

An “alkyl” group refers to an aliphatic hydrocarbon group. The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl and the like. In some embodiments, an alkyl is a C₁-C₆alkyl. In one aspect the alkyl is methyl, ethyl, propyl, iso-propyl, n-butyl, iso-butyl, sec-butyl, or t-butyl. The term “alkylene,” as used herein, alone or in combination, refers to a saturated aliphatic group derived from a straight or branched chain saturated hydrocarbon attached at two or more positions, such as methylene (—CH₂—). In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—. Unless otherwise specified, the term “alkyl” may include “alkylene” groups.

An “alkoxyalkyl” refers to an alkyl in which one hydrogen atom is replaced by an alkoxy group, as defined herein. In some embodiments, an alkoxyalkyl is a (C₁-C₆alkoxy)-C₁-C₆alkyl, which can also be considered a (C₁-C₆alkyl)-O—(C₁-C₆alkyl)−group. In some embodiments, an alkoxyalkyl is a (C₁-C₄alkoxy)-C₁-C₆alkyl, which can also be considered a (C₁-C₄alkyl)-O—(C₁-C₆alkyl)- group. Typical alkoxyalkyl groups include, but are not limited to, —CH₂OCH₃, —CH₂CH₂OCH₃, —CH₂CH₂CH₂OCH₃, —CH₂CH₂CH₂CH₂OCH₃, —CH₂OCH₂CH₃, —CH₂CH₂OCH₂CH₃, —CH₂CH₂CH₂OCH₂CH₃, —CH₂CH₂CH₂CH₂OCH₂CH₃, and the like.

An “alkoxyalkoxyalkyl” refers to an alkoxyalkyl in which one hydrogen atom of the alkoxy group is replaced by a second alkoxy group, as defined herein. In some embodiments, an alkoxyalkoxyalkyl is a (C₁-C₆alkoxy)-(C₁-C₆alkoxy)-C₁-C₆alkyl, which can also be considered a (C₁-C₆alkyl)-O—(C₁-C₆alkyl)-O—(C₁-C₆alkyl)- group. In some embodiments, an alkoxyalkoxyalkyl is a (C₁-C₄alkoxy)-(C₁-C₄alkoxy)-C₁-C₆alkyl, which can also be considered a (C₁-C₄alkyl)-O—(C₁-C₄ₐlkyl)-O—(C₁-C₆alkyl)- group. Typical alkoxyalkoxyalkyl groups include, but are not limited to, —CH₂OCH₂CH₂OCH₃, —CH₂CH₂OCH₂CH₂OCH₃, —CH₂CH₂CH₂OCH₂CH₂OCH₃, —CH₂CH₂CH₂CH₂OCH₂CH₂OCH₃, —CH₂OCH₂CH₂CH₂OCH₃, —CH₂CH₂OCH₂CH₂CH₂OCH₃, —CH₂CH₂CH₂OCH₂CH₂CH₂OCH₃, —CH₂CH₂CH₂CH₂OCH₂CH₂CH₂OCH₃, —CH₂OCH₂CH₂OCH₂CH₃, —CH₂CH₂OCH₂CH₂OCH₂CH₃, —CH₂CH₂CH₂OCH₂CH₂OCH₂CH₃, —CH₂CH₂CH₂CH₂OCH₂CH₂OCH₂CH₃, —CH₂OCH₂CH₂CH₂OCH₂CH₃, —CH₂CH₂OCH₂CH₂CH₂OCH₂CH₃, —CH₂CH₂CH₂OCH₂CH₂CH₂OCH₂CH₃, —CH₂CH₂CH₂CH₂OCH₂CH₂CH₂OCH₂CH₃, and the like.

An “alkoxyalkoxy” refers to an alkoxy in which one hydrogen atom is replaced by an alkoxy group, as defined herein. In some embodiments, an alkoxyalkoxy is a (C₁-C₆alkoxy)-C₁-C₆alkoxy, which can also be considered a (C₁-C₆alkyl)-O—(C₁-C₆alkyl)-O— group. In some embodiments, an alkoxyalkyl is a (C₁-C₄alkoxy)-C₁-C₆alkoxy, which can also be considered a (C₁-C₄ₐlkyl)-O—(C₁-C₆alkyl)-O— group. Typical alkoxyalkyl groups include, but are not limited to, —OCH₂CH₂OCH₃, —OCH₂CH₂CH₂OCH₃, —OCH₂CH₂CH₂CH₂OCH₃, —OCH₂OCH₂CH₃, —OCH₂CH₂OCH₂CH₃, —OCH₂CH₂CH₂OCH₂CH₃, —OCH₂CH₂CH₂CH₂OCH₂CH₃, and the like.

The term “amino,” as used herein, alone or in combination, refers to —NRR′, wherein R and R′ are independently selected from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted. In one aspect, “amino” as used herein refers to an —NH₂ group.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like. In some embodiments, the term “alkylamino” refers to the —N(alkyl)ₓHy group, where x is 0 and y is 2, or where x is 1 and y is 1, or where x is 2 and y is 0. In some embodiments, the term “alkylamino” refers to the —NH(alkyl) group and the term “dialkylamino” refers to the N(alkyl)₂ group. In some embodiments, the alkylamino group is a C₁-C₆alkylamino, which refers to the —NH(C₁-C₆alkyl) group. In some embodiments, the dialkylamino group is a di-(C₁-C₆alkyl)-amino, which refers to the —N(C₁-C₆alkyl)₂ group.

The term “alkylthio,” as used herein, alone or in combination, refers to an alkyl thioether (R-S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like. In some embodiments, the term “alkylthio” refers to the —S-(alkyl) group. In some embodiments, the alkylthio group is a C₁-C₆alkylthio, which refers to the —S—(C₁-C₆alkyl) group.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃, —C≡CCH₂CH₃, —CH₂C≡CH. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)N(RR′) group with R and R′ as defined herein or as defined by the specifically enumerated “R” groups designated. The term “N-amido” as used herein, alone or in combination, refers to a RC(═O)N(R′)-group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(═O)NH—).

The term “aromatic” refers to a planar ring having a delocalized 7r-electron system containing 4n+2 7r electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl. In one aspect, aryl is phenyl or a naphthyl. In some embodiments, an aryl is a phenyl. In some embodiments, an aryl is a phenyl, naphthyl, indanyl, indenyl, or tetrahyodronaphthyl. In some embodiments, an aryl is a C₆-Cioaryl. Depending on the structure, an aryl group is a monoradical or a diradical (i.e., an arylene group).

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy. In some embodiments, aryloxy as used herein refers to the aryl-O— group. In some embodiments, aryloxy is phenoxy, or a phenyl-O— group

The terms “benzo” and “benz,” as used herein refer to fused bicyclic or polyclic ring system that is formed with benzene as one of the rings. Examples include benzofuran, benzothiophene, and benzimidazole.

The term “cycloalkyl,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In some embodiments, cycloalkyl groups include groups having from 3 to 10 ring atoms. In certain embodiments, said cycloalkyl will comprise from 5 to 7 carbon atoms. In certain embodiments, said cycloalkyl will comprise from 3 to 6 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane. In some embodiments, a cycloalkyl is a C₃-C₆cycloalkyl. In some embodiments, a cycloalkyl is a C₃-C_{4c}ycloalkyl.

The term “cycloalkyloxy” as used herein, alone or in combination, refers to a cycloalkyl group attached to the parent molecular moiety through an oxy. In some embodiments, the cycloalkyloxy is a C₃-C₆cycloalkoxy, which refers to a (C₃-C₆cycloalkyl)-0-group.

A “cycloalkylalkyl” refers to an alkyl in which one hydrogen atom is replaced by a cycloalkyl group, as defined herein. In some embodiments, a cycloalkylalkyl is a (C₃-C₆cycloalkyl)-C₁-C₆alkyl.

A “cycloalkoxyalkyl” refers to an alkyl in which one hydrogen atom is replaced by a cycloalkoxy group, as defined herein. In some embodiments, a cycloalkoxyalkyl is a (C₃-C₆cycloalkoxy)-C₁-C₆alkyl, which refers to a (C₃-C₆cycloalkyl)-O—(C₁-C₆alkyl)- group.

A “cycloalkylalkoxy” refers to an alkoxy in which one hydrogen atom is replaced by a cycloalkyl group, as defined herein. In some embodiments, a cycloalkylalkoxy is a (C₃-C₆cycloalkyl)-C₁-C₆alkoxy, which refers to a (C₃-C₆cycloalkyl)-(C₁-C₆alkyl)-O— group.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 1 O atoms in its ring system, and with the proviso that any ring does not contain two adjacent O or S atoms. Non-aromatic heterocyclic groups (also known as heterocycloalkyls) include rings having 3 to 1 O atoms in its ring system and aromatic heterocyclic groups include rings having 5 to 1 O atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. In some embodiments, the term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuranyl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-Csheteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, the term “heterocycloalkyl” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(═O)NRR′, group-with R and R′ as defined herein.

The term “N-carbamyl” as used herein, alone or in combination, refers to a ROC(═O)NR′— group, with R and R′ as defined herein.

The term “carbonyl,” as used herein, when alone includes formyl [—C(═O)H]and in combination is a —C(═O)— group.

The term “carboxyl” or “carboxy,” as used herein, refers to —C(═O)OH or the corresponding “carboxylate” anion, such as is in a carboxylic acid salt. An “O-carboxy” group refers to a RC(═O)O— group, where R is as defined herein. A “C-carboxy” group refers to a —C(═O)OR groups where R is as defined herein.

The term “cyano,” as used herein, alone or in combination, refers to —CN.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine. In some embodiments, halo is fluoro, chloro, or bromo.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like. In one aspect, a haloalkyl is a C₁-C₆haloalkyl. In another aspect, a haloalkyl is a C₁-C₄haloalkyl.

The term “haloalkoxy,” as used herein, alone or in combination, refers to a haloalkyl group attached to the parent molecular moiety through an oxygen atom. In one aspect, the haloalkoxy is a C₁-C₆haloalkoxy, which refers to a (C₁-C₆haloalkyl)-O— group. In another aspect, the haloalkoxy is a C₁-C₄haloalkoxy, which refers to a (C₁-C₄haloalkyl)-O— group.

A “haloalkoxyalkyl” refers to an alkyl in which one hydrogen atom is replaced by a haloalkoxy group, as defined herein. In some embodiments, a haloalkoxyalkyl is a (C₁-C₆haloalkoxy)-C₁-C₆alkyl, which can also be considered a (C₁-C₆haloalkyl)-O—(C₁-C₆alkyl)- group. In some embodiments, an alkoxyalkyl is a (C₁-C₄haloalkoxy)-C₁-C₆alkyl, which can also be considered a (C₁-C₄haloalkyl)-O—(C₁-C₆alkyl)- group. Typical haloalkoxyalkyl groups include, but are not limited to, —CH₂OCF₃, —CH₂CH₂OCF₃, —CH₂CH₂CH₂OCF₃, —CH₂CH₂CH₂CH₂OCF₃, —CH₂OCH₂CF₃, —CH₂CH₂OCH₂CF₃, —CH₂CH₂CH₂OCH₂CF₃, —CH₂CH₂CH₂CH₂OCH₂CF₃, and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms selected from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “hydrazinyl” as used herein, alone or in combination, refers to two amino groups joined by a single bond, i.e., —N-N—.

The term “hydroxy,” or “hydroxyl,” as used herein, alone or in combination, refers to —OH.

The term “hydroxyalkyl,” as used herein, alone or in combination, refers to a hydroxy group attached to the parent molecular moiety through an alkyl group. In some embodiments, a hydroxyalkyl is a C₁-C₄ hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms selected from N, O, and S, or 2) bicyclic heteroaryl, wherein each of the fused rings comprises five or six ring members, comprising between them one to four heteroatoms selected from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from hydrogen and lower alkyl, either of which may be optionally substituted.

The term “mercaptyl” as used herein, alone or in combination, refers to an RS-group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(═O)—.

The term “sulfonyl,” as used herein, alone or in combination, refers to a —S(═O)₂—, —S(═O)₂R, or —S(═O)₂R— group, with R as defined herein.

The term “sulfonamido,” as used herein, alone or in combination, includes both N-sulfonamido and S-sulfonamido. The term “N-sulfonamido” refers to either a RS(═O)₂NR′— or —S(═O)₂NR′— group with R and R′ as defined herein. The term “S-sulfonamido” refers to a —S(═O)₂NRR′ or —S(═O)₂NR— group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(═O)₂NR— group with X is a halogen and R as defined herein.

The term “trihalomethanesulfonyl” refers to a X₃CS(═O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(═O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

In some embodiments, the term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄ fluoroalkyl, C₁-C₄ₕeteroalkyl, C₁-C₄alkoxy, C₁-C₄ fluoroalkoxy, —SC₁-C₄ₐlkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term R or the term R′, appearing by itself and without a number designation, unless otherwise defined, refers to a moiety selected from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(═O)N(R)-may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“GLUT9 inhibitor” is used herein to refer to a compound that exhibits an IC₅₀ with respect to GLUT9 activity of no more than about 10 μM and more typically not more than about 5 μM, as measured in the GLUT9 assay described generally herein. “IC₅₀” is that concentration of inhibitor which reduces the activity of an enzyme (e.g., GLUT9) to half-maximal level. Certain compounds disclosed herein have been discovered to exhibit inhibition against GLUT9. In certain embodiments, compounds will exhibit an EC₅₀ with respect to GLUT9 of no more than about 2 μM; in yet further embodiments, compounds will exhibit an EC₅₀ with respect to GLUT9 of not more than about 1 μM; in yet further embodiments, compounds will exhibit an EC₅₀ with respect to GLUT9 of not more than about 500 nM, as measured in the GLUT9 assay described herein.

The phrase “therapeutically effective” is intended to qualify the amount of active ingredients used in the treatment of a disease or disorder or on the effecting of a clinical endpoint.

The term “pharmaceutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, “treating,” “treatment,” and the like means ameliorating a disease, so as to reduce, ameliorate, or eliminate its cause, its progression, its severity, or one or more of its symptoms, or otherwise beneficially alter the disease in a subject. In certain embodiments, reference to “treating” or “treatment” of a subject at risk for developing a disease, or at risk of disease progression to a worse state, is intended to include prophylaxis. Prevention of a disease may involve complete protection from disease, for example as in the case of prevention of infection with a pathogen, or may involve prevention of disease progression, for example from prediabetes to diabetes. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the terms “subject” and includes all mammals including humans. Examples of mammals include humans, livestock such as cows, goats, sheep, pigs, and rabbits, and companion animals such as dogs, cats, rabbits, and horses. Preferably, the patient or subject or mammal is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003; T. Higuchi and V. Stella, Pro-drugs as Novel Delivery Systems Vol. 14 of the A.C.S. Symposium Series; and Bioreversible Carriers in Drug Design, ed. Edward B. Roche, American Pharmaceutical Association and Pergamon Press, 1987). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as pharmaceutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “pharmaceutically acceptable salt,” as used herein, represents salts or zwitterionic forms of the compounds disclosed herein which are water or oil-soluble or dispersible and pharmaceutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides.

The compounds provided herein may optionally exist as pharmaceutically acceptable salts including pharmaceutically acceptable acid addition salts prepared from pharmaceutically acceptable non-toxic acids including inorganic and organic acids. Representative acids include, but are not limited to, acetic acid, benzenesulfonic acid, benzoic acid, camphorsulfonic acid, citric acid, ethenesulfonic acid, dichloroacetic acid, formic acid, fumaric acid, gluconic acid, glutamic acid, hippuric acid, hydrobromic acid, hydrochloric acid, isethionic acid, lactic acid, maleic acid, malic acid, mandelic acid, methanesulfonic acid, mucic acid, nitric acid, oxalic acid, pamoic acid, pantothenic acid, phosphoric acid, succinic acid, sulfuric acid, tartaric acid, oxalic acid, p-toluenesulfonic acid and the like.

Examples of inorganic acids which can be employed to form pharmaceutically acceptable addition salts include hydrochloric acid, hydrobromic acid, sulfuric acid, and phosphoric acid. Examples of organic acids which can be employed to form pharmaceutically acceptable addition salts include oxalic acid, maleic acid, succinic acid, and citric acid. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

Certain compounds provided herein which contain a carboxylic acid functional group may optionally exist as pharmaceutically acceptable salts containing non-toxic, pharmaceutically acceptable metal cations and cations derived from organic bases. Representative metals include, but are not limited to, aluminium, calcium, lithium, magnesium, potassium, sodium, zinc and the like. In some embodiments the pharmaceutically acceptable metal is sodium. Representative organic bases include, but are not limited to, benzathine (N¹,N2-dibenzylethane-1,2-diamine), chloroprocaine (2-(diethylamino)ethyl 4-(chloroamino)-benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et. al., Journal of Pharmaceutical Sciences, 66:1-19 (1977).

A salt of a compound can be made by reacting the appropriate compound in the form of the free base with the appropriate acid.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds provided herein may form solvates with standard low molecular weight solvents using methods known to the skilled artisan.

In addition, the compounds described herein can exist in unsolvated as well as solvated forms with pharmaceutically acceptable solvents such as water, ethanol, and the like. The solvated forms of the compounds presented herein are also considered to be disclosed herein.

It should be understood that a reference to a pharmaceutically acceptable salt includes the solvent addition forms. In some embodiments, solvates contain either stoichiometric or non-stoichiometric amounts of a solvent, and are formed during the process of isolation or crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, sites on the organic radicals (e.g., alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means. Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O³⁵, ¹⁸F, ³⁶C₁, ¹²³1, ¹²⁴1, ¹²⁵1, 131I. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the compounds described herein possess one or more stereocenters and each stereocenter exists independently in either the R or S configuration. In some embodiments, the compound exists in the R configuration. In some embodiments, the compound exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

### Pharmaceutical Compositions and Formulations

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound provided herein in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins, (Editors: Gennaro et. al.).

While it is possible that, for use in the prophylaxis or treatment, a compound provided herein may, in an alternative use, be administered as a raw or pure chemical, it is preferable however to present the compound or active ingredient as a pharmaceutical formulation or composition further comprising a pharmaceutically acceptable carrier.

Pharmaceutical formulations include those suitable for oral, rectal, nasal, topical (including buccal and sub-lingual), vaginal or parenteral (including intramuscular, subcutaneous and intravenous) administration or in a form suitable for administration by inhalation, insufflation or by a transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds provided herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds provided herein or a salt, solvate, or hydrate thereof can be used as active ingredients in pharmaceutical compositions, specifically as GLUT9 transporter modulators or inhibitors. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds provided herein can vary within wide limits and as is customary and is known to the physician or other clinician, it is to be tailored to the individual conditions in each individual case. It depends, for example, on the nature and severity of the illness to be treated, on the condition of the patient, on the compound employed or on whether an acute or chronic disease state is treated, or prophylaxis conducted, or on whether further active compounds are administered in addition to the compounds provided herein. Representative doses include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3, or 4 doses. Depending on the individual and as deemed appropriate from the healthcare provider it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated, or prophylaxis conducted, or on whether further active compounds are administered in addition to the compounds provided herein and as part of a drug combination. The dosage regimen for treating a disease condition with the compounds and/or compositions provided herein is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods provided herein.

The desired dose may conveniently be presented in a single dose or as divided doses administered at appropriate intervals, for example, as two, three, four, or more sub-doses per day. The sub-dose itself may be further divided, e.g., into a number of discrete loosely spaced administrations. The daily dose can be divided, especially when relatively large amounts are administered as deemed appropriate, into several, for example two, three, or four-part administrations. If appropriate, depending on individual behavior, it may be necessary to deviate upward or downward from the daily dose indicated.

The compounds provided herein can be administrated in a wide variety of oral and parenteral dosage forms. It will be obvious to those skilled in the art that the dosage forms may comprise, as the active component, either a compound provided herein or a pharmaceutically acceptable salt, hydrate, or solvate of a compound provided herein.

For preparing pharmaceutical compositions from the compounds provided herein, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is admixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethyl cellulose, a low melting wax, cocoa butter and the like. The term “preparation” refers to the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams, or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds provided herein may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethyl cellulose, or other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents and the like.

For topical administration to the epidermis the compounds provided herein may be formulated as ointments, creams, or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds provided herein or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds provided herein as an aerosol can be prepared by processes well known to the person skilled in the art. For their preparation, for example, solutions or dispersions of the compounds provided herein in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

Some embodiments include a method of producing a pharmaceutical composition for “combination-therapy” comprising admixing at least one compound according to any of the compound embodiments disclosed herein, together with at least one known pharmaceutical agent and a pharmaceutically acceptable carrier.

### Indications and Methods of Treatment

Also provided herein are methods for treating GLUT9-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of GLUT9-mediated disorders.

A GLUT9 inhibitor is considered to be an agent for the treatment or prophylaxis of pathological conditions that involve high blood uric acid levels; specifically, hyperuricemia, gout (for example, gouty arthritis, gouty kidney, and gouty tophus) and the like. Further, it is considered to have potential to be useful as an agent for the treatment or prophylaxis of pathological conditions which are generally known to have a complication with hyperuricemia and are particularly suggested to have association with high uric acid; specifically, chronic kidney disease (CKD), hypertension, diabetes, cardiac disease (for example, cardiovascular disease, cardiac failure, and atrial fibrillation), arteriosclerotic disease, non-alcoholic fatty liver disease (NAFLD), non-alcoholic steatohepatitis (NASH), psoriasis and the like.

Specific diseases to be treated by the compounds, compositions, and methods disclosed herein include hyperuricemia and gout.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

Accordingly, the present invention also relates to a method of inhibiting at least one GLUT9 function comprising the step of contacting GLUT9 with a compound as described herein. The cell phenotype, cell proliferation, activity of GLUT9, change in biochemical output produced by active GLUT9, expression of GLUT9, or binding of GLUT9 with a natural binding partner may be monitored. Such methods may be modes of treatment of disease, biological assays, cellular assays, biochemical assays, or the like.

Also provided herein is a method of treatment of a GLUT9-mediated disease comprising the administration of a therapeutically effective amount of a compound as disclosed herein, or a salt thereof, to a patient in need thereof.

A GLUT9 inhibitor is considered to be an agent for the treatment or prophylaxis of pathological conditions that involve high blood uric acid levels; specifically, hyperuricemia, gout (for example, gouty arthritis, gouty kidney, and gouty tophus) and the like. Further, it is considered to have potential to be useful as an agent for the treatment or prophylaxis of pathological conditions which are generally known to have a complication with hyperuricemia and are particularly suggested to have association with high uric acid; specifically, chronic kidney disease (CKD), hypertension, diabetes, cardiac disease (for example, cardiovascular disease, cardiac failure, and atrial fibrillation), arteriosclerotic disease, non-alcoholic fatty liver disease (NAFLD), non-alcoholic steatohepatitis (NASH), psoriasis and the like.

In an embodiment, the disease is selected from hyperuricemia, gout, and uncontrolled gout including comorbidities and associated diseases.

In certain embodiments, the disease is selected from hyperuricemia and gout.

Also provided herein is a compound as disclosed herein for use as a medicament.

Also provided herein is a compound as disclosed herein for use as a medicament for the treatment of a GLUT9-mediated disease.

Also provided is the use of a compound as disclosed herein as a medicament.

Also provided is the use of a compound as disclosed herein as a medicament for the treatment of a GLUT9-mediated disease.

Also provided is a compound as disclosed herein for use in the manufacture of a medicament for the treatment of a GLUT9-mediated disease.

Also provided is the use of a compound as disclosed herein for the treatment of a GLUT9-mediated disease.

Also provided herein is a method of inhibition of GLUT9 comprising contacting GLUT9 with a compound as disclosed herein, or a salt thereof.

Also provided herein is a method for achieving an effect in a patient comprising the administration of a therapeutically effective amount of a compound as disclosed herein, or a salt thereof, to a patient, wherein the effect is selected from cognition enhancement.

In certain embodiments, the GLUT9-mediated disease is selected from hyperuricemia and gout.

Also provided herein is a method for achieving an effect in a patient comprising the administration of a therapeutically effective amount of a compound described herein to a patient, wherein the effect is lowering blood uric acid levels.

Also provided is a method of modulation of a GLUT9-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the oral pharmaceutical composition is selected from a tablet and a capsule.

### Combination Therapy

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the invention with inhibitors of uric acid synthesis, uricosurics, and uric acid catabolizing agents.

In an embodiment, said uricosuric is selected from probenecid, lesinurad, benzbromarone, and sulfinpyrazone.

In an embodiment, said inhibitor of uric acid synthesis is selected from allopurinol and febuxostat.

In an embodiment, said uric acid catabolizing agent is pegloticase.

In an embodiment, said other agent is colchichine.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

### General Synthetic Schemes

The following schemes can be used to practice the present invention.

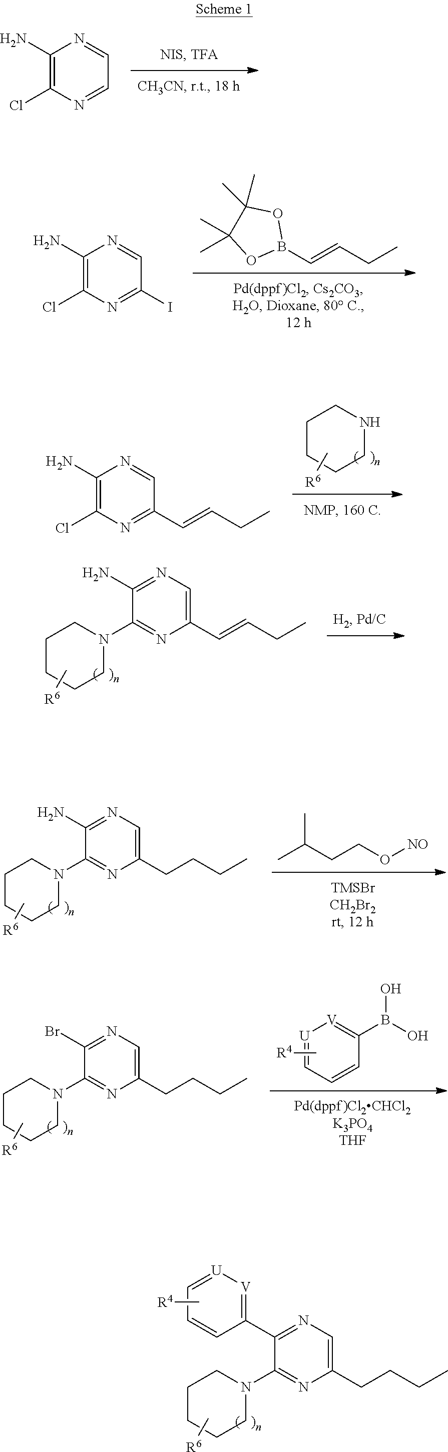

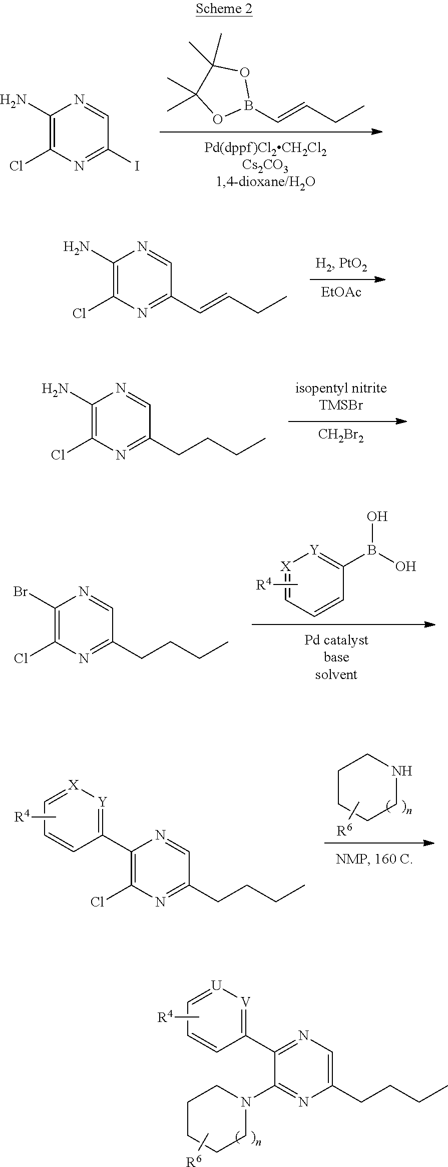

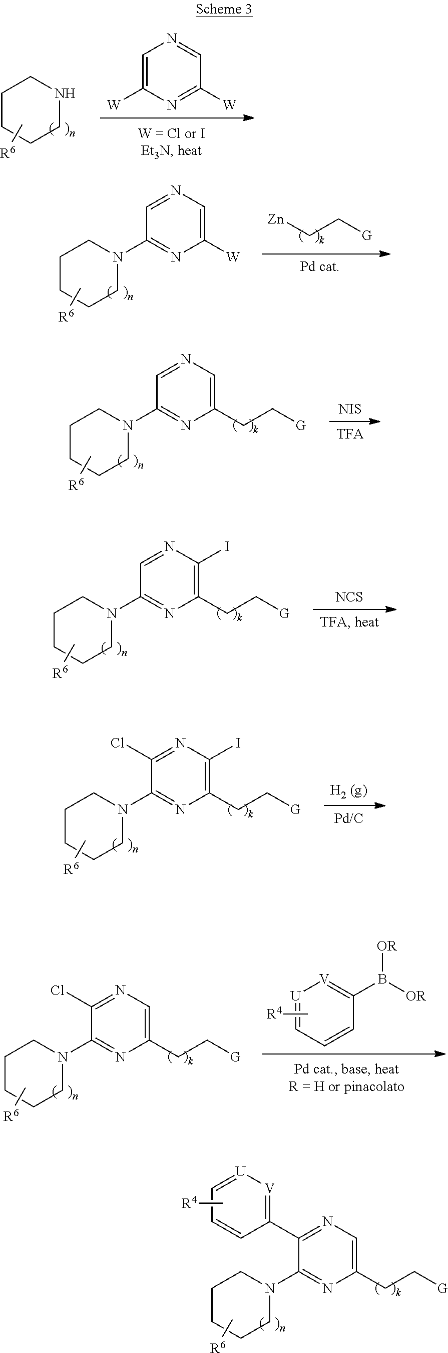

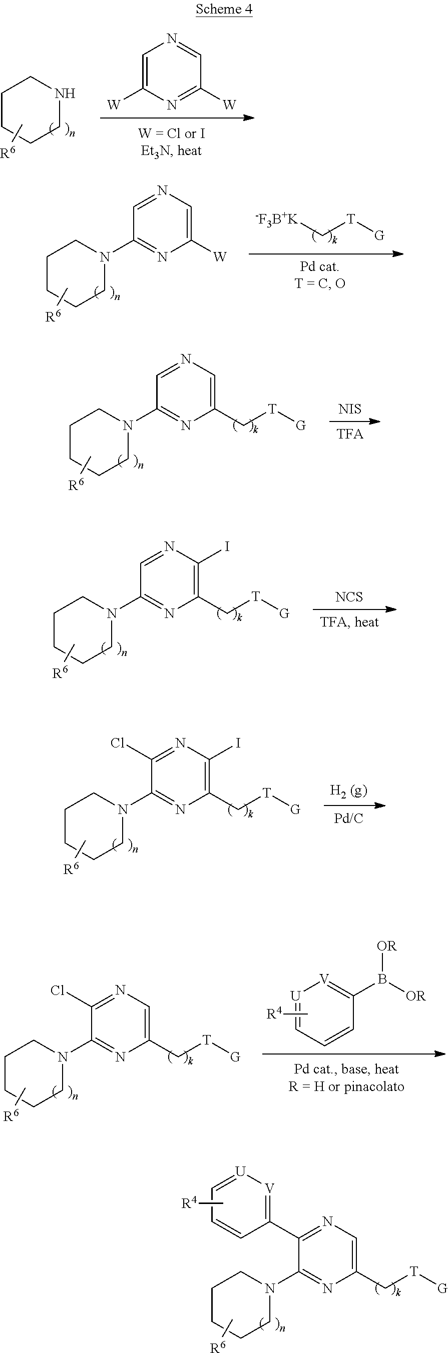

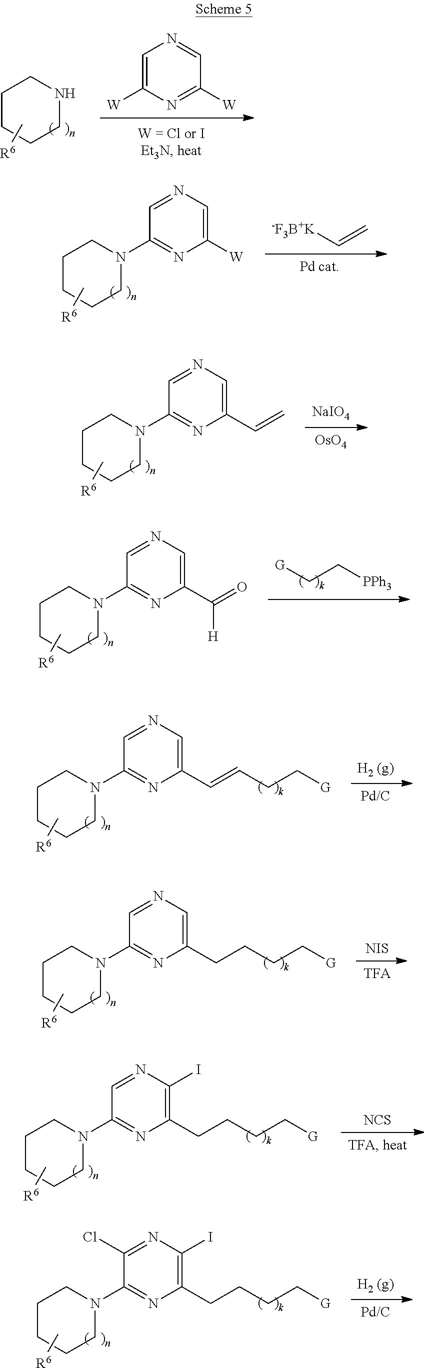

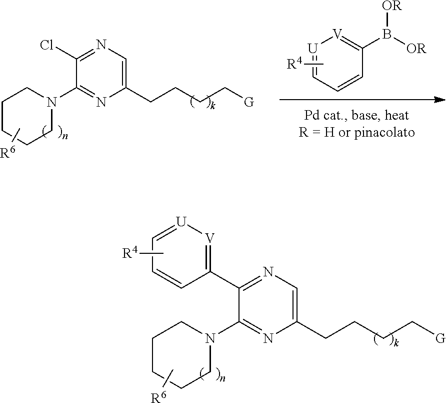

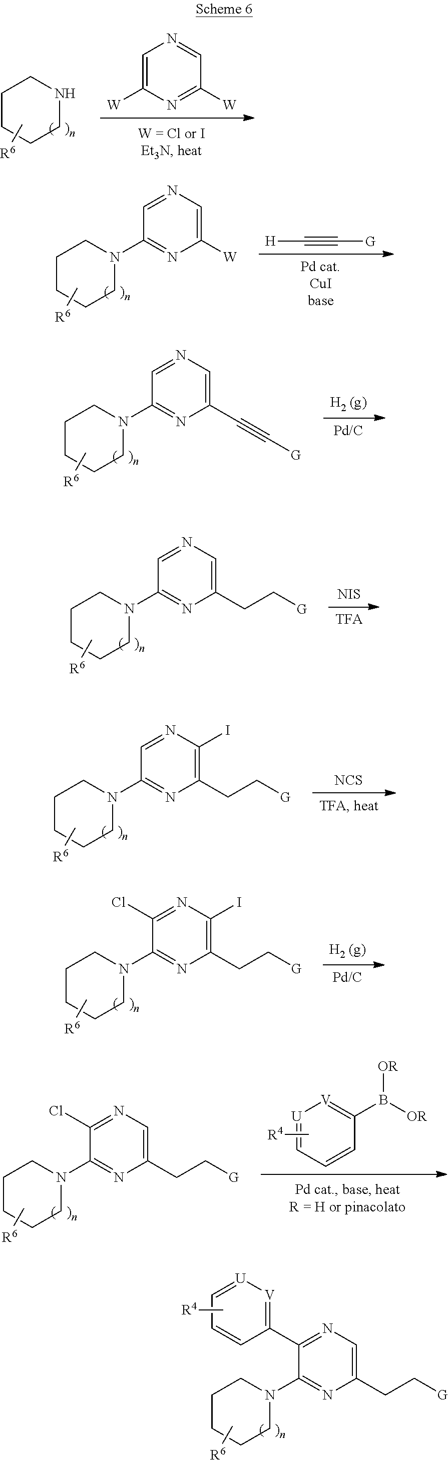

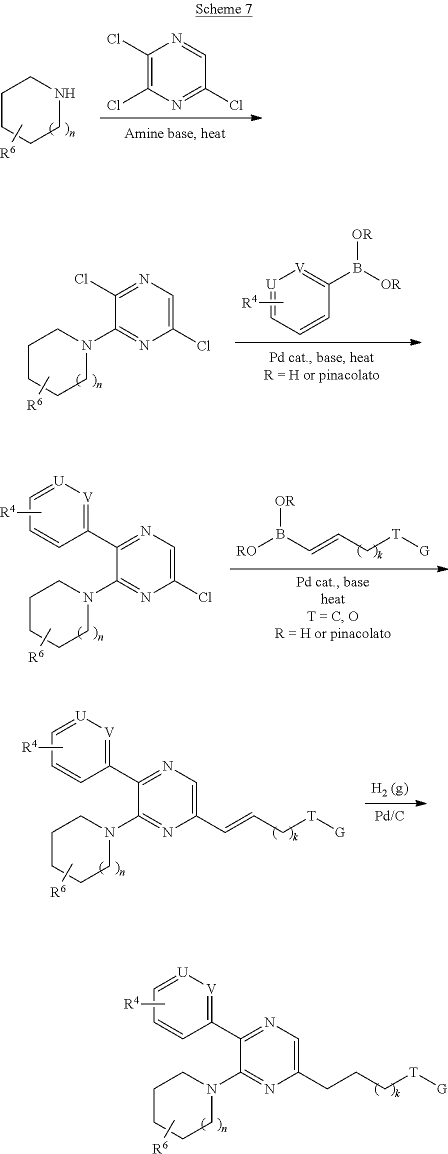

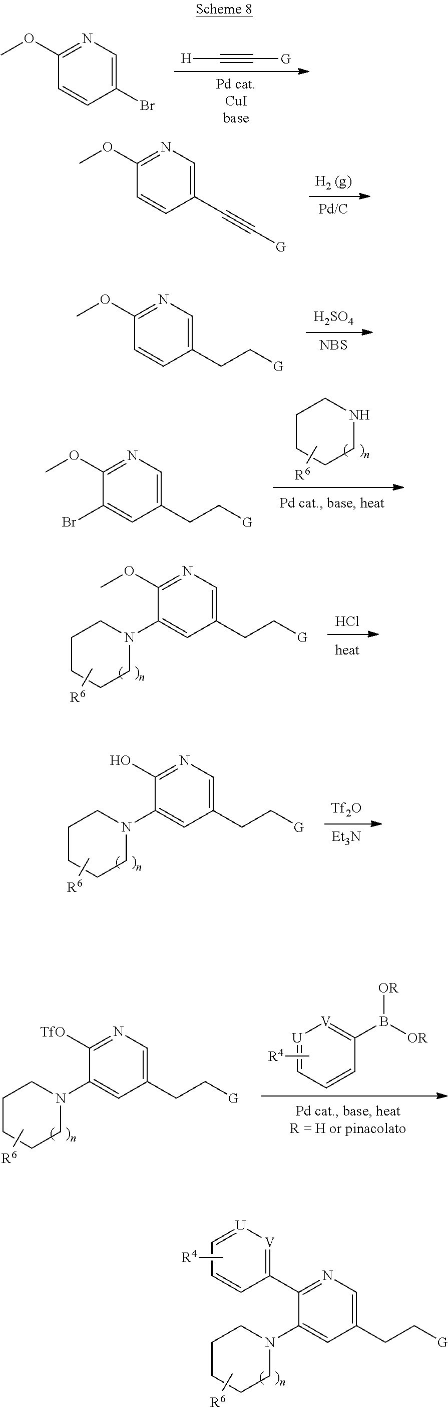

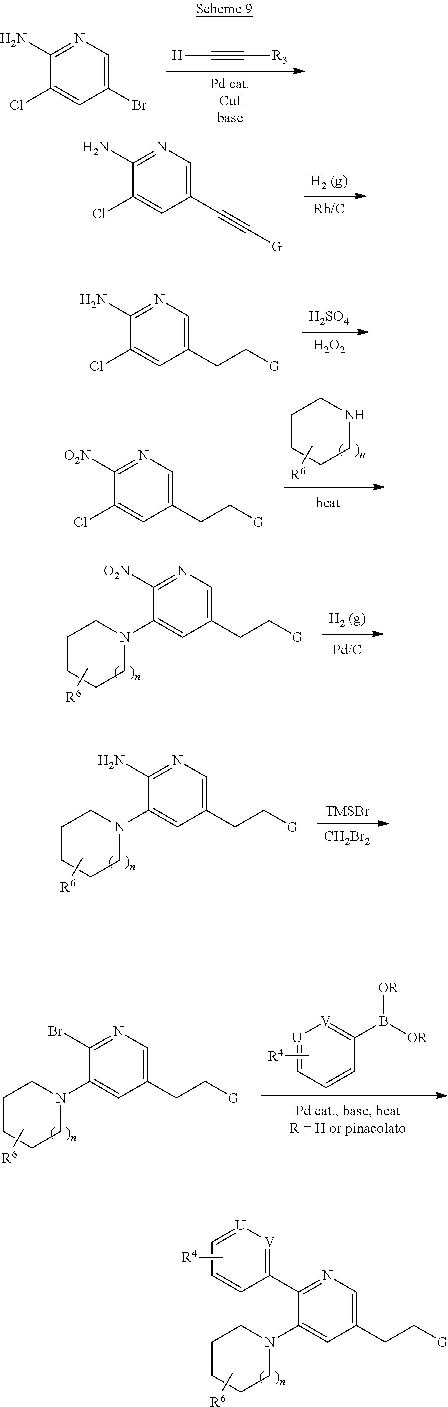

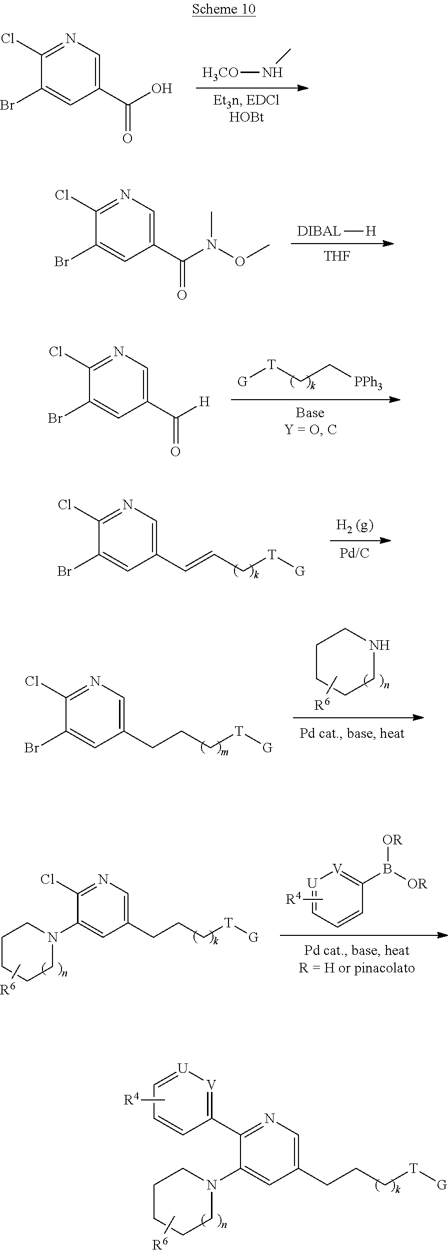

### EXAMPLES

The invention is further illustrated by the following examples. All IUPAC names were generated using CambridgeSoft's ChemDraw Ultra 11.0.1 or CambridgeSoft's ChemDraw Ultra 20.0.0.41.

### I. Chemical Synthesis

### List of Commonly Used Abbreviations

g (grams); mg (milligrams);
L (liters); mL (milliliters); p L (microliters);
psi (pounds per square inch);
M (molar); mM (millimolar); μM (micromolar);
MHz (megahertz);
mol (moles); mmol (millimoles);
rt (room temperature);
hr (hours);
min (minutes);
TLC (thin layer chromatography);
mp (melting point);
THF (tetrahydrofuran);
TFA (trifluoroacetic acid);
TFAA (trifluoroacetic anhydride);
Et₃N (triethylamine);
NMP (N-methyl-2-pyrrolidine)
CDCl₃ (deuterated chloroform);
N2(g) (nitrogen gas)
DMSO (dimethylsulfoxide);
SOC₂ (thionyl chloride)
SiO2 (silica);
atm (atmosphere);
EtOAc (ethyl acetate);
CHCl₃ (chloroform);
HCl (hydrochloric acid);
Ac (acetyl);
DMF (N,N-dimethylformamide);
Me (methyl);
Et (ethyl);
t-Bu (tert-butyl);
Pr (propyl);
i-Pr (isopropyl);
Cs₂CO₃ (cesium carbonate);
EtOH (ethanol);
MeOH (methanol);
CD₃GD (deuterated methanol);
p-TsOH (p-toluenesulfonic acid);
CDI (1,1′-carbonyldiimidazole);
NaHCO₃ (sodium bicarbonate);
DCM (dichloromethane);
DCE (1,2-dichloroethane);
K₂CO₃ (potassium carbonate);
Na₂CO₃ (sodium carbonate);
ACN or CH₃CN (acetonitrile);
PE (petroleum ether);
MTBE (methyl tert-butyl ether);
Hex (hexanes);
H₂SO₄ (sulfuric acid);
HCl (hydrochloric acid);
TMSBr (bromotrimethylsilane);
Na₂SO₄ (sodium sulfate);
Boc (tert-butoxycarbonyl);
DIPEA (diisopropylethylamine);
IPA (isopropanol);
HMDS (hexamethyldisilazane);
NH₄Cl (ammonium chloride);
K₃PO₄ (potassium phosphate tribasic);
(NH₄)HCO₃ (ammonium bicarbonate);
EDCI (1-Ethyl-3-(3-dimethylaminopropyl)carbodiimide);
NaOH (sodium hydroxide);
DMAP (4-dimethylaminopyridine);
TMSCl (chlorotrimethylsilane);
NIS (N-iodosuccinimide);
NCS (N-chlorosuccinimide);
CuI (copper (I) iodide);
KOAc (potassium acetate);
KF (potassium fluoride);
CsF (cesium fluoride);
NaN₃ (sodium azide);
TMP (2,2,6,6-tetramethylpiperidine);
K₂OsO₄ (potassium osmate);
NaIO₄ (sodium metaperiodate);
DAST ((diethylamino)sulfur trifluoride);
Pd(OAc)₂ (palladium acetate);
BF₃-(OEt)₂ (boron trifluoride diethyl etherate);
CS₂ (carbon disulfide);
Tf₂O (trifluoromethanesulfonic anhydride);
TMSCN (trimethylsilyl cyanide);
CDI (1,1′-carbonyldiimidazole);
LiAlH₄ (lithium aluminum hydride);
TMSN₃ (trimethylsilyl azide);
DMA (N,N-dimethylacetamide);
FA (formic acid);
NH₂OH- HCl (hydroxylamine hydrochloride);
DEAD (diethyl azodicarboxylate);
PyBOP (benzotriazol-1-yloxytripyrrolidinophosphonium hexafluorophosphate);
Pd(PPh₃)₂Cl₂ (Bis(triphenylphosphine)palladium chloride);
Pd(dppf)Cl₂ ([1,1′-Bis(diphenylphosphino)ferrocene]dichloropalladium(II));
Pd(dppf)Cl₂—CH₂Cl₂ ([1,1′-Bis(diphenylphosphino)ferrocene]palladium(II) dichloride dichloromethane adduct);
Pd(PPh₃)₄(Tetrakis(triphenylphosphine)palladium(0).

### Synthetic Methods

The following intermediates and examples can be synthesized using the general synthetic procedures set forth in Schemes 1-10.

### Intermediate 1. (E)-5-(but-1-en-1-yl)-3-chloropyrazin-2-amine

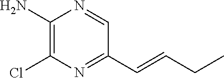

To a mixture of 3-chloro-5-iodo-pyrazin-2-amine (18.0 g, 70.47 mmol, 1.0 equiv) and 2-[(E)-but-1-enyl]-4,4,5,5-tetramethyl-1,3,2-dioxaborolane (15.0 g, 82.39 mmol, 1.2 equiv) in H₂O (90 mL) and 1,4-dioxane (270 mL) at rt was added Pd(dppf)Cl₂-CH₂Cl₂ (5.75 g, 7.05 mmol, 0.1 equiv) and Cs₂CO₃ (68.88 g, 211.40 mmol, 3.0 equiv). The resulting reaction mixture was heated to 80° C. and stirred for 12 hr. The reaction mixture was then cooled to rt, diluted with H₂O (300 mL), and then extracted with EtOAc (2×400 mL). The combined organic layers were washed with brine (100 mL), dried over Na₂SO₄, filtered, and concentrated under reduced pressure to give a residue. The residue was purified by silica gel column chromatography (PE: EtOAc=20:1 to 3:1) to afford (E)-5-(but-1-en-1-yl)-3-chloropyrazin-2-amine (22.4 g, 86% yield) as a yellow solid. ¹H NMR (400 MHz, CDCl₃) δ 7.84 (s, 1H), 6.61 (dt, J=6.8, 15.6, 1H), 6.28 (d, J=15.6, 1H), 4.90 (s, br, 2H), 2.23 (m, 2 H), 1.08 (t, J=7.6, 3H); LCMS calculated for CsHioClN₃: m/z=183; found: m/z=184 (M+H).

### Intermediate 2. Methyl (E)-1-(3-amino-6-(but-1-en-1-yl)pyrazin-2-yl)piperidine-4-carboxylate

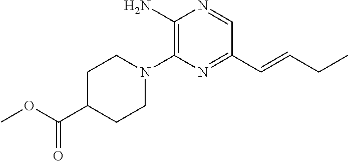 [SMILES: CC/C=C/c1cnc(N)c(N2CCC(C(=O)OC)CC2)n1]

To a solution of (E)-5-(but-1-en-1-yl)-3-chloropyrazin-2-amine (Intermediate 1) (4.0 g, 21.78 mmol, 1.0 equiv) in NMP (80 mL) at rt was added methyl piperidine-4-carboxylate (31.19 g, 217.82 mmol, 10.0 equiv). The resulting reaction mixture was heated at 160° C. for 3 hr. The reaction mixture was cooled to rt, diluted with H₂O (100 mL), and then extracted with EtOAc (2×100 mL). The combined organic layers were washed with brine (1×10 mL), dried over Na₂SO₄, filtered, and concentrated under reduced pressure to afford crude methyl (E)-1-(3-amino-6-(but-1-en-1-yl)pyrazin-2-yl)piperidine-4-carboxylate (ca. 2 g). The aqueous phase was adjusted pH to 5 with saturated aqueous citric acid (5 mL) and then extracted with EtOAc (2×10 mL). The combined organic layers were washed with brine (1×10 mL), dried over Na₂SO₄, filtered, and concentrated under reduced pressure to afford (E)-1-(3-amino-6-(but-1-en-1-yl)pyrazin-2-yl)piperidine-4-carboxylic acid (5 g, crude) were obtained as a yellow oil. The crude acid was dissolved in MeOH (500 mL) and the resulting solution was treated with SOCl₂ (6.46 g, 54.28 mmol, 3.94 mL, 3.0 equiv). The resulting reaction mixture was heated to 70° C. and stirred for 1 hr. The reaction mixture was cooled to rt and treated with saturated aqueous NaHCO₃ (50 mL), and the resulting mixture was extracted with EtOAc (2×10 mL). The combined organic layers were washed with brine (1×10 mL), dried over Na₂SO₄, filtered, and concentrated under reduced pressure to give additional crude methyl (E)-1-(3-amino-6-(but-1-en-1-yl)pyrazin-2-yl)piperidine-4-carboxylate, which was combined with the material obtained above and purified by silica gel column chromatography (PE:EtOAc=50:1 to 15:1) to afford pure methyl (E)-1-(3-amino-6-(but-1-en-1-yl)pyrazin-2-yl)piperidine-4-carboxylate (3.5 g, 58% yield) as a yellow solid. ¹H NMR (400 MHz, CD₃OD) δ 7.44 (s, 1H), 6.57 (m, 1H), 6.28 (d, J=15.6, 1H), 3.72 (s, 3 H), 3.56 (m, 2H), 2.58 (m, 1H), 2.36 (m, 2H), 2.23 (m, 2H), 2.09 (m, 2H), 1.93 (m, 2H), 1.10 (t, J=7.2, 3H); LCMS calculated for C₁₅H₂₂N₄O₂: m/z=290; found: m/z=291 (M+H).

### Intermediate 3. Methyl 1-(3-amino-6-butylpyrazin-2-yl)piperidine-4-carboxylate

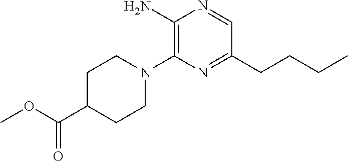 [SMILES: CCCCc1cnc(N)c(N2CCC(C(=O)OC)CC2)n1]

To a solution of methyl (E)-1-(3-amino-6-(but-1-en-1-yl)pyrazin-2-yl)piperidine-4-carboxylate (Intermediate 2) (6.0 g, 20.66 mmol, 1.0 equiv) in MeOH (50 mL) at rt was added Pd/C (2.0 g, 10% weight). The resulting reaction mixture was degassed under vacuum and purged with H₂(g) several times. The reaction mixture was then stirred under H₂ (15 psi) at rt for 12 hr. The reaction mixture was then purged several times with N₂(g), filtered through a pad of celite to remove the Pd catalyst, and concentrated under reduced pressure to give a residue. The residue was purified by silica gel column chromatography (PE:EtOAc=20:1 to 1:1) to afford methyl 1-(3-amino-6-butylpyrazin-2-yl)piperidine-4-carboxylate carboxylate (4.3 g, 71% yield) as a yellow solid. ¹H NMR (400 MHz, CDCl₃) δ 7.47 (s, 1H), 4.50 (s, br, 2H), 3.69 (s, 3H) 3.51 (m, 2H), 2.83 (m, 3H), 2.55 (t, J=7.6, 2H), 2.02 (m, 2 H), 1.85 (m, 2H), 1.60 (m, 2H), 1.33 (m, 2H), 0.91 (t, J=7.2, 3H); LCMS calculated for C₁₅H₂₄N₄O₂: m/z=292; found: m/z=293 (M+H).

### Intermediate 4. Methyl 1-(3-bromo-6-butylpyrazin-2-yl)piperidine-4-carboxylate

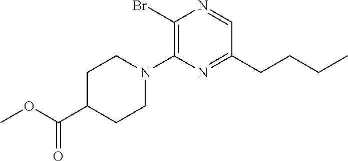 [SMILES: CCCCc1cnc(Br)c(N2CCC(C(=O)OC)CC2)n1]

To a solution of methyl 1-(3-amino-6-butylpyrazin-2-yl)piperidine-4-carboxylate (Intermediate 3) (0.45 g, 1.54 mmol, 1.0 equiv) in dibromomethane (30 mL) at rt under N₂(g) was added isopentyl nitrite (198 mg, 1.69 mmol, 228 μL, 1.1 equiv), followed by a solution of TMSBr (259 mg, 1.69 mmol, 220 μL, 1.1 equiv) in dibromomethane (15 mL) at rt. The resulting reaction mixture was stirred for 16 hr at rt. The reaction mixture was treated with H₂O (10 mL), and then extracted with EtOAc (2×10 mL). The combined organic layers were washed with brine (1×10 mL), dried over Na₂SO₄, filtered, and concentrated under reduced pressure to give a residue. The residue was purified by preparative thin-layer chromatography (PE:EtOAc=8:1 to afford methyl 1-(3-bromo-6-butylpyrazin-2-yl)piperidine-4-carboxylate (0.6 g, 55% yield) as a yellow oil. LCMS calculated for C₁₅H₂₂BrN₃O₂: m/z=355; found: m/z=356 (M+H).

### Intermediate 5.5-butyl-3-chloropyrazin-2-amine

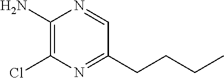

To a solution of (E)-5-(but-1-en-1-yl)-3-chloropyrazin-2-amine (Intermediate 1) (3.0 g, 16.34 mmol, 1.0 equiv) in EtOAc (120 mL) at rt was added PtO₂ (370 mg, 1.63 mmol, 0.1 equiv) under N₂(g). The suspension was degassed under vacuum and purged with H₂ several times. The resulting reaction mixture was stirred under H₂(g) (15 psi) at rt for 3 hr. The reaction mixture was purged with N₂(g) several times then filtered to remove the catalyst and concentrated under reduced pressure to give 5-butyl-3-chloropyrazin-2-amine (3.0 g, crude) as yellow liquid. LCMS calculated for CsH₁₂ClN₃: m/z=185; found: m/z=186 (M+H).

### Intermediate 6. 2-bromo-5-butyl-3-chloropyrazine

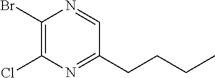

To a solution of crude 5-butyl-3-chloropyrazin-2-amine (Intermediate 5) (3.0 g, 16.16 mmol, 1.0 equiv) in dibromomethane (18 mL) at rt under an N₂(g) atmosphere was added isopentyl nitrite (3.79 g, 32.32 mmol, 4.35 mL, 2.0 equiv) in one portion, followed by addition of a solution of TMSBr (4.95 g, 32.32 mmol, 4.19 mL, 2.0 equiv) in dibromomethane (27 mL). The resulting reaction mixture was stirred at rt for 12 hr. The reaction mixture was then poured into H₂O (100 mL) and extracted with EtOAc (2×200 mL). The combined organic phase was dried with anhydrous Na₂SO₄, filtered, and concentrated under vacuum. The residue was purified by silica gel column chromatography (PE: EtOAc=20:1 to 10:1) to afford 2-bromo-5-butyl-3-chloropyrazine (3.1 g, 78% yield) as a yellow liquid. ¹H NMR: (400 MHz, CD₃OD) δ8.24 (s, 1H), 2.76 (t, J=7.6, 2H), 1.69 (m, 2H), 1.38 (m, 2H), 0.94 (t, J=7.6, 3H); LCMS calculated for CsHioBrClN₂: m/z=249; found: m/z=250 (M+H).

### Intermediate 7. 5-butyl-3-chloro-2-(4-methoxyphenyl)pyrazine

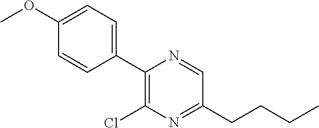 [SMILES: CCCCc1cnc(-c2ccc(OC)cc2)c(Cl)n1]

To a mixture of 2-bromo-5-butyl-3-chloropyrazine (Intermediate 6) (2.5 g, 10.02 mmol, 1.0 equiv) and (4-methoxyphenyl)boronic acid (1.37 g, 9.02 mmol, 0.9 equiv) in dioxane (25 mL) and H₂O (5 mL) at rt under an N₂(g) atmosphere was added Pd(dppf)Cl₂—CH₂Cl₂ (818 mg, 1.00 mmol, 0.1 equiv) and K₂CO₃ (2.77 g, 20.04 mmol, 2.0 equiv). The resulting reaction mixture was heated to 80° C. and stirred for 12 hr. The reaction mixture was cooled to rt, diluted with H₂O (100 mL), and then extracted with EtOAc (2×100 mL). The combined organic layers were dried over Na₂SO₄, filtered, and concentrated under reduced pressure to give a residue. The residue was purified by silica gel column chromatography (PE:EtOAc=20:1 to 10:1) to afford 5-butyl-3-chloro-2-(4-methoxyphenyl)pyrazine (2.3 g, 83% yield) as a yellow oil. ¹H NMR (400 MHz, CD₃OD) δ 8.48 (s, 1H), 7.73 (d, J=8.4, 2H), 7.05 (d, J=8.8, 2H), 3.88 (s, 3H), 2.84 (t, J=8.0, 2H), 1.77 (m, 2H), 1.44 (m, 2H), 1.00 (t, J=7.2, 3H); LCMS calculated for C₁₅H₁₇ClN₂O: m/z=276; found: m/z=277 (M+H).

### Intermediate 8. Ethyl 1-(6-chloropyrazin-2-yl)piperidine-4-carboxylate

To a solution of 2,6-dichloropyrazine (20.00 g, 134.25 mmol, 1.0 eq) and ethyl piperidine-4-carboxylate (22.16 g, 140.96 mmol, 1.1 eq) in 1,4-dioxane (600 mL) at rt was added Et₃N (14.94 g, 147.64 mmol, 1.1 eq). The resulting reaction mixture was heated to 100° C. under an N2 (g) atmosphere with stirring for 12 hr. The reaction mixture was then cooled to rt and concentrated under reduced pressure to remove most of the 1,4-dioxane. The residue was poured into H₂O (700 mL) and extracted with EtOAc (1400 mL). The organic phase was washed with brine (100 mL), dried with anhydrous Na₂SO₄, filtered, and concentrated under vacuum. The residue was purified by silica gel column chromatography (PE: EtOAc=10:1 to 5:1) to afford ethyl 1-(6-chloropyrazin-2-yl)piperidine-4-carboxylate (29.50 g, 74% yield) as yellow oil. ¹H NMR (400 MHz, CDCl₃) δ 7.98 (s, 1H), 7.77 (s, 1H), 4.19 (m, 4H), 3.07 (m, 2H), 2.58 (m, 1H), 2.03 (m, 2H), 1.75 (m, 2H), 1.27 (t, J=7.2, 3H); LCMS calculated for C₁₂H₁₆ClN₃O₂: m/z=269; found: m/z=270 (M+H).

### Intermediate 9. Ethyl 1-(6-(4,4,4-trifluorobutyl)pyrazin-2-yl)piperidine-4-carboxylate

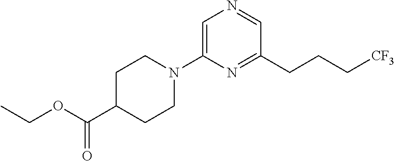 [SMILES: CCOC(=O)C1CCN(c2cncc(CCCC(F)(F)F)n2)CC1]

To a mixture of Zn (16.82 g, 257.23 mmol, 3.0 eq) in THF (180 mL) at rt under an N₂ (g) atmosphere was added 1,2-dibromoethane (3.16 g, 16.81 mmol, 1.27 mL, 0.2 eq). The resulting reaction mixture was heated to 80° C. and stirred for 5 min, then cooled back to rt. This heating-cooling cycle was repeated three additional times. Next, TMSCl (547 mg, 5.04 mmol, 0.06 eq) was added to the mixture and the resulting reaction mixture was stirred at rt for 10 min. The reaction mixture was then cooled to 0° C., and a solution of 1,1,1-trifluoro-4-iodobutane (20 g, 84.04 mmol, 1.0 eq) in THF (180 mL) was added dropwise to the mixture over 15 minutes. The resulting reaction mixture was warmed to rt and stirred for 15 min. Next, a solution of ethyl 1-(6-chloropyrazin-2-yl)piperidine-4-carboxylate (Intermediate 8) (9.56 g, 35.43 mmol, 1.0 eq) in THF (300 mL) was added to the reaction mixture. The reaction mixture was degassed and purged with N₂ (g) 3 times, and then Pd(dppf)Cl₂-CH₂Cl₂ (2.89 g, 3.54 mmol, 0.1 eq) was added. The resulting reaction mixture was heated to 60° C. and stirred for 12 hr. The reaction mixture was then cooled to rt and concentrated under reduced pressure to remove THF. The reaction mixture was poured into H₂O (200 mL) and extracted with EtOAc (3×200 mL). The combined organic phases were washed with brine (400 mL), dried with anhydrous Na₂SO₄, filtered, and concentrated under vacuum. The residue was purified by silica gel column chromatography (PE: EtOAc=5:1 to 1:1) to afford ethyl 1-(6-(4,4,4-trifluorobutyl) pyrazin -2-yl)piperidine-4-carboxylate (14 g, 60% yield) as a brown oil. ¹H NMR (400 MHz, CDCl₃) δ=8.13 (s, 1H), 7.68 (s, 1H), 4.26 (d, J=13.6, 2 H), 4.13 (q, J=7.2, 2H), 3.12 (m, 2H), 2.73 (t, J=7.6, 2H), 2.61 (m, 1H), 2.16 (m, 2H), 2.05 (m, 4H), 1.76 (m, 2H), 1.29 (t, J=7.2, 3H); LCMS calculated for C₁₆H₂₂F3N₃O₂: m/z=345; found: m/z=346 (M+H).

### Intermediate 10. Ethyl 1-(5-iodo-6-(4,4,4-trifluorobutyl)pyrazin-2-yl)piperidine-4-carboxylate

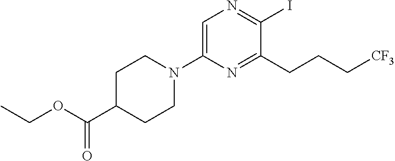 [SMILES: CCOC(=O)C1CCN(c2cnc(I)c(CCCC(F)(F)F)n2)CC1]

To a solution of ethyl 1-(6-(4,4,4-trifluorobutyl)pyrazin-2-yl)piperidine-4-carboxylate (Intermediate 9) (74 g, 214.26 mmol, 1.0 eq) in DMF (1000 mL) at rt was added TFA (24.44 g, 214.26 mmol, 1.0 eq) and NIS (62.66 g, 278.56 mmol, 1.3 eq). The resulting reaction mixture was stirred at rt for 2 hr. The reaction mixture was then adjusted to pH=8 with saturated aqueous NaHCO₃ solution and then the reaction mixture was extracted with EtOAc (3×500 mL). The combined organic layers were washed with brine (1000 mL), dried over Na₂SO₄, filtered, and concentrated under vacuum. The residue was purified by silica gel column chromatography (PE: EtOAc=1:0 to 5:1) to afford ethyl 1-(5-iodo-6-(4,4,4-trifluorobutyl)pyrazin-2-yl)piperidine-4-carboxylate (49 g, 45% yield) as a brown oil. ¹H NMR (400 MHz, CDCl₃) δ=7.77 (s, 1H), 4.19 (m, 4H), 3.05 (m, 2H), 2.83 (t, J=7.4, 2 H), 2.57 (m, 1H), 2.19 (m, 2H), 2.01 (m, 4H), 1.73 (m, 2H), 1.27 (t, J=7.2, 3H); LCMS calculated for C₁₆H₂₁F₃IN₃O₂: m/z=471; found: m/z=472 (M+H).

### Intermediate 11. Ethyl 1-(3-chloro-5-iodo-6-(4,4,4-trifluorobutyl)pyrazin-2-yl)piperidine-4-carboxylate

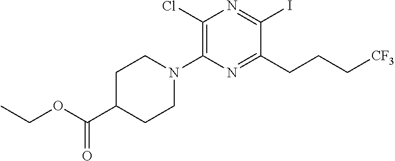 [SMILES: CCOC(=O)C1CCN(c2nc(CCCC(F)(F)F)c(I)nc2Cl)CC1]

To a solution of ethyl 1-(5-iodo-6-(4,4,4-trifluorobutyl)pyrazin-2-yl)piperidine-4-carboxylate (Intermediate 10) (44 g, 93.36 mmol, 1.0 eq) in DMF (500 mL) at rt was added NCS (18.70 g, 140.06 mmol, 1.5 eq) and TFA (10.64 g, 93.36 mmol, 1.0 eq). The resulting reaction mixture was heated to 100° C. and stirred for 1 hr. The reaction mixture was then cooled to rt and adjusted pH=8 with saturated aqueous NaHCO₃, and the resulting mixture was extracted with EtOAc (3×500 mL). The combined organic layers were washed with brine (400 mL), dried over Na₂SO₄, filtered, and concentrated under vacuum. The residue was purified by silica gel column chromatography (PE:EtOAc=1:0 to 5:1) to afford Ethyl 3-(3-chloro-5-iodo-6-(3,3,3-trifluoropropyl)pyrazin-2-yl)-3-azabicyclo[3.1.1]heptane-6-carboxylate (49 g, 93% yield) as a yellow oil. LCMS calculated for C₁₆H₂₀ClF₃IN₃O₂: m/z=489; found: m/z=506 (M+H).

### Intermediate 12. Ethyl 1-(3-chloro-6-(4,4,4-trifluorobutyl)pyrazin-2-yl)piperidine-4-carboxylate

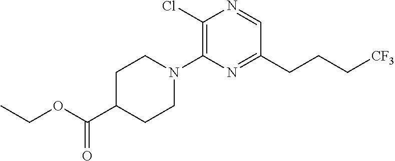 [SMILES: CCOC(=O)C1CCN(c2nc(CCCC(F)(F)F)cnc2Cl)CC1]

To a solution of ethyl 1-(3-chloro-5-iodo-6-(4,4,4-trifluorobutyl)pyrazin-2-yl)piperidine-4-carboxylate (Intermediate 11) (36 g, 73.19 mmol, 1.0 eq) in EtOH (300 mL) at rt under an N₂ (g) atmosphere was added 10% Pd/C (30 g). The resulting suspension was degassed and purged with H₂ (g) for 3 times. The resulting reaction mixture was stirred at rt under an H₂ (g) atmosphere (30 psi) for 12 hr. The mixture was then purged with N₂ (g) three times and then filtered through Celite to remove the catalyst. The filtrate was concentrated under reduced pressure to give a residue. The residue was purified by silica gel column chromatography (PE:EtOAc=1:0 to 3:1) to afford ethyl 1-(3-chloro-6-(4,4,4-trifluorobutyl)pyrazin-2-yl)piperidine-4-carboxylate (13 g, 45% yield) as a yellow oil. ¹H NMR (400 MHz, CDCl₃) δ=7.72 (s, 1H), 4.17 (q, J=7.2, 2H), 3.95 (d, J=13.6, 2H), 2.96 (m, 2H), 2.75 (t, J=7.2, 2H), 2.54 (m, 1H), 2.18 (m, 2H), 2.02 (m, 4H), 1.90 (m, 2H), 1.29 (t, J=7.2, 3H); LCMS calculated for C₁₆H₂₁ClF₃N₃O₂: m/z=379; found: m/z=380 (M+H).

### Intermediate 13. Ethyl 1-(6-((2,2,2-trifluoroethoxy)methyl)pyrazin-2-yl)piperidine-4-carboxylate

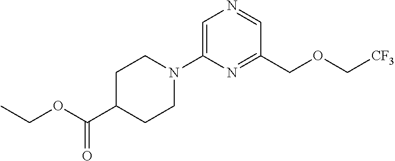 [SMILES: CCOC(=O)C1CCN(c2cncc(COCC(F)(F)F)n2)CC1]

To a slurry of NaH (14.94 g, 373.44 mmol, 60% purity, 3.0 eq) in THF (1125 mL) at 0° C. under an N₂ (g) atmosphere was added 2,2,2-trifluoroethanol (37.36 g, 373.44 mmol, 3.0 eq). The resulting reaction mixture was warmed to rt and stirred for 1 hr. Then the resulting reaction mixture was recooled to 0° C., and potassium trifluoro((2,2,2-trifluoroethoxy)methyl)borate (25.00 g, 124.48 mmol, 1.0 eq) was added. The resulting reaction mixture was warmed to rt and stirred for 11 hr. The reaction mixture was cooled to 0° C. and quenched with potassium hydrogen fluoride (200 mL, 4. μM), and the resulting reaction mixture was stirred for 30 min. The suspension was concentrated under reduced pressure to afford a residue. The residue was triturated with MTBE (2×600 mL) at rt for 30 min. The resulting slurry was filtered and the solid was collected and dried under vacuum. The crude product was triturated with acetonitrile (200 mL) at rt for 30 min. The resulting slurry was filtered and the filtrate was concentrated in vacuo to afford potassium (2,2,2-trifluoroethoxymethyl)trifluoroborate (27.00 g, crude) as a white solid. Next, a solution of ethyl 1-(6-chloropyrazin-2-yl)piperidine-4-carboxylate (Intermediate 8) (10.00 g, 37.07 mmol, 1.0 eq) and potassium (2,2,2-trifluoroethoxymethyl)trifluoroborate (12.23 g, 55.61 mmol, 1.5 eq) in 1,4-dioxane (100 mL) and H₂O (20 mL) at rt under an N₂ (g) atmosphere was added Pd(dppf)Cl₂-CH₂Cl₂ (1.51 g, 1.85 mmol, 0.05 eq) and Cs₂CO₃ (36.24 g, 111.22 mmol, 3.0 eq). The resulting reaction mixture was heated to 110° C. and stirred for 12 hr. The reaction mixture was then cooled to rt and concentrated under reduced pressure to remove most of the dioxane. The residue was poured into H₂O (300 mL) and extracted with EtOAc (3×300 mL). The combined organic phases were washed with brine (100 mL), dried with anhydrous Na₂SO₄, filtered, and concentrated under vacuum. The residue was purified by silica gel column chromatography (PE: EtOAc=1:0 to 1:1) to afford ethyl 1-(6-((2,2,2-trifluoroethoxy)methyl)pyrazin-2-yl)piperidine-4-carboxylate (9.5 g, 73% yield) as a yellow solid. ¹H NMR (400 MHz, CDCl₃) δ 8.02 (s, 1H), 7.85 (s, 1H), 4.58 (s, 2H), 4.18 (m, 2H), 4.07 (m, 2H), 3.90 (q, J=8.8, 2H), 2.97 (m, 2H), 2.51 (m, 1H), 1.96 (m, 3H), 1.68 (m, 2 H), 1.22-1.73 (t, J=8.8, 3H).

### Intermediate 14. Ethyl 1-(5-chloro-6-((2,2,2-trifluoroethoxy)methyl)pyrazin-2-yl)piperidine-4-carboxylate

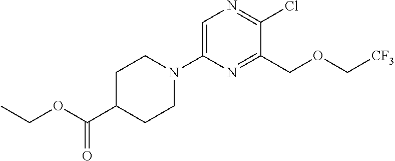 [SMILES: CCOC(=O)C1CCN(c2cnc(Cl)c(COCC(F)(F)F)n2)CC1]

To a solution of ethyl 1-(6-((2,2,2-trifluoroethoxy)methyl)pyrazin-2-yl)piperidine-4-carboxylate (Intermediate 13) (1.50 g, 4.32 mmol, 1.0 eq) in DMF (15 mL) at rt under an N₂ (g) atmosphere was added NCS (519 mg, 3.89 mmol, 0.9 eq) and TFA (49 mg, 431 μmol, 0.1 eq). The resulting reaction mixture was then heated at 60° C. and stirred for 1 hr. The reaction mixture was then cooled to rt. The residue was poured into H₂O (50 mL) and extracted with EtOAc (3×50 mL). The combined organic phases were washed with brine (50 mL), dried with anhydrous Na₂SO₄, filtered, and concentrated under vacuum. The residue was purified by silica gel column chromatography (PE: EtOAc=1:0 to 3:1) to afford ethyl 1-(5-chloro-6-((2,2,2-trifluoroethoxy)methyl)pyrazin-2-yl)piperidine-4-carboxylate (720 mg, 43% yield) as a white solid. LCMS calculated for C₁₅H₁₉ClF₃N₃O₃: m/z=381; found: m/z=382 (M+H).

### Intermediate 15. Ethyl 1-(5-chloro-3-iodo-6-((2,2,2-trifluoroethoxy)methyl)pyrazin-2-yl)piperidine-4-carboxylate

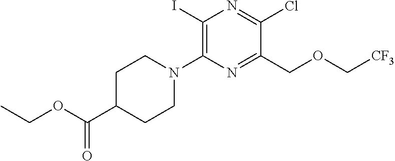 [SMILES: CCOC(=O)C1CCN(c2nc(COCC(F)(F)F)c(Cl)nc2I)CC1]

To a solution of ethyl 1-(5-chloro-6-((2,2,2-trifluoroethoxy)methyl)pyrazin-2-yl)piperidine-4-carboxylate (Intermediate 14) (620 mg, 1.62 mmol, 1.0 eq) in CHCl₃ (10 mL) at rt under an N₂ (g) atmosphere was added NIS (730 mg, 3.25 mmol, 2.0 eq) and TFA (18 mg, 162 μmol, 12 μL, 0.1 eq). The resulting reaction mixture was then heated at 80° C. and stirred for 0.5 hr. The reaction mixture was cooled to rt and was then poured into H₂O (30 mL) and extracted with EtOAc (3×30 mL). The combined organic phase was washed with brine (30 mL), dried with anhydrous Na₂SO₄, filtered, and concentrated under vacuum. The residue was purified by silica gel column chromatography (PE:EtOAc=1:0 to 5:1) to afford ethyl 1-(5-chloro-3-iodo-6-((2,2,2-trifluoroethoxy)methyl)pyrazin-2-yl)piperidine-4-carboxylate (780 mg, 94% yield) as a yellow oil. ¹H NMR (400 MHz, CDCl₃) δ 4.68 (s, 2H), 4.10 (q, J=7.2, 2H), 3.94 (dd, J=8.4, 16.8, 2H), 3.74 (m, 2H), 2.88 (m, 2H), 2.42 (m, 1 H), 1.98 (m, 2H), 1.88 (m, 2H), 1.21 (t, J=7.2, 3H).

### Intermediate 16. Ethyl 1-(6-(3,3,4,4,4-pentafluorobutyl)pyrazin-2-yl)piperidine-4-carboxylate

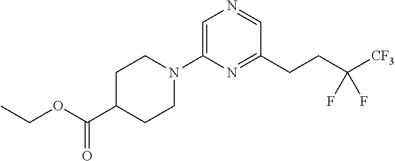 [SMILES: CCOC(=O)C1CCN(c2cncc(CCC(F)(F)C(F)(F)F)n2)CC1]

To a suspension of Zn (10.74 g, 164.25 mmol, 3.0 eq) in THF (45 mL) at rt under an N2 (g) atmosphere 1,2-dibromoethane (2.06 g, 10.95 mmol, 0.2 eq) was added over 5 min. After the addition was complete, the resulting reaction mixture was heated to 80° C. and stirred for 5 min, then the reaction mixture was cooled to rt. This heating-cooling cycle was repeated three additional times. Next, TMSCl (356 mg, 3.29 mmol, 0.1 eq) was added to the mixture at rt and the resulting reaction mixture was stirred for 10 min. The reaction mixture was cooled to at 0° C., and then a solution of 1,1,1,2,2-pentafluoro-4-iodo-butane (15 g, 54.75 mmol, 1.0 eq) in THF (45 mL) was added dropwise over 10 min. The resulting reaction mixture was allowed to warm to rt and stirred for 15 min. Next, ethyl 1-(6-chloropyrazin-2-yl)piperidine-4-carboxylate (Intermediate 8) (7.50 g, 27.81 mmol, 1.0 eq) was added, followed by Pd(dppf)Cl₂—CH₂Cl₂ (2.30 g, 2.78 mmol, 0.1 eq). The resulting reaction mixture was heated to 60° C. and stirred for 12 hr. The reaction mixture was then cooled to rt and poured into H₂O (100 mL) and extracted with EtOAc (2×100 mL). The combined organic phases were washed with brine (100 mL), dried with anhydrous Na₂SO₄, filtered, and concentrated under vacuum. The residue was purified by silica gel column chromatography (PE:EtOAc=15:1 to 1:1) to afford ethyl 1-(6-(3,3,4,4,4-pentafluorobutyl)pyrazine-2-yl)piperidine-4-carboxylate (10.5 g, 38% yield) as a yellow liquid. LCMS calculated for C₁₆H₂₀F₅N₃O₂: m/z=381; found: m/z=382 (M+H).

### Intermediate 17. Ethyl 1-(5-iodo-6-(3,3,4,4,4-pentafluorobutyl)pyrazin-2-yl)piperidine-4-carboxylate

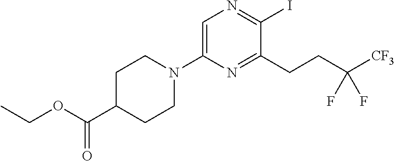 [SMILES: CCOC(=O)C1CCN(c2cnc(I)c(CCC(F)(F)C(F)(F)F)n2)CC1]

To a mixture of ethyl 1-(6-(3,3,4,4,4-pentafluorobutyl)pyrazine-2-yl)piperidine-4-carboxylate (Intermediate 16) (11.00 g, 28.85 mmol, 1.0 eq) in DMF (110 mL) at rt was added NIS (7.79 g, 34.61 mmol, 1.2 eq) and TFA (3.29 g, 28.85 mmol, 1.0 eq). The resulting reaction mixture was stirred at rt for 1 hr. The reaction mixture was quenched by addition of saturated aqueous NH₄Cl (100 mL). The mixture was then diluted with H₂O (300 mL) and extracted with EtOAc (3×400 mL). The combined organic phases were washed with brine (400 mL), dried with anhydrous Na₂SO₄, filtered, and concentrated under vacuum. The residue was purified by silica gel column chromatography (PE:EtOAc=20:1 to 5:1) to afford ethyl 1-(5-iodo-6-(3,3,4,4,4-pentafluorobutyl)pyrazin-2-yl)piperidine-4-carboxylate (11.00 g, 77% yield) as a brown solid. ¹H NMR (400 MHz, CD₃OD) δ7.91 (s, 1H), 4.27 (m, 2H), 4.14 (q, J=7.2, 2H), 3.07 (m, 4H), 2.64 (m, 3H), 2.01 (m, 2H), 1.70 (m, 2H), 1.26 (t, J=7.2, 3H); LCMS calculated for C₁₆H₁₉F₅IN₃O₂: m/z=507; found: m/z=508 (M+H).

### Intermediate 18. Ethyl 1-(3-chloro-5-iodo-6-(3,3,4,4,4-pentafluorobutyl)pyrazin-2-yl)piperidine-4-carboxylate

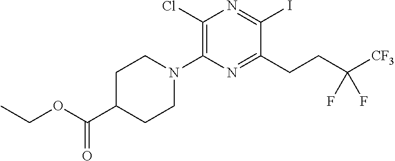 [SMILES: CCOC(=O)C1CCN(c2nc(CCC(F)(F)C(F)(F)F)c(I)nc2Cl)CC1]

To a mixture of ethyl 1-(5-iodo-6-(3,3,4,4,4-pentafluorobutyl)pyrazin-2-yl)piperidine-4-carboxylate (Intermediate 17) (10.00 g, 19.71 mmol, 1.0 eq) in DMF (100 mL) at rt was added NCS (3.42 g, 25.63 mmol, 1.3 eq) and TFA (2.25 g, 19.71 mmol, 1.0 eq). The resulting reaction mixture was heated to 100° C. and stirred for 1 hr. The reaction mixture was then cooled to rt and was quenched by addition of saturated aqueous NH₄Cl (80 mL). The resulting solution was diluted with H₂O (300 mL) and extracted with EtOAc (3×400 mL). The combined organic phase was washed with brine (300 mL), dried with anhydrous Na₂SO₄, filtered, and concentrated under vacuum. The residue was purified by silica gel column chromatography (PE:EtOAc=20:1 to 5:1) to afford ethyl 1-(3-chloro-5-iodo-6-(3,3,4,4,4-pentafluorobutyl)pyrazin-2-yl)piperidine-4-carboxylate (9.11 g, 96% yield) as a yellow liquid. LCMS calculated for C₁₆H₁₅ClF₅IN₃O₂: m/z=541; found: m/z=542 (M+H).

### Intermediate 19. Ethyl 1-(3-chloro-6-(3,3,4,4,4-pentafluorobutyl)pyrazin-2-yl)piperidine-4-carboxylate

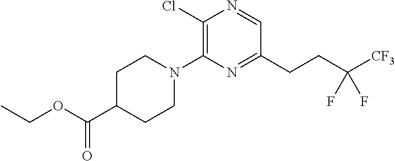 [SMILES: CCOC(=O)C1CCN(c2nc(CCC(F)(F)C(F)(F)F)cnc2Cl)CC1]

To a solution of ethyl 1-(3-chloro-5-iodo-6-(3,3,4,4,4-pentafluorobutyl)pyrazin-2-yl)piperidine-4-carboxylate (Intermediate 18) (2.00, 3.69 mmol, 1.0 eq) in MeOH (80 mL) at rt under an N₂ (g) atmosphere was added Pd/C (0.60 g, 10% weight). The resulting suspension was degassed under vacuum and purged with H₂ (g) three times. The resulting reaction mixture was stirred at rt under an H₂ (g) atmosphere (30 psi) for 12 hr. The reaction mixture was then purged with N₂ (g), filtered through a pad of celite to remove the catalyst, and the filtrate was concentrated under vacuum. The residue was purified by silica gel column chromatography (PE:EtOAc=30:1 to 1:1) to afford ethyl 1-(3-chloro-6-(3,3,4,4,4-pentafluorobutyl)pyrazin-2-yl)piperidine-4-carboxylate (1.67 g, crude) as a yellow solid. ¹H NMR (400 MHz, CDCl₃) δ 7.68 (s, 1H), 4.06 (q, J=7.2, 2H), 3.89 (m, 2H), 2.88 (m, 4H), 2.46 (m, 3H), 1.96 (m, 2H), 1.84 (m, 2H), 1.20 (t, J=7.2, 3H); LCMS calculated for C₁₆H₁₉ClF₅N₃O₂: m/z=415; found: m/z=416 (M+H).

### Intermediate 20. Ethyl 1-(5-chloro-6-(3,3,4,4,4-pentafluorobutyl)pyrazin-2-yl)piperidine-4-carboxylate

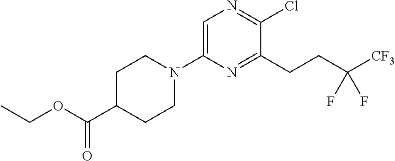 [SMILES: CCOC(=O)C1CCN(c2cnc(Cl)c(CCC(F)(F)C(F)(F)F)n2)CC1]

To a solution of ethyl 1-(6-(3,3,4,4,4-pentafluorobutyl)pyrazin-2-yl}piperidine-4-carboxylate (Intermediate 16) (2.50 g, 6.56 mmol, 1.0 eq) in DMF (10 mL) at rt was added NCS (875 mg, 6.56 mmol, 1.0 eq). The resulting reaction mixture was heated to 60° C. and stirred for 7 hr. The reaction mixture was then cooled to rt. The residue was poured into H₂O (70 mL) and extracted with EtOAc (3×15 mL). The combined organic phase was washed with brine (30 mL), dried with anhydrous Na₂SO₄, filtered, and concentrated under vacuum. The residue was purified by silica gel column chromatography (PE:EtOAc=20:1 to 10:1) to afford ethyl 1-(5-chloro-6-(3,3,4,4,4-pentafluorobutyl)pyrazin-2-yl)piperidine-4-carboxylate (1.5 g, 55% yield) as a yellow solid. ¹H NMR (400 MHz, CDCl₃) δ 7.76 (s, 1H), 4.19 (q, J=6.8, 2H), 3.07 (m, 4H), 2.55 (m, 3H), 2.02 (m, 2H), 1.77 (m, 2H), 1.26 (t, J=6.8, 3H), 0.86 (m, 2H); LCMS calculated for C₁₆H₁₉ClF₅N₃O₂: m/z=415; found: m/z=416 (M+H).

### Intermediate 21. Ethyl 1-(5-chloro-3-iodo-6-(3,3,4,4,4-pentafluorobutyl)pyrazin-2-yl)piperidine-4-carboxylate

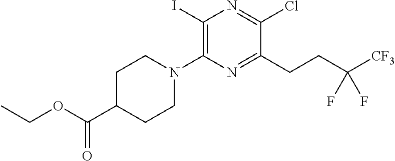 [SMILES: CCOC(=O)C1CCN(c2nc(CCC(F)(F)C(F)(F)F)c(Cl)nc2I)CC1]

To a solution of ethyl 1-(5-chloro-6-(3,3,4,4,4-pentafluorobutyl)pyrazin-2-yl)piperidine-4-carboxylate (Intermediate 20) (1.00 g, 2.41 mmol, 1.0 eq) in CHCl₃ (5 mL) at rt was added NIS (1.08 g, 4.81 mmol, 2.0 eq) and TFA (27 mg, 240 μmol, 0.1 eq). The resulting reaction mixture was heated to 80° C. and stirred for 1.5 hr. The reaction mixture was then cooled to rt. The residue was poured into H₂O (15 mL) and extracted with DCM (2×5 mL). The combined organic phase was washed with brine (10 mL), dried with anhydrous Na₂SO₄, filtered, and concentrated under vacuum. The residue was purified by silica gel column chromatography (PE:EtOAc=20:1 to 10:1) to afford ethyl 1-(5-chloro-3-iodo-6-(3,3,4,4,4-pentafluorobutyl)pyrazin-2-yl)piperidine-4-carboxylate (0.9 g, 70% yield) as a yellow solid. LCMS calculated for C₁₆H₁₅ClF₅IN₃O₂: m/z=541; found: m/z=542 (M+H).

### Intermediate 22. Ethyl 1-(6-(cyclopropylethynyl)pyrazin-2-yl)piperidine-4-carboxylate

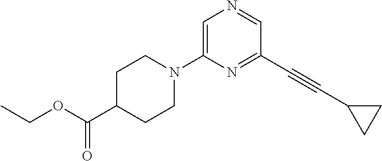 [SMILES: CCOC(=O)C1CCN(c2cncc(C#CC3CC3)n2)CC1]

To a solution of ethyl 1-(6-chloropyrazin-2-yl)piperidine-4-carboxylate (Intermediate 8) (10.00 g, 37.07 mmol, 1.0 eq) in DMA (40 mL) at rt was added CuI (706 mg, 3.71 mmol, 0.1 eq), DIPEA (47.92 g, 370.75 mmol, 10.0 eq), Pd(PPh₃)₂Cl₂ (2.60 g, 3.71 mmol, 0.1 eq) and ethynylcyclopropane (12.25 g, 185.37 mmol, 5.0 eq). The resulting reaction mixture was heated at 60° C. under an N₂ (g) atmosphere and stirred for 12 hr. The reaction mixture was cooled to rt and was then poured into H₂O (400 mL) and extracted with EtOAc (800 mL). The organic phase was washed with brine (100 mL), dried with anhydrous Na₂SO₄, filtered, and concentrated under vacuum. The residue was purified by silica gel column chromatography (PE:EtOAc=15:1 to 10:1) to afford ethyl 1-(6-(cyclopropylethynyl)pyrazin-2-yl)piperidine-4-carboxylate (18 g, 81% yield) as a yellow solid. ¹H NMR (400 MHz, CD₃OD) δ 8.07 (s, 1H), 7.73 (s, 1H), 4.26 (m, 2H), 4.12 (q, J=7.2, 2H), 3.07 (m, 2H), 2.65 (m, 1H), 2.00 (m, 2H), 1.69 (m, 2H), 1.53 (m, 1H), 1.26 (t, J=7.2, 3H), 0.95 (m, 2H), 0.86 (m, 2H); LCMS calculated for C₁₇H₂₁N₃O₂: m/z=299; found: m/z=300 (M+H).

### Intermediate 23. Ethyl 1-(6-(2-cyclopropylethyl)pyrazin-2-yl)piperidine-4-carboxylate

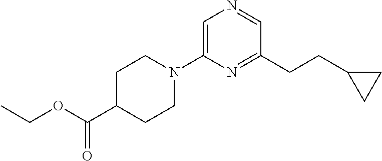 [SMILES: CCOC(=O)C1CCN(c2cncc(CCC3CC3)n2)CC1]

To a solution of ethyl 1-(6-(cyclopropylethynyl)pyrazin-2-yl)piperidine-4-carboxylate (Intermediate 22) (2.00 g, 6.68 mmol, 1.0 eq) in MeOH (100 mL) at rt under an N₂(g) atmosphere was added 10% Pd/C (1.00 g). The resulting suspension was degassed under vacuum and purged with H₂ (g) three times. The resulting reaction mixture was stirred at rt under an H₂ (g) atmosphere (15 psi) for 12 hr. The reaction mixture was then purged with N₂ (g), filtered through a pad of celite to remove the catalyst, and the filtrate was concentrated under reduced pressure. The residue was purified by silica gel column chromatography (PE:EtOAc=20:1 to 10:1) to afford ethyl 1-(6-(2-cyclopropylethyl)pyrazin-2-yl)piperidine-4-carboxylate (2 g, crude) as a yellow solid. LCMS calculated for C₁₇H₂₅N₃O₂: m/z=303; found: m/z=304 (M+H).

### Intermediate 24. Ethyl 1-(6-(2-cyclopropylethyl)-5-iodopyrazin-2-yl)piperidine-4-carboxylate

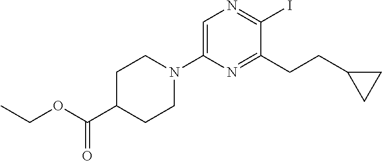 [SMILES: CCOC(=O)C1CCN(c2cnc(I)c(CCC3CC3)n2)CC1]

To a solution of ethyl 1-(6-(2-cyclopropylethyl)pyrazin-2-yl)piperidine-4-carboxylate (Intermediate 23) (10.00 g, 32.96 mmol, 1.0 eq) in DMF (100 mL) at rt was added NIS (9.64 g, 42.85 mmol, 1.3 eq) and TFA (1.88 g, 16.48 mmol, 0.5 eq). The resulting reaction mixture as stirred at rt under an N₂ (g) atmosphere for 1 hr. The reaction mixture was quenched by the slow addition of saturated aqueous NaHCO₃ (200 mL), poured into H₂O (500 mL) and extracted with EtOAc (1000 mL). The organic phase was washed with brine (300 mL), dried with anhydrous Na₂SO₄, filtered, and concentrated under vacuum. The residue was purified by silica gel column chromatography (PE:EtOAc=20:1 to 10:1) to afford ethyl 1-(6-(2-cyclopropylethyl)-5-iodopyrazin-2-yl)piperidine-4-carboxylate (9.52 g, 67%) as yellow oil. LCMS calculated for C₁₇H₂₄IN₃O₂: m/z=429; found: m/z=430 (M+H).

### Intermediate 25. Ethyl 1-(3-chloro-6-(2-cyclopropylethyl)-5-iodopyrazin-2-yl)piperidine-4-carboxylate

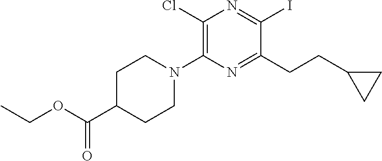 [SMILES: CCOC(=O)C1CCN(c2nc(CCC3CC3)c(I)nc2Cl)CC1]

To a solution of ethyl 1-(6-(2-cyclopropylethyl)-5-iodopyrazin-2-yl)piperidine-4-carboxylate (Intermediate 24) (10.00 g, 23.29 mmol, 1.0 eq) in DMF (100 mL) at rt was added TFA (1.33 g, 11.65 mmol, 0.5 eq) and NCS (3.42 g, 25.62 mmol, 1.1 eq). The resulting reaction mixture was heated to 100° C. under an N₂ (g) atmosphere and stirred for 1 hr. The reaction mixture was cooled to rt and quenched by the slow addition of saturated aqueous NaHCO₃ (100 mL), poured into H₂O (300 mL) and extracted with EtOAc (700 mL). The organic phase was washed with brine (400 mL), dried with anhydrous Na₂SO₄, filtered, and concentrated under vacuum. The residue was purified by silica gel column chromatography (PE:EtOAc=20:1 to 15:1) to afford ethyl 1-(3-chloro-6-(2-cyclopropylethyl)-5-iodopyrazin-2-yl)piperidine-4-carboxylate (6.8 g, 63%) as yellow oil. ¹H NMR (400 MHz, CD₃OD) δ 4.16 (q, J=6.8, 2H), 3.96 (m, 2H), 3.04-2.80 (m, 4H), 2.61 (m, 1H), 2.01 (m, 2H), 1.87-1.59 (m, 4H), 1.40 (m, 1H), 1.28 (t, J=6.8, 3H), 0.98 (m, 1H), 0.80 (m, 1H), 0.46 (m, 2 H).

### Intermediate 26. Ethyl 1-(3-chloro-6-(2-cyclopropylethyl)pyrazin-2-yl)piperidine-4-carboxylate

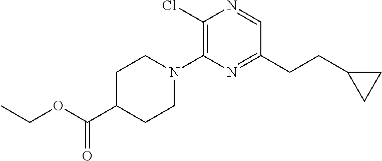 [SMILES: CCOC(=O)C1CCN(c2nc(CCC3CC3)cnc2Cl)CC1]

To a solution of ethyl 1-(3-chloro-6-(2-cyclopropylethyl)-5-iodopyrazin-2-yl)piperidine-4-carboxylate (Intermediate 25) (1.00 g, 2.16 mmol, 1.0 eq) in MeOH (100 mL) at rt under an N₂ (g) atmosphere was added 10% Pd/C (700 mg). The resulting suspension was degassed under vacuum and purged with H₂ (g) three times. The resulting reaction mixture was stirred at rt under an H₂ (g) atmosphere (30 psi) for 2 hr. The reaction mixture was then purged with N₂ (g), filtered through a pad of celite to remove the catalyst, and the filtrate was concentrated under reduced pressure. The residue was purified by silica gel column chromatography (PE:EtOAc=20:1 to 15:1) to afford ethyl 1-(3-chloro-6-(2-cyclopropylethyl)pyrazin-2-yl)piperidine-4-carboxylate (0.6 g, 83%) as yellow oil. LCMS calculated for C₁₇H₂₄C₁N₃O₂: m/z=337; found: m/z=338 (M+H).

### Intermediate 27. Ethyl 1-(5-iodo-6-((2,2,2-trifluoroethoxy)methyl)pyrazin-2-yl)piperidine-4-carboxylate

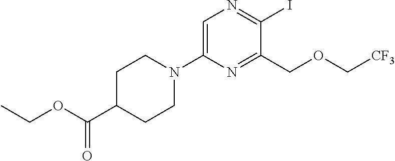 [SMILES: CCOC(=O)C1CCN(c2cnc(I)c(COCC(F)(F)F)n2)CC1]

To a solution of ethyl 1-(6-((2,2,2-trifluoroethoxy)methyl)pyrazin-2-yl)piperidine-4-carboxylate (Intermediate 13) (15.00 g, 43.19 mmol, 1.0 eq) in DMF (150 mL) at rt under an N₂ (g) atmosphere was added NIS (12.63 g, 56.14 mmol, 1.3 eq) and TFA (4.92 g, 43.19 mmol, 1.0 eq). The resulting reaction mixture was stirred for 1 hr. The reaction mixture was poured into H₂O (1000 mL) and extracted with EtOAc (3×300 mL). The combined organic phase was washed with brine (100 mL), dried with anhydrous Na₂SO₄, filtered, and concentrated under vacuum. The residue was purified by silica gel column chromatography (PE:EtOAc=1:0 to 15:1) to afford ethyl 1-(5-iodo-6-((2,2,2-trifluoroethoxy)methyl)pyrazin-2-yl)piperidine-4-carboxylate (30 g, 75% yield). ¹H NMR (400 MHz, CDCl₃) δ 7.99 (s, 1H), 4.71 (s, 2H), 4.31 (m, 2H), 4.25-4.05 (m, 4H), 3.11 (m, 2H), 2.71 (m, 1H), 2.01 (m, 2H), 1.73 (m, 2H), 1.27 (t, J=7.2, 3H).

### Intermediate 28. Ethyl 1-(3-chloro-5-iodo-6-((2,2,2-trifluoroethoxy)methyl)pyrazin-2-yl)piperidine-4-carboxylate

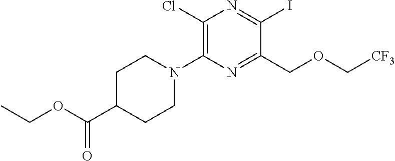 [SMILES: CCOC(=O)C1CCN(c2nc(COCC(F)(F)F)c(I)nc2Cl)CC1]

To a solution of ethyl 1-(5-iodo-6-((2,2,2-trifluoroethoxy)methyl)pyrazin-2-yl)piperidine-4-carboxylate (Intermediate 27) (8.00 g, 16.91 mmol, 1.0 eq) in DMF (80 mL) at rt under an N₂ (g) atmosphere was added NCS (2.93 g, 21.98 mmol, 1.3 eq) and TFA (1.93 mg, 16.91 mmol, 1.0 eq). The resulting reaction mixture was then heated at 90° C. and stirred for 1 hr. The reaction mixture was cooled to rt. The reaction mixture was then poured into H₂O (200 mL) and extracted with EtOAc (3×200 mL). The combined organic phase was washed with brine (100 mL), dried with anhydrous Na₂SO₄, filtered, and concentrated under vacuum. The residue was purified by silica gel column chromatography (PE:EtOAc=1:0 to 1:1) to afford ethyl 1-(3-chloro-5-iodo-6-((2,2,2-trifluoroethoxy)methyl)pyrazin-2-yl)piperidine-4-carboxylate (8.00 g, 93% yield) as a yellow oil. LCMS calculated for C₁₅H₁₈ClF₃IN₃O₃: m/z=507; found: m/z=508 (M+H).

### Intermediate 29. Ethyl 1-(3-chloro-6-((2,2,2-trifluoroethoxy)methyl)pyrazin-2-yl)piperidine-4-carboxylate

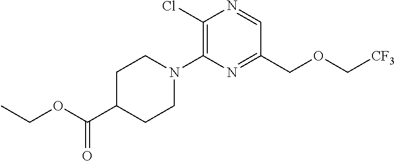 [SMILES: CCOC(=O)C1CCN(c2nc(COCC(F)(F)F)cnc2Cl)CC1]

To a solution of ethyl 1-(3-chloro-5-iodo-6-((2,2,2-trifluoroethoxy)methyl)pyrazin-2-yl)piperidine-4-carboxylate (Intermediate 28) (5.00 g, 9.85 mmol, 1.0 eq) in MeOH (100 mL) at rt under an N₂ (g) atmosphere was added 10% Pd/C (600 mg). The resulting suspension was degassed under vacuum and purged with H₂ (g) three times. The resulting reaction mixture was stirred at rt under an H₂ (g) atmosphere (30 psi) for 1 hr. The reaction mixture was then purged with N₂ (g) filtered through a pad of celite to remove the catalyst, and the filtrate was concentrated under vacuum. The residue was purified by silica gel column chromatography (PE:EtOAc=40:1 to 15:1) to afford ethyl 1-(3-chloro-6-((2,2,2-trifluoroethoxy)methyl)pyrazin-2-yl)piperidine-4-carboxylate (2.20 g, 54% yield) as a yellow oil. ¹H NMR (400 MHz, CDCl₃) δ 7.94 (s, 1H), 4.72 (s, 2H), 4.13 (q, J=7.2, 2H), 3.96 (m, 4H), 2.94 (m, 2H), 2.53 (m, 1H), 2.01 (m, 2H), 1.88 (m, 2H), 1.25 (t, J=7.2,3 H).

### Intermediate 30. Ethyl 1-(6-(3,3,3-trifluoropropyl)pyrazin-2-yl)piperidine-4-carboxylate

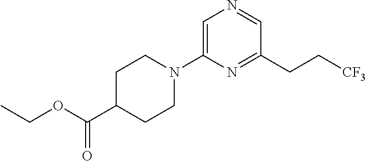 [SMILES: CCOC(=O)C1CCN(c2cncc(CCC(F)(F)F)n2)CC1]

To a solution of ethyl 1-(6-chloropyrazin-2-yl)piperidine-4-carboxylate (Intermediate 8) (2.00 g, 7.41 mmol, 1.0 eq) and potassium trifluoro(3,3,3-trifluoropropyl)borate (1.81 g, 8.90 mmol, 1.2 eq) in 1,4-dioxane (30 mL) and H₂O (6 mL) at rt under an N₂ (g) atmosphere was added Pd(dppf)Cl₂—CH₂Cl₂ (605 mg, 741 μmol, 0.1 eq) and Cs₂CO₃ (7.25 g, 22.24 mmol, 3.0 eq). The resulting reaction mixture was heated to 110° C. and stirred for 12 hr. The reaction mixture was cooled to rt, diluted with H₂O (300 mL), and then extracted with EtOAc (2×300 mL). The combined organic phase was washed with brine (100 mL), dried over Na₂SO₄, filtered, and concentrated under vacuum. The residue was purified by silica gel column chromatography (PE:EtOAc=20:1 to 10:1) to afford ethyl 1-(6-(3,3,3-trifluoropropyl)pyrazin-2-yl)piperidine-4-carboxylate (2.09 g, 85% yield) as yellow liquid. ¹H NMR (400 MHz, CDCl₃) δ 7.97 (s, 1H), 7.69 (s, 1H), 4.24 (m, 2H), 4.13 (q, J=7.2, 2H), 3.03 (m, 2H), 2.84 (m, 2H), 2.53 (m, 3H) 1.99 (m, 2H) 1.74 (m, 5H) 1.25 (t, J=7.2, 3H); LCMS calculated for C₁₅H₂₀F₃N₃O₂: m/z=331; found: m/z=332 (M+H).

### Intermediate 31. Ethyl 1-(5-chloro-6-(3,3,3-trifluoropropyl)pyrazin-2-yl)piperidine-4-carboxylate

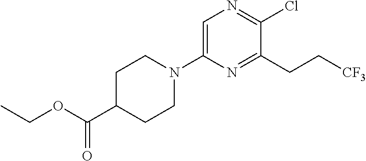 [SMILES: CCOC(=O)C1CCN(c2cnc(Cl)c(CCC(F)(F)F)n2)CC1]

To a solution of ethyl 1-(6-(3,3,3-trifluoropropyl)pyrazin-2-yl)piperidine-4-carboxylate (Intermediate 30) (300 mg, 905 μmol, 1.0 eq) in DMF (2 mL) at rt was added NCS (121 mg, 905 μmol, 1.0 eq). The resulting reaction mixture was heated to 60° C. under an N₂ (g) atmosphere and stirred for 1 hr. The reaction mixture was cooled to rt, poured into H₂O (100 mL), and extracted with EtOAc (3×100 mL). The combined organic phase was washed with brine (50 mL), dried with anhydrous Na₂SO₄, filtered, and concentrated under vacuum. The residue was purified by silica gel column chromatography (PE:EtOAc=20:1 to 10:1) to afford ethyl 1-(5-chloro-6-(3,3,3-trifluoropropyl)pyrazin-2-yl)piperidine-4-carboxylate (170 mg, 51% yield) as a white solid. ¹H NMR (400 MHz, CDCl₃) δ 7.89 (s, 1 H), 4.28 (m, 2H), 4.16 (q, J=7.2, 2H), 3.08 (m, 4H), 2.67 (m, 3H), 1.99 (m, 2H), 1.72 (m, 2H), 1.27 (t, J=7.2, 3H); LCMS calculated for C₁₅H₁₉ClF₃N₃O₂: m/z=365; found: m/z=366 (M+H).

### Intermediate 32. Ethyl 1-(5-chloro-3-iodo-6-(3,3,3-trifluoropropyl)pyrazin-2-yl)piperidine-4-carboxylate

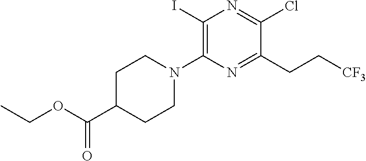 [SMILES: CCOC(=O)C1CCN(c2nc(CCC(F)(F)F)c(Cl)nc2I)CC1]

To a solution of ethyl 1-(5-chloro-6-(3,3,3-trifluoropropyl)pyrazin-2-yl)piperidine-4-carboxylate (Intermediate 31) (150 mg, 410 μmol, 1.0 eq) in CHCl₃ (5 mL) at rt was added NIS (92 mg, 410 μmol, 1.0 eq) and TFA (46 mg, 410 μmol, 1.0 eq). The resulting reaction mixture was heated to 80° C. and stirred for 1 hr. The reaction mixture was cooled to rt, poured into H₂O (100 mL), and extracted with EtOAc (3×100 mL). The combined organic phase was washed with brine (100 mL), dried with anhydrous Na₂SO₄, filtered, and concentrated under vacuum. The residue was purified by silica gel column chromatography (PE:EtOAc=20:1 to 10:1) to afford ethyl 1-(5-chloro-3-iodo-6-(3,3,3-trifluoropropyl)pyrazin-2-yl)piperidine-4-carboxylate (172 mg) as an oily yellow liquid. LCMS calculated for C₁₅H₁₅ClF₃IN₃O₂: m/z=491; found: m/z=492 (M+H).

### Intermediate 33. Ethyl 1-(5-iodo-6-(3,3,3-trifluoropropyl)pyrazin-2-yl)piperidine-4-carboxylate

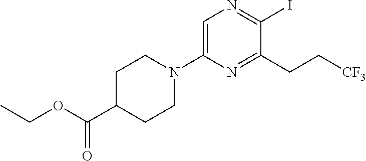 [SMILES: CCOC(=O)C1CCN(c2cnc(I)c(CCC(F)(F)F)n2)CC1]

To a solution of ethyl 1-(6-(3,3,3-trifluoropropyl)pyrazin-2-yl)piperidine-4-carboxylate (Intermediate 30) (10.00 g, 30.18 mmol, 1.0 eq) in DMF (100 mL) at rt was added TFA (344 mg, 3.02 mmol, 0.1 eq) and NIS (6.79 g, 30.18 mmol, 1.0 eq). The resulting reaction mixture was stirred at rt for 1 hr. The reaction mixture was quenched by addition saturated aqueous NaHCO₃ (70 mL), then the reaction mixture was diluted with H₂O (300 mL) and extracted with EtOAc (3×300 mL). The combined organic phase was washed with brine (300 mL), dried with anhydrous Na₂SO₄, filtered, and concentrated under vacuum. The residue was purified by silica gel column chromatography (PE:EtOAc=20:1 to 10:1) to afford ethyl 1-(5-iodo-6-(3,3,3-trifluoropropyl)pyrazin-2-yl)piperidine-4-carboxylate (10.30 g) as a yellow solid. LCMS calculated for C₁₅H₁₉F₃IN₃O₂: m/z=457; found: m/z=458 (M+H).

### Intermediate 34. Ethyl 1-(3-chloro-5-iodo-6-(3,3,3-trifluoropropyl)pyrazin-2-yl)piperidine-4-carboxylate

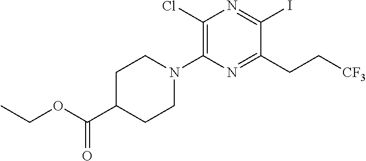 [SMILES: CCOC(=O)C1CCN(c2nc(CCC(F)(F)F)c(I)nc2Cl)CC1]

To a solution of ethyl 1-(5-iodo-6-(3,3,3-trifluoropropyl)pyrazin-2-yl)piperidine-4-carboxylate (Intermediate 33) (4.65 g, 10.17 mmol, 1.0 eq) in DMF (45 mL) at rt was added TFA (1.16 g, 10.17 mmol, 0.1 eq) and NCS (1.63 g, 12.20 mmol, 1.2 eq). The resulting reaction mixture was heated to 100° C. and stirred for 2 hr. The reaction mixture was then cooled to rt and then quenched by addition of saturated aqueous NaHCO₃ (50 mL), diluted with H₂O (300 mL) and extracted with EtOAc (3×400 mL). The combined organic phase was washed with brine (300 mL), dried with anhydrous Na₂SO₄, filtered, and concentrated under vacuum. The residue was purified by silica gel column chromatography (PE:EtOAc=20:1 to 10:1) to afford ethyl 1-(3-chloro-5-iodo-6-(3,3,3-trifluoropropyl)pyrazin-2-yl)piperidine-4-carboxylate (4.65 g) as a yellow solid. LCMS calculated for C₁₅H₁₈ClF₃IN₃O₂: m/z=491; found: m/z=492 (M+H).

### Intermediate 35. Ethyl 1-(3-chloro-6-(3,3,3-trifluoropropyl)pyrazin-2-yl)piperidine-4-carboxylate

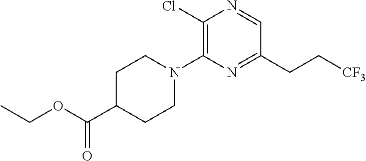 [SMILES: CCOC(=O)C1CCN(c2nc(CCC(F)(F)F)cnc2Cl)CC1]

To a solution of ethyl 1-(3-chloro-5-iodo-6-(3,3,3-trifluoropropyl)pyrazin-2-yl)piperidine-4-carboxylate (Intermediate 34) (1.30 g, 2.64 mmol, 1.0 eq) in MeOH (50 mL) at rt under an N₂ (g) atmosphere was added 10% Pd/C (0.50 g). The suspension was degassed under vacuum and purged with H₂ (g) three times. The resulting reaction mixture was stirred at rt under an H₂ (g) atmosphere (30 psi) for 12 hr. The reaction mixture was then purged with N₂ (g), filtered through a pad of celite to remove the catalyst, and the filtrate was concentrated under vacuum. The residue was purified by silica gel column chromatography (PE:EtOAc=20:1 to 10:1) to afford ethyl 1-(3-chloro-6-(3,3,3-trifluoropropyl)pyrazin-2-yl)piperidine-4-carboxylate (0.73 g, 76%) as yellow liquid. ¹H NMR (400 MHz, CD₃OD) δ 7.79 (s, 1H), 4.14 (q, J=7.2, 2H), 3.93 (m, 2H), 2.94 (m, 4H), 2.59 (m, 3H) 1.99 (m, 2H) 1.81 (m, 2H) 1.24 (t, J=7.2, 3H); LCMS calculated for C₁₅H₁₅ClF₃N₃O₂: m/z=365; found: m/z=366 (M+H).

### Intermediate 36. 1-(6-chloropyrazin-2-yl)piperidine-4-carbonitrile

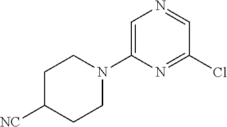 [SMILES: N#CC1CCN(c2cncc(Cl)n2)CC1]

To a solution of 2,6-dichloropyrazine (5.00 g, 33.56 mmol, 1.0 eq) and piperidinium-4-carbonitrile hydrochloride (5.12 g, 34.90 mmol, 1.1 eq,) in 1,4-dioxane (50 mL) at rt was added Et₃N (3.74 g, 36.92 mmol, 1.1 eq). The resulting reaction mixture was heated to 100° C. and stirred for 12 hr. The reaction mixture was then cooled to rt, diluted with H₂O (200 mL), and then extracted with EtOAc (3×200 mL). The combined organic phase was washed with brine (100 mL), dried over Na₂SO₄, filtered, and concentrated under vacuum. The residue was purified by silica gel column chromatography (PE:EtOAc=20:1 to 10:1) to afford 1-(6-chloropyrazin-2-yl)piperidine-4-carbonitrile (3.24 g, 43% yield) as a yellow solid. LCMS calculated for CioHnClN₄: m/z=222; found: m/z=223 (M+H).

### Intermediate 37. 1-(6-((2,2,2-trifluoroethoxy)methyl)pyrazin-2-yl)piperidine-4-carbonitrile

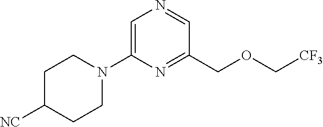 [SMILES: N#CC1CCN(c2cncc(COCC(F)(F)F)n2)CC1]

To a solution of 1-(6-chloropyrazin-2-yl)piperidine-4-carbonitrile (Intermediate 36) (3.00 g, 13.47 mmol, 1.0 eq) and potassium trifluoro((2,2,2-trifluoroethoxy)methyl)borate (4.44 g, 20.21 mmol, 1.5 eq) in H₂O (4 mL) and 1,4-dioxane (20 mL) at rt under an N₂(g) atmosphere was added Pd(dppf)Cl₂—CH₂Cl₂, (1.10 g, 1.35 mmol, 0.1 eq) and Cs₂CO₃ (13.17 g, 40.41 mmol, 3.0 eq). The resulting reaction mixture was heated to 110° C. and stirred for 12 hr. The reaction mixture was cooled to rt, diluted with H₂O (200 mL), and extracted with EtOAc (2×300 mL). The combined organic phase was washed with brine (100 mL), dried over Na₂SO₄, filtered, and concentrated under vacuum. The residue was purified by silica gel column chromatography (PE:EtOAc=20:1 to 10:1) to afford 1-(6-(2,2,2-trifluoroethoxymethyl)pyrazin-2-yl)piperidine-4-carbonitrile (1.3 g, 32% yield) as a white solid. LCMS calculated for C₁₃H₁₅F₃N₄O: m/z=300; found: m/z=301 (M+H).

### Intermediate 38. 1-(5-iodo-6-((2,2,2-trifluoroethoxy)methyl)pyrazin-2-yl)piperidine-4-carbonitrile

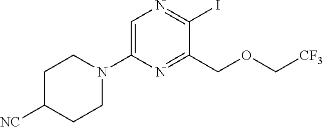 [SMILES: N#CC1CCN(c2cnc(I)c(COCC(F)(F)F)n2)CC1]

To a solution of 1-(6-(2,2,2-trifluoroethoxymethyl)pyrazin-2-yl)piperidine-4-carbonitrile (Intermediate 37) (6.00 g, 19.98 mmol, 1.0 eq) in DMF (40 mL) at rt was added TFA (2.28 g, 19.98 mmol, 1.0 eq) and NIS (5.84 g, 25.98 mmol, 1.3 eq), and the resulting reaction mixture was stirred at rt for 2 hr. The reaction mixture was quenched by addition of saturated aqueous NaHCO₃ (100 mL) at rt, diluted with H₂O (200 mL), and extracted with EtOAc (3×200 mL). The combined organic phase was washed with brine (100 mL), dried with anhydrous Na₂SO₄, filtered, and concentrated under vacuum. The residue was purified by silica gel column chromatography (PE:EtOAc=20:1 to 10:1) to afford 1-(5-iodo-6-(2,2,2-trifluoroethoxymethyl)pyrazin-2-yl)piperidine-4-carbonitrile (4.5 g, 55% yield) as a yellow solid. LCMS calculated for C₁₃H₁₄F₃IN₄O: m/z=426; found: m/z=427 (M+H).

### Intermediate 39. 1-(3-chloro-5-iodo-6-((2,2,2-trifluoroethoxy)methyl)pyrazin-2-yl)piperidine-4-carbonitrile

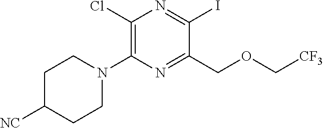 [SMILES: N#CC1CCN(c2nc(COCC(F)(F)F)c(I)nc2Cl)CC1]

To a solution of 1-(5-iodo-6-(2,2,2-trifluoroethoxymethyl)pyrazin-2-yl)piperidine-4-carbonitrile (Intermediate 38) (3.5 g, 8.21 mmol, 1.0 eq) in DMF (35 mL) at rt was added TFA (936 mg, 8.21 mmol, 1.0 eq) and NCS (1.43 g, 10.68 mmol, 1.3 eq). The resulting reaction mixture was heated to 100° C. and stirred for 3 hr. The reaction mixture was then cooled to rt. The reaction mixture was quenched by addition of saturated aqueous NaHCO₃ (50 mL) at rt, diluted with H₂O (200 mL) and extracted with EtOAc (3×200 mL). The combined organic phase was washed with brine (100 mL), dried with anhydrous Na₂SO₄, filtered, and concentrated under vacuum. The residue was purified by silica gel column chromatography (PE:EtOAc=20:1 to 10:1) to afford 1-(3-chloro-5-iodo-6-(2,2,2-trifluoroethoxymethyl)pyrazin-2-yl)piperidine-4-carbonitrile (3.24 g, 86% yield) as a yellow solid. LCMS calculated for C₁₃H₁₃ClF₃IN₄O: m/z=460; found: m/z=461 (M+H).

### Intermediate 40. 1-(3-chloro-6-((2,2,2-trifluoroethoxy)methyl)pyrazin-2-yl)piperidine-4-carbonitrile

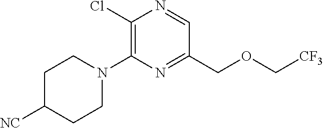 [SMILES: N#CC1CCN(c2nc(COCC(F)(F)F)cnc2Cl)CC1]

To a solution of 1-(3-chloro-5-iodo-6-(2,2,2-trifluoroethoxymethyl)pyrazin-2-yl)piperidine-4-carbonitrile (Intermediate 39) (1.00 g, 2.17 mmol, 1.0 eq) in MeOH (50 mL) at rt was added Pd/C (0.50 g, 10% weight) under an N₂ (g) atmosphere. The suspension was degassed under vacuum and purged with H₂ (g) three times. The resulting reaction mixture was stirred at rt under an H₂ (g) atmosphere (30 psi) for 12 hr. The reaction mixture was then purged with N₂ (g), filtered through a pad of celite to remove the catalyst, and the filtrate was concentrated under vacuum. The residue was purified by silica gel column chromatography (PE:EtOAc=20:1 to 10:1) to afford 1-(3-chloro-6-(2,2,2-trifluoroethoxymethyl)pyrazin-2-yl)piperidine-4-carbonitrile (600 mg, 82% yield) as yellow liquid. LCMS calculated for C₁₃H₁₄ClF₃N₄O: m/z=334; found: m/z=335 (M+H).

### Intermediate 41. 2-(7-fluorobenzofuran-5-yl)-4,4,5,5-tetramethyl-1,3,2-dioxaborolane

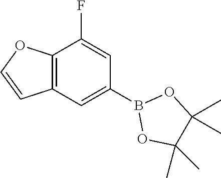 [SMILES: CC1(C)OB(c2cc(F)c3occc3c2)OC1(C)C]

To a solution of 5-bromo-7-fluoro-benzofuran (7.0 g, 32.56 mmol, 1.0 eq) and bis-pinacolatodiborane (13.23 g, 52.09 mmol, 1.6 eq) in 1,4-dioxane (98 mL) at rt under an N₂ (g) atmosphere was added Pd(dppf)Cl₂-CH₂Cl₂ (5.32 g, 6.51 mmol, 0.2 eq) and KOAc (6.39 g, 65.11 mmol, 2.0 eq). The resulting reaction mixture was heated to 100° C. and stirred for 12 hr. The reaction mixture was then cooled to rt, diluted with H₂O (500 mL), and then extracted with EtOAc (2×500 mL). The combined organic layers were washed with brine (200 mL), dried over Na₂SO₄, filtered, and concentrated under reduced pressure to give a residue. The residue was purified by silica gel column chromatography (PE:EtOAc=20:1 to 15:1) to afford 2-(7-fluorobenzofuran-5-yl)-4,4,5,5-tetramethyl-1,3,2-dioxaborolane (3.35 g, 91%) as a white solid. ¹H NMR (400 MHz, CD₃OD) δ7.84 (d, J=2.0, 2H), 7.38 (d, J=11.2, 1H), 6.95 (m, 1H), 1.38 (s, 12H).

### Intermediate 42. 1-(6-(3,3,3-trifluoropropyl)pyrazin-2-yl)piperidine-4-carbonitrile

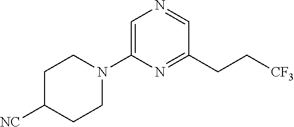 [SMILES: N#CC1CCN(c2cncc(CCC(F)(F)F)n2)CC1]

To a solution of 1-(6-chloropyrazin-2-yl)piperidine-4-carbonitrile (Intermediate 36) (3.00 g, 13.47 mmol, 1.0 eq) and potassium trifluoro(3,3,3-trifluoropropyl)borate (3.02 g, 14.82 mmol, 1.1 eq) in 1,4-dioxane (30 mL) and H₂O (6 mL) at rt under an N₂ (g) atmosphere was added Pd(dppf)Cl₂-CH₂Cl₂ (1.10 g, 1.35 mmol, 0.1 eq) and Cs₂CO₃ (13.17 g, 40.42 mmol, 3.0 eq). The resulting reaction mixture was heated to 110° C. and stirred for 12 hr. The reaction mixture was then cooled to rt, diluted with H₂O (200 mL), and extracted with EtOAc (2×300 mL). The combined organic phase was washed with brine (100 mL), dried over Na₂SO₄, filtered, and concentrated under vacuum. The residue was purified by silica gel column chromatography (PE:EtOAc=20:1 to 5:1) to afford 1-(6-(3,3,3-trifluoropropyl)pyrazin-2-yl)piperidine-4-carbonitrile (3.7 g, 96% yield) as brown liquid. LCMS calculated for C₁₃H₁₅F₃N₄: m/z=284; found: m/z=285 (M+H).

### Intermediate 43. 1-(5-iodo-6-(3,3,3-trifluoropropyl)pyrazin-2-yl)piperidine-4-carbonitrile

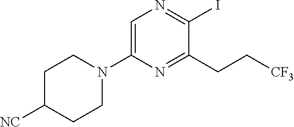 [SMILES: N#CC1CCN(c2cnc(I)c(CCC(F)(F)F)n2)CC1]

To a suspension of 1-(6-(3,3,3-trifluoropropyl)pyrazin-2-yl)piperidine-4-carbonitrile (Intermediate 42) (3.20 g, 11.26 mmol, 1.0 eq) in DMF (32 mL) at rt was added NIS (3.29 g, 14.63 mmol, 1.3 eq) and TFA (1.28 g, 11.26 mmol, 1.0 eq), and the resulting reaction mixture was stirred at rt for 3 hr. The reaction mixture was quenched by addition of saturated aqueous NaHCO₃ (50 mL), diluted with H₂O (200 mL) and extracted with EtOAc (3×200 mL). The combined organic phase was washed with brine (100 mL), dried with anhydrous Na₂SO₄, filtered, and concentrated under vacuum. The residue was purified by silica gel column chromatography (PE:EtOAc=20:1 to 10:1) to afford 1-(5-iodo-6-(3,3,3-trifluoropropyl)pyrazin-2-yl)piperidine-4-carbonitrile (2.3 g, 50% yield) as yellow oil. LCMS calculated for C₁₃H₁₄F₃IN₄: m/z=410; found: m/z=411 (M+H).

### Intermediate 44. 1-(3-chloro-5-iodo-6-(3,3,3-trifluoropropyl)pyrazin-2-yl)piperidine-4-carbonitrile

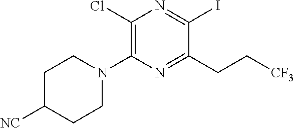 [SMILES: N#CC1CCN(c2nc(CCC(F)(F)F)c(I)nc2Cl)CC1]

To a solution of 1-(5-iodo-6-(3,3,3-trifluoropropyl)pyrazin-2-yl)piperidine-4-carbonitrile (Intermediate 43) (2.00 g, 4.88 mmol, 1.0 eq) in DMF (20 mL) at rt was added TFA (556 mg, 4.88 mmol, 1.0 eq) and NCS (846 mg, 6.34 mmol, 1.3 eq). The resulting reaction mixture was heated to 100° C. and stirred for 3 hr. The reaction mixture was then cooled to rt. The reaction mixture was quenched by addition of saturated aqueous NaHCO₃ (50 mL), diluted with H₂O (200 mL), and extracted with EtOAc (3×200 mL). The combined organic phase was washed with brine (100 mL), dried with anhydrous Na₂SO₄, filtered, and concentrated under vacuum. The residue was purified by silica gel column chromatography (PE:EtOAc=20:1 to 10:1) to afford 1-(3-chloro-5-iodo-6-(3,3,3-trifluoropropyl)pyrazin-2-yl)piperidine-4-carbonitrile (1.9 g, 88% yield) as a yellow solid. LCMS calculated for C₁₃H₁₃ClF₃IN₄: m/z=443; found: m/z=444 (M+H).

### Intermediate 45. 1-(3-chloro-6-(3,3,3-trifluoropropyl)pyrazin-2-yl)piperidine-4-carbonitrile

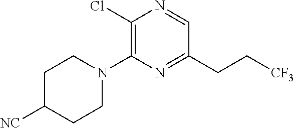 [SMILES: N#CC1CCN(c2nc(CCC(F)(F)F)cnc2Cl)CC1]

To a solution of 1-(3-chloro-5-iodo-6-(3,3,3-trifluoropropyl)pyrazin-2-yl)piperidine-4-carbonitrile (Intermediate 44) (1.80 g, 4.05 mmol, 1.0 eq) in MeOH (100 mL) at rt was added 10% Pd/C (1.00 g) under an N₂ (g) atmosphere. The suspension was degassed under vacuum and purged with H₂ (g) three times. The resulting reaction mixture was stirred at rt under an H₂ atmosphere (g) (30 psi) for 12 hr. The reaction mixture was then purged with N₂ (g), filtered through a pad of celite to remove the catalyst, and the filtrate was concentrated under vacuum. The residue was purified by silica gel column chromatography (PE:EtOAc=20:1 to 10:1) to afford 1-(3-chloro-6-(3,3,3-trifluoropropyl)pyrazin-2-yl)piperidine-4-carbonitrile (1.23 g, 95% yield) as an oily yellow liquid. LCMS calculated for C₁₃H₁₄ClF₃N4: m/z=317; found: m/z=318 (M+H).

### Intermediate 46. Ethyl 1-(6-vinylpyrazin-2-yl)piperidine-4-carboxylate

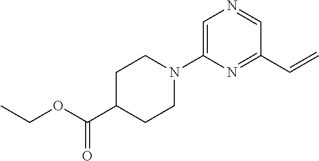 [SMILES: C=Cc1cncc(N2CCC(C(=O)OCC)CC2)n1]

To a solution of ethyl 1-(6-chloropyrazin-2-yl)piperidine-4-carboxylate (Intermediate 8) (20.0 g, 74.15 mmol, 1.0 eq) and potassium trifluoro(vinyl)borate (11.92 g, 88.98 mmol, 1.2 eq) in THF (200 mL) and H₂O (40 mL) at rt was added Pd(dppf)Cl₂—CH₂C₁₂ (3.03 g, 3.71 mmol, 0.05 eq) and K₂CO₃ (20.50 g, 148.30 mmol, 2.0 eq). The resulting reaction mixture was heated at 80° C. under an N₂ (g) atmosphere and stirred for 12 hr. The reaction mixture was cooled to rt and concentrated under reduced pressure to remove most of the THF. The residue was poured into H₂O (700 mL) and extracted with EtOAc (2×700 mL). The combined organic phase was washed with brine (200 mL), dried with anhydrous Na₂SO₄, filtered, and concentrated under vacuum. The residue was purified by silica gel column chromatography (PE:EtOAc=10:1 to 5:1) to afford ethyl 1-(6-vinylpyrazin-2-yl)piperidine-4-carboxylate (16.8 g, 86% yield) as a yellow oil. ¹H NMR (400 MHz, CDCl₃) δ 8.03 (s, 1H), 7.80 (s, 1H), 6.67 (dd, J=10.8, 17.2, 1H), 6.29 (dd, J=2.0, 17.2, 1H), 5.48 (dd, J=2.0, 10.8, 1H), 4.32 (m, 2H), 4.15 (q, J=7.2, 2H), 3.04 (m, 2H), 2.57 (m, 1H), 2.04 (m, 2H), 1.80 (m, 2H), 1.27 (t, J=7.2, 3H); LCMS calculated for C₁₄H₁₉N₃O₂: m/z=261; found: m/z=262 (M+H).

### Intermediate 47. Ethyl 1-(6-formylpyrazin-2-yl)piperidine-4-carboxylate

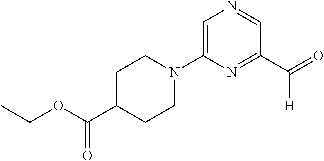 [SMILES: CCOC(=O)C1CCN(c2cncc(C=O)n2)CC1]

To a solution of ethyl 1-(6-vinylpyrazin-2-yl)piperidine-4-carboxylate (Intermediate 46) (10.0 g, 38.27 mmol, 1.0 eq) in THF (100 mL) and H₂O (20 mL) at 0° C. was added KOsO₄ (2.82 g, 7.65 mmol, 0.2 eq), followed by NaIO₄ (24.56 g, 114.80 mmol, 3.0 eq). The resulting reaction mixture was then warmed to rt and stirred for 12 hr. The reaction mixture was poured into H₂O (700 mL) and extracted with EtOAc (2×700 mL). The combined organic phase was washed with brine (100 mL), dried with anhydrous Na₂SO₄, filtered, and concentrated under vacuum. The residue was purified by silica gel column chromatography (PE:EtOAc=10:1 to 2:1) to afford ethyl 1-(6-formylpyrazin-2-yl)piperidine-4-carboxylate (3.00 g, 29% yield) as a yellow oil. ¹H NMR (400 MHz, CDCl₃) δ 9.96 (s, 1H), 8.38 (s, 1H), 8.33 (s, 1H), 4.34 (m, 2H), 4.14 (q, J=7.2,2 H), 3.16 (m, 2 H), 2.62 (m, 1H), 2.08 (m, 2H), 1.81 (m, 2H), 1.29 (t, J=7.2, 3H); LCMS calculated for C₁₃H₁₇N₃O₃: m/z=263; found: m/z=264 (M+H).

### Intermediate 48. Ethyl (E)-1-(6-(5,5,5-trifluoropent-1-en-1-yl)pyrazin-2-yl)piperidine-4-carboxylate

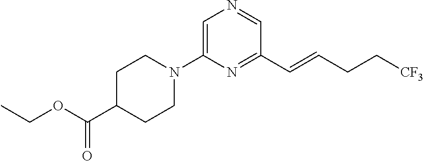 [SMILES: CCOC(=O)C1CCN(c2cncc(/C=C/CCC(F)(F)F)n2)CC1]

To a mixture of ethyl 1-(6-formylpyrazin-2-yl)piperidine-4-carboxylate (Intermediate 47) (2.50 g, 9.50 mmol, 1.0 eq) and triphenyl-(4,4,4-trifluorobutyl)phosphonium iodide (prepared by heating a solution of 1,1,1-trifluoro-4-iodobutane (5.00 g, 21.01 mmol, 1.0 eq) and PPh₃ (5.51 g, 21.01 mmol, 1.0 eq) in toluene (50 mL) at 85° C. for 12 hr) (7.25 g, 14.43 mmol, 1.52 eq) in isopropyl acetate (25 mL) at rt was added K₂CO₃ (2.10 g, 15.19 mmol, 1.6 eq). The resulting reaction mixture was heated to 80° C. and stirred for 12 hr. The reaction mixture was cooled to rt, diluted with H₂O (400 mL) and extracted with EtOAc (2×400 mL). The combined organic phase was washed with brine (200 mL), dried with anhydrous Na₂SO₄, filtered, and concentrated under vacuum. The residue was purified by silica gel column chromatography (PE:EtOAc=8:1 to 2:1) to afford ethyl (E)-1-(6-(5,5,5-trifluoropent-1-en-1-yl)pyrazin-2-yl)piperidine-4-carboxylate (2.83 g) as yellow oil. ¹H NMR (400 MHz, CDCl₃) δ 8.01 (s, 1H), 7.73 (s, 1H), 6.33 (d, J=11.6, 1 H), 5.88 (m, 1H), 4.17 (m, 4H), 3.11 (m, 4H), 2.58 (m, 1H), 2.31 (m, 2H), 2.05 (m, 2H), 1.80 (m, 2H), 1.30 (t, J=7.2, 3H); LCMS calculated for C₁₇H₂₂F₃N₃O₂: m/z=357; found: m/z=358 (M+H).

### Intermediate 49. Ethyl 1-(6-(5,5,5-trifluoropentyl)pyrazin-2-yl)piperidine-4-carboxylate

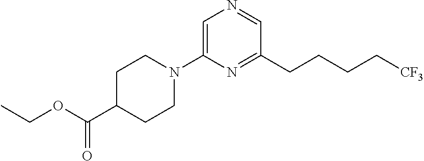 [SMILES: CCOC(=O)C1CCN(c2cncc(CCCCC(F)(F)F)n2)CC1]

To a solution of ethyl (E)-1-(6-(5,5,5-trifluoropent-1-en-1-yl)pyrazin-2-yl)piperidine-4-carboxylate (Intermediate 48) (1.20 g, 3.36 mmol, 1.0 eq) in MeOH (100 mL) at rt was added 10% Pd/C (800 mg). The suspension was degassed under vacuum and purged with H₂ (g) three times. The resulting reaction mixture was stirred at rt under an H₂ atmosphere (g) (15 psi) for 3 hr. The reaction mixture was then purged with N₂ (g) three times and filtered through a pad of celite to remove the catalyst. The filtrate was concentrated under reduced pressure to give a residue. The residue was purified by silica gel column chromatography (PE:EtOAc=10:1 to 5:1) to afford ethyl 1-(6-(5,5,5-trifluoropentyl)pyrazin-2-yl]piperidine-4-carboxylate (0.91 g, 76% yield) as a yellow oil. ¹H NMR (400 MHz, CDCl₃) δ 7.97 (s, 1H), 7.70 (s, 1H), 4.27 (m, 2H), 4.16 (q, J=7.2, 2H), 3.04 (dt, J=2.4, 11.2, 2H), 2.64 (t, J=7.2, 2H), 2.53 (m, 1H), 2.16 (m, 2H), 2.03 (m, 2H), 1.80 (m, 4H), 1.63 (m, 2H), 1.29 (t, J=7.2, 3H); LCMS calculated for C₁₇H₂₄F₃N₃O₂: m/z=359; found: m/z=360 (M+H).

### Intermediate 50. Ethyl 1-(5-iodo-6-(5,5,5-trifluoropentyl)pyrazin-2-yl)piperidine-4-carboxylate

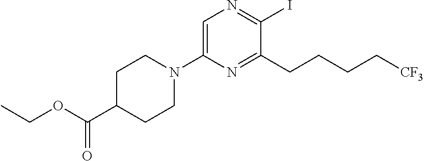 [SMILES: CCOC(=O)C1CCN(c2cnc(I)c(CCCCC(F)(F)F)n2)CC1]

To a solution of ethyl 1-(6-(5,5,5-trifluoropentyl)pyrazin-2-yl)piperidine-4-carboxylate (Intermediate 49) (2.00 g, 5.57 mmol, 1.0 eq) in DMF (20 mL) at rt was added NIS (1.38 g, 6.12 mmol, 1.1 eq) and TFA (63 mg, 556 μmol, 0.1 eq). The resulting reaction mixture was stirred at rt for 1 hr. The reaction mixture was quenched by the slow addition of a saturated aqueous NaHCO₃ (20 mL), then the reaction mixture was poured into H₂O (200 mL) and extracted with EtOAc (400 mL). The combined organic phase was washed with brine (50 mL), dried with anhydrous Na₂SO₄, filtered, and concentrated under vacuum. The residue was purified by silica gel column chromatography (PE:EtOAc=15:1 to 10:1) to afford ethyl 1-(5-iodo-6-(5,5,5-trifluoropentyl)pyrazin-2-yl)piperidine-4-carboxylate (2.3 g, 85% yield) as a yellow oil. ¹H NMR (400 MHz, CDCl₃) δ 7.76 (s, 1H), 4.19 (m, 4H), 3.03 (dt, J=2.4, 11.2, 2H), 2.79 (t, J=7.2, 2H), 2.57 (m, 1H), 2.19-1.98 (m, 4H), 1.78 (m, 4 H), 1.69 (m, 2H), 1.27 (t, J=7.2, 3H); LCMS calculated for C₁₇H23F₃IN₃O₂: m/z=485; found: m/z=486 (M+H).

### Intermediate 51. Ethyl 1-(3-chloro-5-iodo-6-(5,5,5-trifluoropentyl)pyrazin-2-yl)piperidine-4-carboxylate

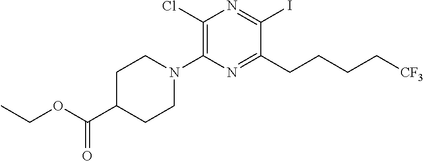 [SMILES: CCOC(=O)C1CCN(c2nc(CCCCC(F)(F)F)c(I)nc2Cl)CC1]

To a solution of ethyl 1-(5-iodo-6-(5,5,5-trifluoropentyl)pyrazin-2-yl)piperidine-4-carboxylate (Intermediate 50) (2.50 g, 5.15 mmol, 1.0 eq) in DMF (25 mL) at rt was added NCS (894 mg, 6.70 mmol, 1.3 eq) and TFA (58 mg, 515 μmol, 0.1 eq). The resulting reaction mixture was heated at 100° C. and stirred for 2 hr. The reaction mixture was cooled to rt, poured into H₂O (200 mL) and extracted with EtOAc (2×200 mL). The combined organic phase was washed with brine (50 mL), dried with anhydrous Na₂SO₄, filtered, and concentrated under vacuum. The residue was purified by silica gel column chromatography (PE:EtOAc=15:1 to 12:1) to afford ethyl 1-(3-chloro-5-iodo-6-(5,5,5-trifluoropentyl)pyrazin-2-yl)piperidine-4-carboxylate (2.5 g, 93% yield) as a yellow oil. ¹H NMR (400 MHz, CDCl₃) δ 4.16 (q, J=7.2, 2H), 3.95 (m, 2H), 3.01 (dt, J=2.4, 11.2, 2H), 2.83 (t, J=7.2, 2H), 2.54 (m, 1H), 2.18 (m, 2H), 2.04 (m, 2H), 1.92-1.76 (m, 4H), 1.66 (m, 2H), 1.27 (t, J=7.2, 3H); LCMS calculated for C₁₇H₂₂ClF₃IN₃O₂: m/z=519; found: m/z=520 (M+H).

### Intermediate 52. Ethyl 1-(3-chloro-6-(5,5,5-trifluoropentyl)pyrazin-2-yl)piperidine-4-carboxylate

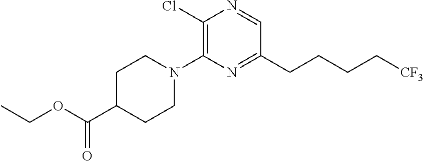 [SMILES: CCOC(=O)C1CCN(c2nc(CCCCC(F)(F)F)cnc2Cl)CC1]

To a solution of ethyl 1-(3-chloro-5-iodo-6-(5,5,5-trifluoropentyl)pyrazin-2-yl)piperidine-4-carboxylate (Intermediate 51) (1.00 g, 1.92 mmol, 1.0 eq) in MeOH (100 mL) at rt was added 10% Pd/C (600 mg). The suspension was degassed under vacuum and purged with H₂ (g) three times. The resulting reaction mixture was stirred at rt under an H₂ (g) atmosphere (30 psi) for 0.5 hr. The reaction mixture was then purged with N₂ (g) and filtered through a pad of celite to remove the catalyst. The filtrate was concentrated under reduced pressure to give a residue. The residue was purified by silica gel column chromatography (PE:EtOAc=15:1 to 10:1) to afford ethyl 1-(3-chloro-6-(5,5,5-trifluoropentyl)pyrazin-2-yl)piperidine-4-carboxylate (900 mg, 79% yield) as a yellow oil. ¹H NMR (400 MHz, CDCl₃) δ 7.71 (s, 1H), 4.16 (q, J=7.2, 2H), 3.93 (m, 2H), 2.99 (dt, J=2.4, 11.2, 2H), 2.69 (t, J=7.2, 2H), 2.53 (m, 1H), 2.16-2.01 (m, 4H), 1.95-1.75 (m, 4H), 1.62 (m, 2H), 1.28 (t, J=7.2, 3H); LCMS calculated for C₁₇H23ClF₃N₃O₂: m/z=393; found: m/z=394 (M+H).

### Intermediate 53. Ethyl 1-(6-(3-hydroxybut-1-yn-1-yl)pyrazin-2-yl)piperidine-4-carboxylate

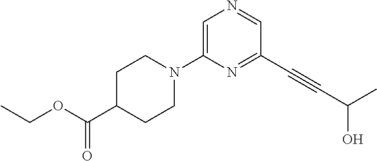 [SMILES: CCOC(=O)C1CCN(c2cncc(C#CC(C)O)n2)CC1]

A mixture of ethyl 1-(6-iodopyrazin-2-yl)piperidine-4-carboxylate (Intermediate 76) (15.0 g, 41.53 mmol, 1.0 eq), but-3-yn-2-ol (8.73 g, 124.59 mmol, 3.0 eq), PPh₃ (2.18 g, 8.31 mmol, 0.2 eq), CuI (1.58 g, 8.31 mmol, 0.2 eq) and Et₃N (21.01 g, 208 mmol, 5.0 eq) in ACN (200 mL) was purged with N₂ (g) three times. Next, Pd(dppf)Cl₂ (3.04 g, 4.15 mmol, 0.1 eq) was added, and the resulting reaction mixture was heated to 60° C. and stirred for 12 hr under an N₂ (g) atmosphere. The reaction mixture was cooled to rt, diluted with H₂O (200 mL) and extracted with EtOAc (3×200 mL). The combined organic layers were washed with brine (300 mL), dried over Na₂SO₄, filtered, and concentrated under reduced pressure to give a residue. The residue was purified by silica gel column chromatography (PE:EtOAc=3:1 to 1:1) to afford ethyl 1-(6-(3-hydroxybut-1-yn-1-yl)pyrazin-2-yl)piperidine-4-carboxylate (12.0 g, 83% yield) as a brown oil. LCMS calculated for C₁₆H₂₁N₃O₃: m/z=303; found: m/z=304 (M+H).

### Intermediate 54. Ethyl 1-(6-(3-hydroxybutyl)pyrazin-2-yl)piperidine-4-carboxylate

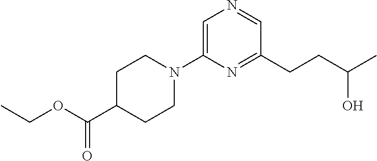 [SMILES: CCOC(=O)C1CCN(c2cncc(CCC(C)O)n2)CC1]

To a solution of ethyl 1-(6-(3-hydroxybut-1-yn-1-yl)pyrazin-2-yl)piperidine-4-carboxylate (Intermediate 53) (15.2 g, 50.11 mmol, 1.0 eq) in MeOH (40 mL) was added 20% Pd(OH)₂/C (1.5 g, 50.11 mmol, 1.0 eq) under an N₂ (g) atmosphere. The suspension was degassed and purged with H₂ (g) three times. The resulting reaction mixture was stirred at rt under an H₂ (g) atmosphere (30 psi) for 4 hr. The reaction mixture was then purged with N₂ (g) and filtered through a pad of celite to remove the catalyst. The filtrate was concentrated under vacuum to afford crude ethyl 1-(6-(3-hydroxybutyl) pyrazin-2-yl)piperidine-4-carboxylate (9.6 g, 55% yield) as a yellow oil. ¹H NMR (400 MHz, CDCl₃) δ 7.99 (s, 1H), 7.73 (s, 1H), 4.18 (m, 4H), 3.88 (m, 1H), 3.04 (m, 2H), 2.78 (t, J=7.2, 2H), 2.56 (m, 1 H), 2.02 (m, 2H), 1.93-1.71 (m, 4H), 1.27 (t, J=7.0, 3H); LCMS calculated for C₁₆H₂₁N₃O₃: m/z=307; found: m/z=308 (M+H).

### Intermediate 55. Ethyl 1-(6-(3-oxobutyl)pyrazin-2-yl)piperidine-4-carboxylate

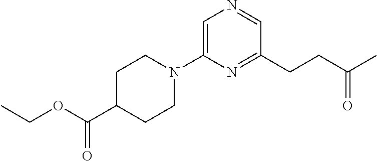 [SMILES: CCOC(=O)C1CCN(c2cncc(CCC(C)=O)n2)CC1]

To a solution of ethyl 1-(6-(3-hydroxybutyl)pyrazin-2-yl)piperidine-4-carboxylate (Intermediate 54) (9.60 g, 31.23 mmol, 1.0 eq) in DCM (120 mL) at rt was added Dess-Martin periodinanae (26.49 g, 62.46 mmol, 2.0 eq). The resulting reaction mixture was stirred at rt for 12 hr. The reaction mixture was then concentrated under reduced pressure to remove DCM. The residue was diluted with saturated aqueous NaHCO₃ (100 mL) and extracted with EtOAc (3×100 mL). The combined organic layers were washed with brine (150 mL), dried over Na₂SO₄, filtered, and concentrated under reduced pressure to give a residue. The residue was purified by silica gel column chromatography (PE:EtOAc=3:1 to 1:1) to afford ethyl 1-(6-(3-oxobutyl)pyrazin-2-yl)piperidine-4-carboxylate (6.20 g, 53% yield) as a brown oil. LCMS calculated for C₁₆H₂₃N₃O₃: m/z=305; found: m/z=306 (M+H).

### Intermediate 56. Ethyl 1-(6-(3,3-difluorobutyl)pyrazin-2-yl)piperidine-4-carboxylate

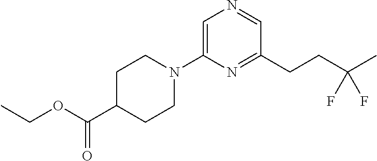 [SMILES: CCOC(=O)C1CCN(c2cncc(CCC(C)(F)F)n2)CC1]

To a solution of ethyl 1-(6-(3-oxobutyl)pyrazin-2-yl)piperidine-4-carboxylate (Intermediate 55) (3.00 g, 9.82 mmol, 1.0 eq) in DCE (30 mL) at rt was added DAST (43.47 g, 196.48 mmol, 20.0 eq). The resulting reaction mixture was stirred at rt for 2 hr. The reaction mixture was then cooled to 0° C. and adjusted pH=8 with saturated aqueous NaHCO₃ (100 mL). The reaction mixture was diluted with H₂O (100 mL), extracted with EtOAc (3×200 mL), dried over Na₂SO₄, filtered, and concentrated under reduced pressure to give a residue. The residue was purified by silica gel column chromatography (PE:EtOAc=5:1 to 2:1) to afford ethyl 1-(6-(3,3-difluorobutyl)pyrazin-2-yl)piperidine-4-carboxylate (1.20 g, 33% yield) as a yellow oil. ¹H NMR (400 MHz, CDCl₃) δ 7.99 (s, 1H), 7.73 (s, 1H), 4.27 (m, 2H), 4.17 (q, J=7.2, 2H), 3.04 (t, J=8.8, 2H), 2.88 (m, 2H), 2.57 (m, 1H), 2.29 (m, 2 H), 2.02 (m, 2H), 1.80 (m, 2H), 1.65 (t, J=18.4, 3H), 1.28 (t, J=7.2, 3H); LCMS calculated for C₁6H23F₂N₃O₂: m/z=327; found: m/z=328 (M+H).

### Intermediate 57. Ethyl 1-(6-(3,3-difluorobutyl)-5-iodopyrazin-2-yl)piperidine-4-carboxylate

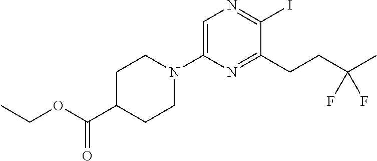 [SMILES: CCOC(=O)C1CCN(c2cnc(I)c(CCC(C)(F)F)n2)CC1]

To a solution of ethyl 1-(6-(3,3-difluorobutyl)pyrazin-2-yl)piperidine-4-carboxylate (Intermediate 56) (1.7 g, 5.19 mmol, 1.0 eq) in DMF (15 mL) at rt was added TFA (593 mg, 5.19 mmol, 1.0 eq) and NIS (1.52 g, 6.75 mmol, 1.3 eq). The resulting reaction mixture was stirred at rt for 1 hr. The reaction mixture was adjusted pH=8 with saturated aqueous NaHCO₃ (30 mL) and then extracted with EtOAc (3×20 mL). The combined organic layers were washed with brine (30 mL), dried over Na₂SO₄, filtered, and concentrated under reduced pressure to give a residue. The residue was purified by silica gel column chromatography (PE:EtOAc=5:1 to 3:1) to afford ethyl 1-(6-(3,3-difluorobutyl)-5-iodopyrazin-2-yl)piperidine-4-carboxylate (1.8 g, 76% yield) as a yellow oil. LCMS calculated for C₁₆H₂₂F₂IN₃O₂: m/z=453; found: m/z=454 (M+H).

### Intermediate 58. Ethyl 1-(3-chloro-6-(3,3-difluorobutyl)-5-iodopyrazin-2-yl)piperidine-4-carboxylate

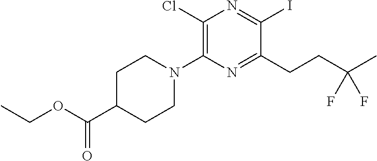 [SMILES: CCOC(=O)C1CCN(c2nc(CCC(C)(F)F)c(I)nc2Cl)CC1]

To a solution of ethyl 1-(6-(3,3-difluorobutyl)-5-iodo-pyrazin-2-yl)piperidine-4-carboxylate (Intermediate 57) (1.5 g, 3.31 mmol, 1.0 eq) in DMF (20 mL) at rt was added TFA (378 mg, 3.31 mmol, 1.0 eq) and NCS (663 mg, 4.96 mmol, 1.5 eq). The resulting reaction mixture was heated to 80° C. and stirred for 1 hr. The reaction mixture was then cooled to rt and adjusted pH=8 with saturated aqueous NaHCO₃ (30 mL) and then extracted with EtOAc (3×20 mL). The combined organic layers were washed with brine (30 mL), dried over Na₂SO₄, filtered, and concentrated under reduced pressure to give a residue. The residue was purified by silica gel column chromatography (PE:EtOAc=1:0 to 4:1) to afford ethyl 1-(3-chloro-6-(3,3-difluorobutyl)-5-iodopyrazin-2-yl)piperidine-4-carboxylate (1.5 g, 77% yield) as a yellow oil. ¹H NMR (400 MHz, CDCl₃) δ 4.18 (q, J=7.0, 2H), 3.94 (m, 2 H), 2.98 (m, 4H), 2.54 (m, 1H), 2.31 (m, 2H), 2.02 (m, 2H), 1.88 (m, 2H), 1.69 (t, J=18.4, 3H), 1.28 (t, J=7.2, 3H).

### Intermediate 59. Ethyl 1-(3-chloro-6-(3,3-difluorobutyl)pyrazin-2-yl)piperidine-4-carboxylate

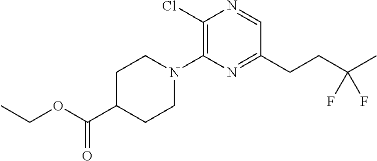 [SMILES: CCOC(=O)C1CCN(c2nc(CCC(C)(F)F)cnc2Cl)CC1]

To a solution of ethyl 1-(3-chloro-6-(3,3-difluorobutyl)-5-iodopyrazin-2-yl)piperidine-4-carboxylate (Intermediate 58) (1.2 g, 2.46 mmol, 1.0 eq) in EtOH (30 mL) was added 10% Pd/C (2.5 g, 2.46 mmol, 1.0 eq). The suspension was degassed and purged with H₂ (g) three times. The resulting reaction mixture was stirred under H₂ (g) (15 psi) at rt for 12 hr. The reaction mixture was then purged with N₂ (g) and filtered through a pad of celite to remove the catalyst. The filtrate was concentrated under vacuum to give a residue. The residue was purified by silica gel column chromatography (PE:EtOAc=0:1 to 3:1) to afford ethyl 1-(3-chloro-6-(3,3-difluorobutyl)pyrazin-2-yl)piperidine-4-carboxylate (920 mg, 83% yield) as a yellow oil. LCMS calculated for C₁₆H₂₂ClF₂N₃O₂: m/z=361; found: m/z=362 (M+H).

### Intermediate 60. Ethyl (E)-1-(6-(4-methoxybut-1-en-1-yl)pyrazin-2-yl)piperidine-4-carboxylate

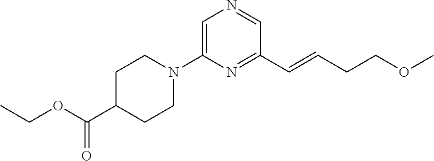 [SMILES: CCOC(=O)C1CCN(c2cncc(/C=C/CCOC)n2)CC1]

To a solution of ethyl 1-(6-formylpyrazin-2-yl)piperidine-4-carboxylate (Intermediate 47) (3.21 g, 12.19 mmol, 1.0 eq) in isopropyl acetate (45 mL) at rt was added (3-methoxypropyl)triphenylphosphonium bromide (6.33 g, 15.24 mmol, 1.3 eq) followed by K₂CO₃ (2.02 g, 14.63 mmol, 1.2 eq). The resulting reaction mixture was heated to 80° C. and stirred for 48 hr. The reaction mixture was then cooled to rt, diluted with H₂O (400 mL) and extracted with EtOAc (2×150 mL). The organic layers were combined, washed with brine (200 mL), dried over Na₂SO₄, filtered, and concentrated under reduced pressure. The residue was purified by silica gel column chromatography (PE:EtOAc=3:1 to 2:1) to afford ethyl (E)-1-(6-(4-methoxybut-1-en-1-yl)pyrazin-2-yl)piperidine-4-carboxylate (3.60 g, 92% yield) as light yellow oil. ¹H NMR (400 MHz, CDCl₃) δ 7.97 (s, 1H), 7.77 (s, 1H), 6.34 (d, J=11.8, 1H), 6.01 (m, 1H), 4.27 (m, 2H), 4.17 (q, J=8.0, 2H), 3.54 (t, J=8.0, 2H), 3.37 (s, 3H), 3.08 (m, 4H), 2.60 (m, 1H), 2.04 (m, 2H), 1.81 (m, 2H), 1.28 (t, J=8.0, 3H); LCMS calculated for C₁₇H₂₅N₃O₃: m/z=319; found: m/z=320 (M+H).

### Intermediate 61. Ethyl 1-(6-(4-methoxybutyl)pyrazin-2-yl)piperidine-4-carboxylate

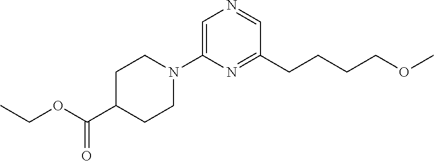 [SMILES: CCOC(=O)C1CCN(c2cncc(CCCCOC)n2)CC1]

To a solution of ethyl (E)-1-(6-(4-methoxybut-1-en-1-yl)pyrazin-2-yl)piperidine-4-carboxylate (Intermediate 60) (1.87 g, 5.85 mmol, 1.0 eq) in EtOH (20 mL) at rt was added 10% Pd/C (1.87 g) under an N₂ (g) atmosphere. The resulting suspension was degassed and purged with H₂ three times, and then the mixture was stirred under an H₂ (g) atmosphere (15 psi) at rt for 2 hr. The reaction mixture was then purged with N₂ (g), filtered through a pad of celite to remove the catalyst, and the filtrate was concentrated under reduced pressure to afford ethyl 1-(6-(4-methoxybutyl)pyrazin-2-yl)piperidine-4-carboxylate (3.65 g, 97% yield) as a light-yellow oil. ¹H NMR (400 MHz, CDCl₃) δ 7.95 (s, 1H), 7.70 (s, 1H), 4.26 (m, 2 H), 4.16 (q, J=8.0, 2H), 3.41 (t, J=6.0, 2H), 3.33 (s, 3H), 3.02 (m, 2H), 2.63 (t, J=6.0, 2 H), 2.55 (m, 1H), 2.01 (m, 2H), 1.80 (m, 4H), 1.66 (m, 2H), 1.27 (t, J=8.0, 3H); LCMS calculated for C₁₇H27N₃O₃: m/z=321; found: m/z=322 (M+H).

### Intermediate 62. Ethyl 1-(5-iodo-6-(4-methoxybutyl)pyrazin-2-yl)piperidine-4-carboxylate

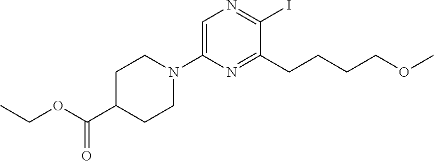 [SMILES: CCOC(=O)C1CCN(c2cnc(I)c(CCCCOC)n2)CC1]

To a solution of ethyl 1-(6-(4-methoxybutyl)pyrazin-2-yl)piperidine-4-carboxylate (Intermediate 61) (3.45 g, 10.73 mmol, 1.0 eq) in DMF (75 mL) at rt was added NIS (3.02 g, 13.42 mmol, 1.2 eq). The resulting reaction mixture was heated to 60° C. and stirred for 2 hr under an N₂ (g) atmosphere in the absence of light. The reaction mixture was then cooled to rt, quenched by the addition of saturated aqueous Na₂SO₃ (10 mL), and then diluted with EtOAc (200 mL) and H₂O (200 mL). The aqueous layer was extracted with EtOAc (3×150 mL). The organic layers were combined, washed with brine (2×150 mL), dried over Na₂SO₄, filtered, and concentrated under reduced pressure to obtain a residue. The residue was purified by silica gel column chromatography (PE:EtOAc=4:1 to 2:1) to afford ethyl 1-(5-iodo-6-(4-methoxybutyl)pyrazin-2-yl)piperidine-4-carboxylate (3.57 g, 74% yield) as a yellow oil. ¹H NMR (400 MHz, CDCl₃) δ 7.73 (s, 1H), 4.19 (m, 2H), 4.15 (q, J=8.0, 2H), 3.42 (t, J=6.0, 2H), 3.34 (s, 3H), 3.01 (m, 2H), 2.77 (t, J=6.0, 2H), 2.54 (m, 1H), 2.01 (m, 2H), 1.76 (m, 4H), 1.68 (m, 2H), 1.26 (t, J=6.0, 3H); LCMS calculated for C₁₇H₂₆IN₃O₃: m/z=447; found: m/z=448 (M+H).

### Intermediate 63. Ethyl 1-(3-chloro-5-iodo-6-(4-methoxybutyl)pyrazin-2-yl)piperidine-4-carboxylate

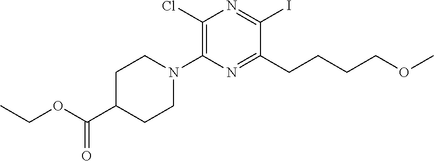 [SMILES: CCOC(=O)C1CCN(c2nc(CCCCOC)c(I)nc2Cl)CC1]

To a solution of ethyl 1-(5-iodo-6-(4-methoxybutyl)pyrazin-2-yl)piperidine-4-carboxylate (Intermediate 62) (2.37 g, 5.30 mmol, 1.0 eq) in DMF (23 mL) at rt was added NCS (707 mg, 5.30 mmol, 1.0 eq). The resulting reaction mixture was heated to 100° C. and stirred for 4 hr under an N₂ (g) atmosphere in the absence of light. The reaction mixture was then cooled to rt and quenched by the addition of saturated aqueous Na₂SO₃ (20 mL). The mixture was then diluted with EtOAc (300 mL) and H₂O (300 mL). The aqueous layer was extracted with EtOAc (2×300 mL). The organic layers were combined, washed with brine (2×200 mL), dried over Na₂SO₄, filtered, and concentrated under reduced pressure to obtain a residue. The residue was purified by silica gel column chromatography (PE:EtOAc=9:1 to 4:1) to afford ethyl 1-(3-chloro-5-iodo-6-(4-methoxybutyl)pyrazin-2-yl)piperidine-4-carboxylate (2.13 g, 83% yield) as a yellow oil. ¹H NMR (400 MHz, CDCl₃) δ 4.16 (q, J=8.0, 2H), 3.95 (m, 2H), 3.43 (t, J=8.0, 2H), 3.35 (s, 3H), 2.98 (m, 2H), 2.83 (t, J=8.0, 2 H), 2.54 (m, 1H), 2.04 (m, 2H), 1.90 (m, 2H), 1.77 (m, 2H), 1.67 (m, 2H), 1.28 (t, J=6.0, 3H); LCMS calculated for C₁₇H₂₅C₁IN₃O₃: m/z=481; found: m/z=482 (M+H).

### Intermediate 64. Ethyl 1-(3-chloro-6-(4-methoxybutyl)pyrazin-2-yl)piperidine-4-carboxylate

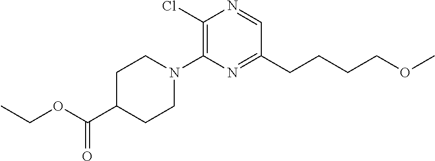 [SMILES: CCOC(=O)C1CCN(c2nc(CCCCOC)cnc2Cl)CC1]

To a suspension of 10% Pd/C (2.81 g, 2.65 mmol) in EtOH (140 mL) at rt was added ethyl 1-(3-chloro-5-iodo-6-(4-methoxybutyl)pyrazin-2-yl)piperidine-4-carboxylate (Intermediate 63) (2.81 g, 5.83 mmol, 1.0 eq) under an N₂ (g) atmosphere. The suspension was degassed and purged with H₂ (g) three times. The mixture was stirred under an H₂ (g) atmosphere (30 psi) at rt for 4 hr. The reaction mixture was then purged with N₂ (g), filtered through a pad of celite to remove the catalyst, and the filtrate was concentrated under reduced pressure to remove the EtOH. The residue was partitioned between H₂O (200 mL) and EtOAc (150 mL). The aqueous layer was extracted with EtOAc (3×150 mL). The organic layers were combined, dried over Na₂SO₄, filtered, and concentrated under reduced pressure to obtain a residue. The residue was purified by silica gel column chromatography (PE:EtOAc=9:1 to 4:1) to afford ethyl 1-(3-chloro-6-(4-methoxybutyl)pyrazin-2-yl)piperidine-4-carboxylate (1.57 g, 76% yield) as a colorless oil. ¹H NMR (400 MHz, CDCl₃) δ 7.71 (s, 1H), 4.17 (q, J=8.0, 2H), 3.93 (m, 2H), 3.40 (t, J=8.0, 2H), 3.33 (s, 3 H), 2.94 (m, 2H), 2.68 (t, J=8.0, 2H), 2.52 (m, 1H), 2.02 (m, 2H), 1.91 (m, 2H), 1.76 (m, 2H), 1.62 (m, 2H), 1.28 (t, J=8.0, 3H); LCMS calculated for C₁₇H₂₆C₁N₃O₃: m/z=355; found: m/z=356 (M+H).

### Intermediate 65. Ethyl 1-(6-(4-hydroxybut-1-yn-1-yl)pyrazin-2-yl)piperidine-4-carboxylate

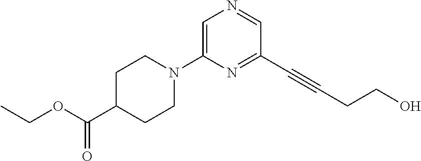 [SMILES: CCOC(=O)C1CCN(c2cncc(C#CCCO)n2)CC1]

To a mixture of ethyl 1-(6-iodopyrazin-2-yl)piperidine-4-carboxylate (Intermediate 76) (15.0 g, 41.53 mmol, 1.0 eq), but-3-yn-1-ol (4.37 g, 62.30 mmol, 4.72 mL, 1.5 eq), CuI (1.58 g, 8.31 mmol, 0.2 eq), PPh₃ (2.18 g, 8.31 mmol, 0.2 eq) and Et₃N (21.01 g, 207.65 mmol, 28.90 mL, 5.0 eq) in ACN (150 mL) at rt under an N₂ (g) atmosphere was added Pd(dppf)Cl₂ (3.04 g, 4.15 mmol, 0.1 eq). The resulting reaction mixture was heated to 80° C. and stirred for 12 hr. The reaction mixture was cooled to rt, diluted with H₂O (200 mL) and extracted with EtOAc (3×200 mL). The combined organic layers were washed with brine (2×200 mL), dried over Na₂SO₄, filtered, and concentrated under reduced pressure to give a residue. The residue was purified by silica gel column chromatography (PE:EtOAc=3:1 to 1:2) to afford ethyl 1-(6-(4-hydroxybut-1-yn-1-yl)pyrazin-2-yl)piperidine-4-carboxylate (11.0 g, 73% yield) as a yellow oil. ¹H NMR (400 MHz, CDCl₃) δ 8.03 (s, 1H), 7.89 (s, 1H), 4.23 (m, 2H), 4.15 (q, J=7.2, 2H), 3.85 (m, 2H), 3.02 (m, 2H), 2.73 (t, J=6.4, 2H), 2.55 (m, 1H), 2.01 (m, 2H), 1.77 (m, 2H), 1.26 (t, J=7.2 Hz, 3H); LCMS calculated for C₁₆H₂₁N₃O₃: m/z=303; found: m/z=304 (M+H).

### Intermediate 66. Ethyl 1-(6-(4-oxobutyl)pyrazin-2-yl)piperidine-4-carboxylate

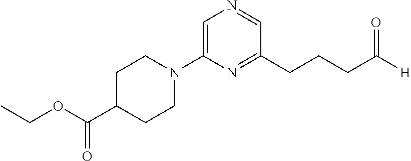 [SMILES: CCOC(=O)C1CCN(c2cncc(CCCC=O)n2)CC1]

To a suspension of 20% Pd(OH)₂ (15.0 g, 21.36 mmol) in EtOH (100 mL) at rt under an N₂ (g) atmosphere was added ethyl 1-(6-(4-hydroxybut-1-yn-1-yl)pyrazin-2-yl)piperidine-4-carboxylate (Intermediate 65) (9.00 g, 29.67 mmol, 1.0 eq). The resulting reaction mixture was degassed under vacuum and purged with H₂ (g) several times and then stirred at rt under an H₂ (g) atmosphere (30 psi) for 96 hr. The reaction mixture was filtered through a pad of Celite, and the filtrate was concentrated under reduced pressure to afford crude ethyl 1-(6-(4-hydroxybutyl) pyrazin-2-yl)piperidine-4-carboxylate (8.00 g, crude) as a yellow oil. In a separate flask, a solution of oxalyl chloride (8.67 g, 68.32 mmol, 5.98 mL, 3.0 eq) in DCM (80 mL) at −78° C. under an N₂ (g) atmosphere was treated with DMSO (8.90 g, 113.86 mmol, 8.90 mL, 5.0 eq) dropwise over 5 min, and the resulting reaction mixture was stirred for 0.5 hr. Next, a solution of crude ethyl 1-(6-(4-hydroxybutyl)pyrazin-2-yl)piperidine-4-carboxylate prepared above in DCM (20 mL) was added dropwise over 10 min and the resulting reaction mixture was stirred at −78° C. for 1 hr. Then Et₃N (18.43 g, 182.18 mmol, 25.36 mL, 8.0 eq) was added dropwise over 5 min and the resulting reaction mixture was stirred at −78° C. for 0.5 hr. The mixture was warmed to rt, diluted with H₂O (50 mL) and extracted with DCM (3×30 mL). The combined organic layers were dried over Na₂SO₄, filtered, and concentrated under reduced pressure to give a residue. The residue was purified by silica gel column chromatography (PE:EtOAc=5:1 to 1:1) to afford ethyl 1-(6-(4-oxobutyl)pyrazin-2-yl)piperidine-4-carboxylate (3.60 g, 52%) as a yellow oil. ¹H NMR (400 MHz, CDCl₃) δ 9.79 (s, 1H), 7.99 (s, 1H), 7.71 (s, 1H), 4.26 (m, 2H), 4.17 (q, J=7.2, 2H), 3.05 (m, 2H), 2.67 (t, J=7.2, 2H), 2.60 (m, 1H), 2.51 (m, 2H), 2.08 (m, 2H), 2.02 (m, 2H), 1.76 (m, 2H), 1.28 (t, J=7.2, 3H).

### Intermediate 67. Ethyl 1-(6-(4,4-difluorobutyl)pyrazin-2-yl)piperidine-4-carboxylate

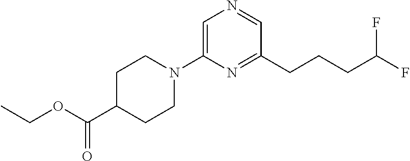 [SMILES: CCOC(=O)C1CCN(c2cncc(CCCC(F)F)n2)CC1]

To a solution of ethyl 1-(6-(4-oxobutyl)pyrazin-2-yl)piperidine-4-carboxylate (Intermediate 66) (3.20 g, 10.48 mmol, 1.0 eq) in DCM (30 mL) at −78° C. was added DAST (4.64 g, 20.96 mmol, 4.59 mL, 2.0 eq). The resulting reaction mixture was warmed to rt and stirred for 3 hr. The reaction mixture was diluted with saturated aqueous NaHCO₃ (50 mL) and then extracted with DCM (3×30 mL). The combined organic layers were dried over Na₂SO₄, filtered, and concentrated under reduced pressure to give a residue. The residue was purified by silica gel column chromatography (PE:EtOAc=5:1 to 1:1) to afford ethyl 1-(6-(4,4-difluorobutyl)pyrazin-2-yl)piperidine-4-carboxylate (1.90 g, 55% yield) as a yellow oil. LCMS calculated for C₁₆H₂₃F₂N₃O₂: m/z=327; found: m/z=328 (M+H).

### Intermediate 68. Ethyl 1-(6-(4,4-difluorobutyl)-5-iodopyrazin-2-yl)piperidine-4-carboxylate

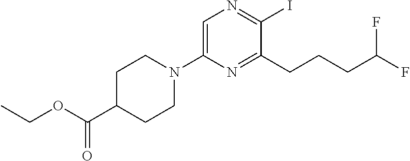 [SMILES: CCOC(=O)C1CCN(c2cnc(I)c(CCCC(F)F)n2)CC1]

A solution of ethyl 1-(6-(4,4-difluorobutyl)pyrazin-2-yl)piperidine-4-carboxylate (Intermediate 67) (2.00 g, 6.11 mmol, 1.0 eq), TFA (696 mg, 6.11 mmol, 452 μL, 1.0 eq), and NIS (1.79 g, 7.94 mmol, 1.3 eq) in DMF (20 mL) at rt was degassed and purged with N₂ (g) three times, and then the resulting reaction mixture was stirred at rt for 1 hr. The reaction mixture was diluted with saturated aqueous NaHCO₃ (30 mL) and then extracted with EtOAc (3×50 mL). The combined organic layers were washed with brine (2×50 mL), dried over Na₂SO₄, filtered, and concentrated under reduced pressure to give a residue. The residue was purified by silica gel column chromatography (PE:EtOAc=30:1 to 10:1) to afford ethyl 1-(6-(4,4-difluorobutyl)-5-iodopyrazin-2-yl)piperidine-4-carboxylate (2.16 g, 78% yield) as a yellow oil. LCMS calculated for C₁₆H₂₂F₂IN₃O₂: m/z=453; found: m/z=454 (M+H).

### Intermediate 69. Ethyl 1-(3-chloro-6-(4,4-difluorobutyl)-5-iodopyrazin-2-yl)piperidine-4-carboxylate

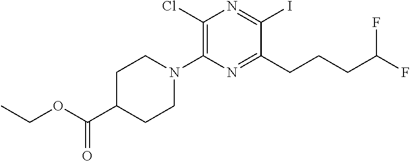 [SMILES: CCOC(=O)C1CCN(c2nc(CCCC(F)F)c(I)nc2Cl)CC1]

A solution of ethyl 1-(6-(4,4-difluorobutyl)-5-iodopyrazin-2-yl)piperidine-4-carboxylate (Intermediate 68) (2.20 g, 4.85 mmol, 1.0 eq), NCS (842 mg, 6.31 mmol, 1.3 eq), TFA (553 mg, 4.85 mmol, 359 μL, 1.0 eq) in DMF (20 mL) at rt was degassed and purged with N₂ (g) three times, and then the resulting reaction mixture was heated to 100° C. and stirred for 1 hr. The reaction mixture was cooled to rt, diluted with saturated aqueous NaHCO₃ (30 mL), and extracted with EtOAc (2×50 mL). The combined organic layers were washed with brine (2×50 mL), dried over Na₂SO₄, filtered, and concentrated under reduced pressure to give a residue. The residue was purified by silica gel column chromatography (PE:EtOAc=1:0 to 10:1) to afford ethyl 1-(3-chloro-6-(4,4-difluorobutyl)-5-iodopyrazin-2-yl)piperidine-4-carboxylate (1.62 g, 68% yield) as a yellow oil. LCMS calculated for C₁₆H₂₁ClF₂IN₃O₂: m/z=487; found: m/z=488 (M+H).

### Intermediate 70. Ethyl 1-(3-chloro-6-(4,4-difluorobutyl)pyrazin-2-yl)piperidine-4-carboxylate

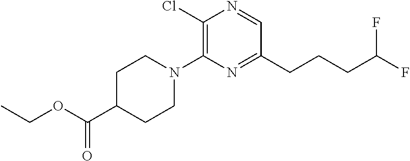 [SMILES: CCOC(=O)C1CCN(c2nc(CCCC(F)F)cnc2Cl)CC1]

To a solution of ethyl 1-(3-chloro-6-(4,4-difluorobutyl)-5-iodopyrazin-2-yl)piperidine-4-carboxylate (Intermediate 69) (1.50 g, 3.08 mmol, 1.0 eq) in EtOH (15 mL) at rt under an N₂ (g) atmosphere was added 10% Pd/C (3.00 g). The resulting suspension was degassed under vacuum and purged with H₂ (g) several times. The resulting reaction mixture was stirred under an H₂ (g) atmosphere (15 psi) at rt for 48 hr. The reaction mixture was purged with N₂ (g) several times, then filtered through a pad of Celite to remove the catalyst. The filtrate was concentrated under reduced pressure to afford a residue. The residue was purified by silica gel column chromatography (PE:EtOAc=30:1 to 10:1) to afford ethyl 1-(3-chloro-6-(4,4-difluorobutyl)pyrazin-2-yl)piperidine-4-carboxylate (975 mg, 87% yield) as a yellow oil. LCMS calculated for C₁₆H₂₂ClF₂N₃O₂: m/z=361; found: m/z=362 (M+H).

### Intermediate 71. 1-(6-(4,4,4-trifluorobutyl)pyrazin-2-yl)piperidine-4-carbonitrile

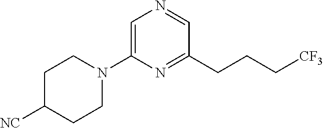 [SMILES: N#CC1CCN(c2cncc(CCCC(F)(F)F)n2)CC1]

To a mixture of zinc (6.44 g, 76.35 mmol, 3.0 eq) in THF (75 mL) at rt under an N₂ (g) atmosphere was added 1,2-dibromoethane (1.18 g, 6.30 mmol, 0.2 eq). The resulting reaction mixture was heated to 80° C. and stirred for 5 min. The reaction mixture was then cooled to rt. This heating and cooling cycle was repeated four times, then TMSCl (205 mg, 1.89 mmol, 0.06 eq) was added to the mixture and the resulting mixture was stirred at rt for 10 min and then cooled to 0° C. Next, a solution of 1,1,1-trifluoro-3-iodo-propane (7.50 g, 31.51 mmol, 1.0 eq) in THF (75 mL) was added to the reaction mixture dropwise over 10 min, and the resulting reaction mixture was stirred at 0° C. for 15 min. Next, 1-(6-chloropyrazin-2-yl)piperidine-4-carbonitrile (Intermediate 36) (3.50 g, 15.72 mmol, 1.0 eq) in 5 mL of THF was added, followed by Pd(dppf)Cl₂-CH₂Cl₂ (1.28 g, 1.57 mmol, 0.1 eq). The resulting reaction mixture was heated at 60° C. with stirring for 12 hr. The reaction mixture was cooled to rt and was then poured into H₂O (300 mL) and extracted with EtOAc (2×300 mL). The combined organic phase was washed with brine (300 mL), dried with anhydrous Na₂SO₄, filtered, and concentrated under vacuum. The residue was purified by silica gel column chromatography (PE:EtOAc=1:1 to 0:1) to afford 1-(6-(4,4,4-trifluorobutyl)pyrazin-2-yl)piperidine-4-carbonitrile (4.1 g, 87%) as a black oil. ¹H NMR (400 MHz, CDCl₃) δ 7.94 (s, 1H), 7.69 (s, 1H), 3.79 (m, 2H), 3.52 (m, 2H), 2.84 (m, 1H), 2.64 (t, J=7.2, 2H), 2.07 (m, 2H), 1.92 (m, 6H).

### Intermediate 72. 1-(5-iodo-6-(4,4,4-trifluorobutyl)pyrazin-2-yl)piperidine-4-carbonitrile

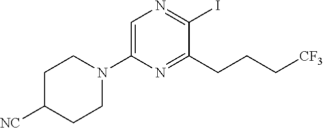 [SMILES: N#CC1CCN(c2cnc(I)c(CCCC(F)(F)F)n2)CC1]

To a solution of 1-(6-(4,4,4-trifluorobutyl)pyrazin-2-yl)piperidine-4-carbonitrile (Intermediate 71) (3.10 g, 10.39 mmol, 1.0 eq) in DMF (30 mL) at rt under an N₂ (g) atmosphere was added NIS (3.04 g, 13.51 mmol, 1.3 eq) and TFA (1.18 g, 10.39 mmol, 1 eq). The resulting reaction mixture was then stirred at rt for 1 hr. The reaction mixture was quenched by saturated aqueous NaHCO₃ (60 mL), poured into H₂O (100 mL) and extracted with EtOAc (2×140 mL). The combined organic phase was washed with brine (150 mL), dried with anhydrous Na₂SO₄, filtered, and concentrated under vacuum. The residue was purified by silica gel column chromatography (PE:EtOAc=1:0 to 4:1) to afford 1-(5-iodo-6-(4,4,4-trifluorobutyl)pyrazin-2-yl)piperidine-4-carbonitrile (3.6 g, 81% yield) as a yellow oil. ¹H NMR (400 MHz, CDCl₃) δ 7.72 (s, 1H), 3.74 (m, 2H), 3.51 (m, 2H), 2.80 (m, 1H), 2.72 (m, 2H), 2.09 (m, 2H), 1.94 (m, 6H).

### Intermediate 73. 1-(3-chloro-5-iodo-6-(4,4,4-trifluorobutyl)pyrazin-2-yl)piperidine-4-carbonitrile

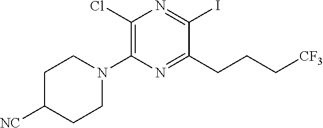 [SMILES: N#CC1CCN(c2nc(CCCC(F)(F)F)c(I)nc2Cl)CC1]

To a solution of 1-(5-iodo-6-(4,4,4-trifluorobutyl)pyrazin-2-yl)piperidine-4-carbonitrile (Intermediate 72) (3.30 g, 7.78 mmol, 1.0 eq) in DMF (30 mL) at rt under an N₂ (g) atmosphere was added NCS (1.14 g, 8.56 mmol, 1.1 eq) and TFA (887 mg, 7.78 mmol, 1.0 eq). The resulting reaction mixture was heated at 100° C. and stirred for 2 hr. The reaction mixture was cooled to rt and was then quenched by saturated aqueous NaHCO₃ (50 mL), poured into H₂O (100 mL) and extracted with EtOAc (3×100 mL). The combined organic phase was washed with brine (300 mL), dried with anhydrous Na₂SO₄, filtered, and concentrated under vacuum. The residue was purified by silica gel column chromatography (PE:EtOAc=1:0 to 6:1) to afford 1-(3-chloro-5-iodo-6-(4,4,4-trifluorobutyl)pyrazin-2-yl)piperidine-4-carbonitrile (3.1 g, 85% yield) as a yellow oil. ¹H NMR (400 MHz, CDCl₃) δ 3.59 (m, 2H), 3.32 (m, 2H), 2.84 (m, 3H), 2.16 (m, 2H), 1.98 (m, 6H).

### Intermediate 74. 1-(3-chloro-6-(4,4,4-trifluorobutyl)pyrazin-2-yl)piperidine-4-carbonitrile

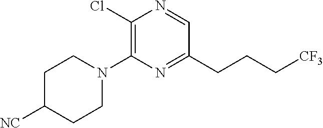 [SMILES: N#CC1CCN(c2nc(CCCC(F)(F)F)cnc2Cl)CC1]

To a solution of ethyl 1-(3-chloro-5-iodo-6-(4,4,4-trifluorobutyl)pyrazin-2-yl)piperidine-4-carbonitrile (2.00 g, 4.36 mmol, 1.0 eq) in MeOH (150 mL) at rt was added 10% Pd/C (1.00 g). The suspension was degassed under vacuum and purged with H₂ (g) three times. The resulting reaction mixture was stirred at rt under an H₂ (g) atmosphere (30 psi) for 12 hr. The reaction mixture was then purged with N₂ (g) three times and filtered through a pad of celite to remove the catalyst. The filtrate was concentrated under reduced pressure to give a residue. The residue was purified by silica gel column chromatography (PE:EtOAc=15:1 to 5:1) to afford 1-(3-chloro-6-(4,4,4-trifluorobutyl)pyrazin-2-yl)piperidine-4-carbonitrile (1.2 g, 80% yield) as a yellow oil. ¹H NMR (400 MHz, CDCl₃) δ 7.70 (s, 1H), 3.59 (m, 2H), 3.31 (m, 2H), 2.83 (m, 1H), 2.70 (m, 2H), 2.01 (m, 8H).

### Intermediate 75. 2,6-diiodopyrazine

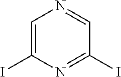

To a solution of 2,6-dichloropyrazine (50.00 g, 335 mmol, 1.0 eq) in HI (250 mL, 48% purity) at rt was added sodium iodide (65.40 g, 436 mmol, 1.3 eq). The resulting reaction mixture was heated to 100° C. and stirred for 1 hr. The mixture was then cooled to rt, diluted with H₂O (500 mL) and extracted with MTBE (3×500 mL). The combined organic layers were washed sequentially with saturated aqueous NaHCO₃ (500 mL), Na₂S20₃ (500 mL) and brine (500 mL), then dried over Na₂SO₄, filtered, and concentrated under reduced pressure to afford 2,6-diiodopyrazine (102 g, crude) as a white solid. ^{1H} NMR (400 MHz, DMSO-d₆) δ 8.91 (s, 2H).

### Intermediate 76. Ethyl 1-(6-iodopyrazin-2-yl)piperidine-4-carboxylate

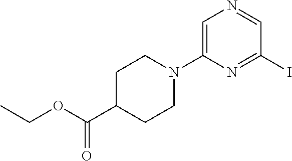 [SMILES: CCOC(=O)C1CCN(c2cncc(I)n2)CC1]

To a solution of 2,6-diiodopyrazine (Intermediate 75) (60 g, 180 mmol, 1.0 eq) in dioxane (800 mL) at rt was added Et₃N (27.44 g, 271 mmol, 37.8 mL, 1.5 eq) and ethyl piperidine-4-carboxylate (42.63 g, 271 mmol, 41.8 mL, 1.5 eq). The resulting reaction mixture was heated to 100° C. and stirred for 12 hr. The mixture was then cooled to rt, diluted with H₂O (500 mL) and extracted with EtOAc (3×500 mL). The combined organic layers were washed with brine (500 mL), dried over Na₂SO₄, filtered, and concentrated under reduced pressure to give a residue. The residue was purified by silica gel column chromatography (PE:EtOAc=1:0 to 10:1) to afford ethyl 1-(6-iodopyrazin-2-yl)piperidine-4-carboxylate (66 g, 96% yield) as a yellow oil. ¹H NMR (400 MHz, CDCl₃) δ 8.04 (s, 1H), 7.99 (s, 1H), 4.23-4.14 (m, 4H), 3.11 (m, 2H), 2.58 (m, 1H), 2.01 (m, 2H), 1.78 (m, 2H), 1.28 (t, J=7.2, 3H); LCMS calculated for C₁₂H₁₆IN₃O₂: m/z=361; found: m/z=362 (M+H).

### Intermediate 77. Ethyl 1-(6-(3-hydroxyprop-1-yn-1-yl)pyrazin-2-yl)piperidine-4-carboxylate

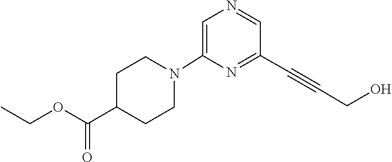 [SMILES: CCOC(=O)C1CCN(c2cncc(C#CCO)n2)CC1]

To a solution of ethyl 1-(6-iodopyrazin-2-yl)piperidine-4-carboxylate (Intermediate 76) (10 g, 28 mmol, 1.00 eq) in ACN (100 mL) at rt was added prop-2-yn-1-ol (3.35 g, 61 mmol, 2.20 eq), CuI (1.05 g, 5.6 mmol, 0.20 eq), PPh₃ (1.45 g, 5.6 mmol, 0.20 eq), Pd(dppf)Cl₂ (1.01 g, 1.4 mmol, 0.05 eq) and Et₃N (14.01 g, 138.5 mmol, 19 mL, 5.00 eq). The resulting reaction mixture was degassed and purged with N₂ (g) three times, then the reaction mixture was heated to 60° C. and stirred for 12 hr under an N₂ (g) atmosphere. The reaction mixture was then cooled to rt and concentrated under reduced pressure to remove most of the ACN. The residue was diluted with H₂O (100 mL) and extracted with EtOAc (3×100 mL). The combined organic layers were washed with brine (500 mL), dried over Na₂SO₄, filtered, and concentrated under reduced pressure to give a residue. The residue was purified by silica gel column chromatography (PE:EtOAc=1:0 to 1:1) to afford ethyl 1-(6-(3-hydroxyprop-1-yn-1-yl)pyrazin-2-yl)piperidine-4-carboxylate (7.50 g, 90% yield) as a brown oil. ¹H NMR (400 MHz, CDCl₃) δ 8.07 (s, 1H), 7.95 (s, 1H), 4.51 (s, 2H), 4.23 (m, 2H), 4.12 (q, J=7.2, 2H), 3.04 (m, 2H), 2.56 (m, 1H), 2.01 (m, 2H), 1.77 (m, 2H), 1.28 (t, J=7.2, 3H); LCMS calculated for C₁₅H₁₉N₃O₃: m/z=289; found: m/z=290 (M+H).

### Intermediate 78. Ethyl 1-(6-(3-hydroxypropyl)pyrazin-2-yl)piperidine-4-carboxylate

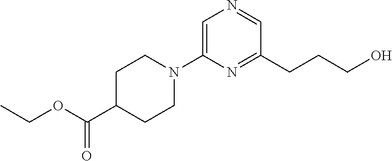 [SMILES: CCOC(=O)C1CCN(c2cncc(CCCO)n2)CC1]

To a solution of ethyl 1-(6-(3-hydroxyprop-1-ynyl)pyrazin-2-yl)piperidine-4-carboxylate (Intermediate 77) (12 g, 41.48 mmol, 1.0 eq) in EtOH (100 mL) at rt was added 20% Pd(OH)₂/C (15.0 g). The resulting suspension was degassed under vacuum and purged with H₂ (g) several times. The reaction mixture was stirred under an H₂ (g) atmosphere (15 psi) at rt for 12 hr. The reaction mixture was then purged with N₂ (g) several times. The reaction mixture was filtered and the filtrate was concentrated to give a residue. The residue was purified by silica gel column chromatography (PE:EtOAc=1:0 to 1:1) to afford ethyl 1-(6-(3-hydroxypropyl)pyrazin-2-yl)piperidine-4-carboxylate (6.75 g, 55% yield) as a yellow oil. ¹H NMR (400 MHz, CDCl₃) δ 8.00 (s, 1H), 7.73 (s, 1H), 4.21 (m, 2H), 4.14 (q, J=7.2, 2H), 3.71 (t, J=6.0, 2H), 3.03 (m, 2H), 2.77 (t, J=7.2, 2H), 2.58 (m, 1H), 2.02 (m, 2H), 1.96 (m, 2H), 1.80 (m, 2H), 1.27 (t, J=7.2, 3H); LCMS calculated for C₁₅H₂₃N₃O₃: m/z=293; found: m/z=294 (M+H).

### Intermediate 79. Ethyl 1-(6-(3-hydroxypropyl)-5-iodopyrazin-2-yl)piperidine-4-carboxylate

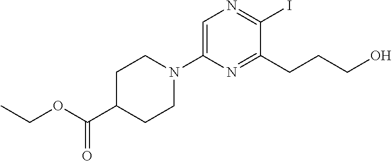 [SMILES: CCOC(=O)C1CCN(c2cnc(I)c(CCCO)n2)CC1]

To a solution of ethyl 1-(6-(3-hydroxypropyl)pyrazin-2-yl)piperidine-4-carboxylate (Intermediate 78) (2.30 g, 8 mmol, 1.0 eq) and NIS (2.29 g, 10 mmol, 1.3 eq) in DMF (30 mL) at rt was added TFA (179 mg, 1.57 mmol, 116 μL, 0.2 eq). The resulting reaction mixture was stirred at rt for 1 hr. The reaction mixture was diluted with saturated aqueous NaHCO₃ (50 mL) and extracted with EtOAc (3×50 mL). The combined organic layers were dried over Na₂SO₄, filtered, and concentrated under reduced pressure to give a residue. The residue was purified by silica gel column chromatography (PE:EtOAc=1:0 to 3:1) to afford ethyl 1-(6-(3-hydroxypropyl)-5-iodopyrazin-2-yl) piperidine-4-carboxylate (1.8 g, 55% yield) as a yellow solid. ¹H NMR (400 MHz, CDCl₃) δ 7.77 (s, 1H), 4.16 (m, 4H), 3.71 (t, J=7.2, 2H), 3.04 (m, 2H), 2.90 (t, J=7.2, 2H), 2.56 (m, 1H), 2.07-1.93 (m, 5H), 1.76 (m, 2H), 1.27 (t, J=7.2, 3H); LCMS calculated for C₁₅H₂₂IN₃O₃: m/z=419; found: m/z=420 (M+H).

### Intermediate 80. Ethyl 1-(3-chloro-6-(3-hydroxypropyl)-5-iodopyrazin-2-yl)piperidine-4-carboxylate

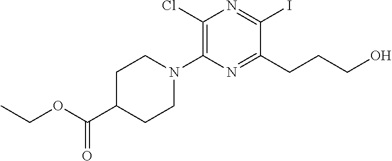 [SMILES: CCOC(=O)C1CCN(c2nc(CCCO)c(I)nc2Cl)CC1]

To a solution of ethyl 1-(6-(3-hydroxypropyl)-5-iodo-pyrazin-2-yl)piperidine-4-carboxylate (Intermediate 79) (1.3 g, 3 mmol, 1 eq) and NCS (497 mg, 3.72 mmol, 1.2 eq) in DMF (20 mL) at rt was added TFA (71 mg, 620 μmol, 46 μL, 0.2 eq). The resulting reaction mixture was heated to 100° C. and stirred for 1 hr. The mixture was then cooled to rt, diluted with H₂O (30 mL) and extracted with EtOAc (2×50 mL). The combined organic layers were dried over Na₂SO₄, filtered, and concentrated under reduced pressure to give a residue. The residue was purified by silica gel column chromatography (PE:EtOAc=1:0 to 3:1) to afford ethyl 1-(3-chloro-6-(3-hydroxypropyl)-5-iodo-pyrazin-2-yl)piperidine-4-carboxylate (1.0 g, 71% yield) as a yellow solid. ¹H NMR (400 MHz, CDCl₃) δ 4.18 (q, J=7.2, 2H), 3.94 (m, 2 H), 3.72 (t, J=6.4, 2H), 3.04-2.88 (m, 4H), 2.53 (m, 1H), 2.02 (m, 4H), 1.89 (m, 2H), 1.28 (t, J=7.2, 3H); LCMS calculated for C₁₅H₂₁C₁IN₃O₃: m/z=453; found: m/z=454 (M+H).

### Intermediate 81. Ethyl 1-(3-chloro-6-(3-hydroxypropyl)pyrazin-2-yl)piperidine-4-carboxylate

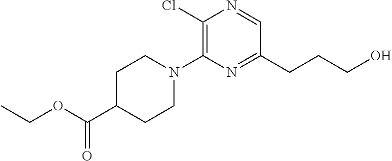 [SMILES: CCOC(=O)C1CCN(c2nc(CCCO)cnc2Cl)CC1]

To a solution of ethyl 1-(3-chloro-6-(3-hydroxypropyl)-5-iodo-pyrazin-2-yl)piperidine-4-carboxylate (Intermediate 80) (1.1 g, 2.42 mmol, 1.0 eq) in EtOH (10 mL) was added 10% Pd/C (2.42 mmol, 1 eq). The resulting suspension was degassed under vacuum and purged with H₂ (g) several times. The reaction mixture was stirred under an H₂ (g) atmosphere (15 psi) at rt for 16 hr. The reaction mixture was then purged with N₂ (g). The reaction mixture was filtered and the filtrate was concentrated to afford a residue. The residue was purified by silica gel column chromatography (PE:EtOAc=1:0 to 2:1) to afford ethyl 1-(3-chloro-6-(3-hydroxypropyl) pyrazin-2-yl) piperidine-4-carboxylate (500 mg, 63% yield) as a yellow oil. ¹H NMR (400 MHz, CDCl₃) δ 7.76 (s, 1H), 4.18 (q, J=7.2, 2H), 3.93 (m, 2 H), 3.71 (m, 2H), 3.00-2.92 (m, 2H), 2.81 (t, J=7.2, 2H), 2.53 (m, 1H), 2.27 (m, 1H), 2.07 (m, 2H), 1.94 (m, 4H), 1.29 (t, J=7.2, 3H); LCMS calculated for C₁₅H₂₂C₁N₃O₃: m/z=327; found: m/z=328 (M+H).

### Intermediate 82. Ethyl 1-(3-chloro-6-(3-oxopropyl)pyrazin-2-yl)piperidine-4-carboxylate

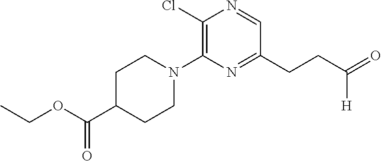 [SMILES: CCOC(=O)C1CCN(c2nc(CCC=O)cnc2Cl)CC1]

A solution of oxalyl chloride (465 mg, 3.66 mmol, 320 μL, 3 eq) in DCM (5 mL) was cooled to −78° C. To this solution was added DMSO (477 mg, 6.10 mmol, 476 μL, 5 eq) and the resulting reaction mixture stirred at −78° C. for 30 min. Next, a solution of ethyl 1-(3-chloro-6-(3-hydroxypropyl)pyrazin-2-yl)piperidine-4-carboxylate (Intermediate 81) (400 mg, 1.22 mmol, 1 eq) in DCM (5 mL) was added to the reaction mixture dropwise over 5 min and the resulting reaction mixture was stirred for 1 hr at −78° C. Then Et₃N (988 mg, 9.76 mmol, 1.36 mL, 8 eq) was added dropwise over 5 min at −78° C. and the resulting reaction mixture was stirred for 30 min., then was warmed to rt and stirred an additional 15 min. The reaction mixture was diluted with H₂O (30 mL) and extracted with DCM (3×30 mL). The combined organic layers were dried over Na₂SO₄, filtered, and concentrated under reduced pressure to give a residue. The residue was purified by silica gel column chromatography (PE:EtOAc=1:0 to 1:1) to afford ethyl 1-(3-chloro-6-(3-oxopropyl)pyrazin-2-yl)piperidine-4-carboxylate (380 mg, 69% yield) as a yellow oil. ¹H NMR (400 MHz, CDCl₃) δ 9.89 (s, 1H), 7.77 (s, 1 H), 4.18 (q, J=7.2, 2H), 3.92 (m, 2H), 3.05 (m, 2H), 2.99-2.84 (m, 4H), 2.53 (m, 1H), 2.03 (m, 2H), 1.90 (m, 2H), 1.29 (t, J=7.2, 3H); LCMS calculated for C₁₅H₂₀ClN₃O₃: m/z=325; found: m/z=326 (M+H).

### Intermediate 83. Ethyl 1-(3-chloro-6-(4,4-difluorobut-3-en-1-yl)pyrazin-2-yl)piperidine-4-carboxylate

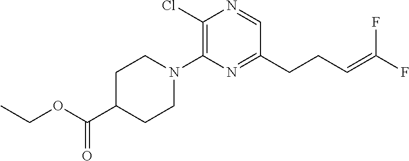 [SMILES: CCOC(=O)C1CCN(c2nc(CCC=C(F)F)cnc2Cl)CC1]

To a mixture of ethyl 1-(3-chloro-6-(3-oxopropyl)pyrazin-2-yl)piperidine-4-carboxylate (Intermediate 82) (911 mg, 2.80 mmol, 1.5 eq), 2-((difluoromethyl)sulfonyl)pyridine (360 mg, 1.86 mmol, 1.0 eq) and CsF (566 mg, 3.73 mmol, 138 μL, 2.0 eq) in DMF (15 mL) at rt under an N₂ (g) atmosphere was added bis(trimethylsilyl)amine (871 mg, 3.73 mmol, 2 eq). The resulting reaction mixture was stirred at rt for 11.5 hr. The mixture was cooled to 0° C. and then saturated aqueous NH₄Cl (100 mg, 1.86 mmol, 1 eq) and HCl (2 M, 13.98 mL, 15 eq) were added to the reaction mixture sequentially, and the resulting reaction mixture was heated to 50° C. and stirred for 30 min. The reaction mixture was cooled to rt, diluted with H₂O (50 mL) and extracted with EtOAc (3×50 mL). The combined organic layers were dried over Na₂SO₄, filtered, and concentrated under reduced pressure to give a residue. The residue was purified by silica gel column chromatography (PE:EtOAc=1:0 to 5:1) to afford ethyl 1-(3-chloro-6-(4, 4-difluorobut-3-en-1-yl) pyrazin-2-yl) piperidine-4-carboxylate (420 mg, 61% yield) as a yellow oil. ¹H NMR (400 MHz, CDCl₃) δ 7.63 (s, 1H), 4.11 (m, 3H), 3.87 (m, 2H), 2.90 (m, 2H), 2.66 (t, J=7.6, 2H), 2.46 (m, 1H), 2.34 (m, 2H), 1.97 (m,), 1.85 (m, 2H), 1.21 (t, J=7.2, 3H); LCMS calculated for C₁₆H₂₀ClF₂N₃O₂: m/z=359; found: m/z=360 (M+H).

### Intermediate 84. 5-bromo-2-chloro-3-(methoxymethoxy)pyridine

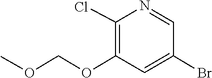

To a solution of NaH (2.49 g, 62.37 mmol, 60% purity, 1.30 eq) in DMF (50 mL) at rt under an N₂ (g) atmosphere was added 5-bromo-2-chloropyridin-3-ol (10.00 g, 47.98 mmol, 1.00 eq) in DMF (50 mL) dropwise over 10 min. The resulting reaction mixture was stirred at rt for 30 min., then MOMCl (4.83 g, 59.99 mmol, 4.56 mL, 1.25 eq) was added. The resulting reaction mixture was stirred at rt for 2 hr. The reaction mixture was diluted with saturated aqueous NaHCO₃ (100 mL) and extracted with EtOAc (2×100 mL). The combined organic layers were washed with brine (2×100 mL), dried over Na₂SO₄, filtered, and concentrated under reduced pressure to give a residue. The residue was purified by silica gel column chromatography (PE:EtOAc=20:1 to 5:1) to afford 5-bromo-2-chloro-3-(methoxymethoxy)pyridine (10.0 g, 81% yield) as a white solid.

### Intermediate 85. 3-(6-chloro-5-(methoxymethoxy)pyridin-3-yl)prop-2-yn-1-ol

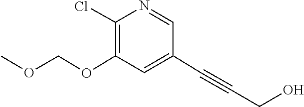

A mixture of 3-methoxyprop-1-yne (12.21 g, 174.26 mmol, 14.37 mL, 2.00 eq), 5-bromo-2-chloro-3-(methoxymethoxy)pyridine (Intermediate 84) (22.00 g, 87.13 mmol, 1.00 eq), CuI (1.66 g, 8.71 mmol, 0.10 eq), [2-(2-aminophenyl)phenyl]palladium(1+); bis(1-adamantyl)-butyl-phosphane; methanesulfonate (CataCXium A Pd G3) (3.17 g, 4.36 mmol, 0.05 eq) and Cs₂CO₃ (85.17 g, 261.39 mmol, 3.00 eq) in ACN (400 mL) was degassed under vacuum and purged with N₂ (g) three times, and then the reaction mixture was heated to 100° C. and stirred for 12 hr. The reaction mixture was cooled to rt and filtered through a pad of Celite. The filtrate was diluted with H₂O (300 mL) and extracted with EtOAc (3×200 mL). The combined organic layers were washed with brine (2×200 mL), dried over Na₂SO₄, filtered, and concentrated under reduced pressure to give a residue. The residue was purified by silica gel column chromatography (PE:EtOAc=50:1 to 10:1) to afford 2-chloro-3-(methoxymethoxy)-5-(3-methoxyprop-1-yn-1-yl)pyridine (17.0 g, 80% yield) as a yellow solid. ¹H NMR (400 MHz, CDCl₃) δ 8.12 (d, J=1.6, 1H), 7.53 (d, J=1.6, 1H), 5.27 (s, 2 H), 4.32 (s, 2H), 3.52 (s, 3H), 3.46 (s, 3H); LCMS calculated for C₁₁H₁₂ClNO₃: m/z=241; found: m/z=242 (M+H).

### Intermediate 86. 2-chloro-3-(methoxymethoxy)-5-(3-methoxypropyl)pyridine

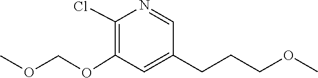

To a solution of 2-chloro-3-(methoxymethoxy)-5-(3-methoxyprop-1-ynyl)pyridine (13.00 g, 53.79 mmol, 1.0 eq) in MeOH (10 mL) at rt under an N₂ (g) atmosphere was added 5% Rh/C (6.00 g). The resulting suspension was degassed under vacuum and purged with H₂ (g) three times. The reaction mixture was stirred under H₂ (g) (15 psi) at rt for 8 h. The reaction mixture was then purged with N₂(g) three times, filtered through a pad of Celite, and the filtrate was concentrated under reduced pressure to give a residue. The residue was purified by silica gel column chromatography (PE:EtOAc=30:1 to 10:1) to afford 2-chloro-3-(methoxymethoxy)-5-(3-methoxypropyl)pyridine (10.00 g, 75% yield) as a yellow oil. LCMS calculated for C₁₁H₁₆ClNO₃: m/z=245; found: m/z=246 (M+H).

### Intermediate 87. 6-bromo-4-fluorobenzo[d][1,3]dioxole

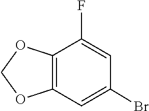 [SMILES: Fc1cc(Br)cc2c1OCO2]

To a solution of 5-bromo-3-fluoro-benzene-1,2-diol (200 mg, 966 μmol, 1.0 eq) and bromo(chloro)methane (375 mg, 2.90 mmol, 3.0 eq) in DMF (3 mL) at rt under an N₂ (g) atmosphere was added Cs2CO₃ (629 mg, 1.93 mmol, 2.0 eq). The resulting reaction mixture was heated to 60° C. and stirred for 3 hr. The reaction mixture was then cooled to rt. The residue was poured into H₂O (60 mL) and extracted with EtOAc (3×60 mL). The combined organic phase was washed with brine (60 mL), dried with anhydrous Na₂SO₄, filtered, and concentrated under vacuum. The residue was purified by silica gel column chromatography (PE:EtOAc=1:0 to 10:1) to afford 6-bromo-4-fluorobenzo[d][1,3]dioxole (60 mg, 18% yield) as a clear liquid. ¹H NMR (400 MHz, CDCl₃) δ 6.77 (dd, J=9.2, 1H), 6.72 (s, 1H), 5.95 (s, 2H).

### Intermediate 88. 2-(7-fluorobenzo[d][1,3]dioxol-5-yl)-4,4,5,5-tetramethyl-1,3,2-dioxaborolane

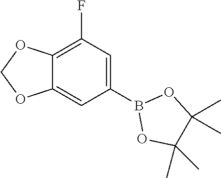 [SMILES: CC1(C)OB(c2cc(F)c3c(c2)OCO3)OC1(C)C]

To a solution of 6-bromo-4-fluoro-1,3-benzodioxole (340 mg, 1.55 mmol, 1.0 eq) and (bis)pinacolatodiborane (591 mg, 2.33 mmol, 1.5 eq) in dioxane (10 mL) at rt under an N₂ (g) atmosphere was added Pd(dppf)Cl₂-CH₂Cl₂ (253 mg, 310 μmol, 0.2 eq) and KOAc (304 mg, 3.10 mmol, 2.0 eq). The resulting reaction mixture was heated to 100° C. and stirred for 12 hr. The reaction mixture was cooled to rt, poured into H₂O (50 mL), and extracted with EtOAc (3×50 mL). The combined organic phase was washed with brine (50 mL), dried with anhydrous Na₂SO₄, filtered, and concentrated under vacuum. The residue was purified by silica gel column chromatography (PE:EtOAc=1:0 to 10:1) to afford 2-(7-fluoro-1,3-benzodioxol-5-yl)-4,4,5,5-tetramethyl-1,3,2-dioxaborolane (240 mg, 58% yield) as a colorless clear liquid. ¹H NMR (400 MHz, CDCl₃) δ 7.19 (s, 1H), 7.09 (s, 1H), 5.95 (s, 2H), 1.27 (s, 12H).

### Intermediate 89. Ethyl 1-(6-(3-methoxyprop-1-yn-1-yl)pyrazin-2-yl)piperidine-4-carboxylate

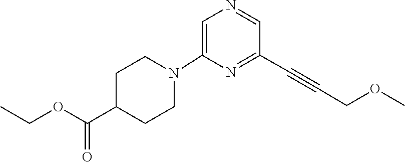 [SMILES: CCOC(=O)C1CCN(c2cncc(C#CCOC)n2)CC1]

To a solution of ethyl 1-(6-iodopyrazin-2-yl)piperidine-4-carboxylate (Intermediate 76) (25.0 g, 69.22 mmol, 1.0 eq) and 3-methoxyprop-1-yne (9.7 g, 138.44 mmol, 2.0 eq) in ACN (250 mL) at rt under an N₂(g) atmosphere was added chloro[(di(1-adamantyl)-N-butylphosphine)-2-(2-aminobiphenyl)]palladium(II) (cataCXium A-Pd-G2) (2.3 g, 3.46 mmol, 0.05 eq), Cs₂CO₃ (67.7 g, 207.65 mmol, 3.0 eq) and CuI (1.3 g, 6.92 mmol, 0.1 eq). The resulting reaction mixture was heated to 80° C. and stirred for 12 hr. The reaction mixture was then cooled to rt and concentrated under reduced pressure to remove most of the ACN. The residue was poured into H₂O (1 L) and extracted with EtOAc (3×1 L). The combined organic phase was washed with brine (500 mL), dried with anhydrous Na₂SO₄, filtered, and concentrated under vacuum. The residue was purified by silica gel column chromatography (PE:EtOAc=5:1 to 3:1) to afford ethyl 1-(6-(3-methoxyprop-1-ynyl)pyrazin-2-yl)piperidine-4-carboxylate (13.1 g, 62% yield) as a brown oil. ¹H NMR (400 MHz, CDCl3) δ 8.10 (s, 1H), 7.97 (s, 1H), 4.37 (s, 2H), 4.27 (m, 2H), 4.19 (q, J=7.2, 2 H), 3.49 (s, 3H), 3.09 (m, 2H), 2.56 (m, 1H), 2.06 (m, 2H), 1.81 (m, 2H), 1.29 (t, J=7.2, 3H).

### Intermediate 90. Ethyl 1-(6-(3-methoxypropyl)pyrazin-2-yl)piperidine-4-carboxylate

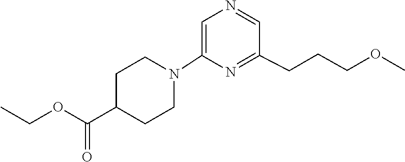 [SMILES: CCOC(=O)C1CCN(c2cncc(CCCOC)n2)CC1]

To a solution of ethyl 1-(6-(3-methoxyprop-1-ynyl)pyrazin-2-yl)piperidine-4-carboxylate (10.0 g, 32.96 mmol, 1.0 eq) in EtOH (500 mL) at rt was added 20% Pd(OH)₂/C (4.4 g). The suspension was degassed under vacuum and purged with H₂ (g) three times. The resulting reaction mixture was stirred at rt under H₂(g) (15 psi) for 3 hr. The reaction mixture was then degassed under vacuum and purged with N₂(g) three times, and then filtered through a pad of celite to remove the catalyst. The filtrate was concentrated under r4educed pressure to afford a residue. The residue was purified by silica gel column chromatography (PE:EtOAc=5:1 to 3:1) to afford ethyl 1-(6-(3-methoxypropyl)pyrazin-2-yl)piperidine-4-carboxylate (9.6 g, 91% yield) as a brown oil. ¹H NMR (400 MHz, CDCl₃) δ 7.89 (s, 1H), 7.64 (s, 1H), 4.19 (m, 2H), 4.10 (q, J=7.2, 2H), 3.35 (t, J=6.4, 2H), 3.28 (s, 3H), 2.95 (m, 2H), 2.64 (t, J=11.2, 2H), 2.47 (m, 1H), 1.95 (m, 4H), 1.71 (m, 2H), 1.20 (t, J=7.2, 3H).

### Intermediate 91. Ethyl 1-(5-iodo-6-(3-methoxypropyl)pyrazin-2-yl)piperidine-4-carboxylate

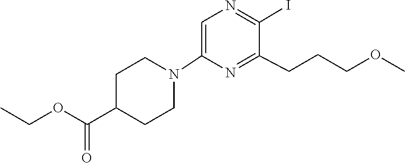 [SMILES: CCOC(=O)C1CCN(c2cnc(I)c(CCCOC)n2)CC1]

To a solution of ethyl 1-(6-(3-methoxypropyl)pyrazin-2-yl)piperidine-4-carboxylate (Intermediate 90) (11.1 g, 36.18 mmol, 1.0 eq) in DMF (100 mL) at rt under an N₂(g) atmosphere was added NIS (8.1 g, 36.18 mmol, 1.0 eq) and TFA (4.1 g, 36.18 mmol, 1.0 eq). The resulting reaction mixture was then stirred for 1 hr at rt. The reaction mixture was poured into H₂O (500 mL) and extracted with EtOAc (3×500 mL). The combined organic phase was washed with brine (300 mL), dried with anhydrous Na₂SO₄, filtered, and concentrated under vacuum. The residue was purified by silica gel column chromatography (PE:EtOAc=5:1 to 3:1) to afford 1-95-iodo-6-(3-methoxypropyl)pyrazin-2-yl)piperidine-4-carboxylate (13.1 g, 84% yield) as a brown oil. ¹H NMR (400 MHz, CDCl₃) δ 7.67 (s, 1H), 4.11 (m, 4H), 3.40 (t, J=6.4, 2H), 3.30 (s, 3H), 2.97 (m, 2H), 2.76 (t, J=7.6, 2H), 2.50 (m, 1H), 1.94 (m, 4H), 1.71 (m, 2H), 1.18 (t, J=4.4, 3H).

### Intermediate 92. Ethyl 1-(3-chloro-5-iodo-6-(3-methoxypropyl)pyrazin-2-yl)piperidine

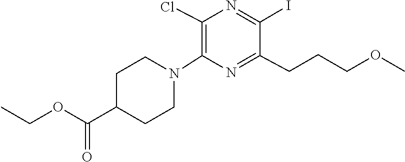 [SMILES: CCOC(=O)C1CCN(c2nc(CCCOC)c(I)nc2Cl)CC1]

To a solution of ethyl 1-(5-iodo-6-(3-methoxypropyl)pyrazin-2-yl)piperidine-4-carbonitrile (Intermediate 91) (32.0 g, 73.85 mmol, 1.0 eq) and NCS (9.9 g, 73.85 mmol, 1.0 eq) in DMF (320 mL) at rt under an N₂ (g) atmosphere was added TFA (8.4 g, 73.85 mmol, 1.0 eq). The resulting reaction mixture was heated to 100° C. and stirred for 3 hr. The reaction mixture was then cooled to rt, poured into H₂O (1 L), and extracted with EtOAc (3×1 L). The combined organic phase was washed with brine (500 mL), dried with anhydrous Na₂SO₄, filtered, and concentrated under vacuum. The residue was purified by silica gel column chromatography (PE:EtOAc=5:1 to 3:1) to afford ethyl 1-(3-chloro-5-iodo-6-(3-methoxypropyl)pyrazin-2-yl]piperidine-4-carboxylate (32.0 g, 92% yield) as a brown oil. ¹H NMR (400 MHz, CDCl₃) δ 4.11 (q, J=7.2, 2H), 3.88 (m, 2H), 3.39 (t, J=6.4, 2H), 3.29 (s, 3H), 2.82 (m, 2H), 2.79 (m, 2H), 2.51 (m, 1H), 1.98 (m, 4H), 1.92 (m, 2H), 1.22-1.17 (t, J=7.2, 3H).

### Intermediate 93. Ethyl 1-(3-chloro-6-(3-methoxypropyl)pyrazin-2-yl)piperidine-4-carboxylate

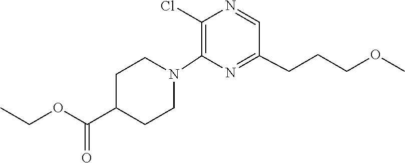 [SMILES: CCOC(=O)C1CCN(c2nc(CCCOC)cnc2Cl)CC1]

To a solution of ethyl 1-(3-chloro-5-iodo-6-(3-methoxypropyl)pyrazin-2-yl)piperidine-4-carboxylate (Intermediate 92) (1.0 g, 2.1 mmol, 1.0 eq) in MeOH (100 mL) at rt was added 10% Pd/C (500 mg). The resulting suspension was degassed under vacuum and purged with H₂ (g) three times. The resulting reaction mixture was stirred at rt under an H₂ (g) atmosphere (30 psi) for 3 hr. The reaction mixture was then degassed under vacuum and purged with N₂ (g) three times, filtered through a pad of celite to remove the catalyst, and the filtrate was concentrated under reduced pressure. The residue was purified by silica gel column chromatography (PE:EtOAc=5:1 to 3:1) to afford ethyl 1-(3-chloro-6-(3-methoxypropyl) pyrazin-2-yl)piperidine-4-carboxylate (750 mg) as a brown oil. ¹H NMR (400 MHz, CDCl₃) δ 7.65 (s, 1H), 4.13 (q, J=7.2, 2H), 3.87 (m, 2H), 3.34 (t, J=6.4, 2H), 3.27 (s, 3H), 2.98 (m, 2H), 2.68 (m, 2H), 2.50 (m, 1H), 1.98-1.84 (m, 6H), 1.21 (t, J=7.2, 3H).

### Intermediate 94. 1-(6-iodopyrazin-2-yl)piperidine-4-carbonitrile

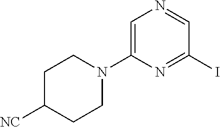 [SMILES: N#CC1CCN(c2cncc(I)n2)CC1]

To a solution of 2,6-diiodopyrazine (Intermediate 75) (10.00 g, 30.13 mmol, 1.0 eq) and piperidine-4-carbonitrile (4.99 g, 34.05 mmol, 1.1 eq) in dioxane (100 mL) at rt was added Et₃N (4.57 g, 45.20 mmol, 1.5 eq). The resulting reaction mixture was heated to 100° C. and stirred for 12 hr. The reaction mixture was then cooled to rt and concentrated under reduced pressure to remove most of the dioxane. The residue was poured into H₂O (400 mL) and extracted with EtOAc (3×200 mL). The combined organic phase was washed with brine (100 mL), dried with anhydrous Na₂SO₄, filtered, and concentrated under vacuum. The residue was purified by silica gel column chromatography (PE:EtOAc=50:1 to 2:1) to 1-(6-iodopyrazin-2-yl)piperidine-4-carbonitrile (17.0 g, 60% yield) as a black oil. ¹H NMR (400 MHz, CDCl₃) δ 8.02 (s, 1H), 7.94 (s, 1H), 3.76 (m, 2H), 3.54 (m, 2H), 2.87 (m, 1H), 1.91 (m, 4H).

### Intermediate 95. 1-(6-(3-methoxyprop-1-yn-1-yl)pyrazin-2-yl)piperidine-4-carbonitrile

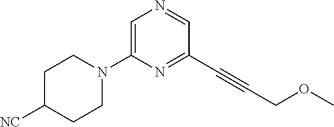 [SMILES: COCC#Cc1cncc(N2CCC(C#N)CC2)n1]

To a solution of 1-(6-iodopyrazin-2-yl)piperidine-4-carbonitrile (Intermediate 94) (4.50 g, 14.33 mmol, 1.0 eq) and 3-methoxyprop-1-yne (2.01 g, 28.65 mmol, 2.0 eq) in ACN (45 mL) at rt under an N₂ (g) atmosphere was added chloro[(di(1-adamantyl)-N-butylphosphine)-2-(2-aminobiphenyl)]palladium(II) (cataCXium A-Pd-G2) (479 mg, 720 μmol, 0.05 eq), Cs₂CO₃ (14.00 g, 42.98 mmol, 3.0 eq) and CuI (270 mg, 1.3 mmol, 0.1 eq). The resulting reaction mixture was heated to 100° C. and stirred for 12 hr. The reaction mixture was then cooled to rt and concentrated under reduced pressure to remove most of the dioxane. The residue was poured into H₂O (100 mL) and extracted with EtOAc (3×50 mL). The combined organic phase was washed with brine (50 mL), dried with anhydrous Na₂SO₄, filtered, and concentrated under vacuum. The residue was purified by silica gel column chromatography (PE:EtOAc=50:1 to 2:1) to afford ethyl 1-(6-(3-methoxyprop-1-yn-1-yl)pyrazin-2-yl)piperidine-4-carbonitrile (2.4 g, 22% yield) as a black oil. ¹H NMR (400 MHz, CDCl₃) δ 8.11 (s, 1H), 8.02 (s, 1H), 4.38 (m, 2H), 3.91 (m, 2H), 3.58 (m, 2H), 3.50 (s, 3H), 2.97 (m, 1H), 2.02 (m, 4H).

### Intermediate 96. 1-(6-(3-methoxypropyl)pyrazin-2-yl)piperidine-4-carbonitrile

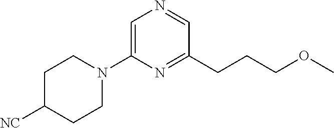 [SMILES: COCCCc1cncc(N2CCC(C#N)CC2)n1]

To a solution of ethyl 1-(6-(3-methoxyprop-1-yn-1-yl)pyrazin-2-yl)piperidine-4-carbonitrile (1.20 g, 4.68 mmol, 1.0 eq) in MeOH (30 mL) at rt was added 10% Pd/C (1.00 g). The suspension was degassed under vacuum and purged with H₂ (g) three times. The resulting reaction mixture was stirred at rt under an H₂ (g) atmosphere (15 psi) for 2 hr. The reaction mixture was then degassed under vacuum and purged with N₂ (g) and filtered through a pad of celite to remove the catalyst. The filtrate was concentrated under reduced pressure to afford ethyl 1-(6-(3-methoxypropyl)pyrazin-2-yl)piperidine-4-carbonitrile (3.2 g, 87% yield) as yellow oil. LCMS calculated for C₁₄H₂₀N₄O: m/z=260; found: m/z=261 (M+H).

### Intermediate 97. 1-(5-iodo-6-(3-methoxypropyl)pyrazin-2-yl)piperidine-4-carbonitrile

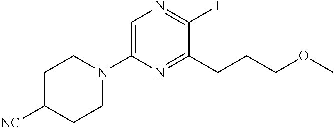 [SMILES: COCCCc1nc(N2CCC(C#N)CC2)cnc1I]

To a solution of ethyl 1-(6-(3-methoxypropyl)pyrazin-2-yl)piperidine-4-carbonitrile (Intermediate 96) (2.70 g, 10.37 mmol, 1.0 eq) in DMF (27 mL) at rt under an N₂ (g) atmosphere was added NIS (2.80 g, 12.45 mmol, 1.2 eq) and TFA (1.18 g, 10.37 mmol, 1.0 eq). The resulting reaction mixture was then stirred for 1 hr at rt. The reaction mixture was poured into H₂O (300 mL) and extracted with EtOAc (3×100 mL). The combined organic phase was washed with brine (30 mL), dried with anhydrous Na₂SO₄, filtered, and concentrated under vacuum. The residue was purified by silica gel column chromatography (PE:EtOAc=10:1 to 0:1) to afford ethyl 1-(5-iodo-6-(3-methoxypropyl)pyrazin-2-yl)piperidine-4-carbonitrile (3.8 g, 80% yield) as a yellow oil. ¹H NMR (400 MHz, CDCl₃) δ 7.69 (s, 1H), 3.73 (m, 2H), 3.49 (m, 2H), 3.40 (t, J=6.8, 2H), 3.30 (s, 3H), 2.86 (m, 1H), 2.77 (t, J=7.6, 2H), 1.91 (m, 6H).

### Intermediate 98. 1-(3-chloro-5-iodo-6-(3-methoxypropyl)pyrazin-2-yl)piperidine-4-carbonitrile

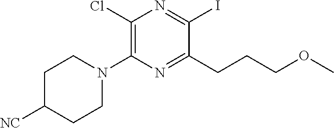 [SMILES: COCCCc1nc(N2CCC(C#N)CC2)c(Cl)nc1I]

To a solution of ethyl 1-(5-iodo-6-(3-methoxypropyl)pyrazin-2-yl)piperidine-4-carbonitrile (Intermediate 97) (2.80 g, 7.25 mmol, 1.0 eq) and NCS (1.16 mg, 8.70 mmol, 1.2 eq) in DMF (28 mL) at rt under an N₂ (g) atmosphere was added TFA (827 mg, 7.25 mmol, 1.0 eq). The resulting reaction mixture was heated to 90° C. and stirred for 1 hr. The reaction mixture was then cooled to rt, poured into H₂O (200 mL), and extracted with EtOAc (3×100 mL). The combined organic phase was washed with brine (50 mL), dried with anhydrous Na₂SO₄, filtered, and concentrated under vacuum. The residue was purified by silica gel column chromatography (PE:EtOAc=10:1 to 0:1) to afford ethyl 1-(3-chloro-5-iodo-6-(3-methoxypropyl)pyrazin-2-yl)piperidine-4-carbonitrile (3.0 g, 62% yield) as a yellow oil. ¹H NMR (400 MHz, CDCl₃) δ 3.68 (m, 2H), 3.46 (t, J=6.4, 2H), 3.37 (m, 5H), 2.90 (m, 3H), 2.06-1.97 (m, 6H).

### Intermediate 99. 1-(3-chloro-6-(3-methoxypropyl)pyrazin-2-yl)piperidine-4-carbonitrile

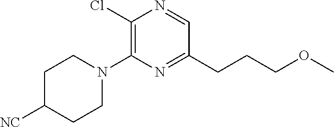 [SMILES: COCCCc1cnc(Cl)c(N2CCC(C#N)CC2)n1]

To a solution of ethyl 1-(3-chloro-5-iodo-6-(3-methoxypropyl)pyrazin-2-yl)piperidine-4-carbonitrile (Intermediate 98) (1.00 g, 2.38 mmol, 1.0 eq) in MeOH (100 mL) at rt was added 10% Pd/C (100 mg). The suspension was degassed under vacuum and purged with H₂ (g) three times. The resulting reaction mixture was stirred at rt under an H₂ (g) atmosphere (30 psi) for 1 hr. The reaction mixture was then degassed under vacuum and purged with N₂ (g) and filtered through a pad of celite to remove the catalyst. The filtrate was concentrated under reduced pressure to give a residue. The residue was purified by silica gel column chromatography (PE:EtOAc=15:1 to 10:1) to afford 1-(3-chloro-6-(3-methoxypropyl)pyrazin-2-yl)piperidine-4-carbonitrile (250 mg, 73% yield) as yellow oil. ¹H NMR (400 MHz, CDCl₃) δ 7.82 (s, 1H), 3.71 (m, 2H), 3.46 (t, J=6.4, 2H), 3.40 (m, 5H), 2.93 (m, 1H), 2.79 (t, J=7.6, 2H), 2.11-2.00 (m, 6H).

### Intermediate 100. Ethyl 1-(6-(3-cyanopropyl)pyrazin-2-yl)piperidine-4-carboxylate

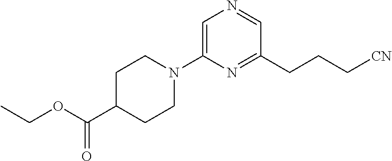 [SMILES: CCOC(=O)C1CCN(c2cncc(CCCC#N)n2)CC1]

To a suspension of Zn (1.35 g, 20.65 mmol, 1.38 eq) in N,N-dimethylacetamide (10 mL) at rt was added 1₂ (190 mg, 748 μmol, 0.05 eq). The mixture was stirred until the yellow color disappeared (1-2 min), then 4-bromobutanenitrile (2.22 g, 15.00 mmol, 1.00 eq) was added. The resulting reaction mixture was heated to 70° C. and stirred for 12 hr. The reaction mixture was cooled to rt, and then ethyl 1-(6-chloropyrazin-2-yl)piperidine-4-carboxylate (Intermediate 8) (910 mg, 3.37 mmol, 1.00 eq) was added. The resulting reaction mixture was degassed under vacuum and purged with N₂ (g) three times, and then Pd(dppf)Cl₂-CH₂Cl₂ (552 mg, 674 μmol, 0.20 eq) was added. The resulting reaction mixture was heated to 60° C. and stirred for 12 hr under an N₂ (g) atmosphere. The reaction mixture was then cooled to rt and filtered through a pad of Celite to remove the catalyst. The filtrate was diluted with H₂O (10 mL) and extracted with EtOAc (3×10 mL). The combined organic layers were washed with brine (20 mL), dried over Na₂SO₄, filtered, and concentrated under vacuum to afford ethyl 1-(6-(3-cyanopropyl)pyrazin-2-yl)piperidine-4-carboxylate (530 mg, 28% yield) as a brown oil. ¹H NMR (400 MHz, CD₃OD) δ8.02 (s, 1H), 7.66 (s, 1H), 4.33 (m, 2H), 4.15 (q, J=7.2, 2H), 3.12 (m, 2H), 2.77 (t, J=7.2, 2H), 2.65 (m, 1H), 2.51 (m, 2 H), 2.23, (m, 2H), 2.00 (m, 2H), 1.69 (m, 2H), 1.26 (t, J=7.2, 3H).

### Intermediate 101. Ethyl 1-(6-(3-cyanopropyl)-5-iodopyrazin-2-yl)piperidine-4-carboxylate

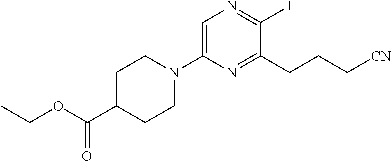 [SMILES: CCOC(=O)C1CCN(c2cnc(I)c(CCCC#N)n2)CC1]

To a solution of ethyl 1-(6-(3-cyanopropyl)pyrazin-2-yl)piperidine-4-carboxylate (Intermediate 100) (1.50 g, 4.96 mmol, 1.0 eq) in DMF (15 mL) at rt was added TFA (566 mg, 4.96 mmol, 1.0 eq) and NIS (1.45 g, 6.45 mmol, 1.3 eq). The resulting reaction mixture was stirred at rt for 1 hr. The reaction mixture was adjusted pH=8 with saturated aqueous NaHCO₃ (20 mL) and extracted with EtOAc (3×20 mL). The combined organic layers were washed with brine (30 mL), dried over Na₂SO₄, filtered, and concentrated under reduced pressure to give a residue. The residue was purified by silica gel column chromatography (PE:EtOAc=5:1 to 2:1) to afford ethyl 1-(6-(3-cyanopropyl)-5-iodopyrazin-2-yl) piperidine-4-carboxylate (1.78 g, 80% yield) as a yellow solid. ¹H NMR (400 MHz, CDCl₃) δ 7.79 (s, 1 H), 4.30-4.08 (m, 4H), 3.05 (t, J=11.2, 2H), 2.92 (t, J=7.2, 2H), 2.58 (m, 1H), 2.47 (t, J=7.2, 2H), 2.14 (m, 2H), 2.02 (m, 2H), 1.76 (m, 2H), 1.28 (t, J=7.2, 3H); LCMS calculated for C₁₆H₂₁IN₄O₂: m/z=428; found: m/z=429 (M+H).

### Intermediate 102. Ethyl 1-(3-chloro-6-(3-cyanopropyl)-5-iodopyrazin-2-yl)piperidine-4-carboxylate

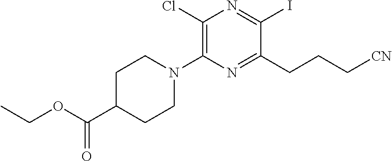 [SMILES: CCOC(=O)C1CCN(c2nc(CCCC#N)c(I)nc2Cl)CC1]

To a mixture of ethyl 1-(6-(3-cyanopropyl)-5-iodopyrazin-2-yl)piperidine-4-carboxylate (Intermediate 101) (1.78 g, 4.16 mmol, 1.0 eq) in DMF (20 mL) at rt was added TFA (474 mg, 4.16 mmol, 1.0 eq) and NCS (833 mg, 6.23 mmol, 1.5 eq). The resulting reaction mixture was heated to 80° C. and stirred for 1 hr. The reaction mixture was then cooled to rt and adjusted pH=8 with saturated aqueous NaHCO₃ (30 mL), and then extracted with EtOAc (3×30 mL). The combined organic layers were washed with brine (50 mL), dried over Na₂SO₄, filtered, and concentrated under reduced pressure to give a residue. The residue was purified by silica gel column chromatography (PE:EtOAc=5:1 to 2:1) to afford ethyl 1-(3-chloro-6-(3-cyanopropyl)-5-iodopyrazin-2-yl)piperidine-4-carboxylate (1.56 g, 80% yield) as a yellow oil. LCMS calculated for C₁₆H₂₀ClIN₄O₂: m/z=462; found: m/z=463 (M+H).

### Intermediate 103. Ethyl 1-(3-chloro-6-(3-cyanopropyl)pyrazin-2-yl)piperidine-4-carboxylate

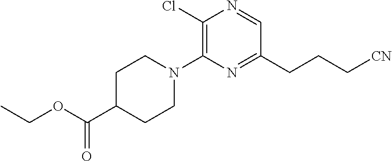 [SMILES: CCOC(=O)C1CCN(c2nc(CCCC#N)cnc2Cl)CC1]

To a mixture of ethyl 1-(3-chloro-6-(3-cyanopropyl)-5-iodopyrazin-2-yl)piperidine-4-carboxylate (Intermediate 102) (780 mg, 1.69 mmol, 1.0 eq) in EtOH (20 mL) at rt was added 10% Pd/C (1.60 g, 1.69 mmol, 1.0 eq). The resulting suspension was degassed under vacuum and purged with H₂ (g) three 3 times. The resulting reaction mixture was stirred under an H₂ (g) atmosphere (15 psi) at rt for 12 hr. The reaction mixture was then degassed under vacuum and purged with N₂ (g). The reaction mixture was then filtered and the filtrate was concentrated under reduced pressure to give a residue. The residue was purified by silica gel column chromatography (PE:EtOAc=4:1 to 2:1) to afford ethyl 1-(3-chloro-6-(3-cyanopropyl)pyrazin-2-yl)piperidine-4-carboxylate (600 mg, 48% yield) as a yellow oil. ¹H NMR (400 MHz, CDCl₃) δ 7.74 (s, 1H), 4.18 (q, J=7.2, 2H), 3.95 (m, 2H), 3.04-2.91 (m, 2H), 2.83 (t, J=7.2, 2H), 2.54 (m, 1H), 2.45 (t, J=7.2, 2H), 2.15 (m, 2H), 2.04 (m, 2H), 1.91 (m, 2H), 1.29 (t, J=7.2, 3H).

### Intermediate 104. 5-(4,4,5,5-tetramethyl-1,3,2-dioxaborolan-2-yl)furo[2,3-b]pyridine

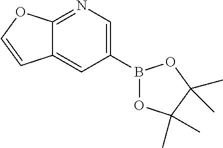 [SMILES: CC1(C)OB(c2cnc3occc3c2)OC1(C)C]

To a solution of 5-bromofuro[2,3-b]pyridine (4.00 g, 20.20 mmol, 1.0 eq) and bis(pinacolato)diboron (6.16 g, 24.24 mmol, 1.2 eq) in dioxane (40 mL) at rt under an N₂ (g) atmosphere was added Pd(dppf)Cl₂-CH₂Cl₂ (1.65 g, 2.02 mmol, 0.1 eq) and KOAc (5.95 g, 60.60 mmol, 3.0 eq). The resulting reaction mixture was heated to 85° C. and stirred for 2 hr. The reaction mixture was cooled to rt, poured into H₂O (150 mL) and extracted with EtOAc (3×150 mL). The combined organic phase was washed with brine (200 mL), dried with anhydrous Na₂SO₄, filtered, and concentrated under vacuum. The residue was purified by silica gel column chromatography (PE:EtOAc=20:1 to 5:1) to afford 5-(4,4,5,5-tetramethyl-1,3,2-dioxaborolan-2-yl)furo[2,3-b]pyridine (6.4 g) as a yellow solid. ¹H NMR (400 MHz, CDCl₃) δ 8.64 (s, 1H), 8.29 (s, 1H), 7.62 (d, J=2.8, 1H), 6.70 (d, J=2.8, 1H), 1.30 (s, 12 H).

### Intermediate 105. 2-methoxy-5-(3-methoxyprop-1-yn-1-yl)pyridine

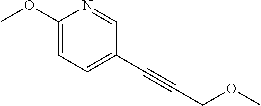

To a solution of 5-bromo-2-methoxypyridine (15.0 g, 79.78 mmol, 1.0 eq) and 3-methoxyprop-1-yne (11.18 g, 159.56 mmol, 2.0 eq) in acetone (300 mL) at rt under an N₂ (g) atmosphere was added CuI (1.52 g, 7.98 mmol, 0.1 eq) and Cs₂CO₃ (77.98 g, 239.33 mmol, 3.0 eq). The resulting reaction mixture was heated to 100° C. and stirred for 12 hr. The reaction mixture was cooled to rt, poured into H₂O (500 mL), and extracted with EtOAc (3×300 mL). The combined organic phase was washed with brine (100 mL), dried with anhydrous Na₂SO₄, filtered, and concentrated under vacuum. The residue was purified by silica gel column chromatography (PE:EtOAc=10:1 to 0:1) to afford methyl 2-methoxy-5-(3-methoxyprop-1-yn-1-yl)pyridine (32.0 g) as a yellow oil. ¹H NMR (400 MHz, CDCl₃) δ 8.20 (d, J=2.4, 1H), 7.53 (dd, J=2.4, 8.4, 1H), 6.62 (d, J=8.4, 1H), 4.24 (s, 2H), 3.87 (s, 3H), 3.38 (s, 3H).

### Intermediate 106. 2-methoxy-5-(3-methoxypropyl)pyridine

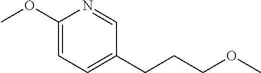

To a solution of 2-methoxy-5-(3-methoxyprop-1-yn-1-yl)pyridine (Intermediate 105) (10.0 g, 56.43 mmol, 1.0 eq) in MeOH (300 mL) at rt under an N₂(g) atmosphere was added 10% Pd/C (10.0 g). The resulting suspension was degassed under vacuum and purged with H₂ (g) three times. The resulting reaction mixture was stirred at rt under an H₂ (g) atmosphere (15 psi) for 12 hr. The reaction mixture was then purged under vacuum and flushed with N₂ (g), filtered through a pad of celite to remove the catalyst, and the filtrate was concentrated under vacuum to afford crude ethyl 2-methoxy-5-(3-methoxypropyl)pyridine (19.0 g, 93% yield) as a yellow oil. ¹H NMR (400 MHz, CDCl₃) δ 7.91 (d, J=2.0, 1H), 7.34 (dd, J=2.0, 8.4, 1H), 6.61 (d, J=8.4, 1H), 3.84 (s, 3H), 3.30 (t, J=8.2, 2H), 3.26 (s, 3H), 2.56 (m, 2H), 1.77 (m, 2H).

### Intermediate 107. 3-bromo-2-methoxy-5-(3-methoxypropyl)pyridine

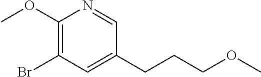

To a solution of 2-methoxy-5-(3-methoxypropyl)pyridine (Intermediate 106) (6.50 g, 35.87 mmol, 1.0 eq) in conc. H₂SO₄ (65 mL) at rt was added NBS (19.15 g, 107.60 mmol, 3.0 eq). The resulting reaction mixture was heated to 60° C. and stirred for 2 hr. The reaction was cooled to 0° C. and was then quenched by addition of ice water (500 mL). The resulting mixture was extracted with DCM (3×200 mL). The combined organic phase was washed with brine (200 mL), dried with anhydrous Na₂SO₄, filtered, and concentrated under vacuum. The residue was purified by silica gel column chromatography (PE:EtOAc=20:1 to 10:1) to afford 3-bromo-2-methoxy-5-(3-methoxypropyl)pyridine (2.80 g, 30% yield) as a colorless liquid. ¹H NMR (400 MHz, CD₃OD) δ7.82 (d, J=2.0, 1H), 7.68 (d, J=2.0, 1H) 3.84 (s, 3 H), 3.28 (t, J=8.2, 2H), 3.22 (s, 3H), 2.51 (t, J=8.2, 2H), 1.72 (m, 2H).

### Intermediate 108. 1-(2-methoxy-5-(3-methoxypropyl)pyridin-3-yl)piperidine-4-carbonitrile

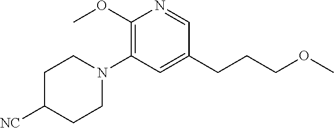 [SMILES: COCCCc1cnc(OC)c(N2CCC(C#N)CC2)c1]

To a solution of 3-bromo-2-methoxy-5-(3-methoxypropyl)pyridine (Intermediate 107) (3.50 g, 13.45 mmol, 1.0 eq) and piperidine-4-carbonitrile (1.97 g, 13.45 mmol, 1.0 eq) in THF (35 mL) at rt under an N₂ (g) atmosphere was added Pd₂(dba)₃ (778 mg, 1.35 mmol, 0.1 eq), t-BuONa (3.88 g, 40.36 mmol, 3.0 eq) and 9,9-dimethyl-9H-xanthene-4,5-diyl)bis(diphenylphosphane) (Xantphos) (1.23 g, 1.35 mmol, 0.1 eq). The resulting reaction mixture was heated to 80° C. and stirred for 12 hr. The reaction mixture was cooled to rt and combined with another batch (500 mg). The mixture was then poured into H₂O (100 mL) and extracted with EtOAc (3×100 mL). The combined organic phase was washed with brine (50 mL), dried with anhydrous Na₂SO₄, filtered, and concentrated under vacuum. The residue was purified by silica gel column chromatography (PE:EtOAc=10:1 to 1:1) to afford 1-(2-methoxy-5-(3-methoxypropyl)pyridin-3-yl)piperidine-4-carbonitrile (3.20 g, 72% yield) as a yellow liquid. LCMS calculated for C₁6H23N₃O₂: m/z=289; found: m/z=290 (M+H).

### Intermediate 109. 1-(2-hydroxy-5-(3-methoxypropyl)pyridin-3-yl)piperidine-4-carbonitrile

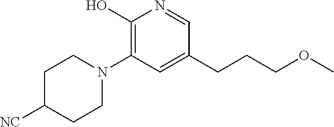 [SMILES: COCCCc1cnc(O)c(N2CCC(C#N)CC2)c1]

A mixture of 1-(2-methoxy-5-(3-methoxypropyl)pyridin-3-yl)piperidine-4-carbonitrile (Intermediate 108) (2.90 g, 10.02 mmol, 1.0 eq) in HCl (3.0 M, 29 ml, 8.7 eq) was heated to 70° C. and stirred for 3 hr. The reaction mixture was cooled to rt and quenched by careful addition of saturated aqueous NaHCO₃ (50 ml), poured into H₂O (400 ml), and extracted with EtOAC (3×200 ml). The combined organic phase was washed with brine (100 ml), dried with anhydrous Na₂SO₄, filtered, and concentrated under vacuum. The residue was purified by silica gel column chromatography (DCM:MeOH=7:1 to 0:1) to afford 1-(2-hydroxy-5-(3-methoxypropyl)pyridin-3-yl)piperidine-4-carbonitrile (2.00 g, 66% yield) as a white solid. ¹H NMR (400 MHz, CDCl₃) δ 8.07 (s, 1H), 6.62 (s, 1H), 3.30 (m, 7 H), 3.02 (m, 2H), 2.78 (m, 1H), 2.40 (m, 2H), 2.06 (m, 4H), 1.74 (m, 2H).

### Intermediate 110. 3-(4-cyanopiperidin-1-yl)-5-(3-methoxypropyl)pyridin-2-yl trifluoromethanesulfonate

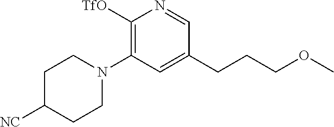 [SMILES: COCCCc1cnc(OS(=O)(=O)C(F)(F)F)c(N2CCC(C#N)CC2)c1]

To a solution of 1-(2-hydroxy-5-(3-methoxypropyl)pyridin-3-yl)piperidine-4-carbonitrile (Intermediate 109) (1.70 g, 6.17 mmol, 1.0 eq) in DCM (17 mL) at 0° C. under an N₂ (g) atmosphere was added Et₃N (2.50 mg, 24.70 mmol, 4.0 eq) and Tf₂O(4.35 g, 15.44 mmol, 2.5 eq). The resulting reaction mixture was warmed to rt and stirred for 16 hr. The reaction mixture was poured into H₂O (200 mL) and extracted with DCM (3×200 mL). The combined organic phase was washed with brine (100 mL), dried with anhydrous Na₂SO₄, filtered, and concentrated under vacuum. The residue was purified by silica gel column chromatography (PE: EtOAc=10:1 to 0:1) to afford 3-(4-cyanopiperidin-1-yl)-5-(3-methoxypropyl)pyridin-2-yl trifluoromethanesulfonate (1.90 g, 64% yield) as a yellow oil. ¹H NMR (400 MHz, CDCl₃) δ 7.77 (d, J=2.0, 1H), 7.23 (d, J=2.0, 1H), 3.33 (t, J=6.0, 2H), 3.28 (s, 3H), 3.17 (m, 2H), 2.98 (m, 2H), 2.81 (m, 1H), 2.65 (t, J=7.6, 2H), 2.02 (m, 4 H), 1.81 (m, 2H).

### Intermediate 111. Ethyl 1-(3,6-dichloropyrazin-2-yl)piperidine-4-carboxylate

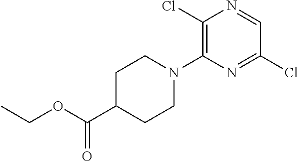 [SMILES: CCOC(=O)C1CCN(c2nc(Cl)cnc2Cl)CC1]

To a solution of 2,3,5-trichloropyrazine (2.00 g, 10.90 mmol, 1.0 eq) and ethyl piperidine-4-carboxylate (1.71 g, 10.90 mmol, 1.68 mL, 1.0 eq) in ACN (37 mL) at rt was added DIEA (4.23 g, 32.71 mmol, 5.70 mL, 3.0 eq). The resulting reaction mixture was heated to 80° C. and stirred for 12 hr. The reaction mixture was then cooled to rt and concentrated under reduced pressure to remove most of the ACN. The reaction mixture was diluted with H₂O (50 mL) and extracted with EtOAc (50 mL×3). The combined organic layers were dried over Na₂SO₄, filtered, and concentrated under reduced pressure to give a residue. The residue was purified by silica gel column chromatography (PE:EtOAc=1:0 to 10:1) to afford ethyl 1-(3,6-dichloropyrazin-2-yl)piperidine-4-carboxylate (1.30 g, 78% yield) as a white solid. LCMS calculated for C₁₂H₁₅C₂N₃O₂: m/z=303; found: m/z=304 (M+H).

### Intermediate 112. Ethyl 1-(3-(benzofuran-5-yl)-6-chloropyrazin-2-yl)piperidine-4-carboxylate

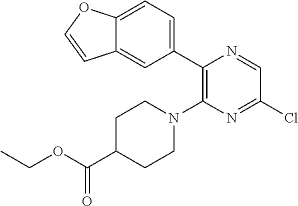 [SMILES: CCOC(=O)C1CCN(c2nc(Cl)cnc2-c2ccc3occc3c2)CC1]

To a solution of ethyl 1-(3,6-dichloropyrazin-2-yl)piperidine-4-carboxylate (Intermediate 111) (3.10 g, 10.19 mmol, 1.0 eq) and benzofuran-5-ylboronic acid (1.65 g, 10.19 mmol, 1.0 eq) in DME (90 mL) at rt was added Pd₂(dba)₃ (933 mg, 1.02 mmol, 0.1 eq), PPh₃ (534 mg, 2.04 mmol, 0.2 eq) and Na₂CO₃ (1 M, 20.38 mL, 2.0 eq). The resulting reaction mixture was heated to 80° C. under an N₂ (g) atmosphere and stirred for 2 hr. The reaction mixture was then cooled to rt, diluted with H₂O (100 mL), and extracted with EtOAc (3×100 mL). The combined organic phase was washed with brine (100 mL), dried with anhydrous Na₂SO₄, filtered, and concentrated under vacuum. The residue was purified by silica gel column chromatography (PE: EtOAc=1:0 to 5:1) to afford ethyl 1-(3-(benzofuran-5-yl)-6-chloropyrazin-2-yl)piperidine-4-carboxylate (3.80 g, 96% yield) as a white solid. LCMS calculated for C₂₀H₂₀ClN₃O₃: m/z=385; found: m/z=386 (M+H).

### Intermediate 113. Ethyl 1-(3-(benzo[d][1,3]dioxol-5-yl)-6-chloropyrazin-2-yl)piperidine-4-carboxylate

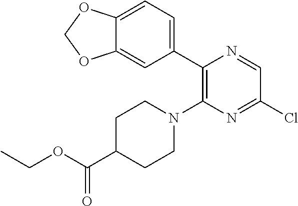 [SMILES: CCOC(=O)C1CCN(c2nc(Cl)cnc2-c2ccc3c(c2)OCO3)CC1]

To a solution of ethyl 1-(3,6-dichloropyrazin-2-yl)piperidine-4-carboxylate (Intermediate 111) (300 mg, 986 μmol, 1.0 eq) and benzo[d][1,3]dioxol-5-ylboronic acid(163 mg, 986 μmol, 1.0 eq) in DME (8 mL) at rt under am N₂ (g) atmosphere was added Pd₂(dba)₃ (90 mg, 98 μmol, 0.1 eq), PPh₃ (51 mg, 197 μmol, 0.2 eq) and Na₂CO₃ (1 M, 1.97 mL, 2.0 eq). The resulting reaction mixture was heated to 80° C. and stirred for 2 hr. The reaction mixture was then cooled to rt, diluted with H₂O (10 mL), and extracted with EtOAc (3×15 mL). The combined organic phase was washed with brine (20 mL), dried with anhydrous Na₂SO₄, filtered, and concentrated under vacuum. The residue was purified by silica gel column chromatography (PE:EtOAc=1:0 to 10:1) to afford ethyl 1-(3-(benzo[d][1,3]dioxol-5-yl)-6-chloropyrazin-2-yl)piperidine-4-carboxylate (340 mg, 88% yield) as a white solid. ¹H NMR (400 MHz, CDCl₃) δ 8.07 (s, 1H), 7.39 (dd, J=1.6, 8.2, 1H), 7.34 (d, J=1.6, 1H), 6.89 (d, J=8.2, 1H), 6.02 (s, 2H), 4.17 (q, J=7.2, 2H), 3.68 (m, 2H), 2.81 (m, 2H), 2.43 (m, 1H), 1.90 (m, 2H), 1.76 (m, 2H), 1.27 (t, J=7.2, 3H); LCMS calculated for C₁₉H₂₀ClN₃O₄: m/z=389; found: m/z=390 (M+H).

### Intermediate 114. Methyl 2-(1-(3,6-dichloropyrazin-2-yl)piperidin-4-yl)acetate

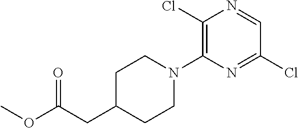 [SMILES: COC(=O)CC1CCN(c2nc(Cl)cnc2Cl)CC1]

To a solution of 2,3,5-trichloropyrazine (500 mg, 2.73 mmol, 1.0 eq) and methyl 2-(piperidin-4-yl)acetate hydrochloride (791 mg, 4.09 mmol, 1.5 eq, HCl salt) in dioxane (10 mL) at rt under an N₂ (g) atmosphere was added Et₃N (414 mg, 4.09 mmol, 1.5 eq). The resulting reaction mixture was heated to 100° C. and stirred for 12 hr. The reaction mixture was cooled to rt and combined with another batch (100 mg scale). The combined mixture was then poured into H₂O (50 mL) and extracted with EtOAc (3×40 mL). The combined organic phase was washed with brine (30 mL), dried with anhydrous Na₂SO₄, filtered, and concentrated under vacuum. The residue was purified by silica gel column chromatography (PE:EtOAc=10:1 to 5:1) to afford methyl 2-(1-(3,6-dichloropyrazin-2-yl)piperidin-4-yl)acetate (650 mg, 65% yield) as a yellow oil. ¹H NMR (400 MHz, CDCl₃) δ 7.72 (s, 1H), 4.03 (m, 2H), 3.63 (s, 3H), 2.85 (dt, J=2.0, 12.8, 2H), 2.24 (d, J=7.2, 2H), 1.98 (m, 1H), 1.18 (m, 2H), 1.36 (m, 2H).

### Intermediate 115. Methyl 2-(1-(3-(benzo[d][1,3]dioxol-5-yl)-6-chloropyrazin-2-yl)piperidin-4-yl)acetate

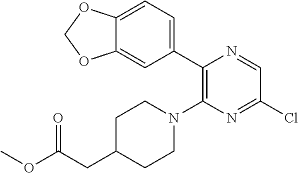 [SMILES: COC(=O)CC1CCN(c2nc(Cl)cnc2-c2ccc3c(c2)OCO3)CC1]

To a solution of methyl 2-(1-(3,6-dichloropyrazin-2-yl)piperidin-4-yl)acetate (Intermediate 114) (550 mg, 1.81 mmol, 1.0 eq) and benzo[d][1,3]dioxol-5-ylboronic acid (300 mg, 1.81 mmol, 1.0 eq) in DME (11 mL) at rt under an N₂(g) atmosphere was added Pd₂(dba)₃ (166 mg, 180 μmol, 0.1 eq) and PPh₃ (95 mg, 361 μmol, 0.2 eq) and Na₂CO₃ (1 M, 3.6 mL, 2.0 eq). The resulting reaction mixture was heated to 80° C. and stirred for 3 hr. The reaction mixture was cooled to rt and combined with another batch (100 mg scale). The combined mixture was then poured into H₂O (50 mL) and extracted with EtOAc (3×40 mL). The combined organic phase was washed with brine (30 mL), dried with anhydrous Na₂SO₄, filtered, and concentrated under vacuum. The residue was purified by silica gel column chromatography (PE:EtOAc=10:1 to 5:1) to afford methyl 2-(1-(3-(benzo[d][1,3]dioxol-5-yl)-6-chloropyrazin-2-yl)piperidin-4-yl)acetate (800 mg, 96% yield) as a yellow oil. ¹H NMR (400 MHz, CDCl₃) δ 7.97 (s, 1H), 7.28 (dd, J=1.6, 8.4, 1H), 7.25 (s, 1H), 6.80 (d, J=8.4, 1H), 5.95 (s, 2H), 3.63 (m, 5H), 2.65 (dt, J=2.0, 12.0, 2H), 2.20 (d, J=7.2, 2H), 1.98 (m, 1H), 1.64 (m, 2H), 1.20 (m, 2H).

### Intermediate 116. Ethyl 3-(1-(3,6-dichloropyrazin-2-yl)piperidin-4-yl)propanoate

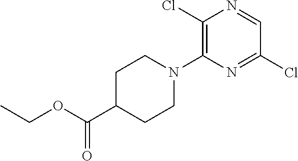 [SMILES: CCOC(=O)C1CCN(c2nc(Cl)cnc2Cl)CC1]

To a solution of 2,3,5-trichloropyrazine (743 mg, 4.05 mmol, 1.0 eq) in ACN (7 mL) at rt was added ethyl 3-(piperidin-4-yl)propanoate (750 mg, 4.05 mmol, 1.0 eq) and Et₃N (2.05 g, 20.24 mmol, 5.0 eq). The resulting reaction mixture was then heated to 80° C. and stirred for 12 hr. The reaction mixture was then cooled to rt, diluted with H₂O (15 mL), and extracted with EtOAc (3×10 mL). The combined organic layers were washed with brine (20 mL), dried over Na₂SO₄, filtered, and concentrated under reduced pressure to give a residue. The residue was purified by silica gel column chromatography (PE:EtOAc=10:1 to 1:1) to afford ethyl 3-(1-(3,6-dichloropyrazin-2-yl)piperidin-4-yl)propanoate (920 mg, 65% yield) as a yellow oil. ¹H NMR (400 MHz, CDCl₃) δ 7.79 (s, 1H), 4.17 (q, J=7.2, 2H), 4.09 (m, 2 H), 2.90 (dt, J=2.0, 8.8, 2H), 2.37 (t, J=7.6, 2H), 1.82 (m, 2H), 1.66 (m, 2H), 1.54 (m, 1 H), 1.37 (m, 2H), 1.27 (t, J=7.2, 3H).

### Intermediate 117. Ethyl 3-(1-(3-(benzo[d][1,3]dioxol-5-yl)-6-chloropyrazin-2-yl)piperidin-4-yl)propanoate

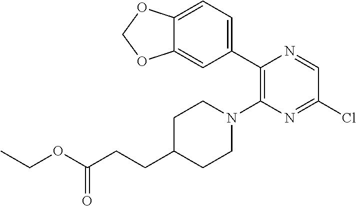 [SMILES: CCOC(=O)CCC1CCN(c2nc(Cl)cnc2-c2ccc3c(c2)OCO3)CC1]

To a solution of ethyl 3-(1-(3,6-dichloropyrazin-2-yl)piperidin-4-yl)propanoate (Intermediate 116) (880 mg, 2.65 mmol, 1.0 eq) in DMF (10 mL) at rt under an N₂ (g) atmosphere was added benzo[d][1,3]dioxol-5-ylboronic acid (440 mg, 2.65 mmol, 1.0 eq), Cs₂CO₃ (1.73 g, 5.30 mmol, 2.0 eq) and Pd(PPh₃)₄(307 mg, 265 μmol, 0.1 eq). The resulting reaction mixture was then heated to 100° C. and stirred for 3 hr. The reaction mixture was cooled to rt, diluted with H₂O (15 mL), and extracted with EtOAc (3×10 mL). The combined organic layers were washed with brine (20 mL), dried over Na₂SO₄, filtered, and concentrated under reduced pressure to give a residue. The residue was purified by silica gel column chromatography (PE:EtOAc=10:1 to 1:1) to afford ethyl 3-(1-(3-(benzo[d][1,3]dioxol-5-yl)-6-chloropyrazin-2-yl)piperidin-4-yl)propanoate (600 mg, 53% yield) as a yellow oil. LCMS calculated for C₂₁H₂₄ClN₃O₄: m/z=417; found: m/z=418 (M+H).

### Intermediate 118. 3-Chloro-5-(3-methoxyprop-1-yn-1-yl)pyridin-2-amine

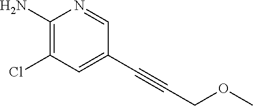

To a solution of 5-bromo-3-chloro-pyridin-2-amine (10.0 g, 48.20 mmol, 1.0 eq) and 3-methoxyprop-1-yne (3.72 g, 53.02 mmol, 1.1 eq) in ACN (100 mL) at rt under an N₂(g) atmosphere was added chloro[(di(1-adamantyl)-N-butylphosphine)-2-(2-aminobiphenyl)]palladium(II) (cataCXium A-Pd-G2) (1.61 g, 2.41 mmol, 0.05 eq), CuI (918 mg, 4.82 mmol, 0.1 eq) and Cs₂CO₃ (47.12 g, 144.61 mmol, 3.0 eq). The resulting reaction mixture was heated to 100° C. and stirred for 2 hr. The reaction mixture was cooled to rt, poured into H₂O (500 mL), and extracted with EtOAc (3×500 mL). The combined organic phase was washed with brine (1000 mL), dried with anhydrous Na₂SO₄, filtered, and concentrated under vacuum. The residue was purified by silica gel column chromatography (PE:EtOAc=1:0 to 3:1) to afford 3-chloro-5-(3-methoxyprop-1-ynyl)pyridin-2-amine (6.2 g, 65% yield) as a white solid. LCMS calculated for C₉H9ClN₂O: m/z=196; found: m/z=197 (M+H).

### Intermediate 119. 3-Chloro-5-(3-methoxypropyl)pyridin-2-amine

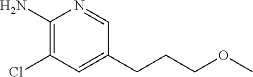

To a solution of 3-chloro-5-(3-methoxyprop-1-ynyl)pyridin-2-amine (Intermediate 118) (6.90 g, 35.09 mmol, 1.0 eq) in MeOH (150 mL) at rt under an N₂ (g) atmosphere was added 5% Rh/C (5.00 g). The resulting suspension was degassed under vacuum and purged with H₂ (g) three times. The resulting reaction mixture was stirred at rt under H₂ (g) (15 Psi) for 2 hr. The reaction mixture was then purged under vacuum and flushed with N₂(g) and filtered through a pad of celite to remove the catalyst. The filtrate was concentrated under reduced pressure to afford crude 3-chloro-5-(3-methoxypropyl)pyridin-2-amine (4.40 g, 62% yield) as a white solid, which was used without further purification. LCMS calculated for C₉H₁₃ClN₂O: m/z=200; found: m/z=201 (M+H).

### Intermediate 120. 3-Chloro-5-(3-methoxypropyl)-2-nitropyridine

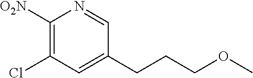

To a solution of 3-chloro-5-(3-methoxypropyl)pyridin-2-amine (Intermediate 119) (4.30 g, 21.43 mmol, 1.0 eq) in conc. H₂SO₄ (43 mL) at −10° C. was added 30% H₂O₂(24.92 g, 219.79 mmol, 10.3 eq). The resulting reaction mixture was stirred at rt for 12 hr. The reaction mixture was then poured into ice water (400 mL) and then extracted with EtOAc (3×300 mL). The combined organic phase was washed with brine (300 mL), dried with anhydrous Na₂SO₄, filtered, and concentrated under vacuum. The residue was purified by silica gel column chromatography (PE:EtOAc=1:0 to 3:1) to afford 3-chloro-5-(3-methoxypropyl)-2-nitropyridine (1.70 g, 34% yield) as a white solid. ¹H NMR (400 MHz, CDCl₃) δ 8.28 (s, 1H), 7.82 (s, 1H), 3.42 (t, J=6.0, 2H), 3.37 (s, 3H), 2.85 (t, J=8.0, 2 H), 1.94 (m, 2H); LCMS calculated for C₉HiiClN₂O₃: m/z=230; found: m/z=231 (M+H).

### Intermediate 121. Ethyl 1-(5-(3-methoxypropyl)-2-nitropyridin-3-yl)piperidine-4-carboxylate

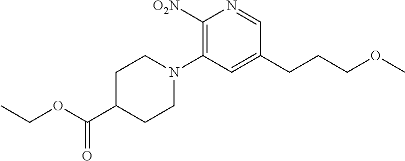 [SMILES: CCOC(=O)C1CCN(c2cc(CCCOC)cnc2[N+](=O)[O-])CC1]

To a solution of 3-chloro-5-(3-methoxypropyl)-2-nitropyridine (Intermediate 120) (300 mg, 1.30 mmol, 1.0 eq) in MeOH (3 mL) at rt was added ethyl piperidine-4-carboxylate (3.06 g, 19.46 mmol, 15.0 eq). The resulting reaction mixture was heated at 80° C. under an N₂ (g) atmosphere and stirred for 3 hr. The reaction mixture was cooled to rt, poured into H₂O (60 mL), and extracted with EtOAc (3×60 mL). The combined organic phase was washed with brine (60 mL), dried with anhydrous Na₂SO₄, filtered, and concentrated under vacuum. The residue was purified by preparative TLC (SiO2, PE:EtOAc=1:1) to afford ethyl 1-(5-(3-methoxypropyl)-2-nitropyridin-3-yl)piperidine-4-carboxylate (380 mg, 83% yield) as a yellow oil. LCMS calculated for C₁₇H₂₅N₃O₅: m/z=351; found: m/z=352 (M+H).

### Intermediate 122. Ethyl 1-(2-amino-5-(3-methoxypropyl)pyridin-3-yl)piperidine-4-carboxylate

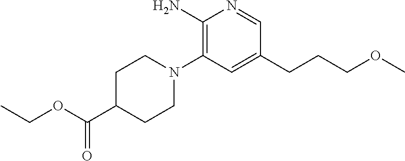 [SMILES: CCOC(=O)C1CCN(c2cc(CCCOC)cnc2N)CC1]

To a solution of ethyl 1-(5-(3-methoxypropyl)-2-nitro-3-pyridyl)piperidine-4-carboxylate (Intermediate 121) (380 mg, 1.08 mmol, 1.0 eq) in MeOH (10 mL) at rt was added 10% Pd/C (0.4 g). The resulting suspension was degassed under vacuum and purged with H₂ (g) three times. The resulting reaction mixture was stirred at rt under an H₂ (g) atmosphere (15 psi) for 2 hr. The reaction mixture was then purged under vacuum and flushed with N₂ (g) and filtered through a pad of celite to remove the catalyst. The filtrate was concentrated under reduced pressure to afford crude ethyl 1-(2-amino-5-(3-methoxypropyl)-3-pyridyl)piperidine-4-carboxylate (380 mg) as a yellow oil, which was used without further purification. ¹H NMR (400 MHz, CD₃OD) δ7.38 (s, 1H), 7.04 (s, 1H), 4.05 (q, J=7.2, 2 H), 3.27 (t, J=6.4, 2H), 3.21 (s, 3H), 3.05 (m, 2H), 2.54 (m, 2H), 2.43 (m, 2H), 2.37 (m, 1H), 1.91 (m, 2H), 1.80 (m, 2H), 1.68 (m, 2H), 1.16 (t, J=7.2, 3H); LCMS calculated for C₁₇H27N₃O₃: m/z=321; found: m/z=322 (M+H).

### Intermediate 123. Ethyl 1-(2-bromo-5-(3-methoxypropyl)pyridin-3-yl)piperidine-4

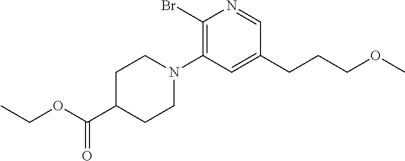 [SMILES: CCOC(=O)C1CCN(c2cc(CCCOC)cnc2Br)CC1]

To a solution of ethyl 1-(2-amino-5-(3-methoxypropyl)-3-pyridyl)piperidine-4-carboxylate (Intermediate 122) (150 mg, 466 μmol, 1.0 eq) in dibromomethane (1.6 mL) at rt under an N₂ (g) atmosphere was added isopentyl nitrite (60 mg, 513 μmol, 1.1 eq). Then to the reaction was added a solution of TMSBr (78 mg, 513 μmol, 1.1 eq) in dibromomethane (1.5 mL). The resulting reaction mixture was stirred at rt for 12 hr. The reaction mixture was then poured into H₂O (40 mL) and extracted with EtOAc (3×40 mL). The combined organic phase was washed with brine (40 mL), dried with anhydrous Na₂SO₄, filtered, and concentrated under vacuum. The residue was purified by preparative TLC (SiO2, PE:EtOAc=2:1) to afford ethyl 1-(2-bromo-5-(3-methoxypropyl)-3-pyridyl)piperidine-4-carboxylate (70 mg, 38% yield) as a yellow oil. ¹H NMR (400 MHz, CD₃OD) δ7.39 (s, 1H), 7.27 (s, 1 H), 4.06 (q, J=7.2, 2H), 3.29 (t, J=6.4, 2H), 3.23 (s, 3H), 2.67 (m, 2H), 2.59 (m, 2H), 2.41 (m, 1H), 1.94 (m, 2H), 1.79 (m, 6H), 1.17 (t, J=7.2, 3H); LCMS calculated for C₁₇H₂₅BrN₂O₃: m/z=384; found: m/z=387 (M+H).

### Intermediate 124. Ethyl 1-(3-(4-hydroxyphenyl)-6-(3-methoxypropyl)pyrazin-2-yl)piperidine-4-carboxylate

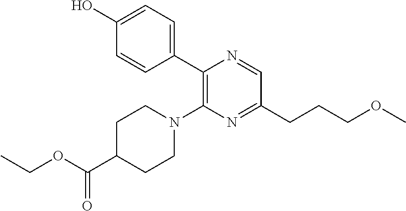 [SMILES: CCOC(=O)C1CCN(c2nc(CCCOC)cnc2-c2ccc(O)cc2)CC1]

To a solution of ethyl 1-(3-chloro-6-(3-methoxypropyl)pyrazin-2-yl)piperidine-4-carboxylate (Intermediate 93) (2.00 g, 5.85 mmol, 1.0 eq) and (4-hydroxyphenyl)boronic acid (1.21 g, 8.78 mmol, 1.5 eq) in THF (40 mL) and H₂O (8 mL) at rt under an N₂ (g) atmosphere was added chloro[(di(1-adamantyl)-N-butylphosphine)-2-(2-aminobiphenyl)]palladium(II) (cataCXium A-Pd-G2) (391 mg, 585 μmol, 0.1 eq) and K₃PO₄ (3.10 g, 14.63 mmol, 2.5 eq). The resulting reaction mixture was heated to 80° C. and stirred for 3 hr. The reaction mixture was cooled to rt, poured into H₂O (100 mL), and extracted with EtOAc (3×60 mL). The combined organic phase was washed with brine (50 mL), dried with anhydrous Na₂SO₄, filtered, and concentrated under vacuum. The residue was purified by silica gel column chromatography (PE:EtOAc=20:1 to 3:1) to afford ethyl 1-(3-(4-hydroxyphenyl)-6-(3-methoxypropyl)pyrazin-2-yl)piperidine-4-carboxylate (2.00 g, 85% yield) as a white solid. ¹H NMR (400 MHz, CDCl₃) δ 7.90 (s, 1H), 7.68 (d, J=6.8, 2H), 6.72 (m, 2H), 6.29 (m, 1H), 4.10 (m, 2H), 3.63 (m, 2H), 3.40 (m, 2H), 3.29 (s, 3H), 2.67 (m, 4H), 2.33 (m, 1H), 1.98 (m, 2H), 1.81 (m, 2H), 1.66 (m, 2H), 1.20 (m, 3H).

### Intermediate 125. tert-Butyl 1-(3,6-dichloropyrazin-2-yl)piperidine-4-carboxylate

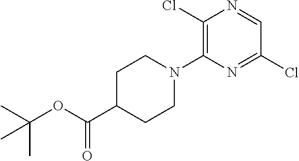 [SMILES: CC(C)(C)OC(=O)C1CCN(c2nc(Cl)cnc2Cl)CC1]

To a solution of 2,3,5-trichloropyrazine (5.00 g, 27.26 mmol, 1.0 eq) and tert-butyl piperidine-4-carboxylate (6.65 g, 29.99 mmol, 1.1 eq) in dioxane (60 mL) was added Et₃N (8.28 g, 81.78 mmol, 11.38 mL, 3.0 eq). The resulting reaction mixture was heated to 100° C. and stirred for 12 hr under an N₂ (g) atmosphere. The reaction mixture was cooled to rt, diluted with H₂O (100 mL), and extracted with EtOAc (3×100 mL). The combined organic layers were washed with brine (100 mL), dried over Na₂SO₄, filtered, and concentrated under reduced pressure to give a residue. The residue was purified by silica gel column chromatography (PE:EtOAc=50:1 to 10:1) to afford tert-butyl 1-(3,6-dichloropyrazin-2-yl)piperidine-4-carboxylate (9.00 g, 98% yield) as a yellow oil. LCMS calculated for C₁₄H₁₉Cl₂N₃O₂: m/z=331; found: m/z=332 (M+H).

### Intermediate 126. tert-Butyl 1-(3-(benzo[d][1,3]dioxol-5-yl)-6-chloropyrazin-2-yl)piperidine-4-carboxylate

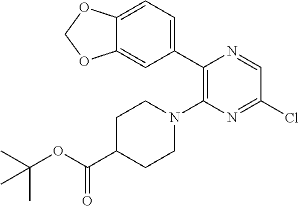 [SMILES: CC(C)(C)OC(=O)C1CCN(c2nc(Cl)cnc2-c2ccc3c(c2)OCO3)CC1]

To a solution of tert-butyl 1-(3,6-dichloropyrazin-2-yl)piperidine-4-carboxylate (Intermediate 125) (4.00 g, 12.04 mmol, 1.0 eq) and benzo[d][1,3]dioxol-5-ylboronic acid (1.95 g, 12.04 mmol, 1.0 eq) in DMF (50 mL) at rt was added Cs₂CO₃ (11.77 g, 36.12 mmol, 3.0 eq) and Pd(PPh₃)₄(1.39 g, 1.20 mmol, 0.1 eq). The resulting reaction mixture was degassed under vacuum and purged with N₂ (g) three times, and then the reaction mixture was heated to 80° C. and stirred for 16 hr under an N₂(g) atmosphere. The reaction mixture was cooled to rt, diluted with H₂O (50 mL), and extracted with EtOAc (3×50 mL). The combined organic layers were dried over Na₂SO₄, filtered, and concentrated under reduced pressure to give a residue. The residue was purified by silica gel column chromatography (PE:EtOAc=1:0 to 5:1) to afford tert-butyl 1-(3-(benzo[d][1,3]dioxol-5-yl)-6-chloropyrazin-2-yl)piperidine-4-carboxylate(4.00 g, 75% yield) as a yellow solid. LCMS calculated for C₂₁H₂₄ClN₃O₄: m/z=417; found: m/z=418 (M+H).

### Intermediate 127. tert-Butyl (E)-1-(3-(benzo[d][1,3]dioxol-5-yl)-6-(3-((tert-butyldimethylsilyl)oxy)prop-1-en-1-yl)pyrazin-2-yl)piperidine-4-carboxylate

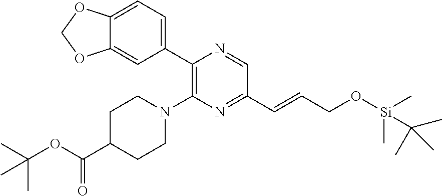 [SMILES: CC(C)(C)OC(=O)C1CCN(c2nc(/C=C/CO[Si](C)(C)C(C)(C)C)cnc2-c2ccc3c(c2)OCO3)CC1]

To a solution of tert-butyl 1-(3-(benzo[d][1,3]dioxol-5-yl)-6-chloropyrazin-2-yl)piperidine-4-carboxylate (Intermediate 126) (8.00 g, 19.14 mmol, 1.0 eq) and (E)-tert-butyldimethyl((3-(4,4,5,5-tetramethyl-1,3,2-dioxaborolan-2-yl)allyl)oxy)silane (6.85 g, 22.97 mmol, 1.2 eq) in dioxane (80 mL) and H₂O (8 mL) at rt was chloro[(di(1-adamantyl)-N-butylphosphine)-2-(2-aminobiphenyl)]palladium(II) (cataCXium A-Pd-G2) (640 mg, 957 μmol, 0.05 eq) and Cs₂CO₃ (18.71 g, 57.43 mmol, 3.0 eq). The resulting reaction mixture was degassed under vacuum and purged with N₂ (g) three times, and then the reaction mixture was heated to 110° C. and stirred for 1 hr under an N₂ (g) atmosphere. The mixture was cooled to rt, diluted with H₂O (100 mL), and extracted with EtOAc (3×100 mL). The combined organic layers were washed with brine (2×100 mL), dried over Na₂SO₄, filtered, and concentrated under reduced pressure to give a residue. The residue was purified by silica gel column chromatography (PE:EtOAc=50:1 to 10:1) to afford tert-butyl (E)-1-(3-(benzo[d][1,3]dioxol-5-yl)-6-(3-((tert-butyldimethylsilyl)oxy)prop-1-en-1-yl)pyrazin-2-yl)piperidine-4-carboxylate (9.00 g, 84% yield) as a yellow oil. ¹H NMR (400 MHz, CDCl₃) δ 8.01 (s, 1H), 7.62 (m, H), 7.49 (m, H), 7.43 (d, J=1.6, H), 6.90 (m, 1H), 6.53 (d, J=12.6, 1H), 6.01 (s, 2H), 4.44 (dd, J=2.0, 4.4, 2H), 3.68 (m, 2H), 2.79 (m, 2H), 2.33 (m, 1H), 1.86 (m, 2H), 1.77-1.68 (m, 2H), 1.46 (s, 9H), 0.98 (s, 9H), 0.14 (s, 6H); LCMS calculated for C₃₀H₄₃N₃O₅Si: m/z=553; found: m/z=554 (M+H).

### Intermediate 128. tert-Butyl 1-(3-(benzo[d][1,3]dioxol-5-yl)-6-(3-((tert-butyldimethylsilyl)oxy)propyl)pyrazin-2-yl)piperidine-4-carboxylate

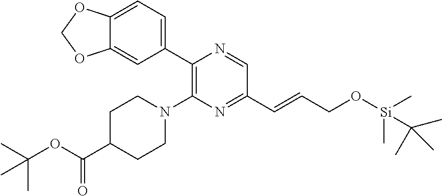 [SMILES: CC(C)(C)OC(=O)C1CCN(c2nc(/C=C/CO[Si](C)(C)C(C)(C)C)cnc2-c2ccc3c(c2)OCO3)CC1]

To a solution of tert-butyl (E)-1-(3-(benzo[d][1,3]dioxol-5-yl)-6-(3-((tert-butyldimethylsilyl)oxy)prop-1-en-1-yl)pyrazin-2-yl)piperidine-4-carboxylate (Intermediate 127) (9.00 g, 16.25 mmol, 1.0 eq) in MeOH (50 mL) was added 10% Pd/C (5.00 g). The resulting suspension was degassed under vacuum and purged with H₂ (g) three times, and then the reaction mixture was stirred at rt for 2 hr under an H₂ (g) atmosphere (15 psi). The reaction mixture was then degassed under vacuum and purged with N₂ (g), filtered through a pad of Celite, and the filtrate was concentrated under reduced pressure to give a residue. The residue was purified by silica gel column chromatography (PE:EtOAc=50:1 to 10:1) to afford tert-butyl 1-(3-(benzo[d][1,3]dioxol-5-yl)-6-(3-((tert-butyldimethylsilyl)oxy)propyl) pyrazin-2-yl)piperidine-4-carboxylate (7.00 g, 66% yield) as a yellow oil. LCMS calculated for C₃₀H₄₅N₃O₅Si: m/z=555; found: m/z=556 (M+H).

### Intermediate 129. tert-Butyl 1-(3-(benzo[d][1,3]dioxol-5-yl)-6-(3-hydroxypropyl)pyrazin-2-yl)piperidine-4-carboxylate

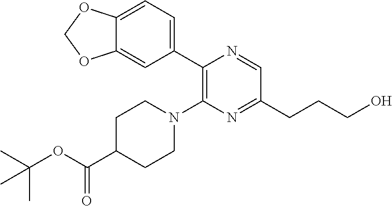 [SMILES: CC(C)(C)OC(=O)C1CCN(c2nc(CCCO)cnc2-c2ccc3c(c2)OCO3)CC1]

To a solution of tert-butyl 1-(3-(benzo[d][1,3]dioxol-5-yl)-6-(3-((tert-butyldimethylsilyl)oxy)propyl) pyrazin-2-yl)piperidine-4-carboxylate (Intermediate 128) (14 g, 25.19 mmol, 1.0 eq) in THF (100 mL) at 0° C. was added TBAF (1 M, 37.78 mL, 1.5 eq). The resulting reaction mixture was warmed to rt and stirred for 2 hr. The reaction mixture was diluted with saturated aqueous Na₂CO₃ (100 mL) and then extracted with EtOAc (3×200 mL). The combined organic layers were washed with saturated Na₂CO₃ (2×100 mL), dried over Na₂SO₄, filtered, and concentrated under reduced pressure to give a residue. The residue was purified by silica gel column chromatography (PE:EtOAc=20:1 to 5:1) to afford tert-butyl 1-(3-(benzol[d][1,3]dioxol-5-yl)-6-(3-hydroxypropyl)pyrazin-2-yl)piperidine-4-carboxylate (8 g, 68% yield) as a yellow solid. ¹H NMR (400 MHz, CDCl₃) δ 8.00 (s, 1H), 7.45 (d, J=8.4, 1H), 7.40 (d, J=1.6, 1H), 6.89 (d, J=8.4, 1H), 6.01 (s, 2H), 3.75 (t, J=6.0, 2H), 3.62 (m, 2H), 2.86 (t, J=6.8, 2H), 2.73 (m, 2H), 2.31 (m, 1H), 2.01 (m, 2H), 1.86 (m, 2H), 1.69 (m, 2H), 1.45 (s, 9H); LCMS calculated for C₂₄H3₁N₃O₅: m/z=441; found: m/z=442 (M+H).

### Intermediate 130. Ethyl 1-(6-chloro-3-(7-fluorobenzofuran-5-yl)pyrazin-2-yl)piperidine-4-carboxylate

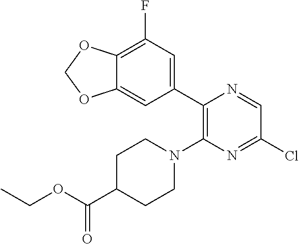 [SMILES: CCOC(=O)C1CCN(c2nc(Cl)cnc2-c2cc(F)c3c(c2)OCO3)CC1]

To a solution of ethyl 1-(3,6-dichloropyrazin-2-yl)piperidine-4-carboxylate (Intermediate 111) (200 mg, 658 μmol, 1.0 eq) and 2-(7-fluorobenzofuran-5-yl)-4,4,5,5-tetramethyl-1,3,2-dioxaborolane (Intermediate 41) (173 mg, 657 μmol, 1.0 eq) in DME (4 mL) and H₂O (0.8 mL) at rt under an N₂ (g) atmosphere was added Pd₂(dba)₃ (60 mg, 65 μmol, 0.1 eq), PPh₃ (35 mg, 131 μmol, 0.2 eq), and Na₂CO₃ (139 mg, 1.32 mmol, 2.0 eq). The resulting reaction mixture was heated to 80° C. and stirred for 2 hr. The reaction mixture was cooled to rt, poured into H₂O (30 mL), and extracted with EtOAc (3×20 mL). The combined organic phase was washed with brine (20 mL), dried with anhydrous Na₂SO₄, filtered, and concentrated under vacuum. The residue was purified by silica gel column chromatography (PE:EtOAc=10:1 to 5:1) to afford ethyl 1-(6-chloro-3-(7-fluorobenzofuran-5-yl)pyrazin-2-yl)piperidine-4-carboxylate (300 mg, 90% yield) as a yellow oil. ¹H NMR (400 MHz, CDCl₃) δ 8.04 (s, 1H), 7.82 (s, 1H), 7.53 (s, 1H), 7.36 (m, 1H), 6.81 (m, 1H), 4.07 (q, J=7.2, 2H), 3.60 (m, 2H), 2.74 (t, J=11.2, 2H), 2.32 (m, 1H), 1.82 (m, 2H), 1.67 (m, 2H), 1.19 (t, J=7.2, 3H).

### Intermediate 131. tert-Butyl 1-(6-chloro-3-(7-fluorobenzofuran-5-yl)pyrazin-2-yl)piperidine-4-carboxylate

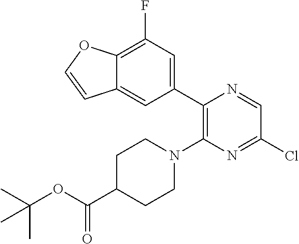 [SMILES: CC(C)(C)OC(=O)C1CCN(c2nc(Cl)cnc2-c2cc(F)c3occc3c2)CC1]

To a solution of tert-butyl 1-(3,6-dichloropyrazin-2-yl)piperidine-4-carboxylate (Intermediate 125) (1.00 g, 3.01 mmol, 1.0 eq) and 2-(7-fluorobenzofuran-5-yl)-4,4,5,5-tetramethyl-1,3,2-dioxaborolane (Intermediate 41) (788 mg, 3.01 mmol, 1.0 eq) in DME (20 mL) at rt under an N₂ (g) atmosphere was added Pd₂(dba)₃ (275 mg, 301 μmol, 0.1 eq), PPh₃ (157 mg, 602 μmol, 0.2 eq) and Na₂CO₃ (1M, 6.02 mL, 2.0 eq). The resulting reaction mixture was heated to 80° C. and stirred for 3 hr. The reaction mixture was cooled to rt, poured into H₂O (60 mL), and extracted with EtOAc (3×50 mL). The combined organic layers were washed with brine (20 mL), dried over Na₂SO₄, filtered, and concentrated under reduced pressure to give a residue. The residue was purified by silica gel column chromatography (PE:EtOAc=1:0 to 19:1) to afford tert-butyl 1-(6-chloro-3-(7-fluorobenzofuran-5-yl)pyrazin-2-yl)piperidine-4-carboxylate (850 mg, 65% yield) as a yellow oil. ¹H NMR (400 MHz, CDCl₃) δ 8.03 (s, 1H), 7.81 (s, 1H), 7.66 (m, 1H), 7.02 (d, J=15.6,1 H), 6.80 (s, 1H), 3.59 (m, 2H), 2.74 (m, 2H), 2.29 (m, 1H), 1.75 (m, 2H), 1.58 (m, 2H), 1.36 (s, 9H).

### Intermediate 132. 1-(6-chloro-3-(7-fluorobenzofuran-5-yl)pyrazin-2-yl)piperidine-4-carbonitrile

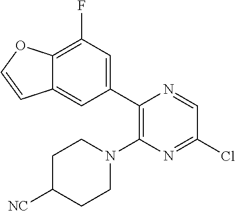 [SMILES: N#CC1CCN(c2nc(Cl)cnc2-c2cc(F)c3occc3c2)CC1]

To a mixture of 1-(3,6-dichloropyrazin-2-yl)piperidine-4-carbonitrile (1.00 g, 3.89 mmol, 1.0 eq) and 2-(7-fluorobenzofuran-5-yl)-4,4,5,5-tetramethyl-1,3,2-dioxaborolane (Intermediate 41) (1.02 g, 3.89 mmol, 1.0 eq) in DME (10 mL) and H₂O (2 mL) at rt under an N₂ (g) atmosphere was added Pd₂(dba)₃ (356 mg, 389 μmol, 0.1 eq), PPh₃ (204 mg, 778 μmol, 0.2 eq) and Na₂CO₃ (824 mg, 7.78 mmol, 2.0 eq). The resulting reaction mixture was heated to 80° C. and stirred for 3 hr. The reaction mixture was then cooled to rt, poured into H₂O (50 mL), and extracted with EtOAc (3×50 mL). The combined organic phase was washed with brine (20 mL), dried with anhydrous Na₂SO₄, filtered, and concentrated under vacuum. The residue was purified by silica gel column chromatography (PE:EtOAc=10:1 to 5:1) to afford 1-(6-chloro-3-(7-fluorobenzofuran-5-yl)pyrazin-2-yl)piperidine-4-carbonitrile (700 mg) as a yellow solid. LCMS calculated for C₁₈H₁₄ClFN₄O: m/z=356; found: m/z=357 (M+H).

### Example 1: 2-(4-(6-Butyl-3-(4-Methoxyphenyl)Pyrazin-2-yl)Piperazin-1-yl)Acetic Acid (Compound 1)

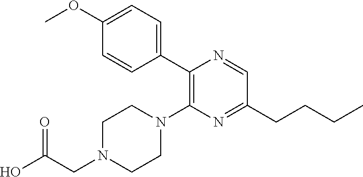 [SMILES: CCCCc1cnc(-c2ccc(OC)cc2)c(N2CCN(CC(=O)O)CC2)n1]

### Step 1: 5-Butyl-2-(4-methoxyphenyl)-3-(piperazin-1-yl)pyrazine

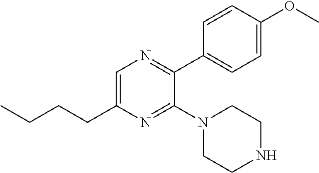 [SMILES: CCCCc1cnc(-c2ccc(OC)cc2)c(N2CCNCC2)n1]

To a mixture of 5-butyl-3-chloro-2-(4-methoxyphenyl)pyrazine (Intermediate 7) (200 mg, 722 mol, 1.0 equiv) in NMP (2 mL) at rt was added piperazine (2.24 g, 26.02 mmol, 36 equiv). The resulting reaction mixture was heated to 120° C. and stirred for 5 hr. The mixture was cooled to rt and then diluted with H₂O (50 mL) and extracted with EtOAc (2×50 mL). The combined organic phase was washed with brine (100 mL), dried with anhydrous Na₂SO₄, filtered, and concentrated under vacuum to afford 5-butyl-2-(4-methoxyphenyl)-3-piperazin-1-yl-pyrazine (250 mg, crude) as yellow liquid which was used without further purification. LCMS calculated for C₁₉H₂₆N₄O: m/z=326; found: m/z=327 (M+H).

### Step 2: Methyl 2-(4-(6-butyl-3-(4-methoxyphenyl)pyrazin-2-yl)piperazin-1-yl)acetate

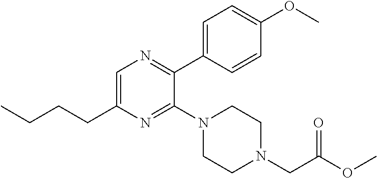 [SMILES: CCCCc1cnc(-c2ccc(OC)cc2)c(N2CCN(CC(=O)OC)CC2)n1]

To a mixture of 5-butyl-2-(4-methoxyphenyl)-3-piperazin-1-yl-pyrazine (230 mg, 704 mol, 1.0 equiv) and methyl 2-chloroacetate (765 mg, 7.05 mmol, 10.0 equiv) in CH₃CN (10 mL) at rt was added Na₂CO₃ (194 mg, 1.83 mmol, 2.6 equiv). The resulting reaction mixture was heated to 60° C. and stirred for 1 hr. The mixture was cooled to rt and then diluted with H₂O (50 mL) and extracted with EtOAc (2×50 mL). The combined organic phase was washed with brine (100 mL), dried with anhydrous Na₂SO₄, filtered, and concentrated under vacuum to afford methyl 2-(4-(6-butyl-3-(4-methoxyphenyl)pyrazin-2-yl)piperazin-1-yl)acetate (250 mg, crude) as yellow oily liquid which was used without further purification. LCMS calculated for C₂₂H₃₀N₄O₃: m/z=398; found: m/z=399 (M+H).

### Step 3: 2-(4-(6-Butyl-3-(4-methoxyphenyl)pyrazin-2-yl)piperazin-1-yl)acetic acid (Compound 1)

To a mixture methyl 2-(4-(6-butyl-3-(4-methoxyphenyl)pyrazin-2-yl)piperazin-1-yl)acetate (200 mg, 502 mol, 1.0 equiv) in MeOH (4 mL) at rt was added aqueous NaOH (4 M, 1.25 mL, 10.0 equiv). The resulting reaction mixture was heated to 50° C. and stirred for 3 hr. The mixture was then cooled to rt, filtered, and concentrated under reduced pressure to give a residue. The residue was purified by reverse-phase preparative HPLC (column: Waters Xbridge BEH C18 100×30×mm10 μm; mobile phase: [H₂O (10 mM NH₄HCO₃)-ACN]; B %: 15%-45%, 8 min run time) to afford 2-(4-(6-butyl-3-(4-methoxyphenyl)pyrazin-2-yl)piperazin-1-yl)acetic acid (105 mg, 52% yield, 100% purity by LC/MS) as a white solid. ¹H NMR (400 MHz, CD₃OD) δ8.06 (s, 1H), 7.83 (d, J=8.8, 2H), 7.06 (d, J=8.8, 2H), 3.87 (s, 3H), 3.56 (s, 2H), 3.42 (m, 4H), 3.25 (m, 4H), 2.77 (t, J=7.6, 2H), 1.76 (m, 2H), 1.42 (m, 2H), 0.99 (t, J=7.6, 3H); LCMS calculated for C₂₁H₂₈N₄O₃: m/z=384; found: m/z=385 (M+H).

### Example 2: 1-(6-Butyl-3-(4-Fluoro-3-Methoxyphenyl)Pyrazin-2-yl)Piperidine-4-carboxylic acid (Compound 2)

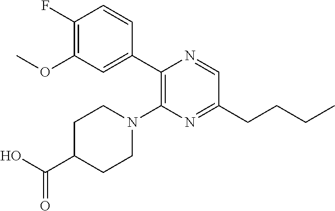 [SMILES: CCCCc1cnc(-c2ccc(F)c(OC)c2)c(N2CCC(C(=O)O)CC2)n1]

To a mixture of methyl 1-(3-bromo-6-butyl-pyrazin-2-yl)piperidine-4-carboxylate (Intermediate 4)(20 mg, 56 mol, 1.0 equiv) and (4-fluoro-3-methoxy-phenyl)boronic acid (11 mg, 62 mol, 1.1 equiv) in THF (1 mL) and H₂O (0.2 mL) at rt was added [1,1′-Bis(di-tert-butylphosphino)ferrocene]dichloropalladium(II) (7 mg, 11 mol, 0.2 equiv) and K₂CO₃(15 mg, 112 mol, 2.0 equiv) under N₂(g). The resulting reaction mixture was heated to 80° C. and stirred for 12 hr. The reaction mixture was then cooled to rt, and MeOH (3 mL) was added, followed by aqueous NaOH (4 M, 374 L, 6.0 equiv). The resulting reaction mixture was heated to 50° C. and stirred for 3 hr. The reaction mixture was then cooled to rt and concentrated to give a residue. The residue was purified by reverse-phase preparative HPLC (column: Waters Xbridge BEH C18 100×30 mm x10 μm; mobile phase: [H₂O (NH₄HCO₃)-ACN]; B %: 25%-55%, 10 min run time) to afford 1-(6-butyl-3-(4-fluoro-3-methoxy-phenyl)pyrazin-2-yl)piperidine-4-carboxylic acid (52 mg, 49% yield, 96% purity by LC/MS) as a yellow solid. ¹H NMR (400 MHz, CD₃OD) δ7.96 (s, 1H), 7.61 (dd, J=2.0, 8.4, 1H), 7.43 (m, 1H), 7.21 (dd, J=8.8, 11.2, 1H), 3.95 (s, 3H), 3.62 (m, 2H), 2.77 (m, 4H), 2.44 (m, 1H), 1.86 (m, 2H), 1.72 (m, 4H), 1.43 (m, 2H), 0.99 (t, J=7.2, 3H); LCMS calculated for C₂₁H₂₆FN₃O₃: m/z=387; found: m/z=388 (M+H).

### Example 3: 1-(6-Butyl-3-(4-Methoxyphenyl)Pyrazin-2-yl)Piperidine-4-Carboxylic Acid (Compound 3)

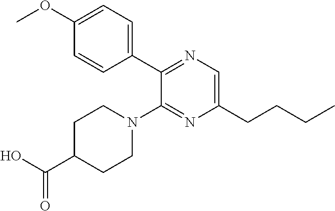 [SMILES: CCCCc1cnc(-c2ccc(OC)cc2)c(N2CCC(C(=O)O)CC2)n1]

### Step 1: Methyl 1-(6-butyl-3-(4-methoxyphenyl)pyrazin-2-yl)piperidine-4-carboxylate

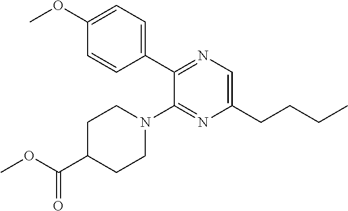 [SMILES: CCCCc1cnc(-c2ccc(OC)cc2)c(N2CCC(C(=O)OC)CC2)n1]

To a mixture of methyl 1-(3-bromo-6-butylpyrazin-2-yl)piperidine-4-carboxylate (Intermediate 4) (0.2 g, 561 mol, 1.0 equiv) and (4-methoxyphenyl)boronic acid (102 mg, 674 mol, 1.2 equiv) in THF (3 mL) and H₂O (0.6 mL) at rt was added [2-(2-aminophenyl)phenyl]-chloro-palladium; bis(1-adamantyl)-butyl-phosphane (38 mg, 56 mol, 0.1 equiv) and K₃PO₄ (238 mg, 1.12 mmol, 2.0 equiv). The resulting reaction mixture was heated to 80° C. and stirred for 12 hr. The reaction mixture was cooled to rt, diluted with H₂O (20 mL), and then extracted with EtOAc (2×20 mL). The combined organic layers were washed with brine (10 mL), dried over Na₂SO₄, filtered, and concentrated under reduced pressure to give a residue. The residue was purified by silica gel column chromatography (PE:EtOAc=15:1 to 5:1) to afford methyl 1-(6-butyl-3-(4-methoxyphenyl)pyrazin-2-yl)piperidine-4-carboxylate (130 mg, 60% yield) as a yellow oil. LCMS calculated for C₂₃H₃₀N₂O₃: m/z=383; found: m/z=384 (M+H).

### Step 2: 1-(6-butyl-3-(4-methoxyphenyl)pyrazin-2-yl)piperidine-4-carboxylic acid (Compound 3)

To a solution of methyl 1-(6-butyl-3-(4-methoxyphenyl)pyrazin-2-yl)piperidine-4-carboxylate (100 mg, 261 mol, 1.0 equiv) in MeOH (10 mL) at rt was added aqueous NaOH (4 M, 391 L, 6.0 equiv). The resulting reaction mixture was heated to 50° C. and stirred for 12 hr. The reaction mixture was filtered to remove particulates and concentrated under reduced pressure to give a residue. The residue was purified by reverse-phase preparative HPLC (column: Waters Xbridge BEH C18 100×30 mm×10 m; mobile phase: [H₂O (10 mM NH₄HCO₃)-ACN]; B %: 20%-50%, 8 min run time) to afford 1-(6-butyl-3-(4-methoxyphenyl)pyrazin-2-yl)piperidine-4-carboxylic acid (36 mg, 36% yield, 100% purity by LC/MS) as a white solid. ¹H NMR (400 MHz, CD₃OD) δ7.89 (s, 1H), 7.80 (d, J=8.8, 2H), 7.05 (d, J=8.8, 2H), 3.86 (s, 3H), 3.64 (d, J=8.8, 2H), 2.71 (m, 4H), 2.31 (m, 1H), 1.80-1.71 (m, 6H), 1.44 (m, 2H,) 1.00 (t, J=7.2, 3H); LCMS calculated for C₂₁H₂₇N₃O₃: m/z=369; found: m/z=370 (M+H).

### Example 4: 3-(6-Butyl-3-(4-Methoxyphenyl)Pyrazin-2-yl)-3-Azabicyclo[3.1.0]Hexane-6-carboxylic acid (Compound 4)

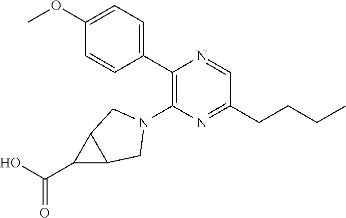 [SMILES: CCCCc1cnc(-c2ccc(OC)cc2)c(N2CC3C(C2)C3C(=O)O)n1]

### Step 1: Methyl 3-(6-butyl-3-(4-methoxyphenyl)pyrazin-2-yl)-3-azabicyclo[3.1.0]hexane-6-carboxylate

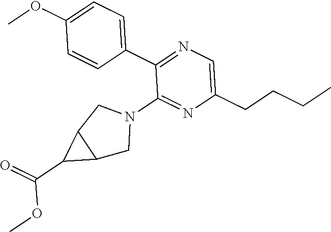 [SMILES: CCCCc1cnc(-c2ccc(OC)cc2)c(N2CC3C(C2)C3C(=O)OC)n1]

To a mixture of 5-butyl-3-chloro-2-(4-methoxyphenyl)pyrazine (Intermediate 7) (150 mg, 541 mol, 1.0 equiv) and methyl 3-azabicyclo[3.1.0]hexane-6-carboxylate (114 mg, 812 mol, 1.5 equiv) in THF (3 mL) at rt was added t-BuONa (182 mg, 1.90 mmol, 3.5 equiv), Xantphos Pd G4 (46 mg, 54 mol, 0.1 equiv) and Xantphos (31 mg, 54 mol, 0.1 equiv). The resulting reaction mixture was heated to reflux and stirred for 12 hr. The mixture was then cooled to rt, poured into H₂O (100 mL) and extracted with EtOAc (3×100 mL). The combined organic phase was washed with brine (60 mL), dried with anhydrous Na₂SO₄, filtered, and concentrated under vacuum. The residue was purified by prep-TLC (PE:EtOAc=7:1) to afford methyl 3-(6-butyl-3-(4-methoxyphenyl)pyrazin-2-yl)-3-azabicyclo[3.1.0]hexane-6-carboxylate (50 mg, 12% yield) as a yellow solid, which was used directly in next step. LCMS calculated for C₂₂H₂₇N₃O₃: m/z=381; found: m/z=382 (M+H).

### Step 2: 3-(6-Butyl-3-(4-methoxyphenyl)pyrazin-2-yl)-3-azabicyclo[3.1.0]hexane-6-carboxylic acid (Compound 4)

To a solution of methyl 3-(6-butyl-3-(4-methoxyphenyl)pyrazin-2-yl)-3-azabicyclo[3.1.0]hexane-6-carboxylate (50 mg, 131 mol, 1.0 equiv) in MeOH (1 mL) at rt was added aqueous NaOH (4 M, 196 L, 6.0 equiv). The resulting reaction mixture was heated to 50° C. and stirred for 3 hr. The reaction mixture was cooled to rt and the volatiles were removed under reduced pressure. The residue was purified by reverse-phase preparative HPLC (column: Waters Xbridge BEH C18 100×30 mm x10 m; mobile phase: [H₂O(NH₄HCO₃)-ACN];B %: 5%-50%, 8 min run time) to afford 3-(6-butyl-3-(4-methoxyphenyl)pyrazin-2-yl)-3-azabicyclo[3.1.0]hexane-6-carboxylic acid (34 mg, 70% yield, 98% purity by LC/MS) as a white solid. ¹H NMR (400 MHz, CD₃OD) δ7.78 (s, 1H), 7.44 (d, J=8.4, 2H), 7.04 (d, J=8.4, 2H), 3.87 (s, 3H), 3.57 (d, J=10.8, 2H), 3.23 (d, J=11.2, 2H), 2.71 (t, J=8.0, 2H), 2.00 (m, 2H), 1.75 (m, 2H), 1.42 (m, 3H), 0.99 (t, J=7.6, 3H). LCMS calculated for C₂₁H₂₅N₃O₃: m/z=367; found: m/z=368 (M+H).

### Example 5: 1-(6-Butyl-3-(1H-Indol-5-yl)Pyrazin-2-yl)Piperidine-4-Carboxylic Acid (Compound 5)

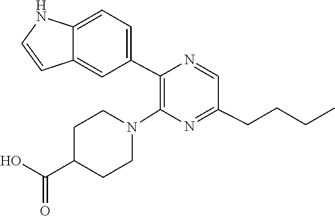 [SMILES: CCCCc1cnc(-c2ccc3[nH]ccc3c2)c(N2CCC(C(=O)O)CC2)n1]

### Step 1: Methyl 1-(6-butyl-3-(1H-indol-5-yl)pyrazin-2-yl)piperidine-4-carboxylate

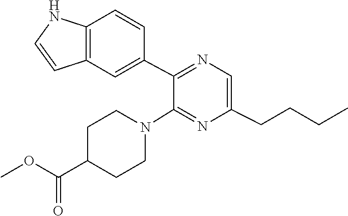 [SMILES: CCCCc1cnc(-c2ccc3[nH]ccc3c2)c(N2CCC(C(=O)OC)CC2)n1]

To a mixture of methyl 1-(3-bromo-6-butylpyrazin-2-yl)piperidine-4-carboxylate (Intermediate 4) (0.1 g, 281 mol, 1.0 equiv) and 1H-indol-5-ylboronic acid (90 mg, 505 mol, 1.8 equiv) in THF (2 mL) and H₂O (0.4 mL) at rt was added Chloro[(di(1-adamantyl)-N-butylphosphine)-2-(2-aminobiphenyl)]palladium(II) (19 mg, 28 mol, 0.1 equiv) and K₃PO₄ (119 mg, 562 mol, 2.0 equiv). The resulting reaction mixture was heated to 80° C. and stirred for 12 hr. The reaction mixture was cooled to rt, filtered to remove solids, and the filtrate was concentrated under reduced pressure to give crude methyl 1-(6-butyl-3-(1H-indol-5-yl)pyrazin-2-yl)piperidine-4-carboxylate, which was used directly without further purification. LCMS calculated for C₂₃H₂₈N₄O₂: m/z=392; found: m/z=393 (M+H).

### Step 2: 1-(6-butyl-3-(1H-indol-5-yl)pyrazin-2-yl)piperidine-4-carboxylic acid (Compound 5)

To a solution of methyl 1-(6-butyl-3-(1H-indol-5-yl)pyrazin-2-yl)piperidine-4-carboxylate (120 mg, 306 mol, 1.0 equiv) in MeOH (1 mL) at rt was added aqueous NaOH (4 M, 459 L, 6.0 equiv). The resulting reaction mixture was heated to 50° C. and stirred for 3 hr. The reaction mixture was cooled to rt, filtered to remove solids, and the filtrate was concentrated under reduced pressure to give a residue. The residue was purified by reverse-phase preparative HPLC (column: Waters Xbridge Prep OBD C18 100×30 mm x10 m; mobile phase: [H₂O (10 mM NH₄HCO₃)-ACN]; B %: 20%-50%, 10 min run time) to afford 1-(6-butyl-3-(1H-indol-5-yl)pyrazin-2-yl)piperidine-4-carboxylic acid (44 mg, 37% yield, 99% purity by LC/MS) as a yellow solid. ¹H NMR (400 MHz, CD₃OD) δ7.98 (s, 1H), 7.87 (s, 1 H), 7.56 (d, J=8.8, 1H), 7.47 (d, J=8.8, 1H), 7.28 (d, J=3.2, 1H), 6.51 (d, J=3.2, 1H), 3.65 (m, 2H), 2.69 (m, 4H), 2.35 (m, 1H), 1.78-1.62 (m, 6H), 1.43 (m, 2H), 0.98 (t, J=7.2, 3H); LCMS calculated for C₂₂H₂₆N₄O₂: m/z=378; found: m/z=379 (M+H).

### Example 6: 1-(6-Butyl-3-(4-(2-(Dimethylamino)Ethoxy)Phenyl)Pyrazin-2-yl)piperidine-4-carboxylic acid (Compound 6)

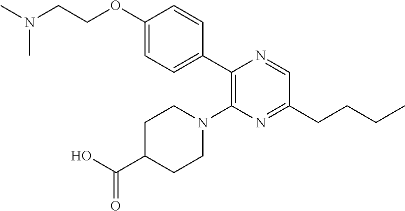 [SMILES: CCCCc1cnc(-c2ccc(OCCN(C)C)cc2)c(N2CCC(C(=O)O)CC2)n1]

To a mixture of methyl 1-(3-bromo-6-butyl-pyrazin-2-yl)piperidine-4-carboxylate (Intermediate 4) (100 mg, 280 mol, 1.0 equiv) and N,N-dimethyl-2-[4-(4,4,5,5-tetramethyl-1,3,2-dioxaborolan-2-yl)phenoxy]ethanamine (82 mg, 280 mol, 1.0 equiv) in THF (2.5 mL) and H₂O (0.5 mL) at rt under N₂(g) was added [1,1′-Bis(di-tert-butylphosphino)ferrocene]dichloropalladium(II) (27 mg, 42 mol, 0.15 equiv) and K₂CO₃(77 mg, 561 mol, 2.0 equiv). The resulting reaction mixture was heated to 80° C. and stirred for 12 hr. The reaction mixture was then cooled to rt and then MeOH (2 mL) was added, followed by aqueous NaOH (4 M, 340 L, 6.0 equiv). The resulting reaction mixture was heated to 50° C. and stirred for 3 hr. The reaction mixture was then cooled to rt and filtered. The filtrate was concentrated under reduced pressure to provide a residue. The residue was purified by reverse-phase preparative HPLC (column: Waters Xbridge BEH C18 100×30 mm x10 μm; mobile phase: [H₂O(NH₄HCO₃)-ACN]; B %: 20%-50%, 10 min run time) to afford 1-(6-butyl-3-(4-(2-(dimethylamino)ethoxy)phenyl)pyrazin-2-yl)piperidine-4-carboxylic acid (10 mg, 10% yield, 91% purity by LC/MS) as a yellow solid. ¹H NMR (400 MHz, CD₃OD) δ 7.91 (s, 1H), 7.83 (d, J=8.8, 2H), 7.11 (d, J=8.8, 2H), 4.33 (m, 2H), 3.63 (d, J=12.8, 2H), 3.31 (m, 2H), 2.71 (m, 10H), 2.28 (m, 1H), 1.72 (m, 6H), 1.42 (m, 2H), 0.99 (t, J=7.2, 3H); LCMS calculated for C₂₄H3₄N₄O₃: m/z=426; found: m/z=427 (M+H).

### Example 7: 1-(6-Butyl-3-(4-(Methylsulfonyl)Phenyl)Pyrazin-2-yl)Piperidine-4-carboxylic acid (Compound 7)

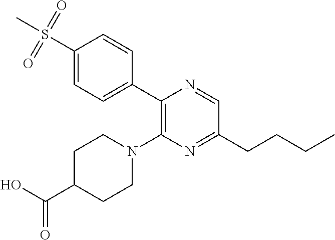 [SMILES: CCCCc1cnc(-c2ccc(S(C)(=O)=O)cc2)c(N2CCC(C(=O)O)CC2)n1]

### Step 1: Methyl 1-(6-butyl-3-(4-(methylsulfonyl)phenyl)pyrazin-2-yl)piperidine-4-carboxylate

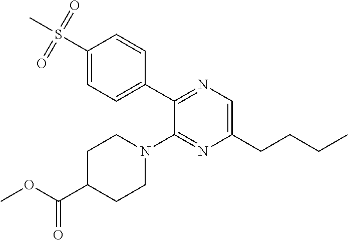 [SMILES: CCCCc1cnc(-c2ccc(S(C)(=O)=O)cc2)c(N2CCC(C(=O)OC)CC2)n1]

To a mixture of methyl 1-(3-bromo-6-butylpyrazin-2-yl)piperidine-4-carboxylate (Intermediate 4) (0.1 g, 281 mol, 1.0 equiv) and (4-methylsulfonylphenyl)boronic acid (101 mg, 505 mol, 1.8 equiv) in THF (2 mL) and H₂O (0.4 mL) at rt was added chloro[(di(1-adamantyl)-N-butylphosphine)-2-(2-aminobiphenyl)]palladium(II) (19 mg, 28 mol, 0.1 equiv) and K₃PO₄ (119 mg, 562 mol, 2.0 equiv). The resulting reaction mixture was heated to 80° C. and stirred for 12 hr. The reaction mixture was cooled to rt, filtered to remove solids, and the filtrate was concentrated under reduced pressure to give crude methyl 1-(6-butyl-3-(4-(methylsulfonyl)phenyl)pyrazin-2-yl)piperidine-4-carboxylate, which was used directly without further purification. LCMS calculated for C₂₂H₂₉N₃O₄S: m/z=431; found: m/z=432 (M+H).

### Step 2: 1-(6-Butyl-3-(4-(methylsulfonyl)phenyl)pyrazin-2-yl)piperidine-4-carboxylic acid (Compound 7)

To a solution of crude methyl 1-(6-butyl-3-(4-(methylsulfonyl)phenyl)pyrazin-2-yl)piperidine-4-carboxylate (120 mg, 278 mol, 1.0 equiv) in MeOH (1 mL) at rt was added aqueous NaOH (4 M, 417 L, 6.0 equiv). The resulting reaction mixture was heated to 50° C. and stirred for 3 hr. The reaction mixture was cooled to rt, filtered to remove solids, and the filtrate was concentrated under reduced pressure to give a residue. The residue was purified by reverse-phase preparative HPLC (column: Waters Xbridge Prep OBD C18 150×40 mm x m; mobile phase: [H₂O (10 mM NH₄HCO₃)-ACN]; B %: 15%-45%, 8 min run time) to afford 1-(6-butyl-3-(4-(methylsulfonyl)phenyl)pyrazin-2-yl)piperidine-4-carboxylic acid (62 mg, 53% yield, 99% purity by LC/MS) as a yellow solid. ¹H NMR (400 MHz, CD₃OD) δ 8.12 (d, J=7.6, 2H), 8.04 (m, 3H), 3.58 (m, 2H), 3.16 (s, 3H), 2.80-2.72 (m, 4H), 2.30 (m, 1H), 1.81-1.68 (m, 6H), 1.41 (m, 2H), 0.97 (t, J=7.2, 3H); LCMS calculated for C₂₁H27N₃O₄S: m/z=417; found: m/z=418 (M+H).

### Example 8: 5-Butyl-2-(4-Methoxyphenyl)-3-(4-(Methylsulfonyl)Piperidin-1-yl)pyrazine (Compound 8)

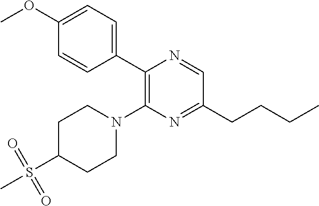 [SMILES: CCCCc1cnc(-c2ccc(OC)cc2)c(N2CCC(S(C)(=O)=O)CC2)n1]

To a mixture of 5-butyl-3-chloro-2-(4-methoxyphenyl)pyrazine (Intermediate 7) (110 mg, 396 mol, 1.0 equiv) and 4-methylsulfonylpiperidine (972 mg, 5.96 mmol, 15.0 equiv) in NMP (5 mL) at rt was added DIEA (770 mg, 5.96 mmol, 15.0 equiv). The resulting reaction mixture was heated to 160° C. and stirred for 4.5 hr. The reaction mixture was then cooled to rt and purified directly by reverse-phase preparative HPLC (column: Waters Xbridge BEH C18 100×30 mm x10 μm; mobile phase: [H₂O (NH₄HCO₃)-ACN];B %: 35%-85%, 10 min run time) to afford 5-butyl-2-(4-methoxyphenyl)-3-(4-methylsulfonyl-1-piperidyl)pyrazine (36 mg, 41% yield, 99% purity by LC/MS) as yellow oil. ¹H NMR (400 MHz, CD₃OD) δ7.96 (s, 1H), 7.79 (d, J=8.8, 2H), 7.05 (d, J=8.8, 2H), 3.87 (s, 3H), 3.81 (m, 1H), 3.21 (m, 1H), 2.92 (s, 3H), 2.76 (m, 5H), 2.05 (m, 2H), 1.78 (m, 4H), 1.42 (m, 2H), 0.99 (t, J=7.6, 3H); LCMS calculated for C₂₁H₂₉N₃O₃S: m/z=403; found: m/z=404 (M+H).

### Example 9: 1-(6-Butyl-3-(7-Fluorobenzofuran-5-yl)Pyrazin-2-yl)Piperidine-4-carboxylic acid (Compound 9)

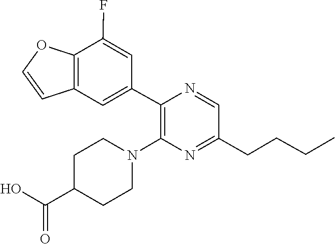 [SMILES: CCCCc1cnc(-c2cc(F)c3occc3c2)c(N2CCC(C(=O)O)CC2)n1]

### Step 1: Methyl 1-(6-butyl-3-(7-fluorobenzofuran-5-yl)pyrazin-2-yl)piperidine-4-carboxylate

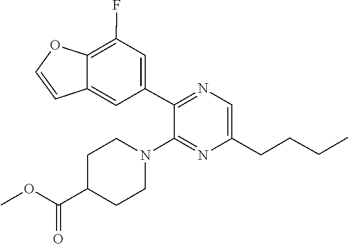 [SMILES: CCCCc1cnc(-c2cc(F)c3occc3c2)c(N2CCC(C(=O)OC)CC2)n1]

To a mixture of methyl 1-(3-bromo-6-butylpyrazin-2-yl)piperidine-4-carboxylate (Intermediate 4)(100 mg, 281 mol, 1.0 equiv) and 2-(7-fluorobenzofuran-5-yl)-4,4,5,5-tetramethyl-1,3,2-dioxaborolane (81 mg, 309 mol, 1.1 equiv) in THF (2 mL) and H₂O (0.4 mL) at rt under N₂(g) was added chloro[(di(1-adamantyl)-N-butylphosphine)-2-(2-aminobiphenyl)]palladium(II) (19 mg, 28 mol, 0.1 equiv) and K₃PO₄ (119 mg, 561 mol, 2.0 equiv). The resulting reaction mixture was heated to 80° C. and stirred for 12 hr. The mixture was then cooled to rt and then was partitioned between H₂O (50 mL) and EtOAc (100 mL) and extracted. The organic phase was washed with brine (100 mL), dried with anhydrous Na₂SO₄, filtered, and concentrated under vacuum. The residue was purified by silica gel column chromatography (PE:EtOAc=20:1 to 10:1) to afford methyl 1-(6-butyl-3-(7-fluorobenzofuran-5-yl)pyrazin-2-yl)piperidine-4-carboxylate (120 mg) as a yellow solid. LCMS calculated for C₂₃H₂₆FN₃O₃: m/z=411; found: m/z=412 (M+H).

### Step 2: 1-(6-butyl-3-(7-fluorobenzofuran-5-yl)pyrazin-2-yl)piperidine-4-carboxylic acid (Compound 9)

To a solution of methyl 1-(6-butyl-3-(7-fluorobenzofuran-5-yl)pyrazin-2-yl)piperidine-4-carboxylate (110 mg, 267 mol, 1.0 equiv) in MeOH (5 mL) at rt was added aqueous NaOH (4 M, 400 L, 6.0 equiv). The resulting reaction mixture was heated to 50° C. and stirred for 3 hr. The mixture was then cooled to rt and then purified by prep-HPLC (column: Phenomenex C18 80×40 mm×3 μm; mobile phase: [H₂O (NH₄HCO₃)-ACN]; B %: 20%-50%, 8 min run time) to afford 1-(6-butyl-3-(7-fluorobenzofuran-5-yl)pyrazin-2-yl)piperidine-4-carboxylic acid (39 mg, 36% yield, 99% purity by LC/MS) as a yellow solid. ¹H NMR (400 MHz, CD₃OD) δ7.96 (m, 2H), 7.90 (d, J=2.4, 1H), 7.61 (dd, J=1.2, 12.0, 1 H), 7.04 (dd, J=2.4, 2.4, 1H), 3.64 (m, 2H), 2.77 (m, 4H), 2.34 (m, 1H), 1.73 (m, 6H), 1.44 (m, 2H), 1.00 (t, J=7.2, 3H); LCMS calculated for C₂₂H₂₄FN₃O₃: m/z=397; found: m/z=398 (M+H).

### Example 10: 1-(6-Butyl-3-(4-Methoxyphenyl)Pyrazin-2-yl)-4-Fluoropiperidine-4-carboxylic acid (Compound 10)

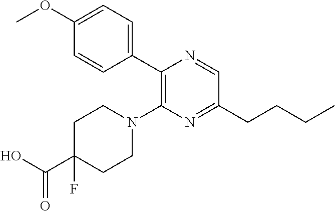 [SMILES: CCCCc1cnc(-c2ccc(OC)cc2)c(N2CCC(F)(C(=O)O)CC2)n1]

### Step 1: Ethyl 1-(6-butyl-3-(4-methoxyphenyl)pyrazin-2-yl)-4-fluoropiperidine-4-carboxylate

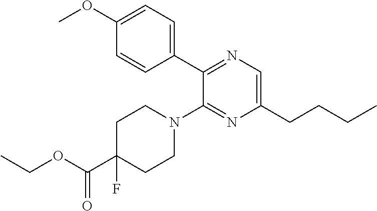 [SMILES: CCCCc1cnc(-c2ccc(OC)cc2)c(N2CCC(F)(C(=O)OCC)CC2)n1]

To a mixture of 5-butyl-3-chloro-2-(4-methoxyphenyl)pyrazine (Intermediate 7) (100 mg, 361 mol, 1.0 equiv) and ethyl 4-fluoropiperidine-4-carboxylate (764 mg, 3.61 mmol, 10.0 equiv, HCl salt) in NMP (2 mL) at rt was added DIEA (1.40 g, 10.84 mmol, 30 equiv). The resulting reaction mixture was heated to 160° C. and stirred for 5 hr. The reaction mixture was then cooled to rt, poured into H₂O (40 mL) and extracted with EtOAc (3×40 mL). The combined organic phase was washed with brine (30 mL), dried with anhydrous Na₂SO₄, filtered, and concentrated under vacuum. The residue was purified by silica gel column chromatography (PE:EtOAc=50:1 to 1:1) to afford ethyl 1-(6-butyl-3-(4-methoxyphenyl)pyrazin-2-yl)-4-fluoro-piperidine-4-carboxylate (30 mg, 19% yield) as a yellow oil. LCMS calculated for C₂₃H₃₀FN₃O₃: m/z=415; found: m/z=416 (M+H).

### Step 2: 1-(6-Butyl-3-(4-methoxyphenyl)pyrazin-2-yl)-4-fluoropiperidine-4-carboxylic acid (Compound 10)

To a solution of ethyl 1-(6-butyl-3-(4-methoxyphenyl)pyrazin-2-yl)-4-fluoro-piperidine-4-carboxylate (30 mg, 73 mol, 1.0 equiv) in MeOH (2 mL) at rt was added aqueous NaOH (4 M, 117 L, 6.0 equiv). The resulting reaction mixture was heated to 50° C. and stirred for 1.5 hr. The reaction mixture was cooled to rt and volatiles were removed under vacuum. The residue was purified directly by reverse-phase preparative HPLC (column: Waters Xbridge BEH C18 100×30 mm×10 μm; mobile phase: [H₂O (NH₄HCO₃)-ACN]; B %: 25%-55%, 10 min run time) to afford 1-(6-butyl-3-(4-methoxyphenyl)pyrazin-2-yl)-4-fluoro-piperidine-4-carboxylic acid (11 mg, 57% yield) as a white solid. ¹H NMR (400 MHz, CD₃OD) δ7.92 (s, 1H), 7.82 (d, J=8.8, 2H), 7.05 (d, J=8.8, 2H), 3.86 (s, 3H), 3.59 (d, J=11.2, 2H), 2.97 (m, 2H), 2.74 (t, J=7.6, 2H), 2.20 (m, 2H), 1.77 (m, 4H), 1.44 (m, 2H), 0.99 (t, J=7.6, 3H); LCMS calculated for C₂₁H₂₆FN₃O₃: m/z=387; found: m/z=388 (M+H).

### Example 11: 1-(6-Butyl-3-(4-Methoxyphenyl)Pyrazin-2-yl)-N-(Methylsulfonyl)Piperidine-4-carboxamide (Compound 11)

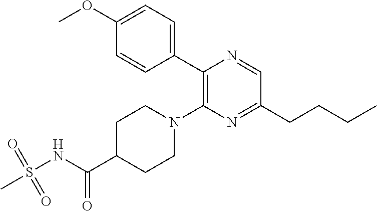 [SMILES: CCCCc1cnc(-c2ccc(OC)cc2)c(N2CCC(C(=O)NS(C)(=O)=O)CC2)n1]

To a mixture of 1-(6-butyl-3-(4-methoxyphenyl)pyrazin-2-yl)piperidine-4-carboxylic acid (Example 3) (100 mg, 270 mol, 1.0 equiv) and methanesulfonamide (31 mg, 324 mol, 1.2 equiv) in DCM (10 mL) at rt under N₂(G) was added DMAP (99 mg, 812 mol, 3.0 equiv) and EDCI (103 mg, 541 mol, 2.0 equiv). The resulting reaction mixture was stirred at rt for 3 hr. The reaction mixture was then concentrated under reduced pressure to remove the DCM, and the residue was diluted with CH₃CN (3 mL) and purified by reverse-phase preparative HPLC (column: Waters Xbridge BEH C18 100×30 mm×10 μm; mobile phase: [H₂O (NH₄HCO₃)-ACN]; B %: 20%-50%, 8 min run time) to afford 1-(6-butyl-3-(4-methoxyphenyl)pyrazin-2-yl)-N-methylsulfonyl-piperidine-4-carboxamide (17 mg, 13% yield, 96% purity by LC/MS) as a white solid. ¹H NMR (400 MHz, CD₃OD) δ7.90 (s, 1H), 7.75 (d, J=8.8, 2H), 7.01 (d, J=8.8, 2H), 3.83 (s, 3H), 3.67 (d, J=12.0, 2H), 3.18 (s, 3 H), 2.68 (m, 4H), 2.35 (m, 1H), 1.72 (m, 6H), 1.39 (m, 2H), 0.96 (t, J=7.2, 3H); LCMS calculated for C₂₂H₃₀N₄O₄S: m/z=446; found: m/z=447(M+H).

### Example 12: 1-(6-Butyl-3-(4-Methoxyphenyl)Pyrazin-2-yl)Pyrrolidine-3-Carboxylic acid (Compound 12)

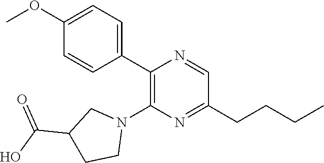 [SMILES: CCCCc1cnc(-c2ccc(OC)cc2)c(N2CCC(C(=O)O)C2)n1]

### Step 1: Methyl 1-(6-butyl-3-(4-methoxyphenyl)pyrazin-2-yl)pyrrolidine-3-carboxylate

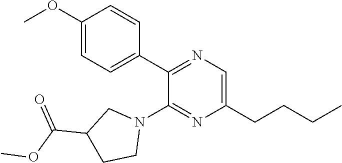 [SMILES: CCCCc1cnc(-c2ccc(OC)cc2)c(N2CCC(C(=O)OC)C2)n1]

To a mixture of 5-butyl-3-chloro-2-(4-methoxyphenyl)pyrazine (Intermediate 7) (100 mg, 361 mol, 1.0 equiv) and methyl pyrrolidine-3-carboxylate (1.80 g, 10.84 mmol, 30.0 equiv) in NMP (3 mL) at rt was added DIEA (1.40 g, 10.84 mmol, 1.89 mL, 30.0 equiv). The resulting reaction mixture was heated to 160° C. and stirred for 3 hr. The reaction mixture was cooled to rt, diluted with H₂O (100 mL), and then extracted with EtOAc (2×100 mL). The combined organic layers were dried over Na₂SO₄, filtered, and concentrated under reduced pressure to give crude methyl 1-(6-butyl-3-(4-methoxyphenyl)pyrazin-2-yl)pyrrolidine-3-carboxylate (100 mg) which was used without further purification. LCMS calculated for C₂₁H₂₇N₃O₃: m/z=369; found: m/z=370 (M+H).

### Step 2: 1-(6-Butyl-3-(4-methoxyphenyl)pyrazin-2-yl)pyrrolidine-3-carboxylic acid (Compound 12)

To a solution of methyl 1-(6-butyl-3-(4-methoxyphenyl)pyrazin-2-yl)pyrrolidine-3-carboxylate (80 mg, 216 mol, 1.0 equiv) in MeOH (3 mL) at rt was added aqueous NaOH (4 M, 325 L, 6.0 equiv). The resulting reaction mixture was heated to 50° C. and stirred for 3 hr. The reaction mixture was cooled to rt, filtered to remove solids, and the filtrate was concentrated under reduced pressure to give a residue. The residue was purified by reverse-phase preparative HPLC (column: Phenomenex Gemini-NX C18 75×30 mm×3 m; mobile phase: [H₂O (10 mM NH₄HCO₃)-ACN]; B %: 15%-45%, 10 min run time) to afford 1-(6-butyl-3-(4-methoxyphenyl)pyrazin-2-yl)pyrrolidine-3-carboxylic acid (56 mg, 65% yield, 93% purity by LC/MS) as a yellow solid. ¹H NMR (400 MHz, CD₃OD) δ7.72 (s, 1H), 7.46 (d, J=8.8, 2H), 6.99 (d, J=8.8, 2H), 3.86 (s, 3H), 3.43 (m, 2H), 3.24 (m, 2H) 2.92 (m, 1 H), 2.69 (t, J=7.6, 2H), 2.06 (m, 2H), 1.76 (m, 2H), 1.43 (m, 2H), 0.98 (t, J=7.2, 3H); LCMS calculated for C₂₀H₂₅N₃O₃: m/z=355; found: m/z=356 (M+H).

### Example 13: 1-(6-Butyl-3-(4-(2-Methoxyethoxy)Phenyl)Pyrazin-2-yl)Piperidine-4-carboxylic acid (Compound 13)

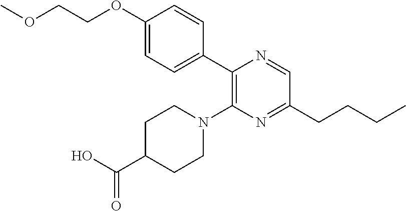 [SMILES: CCCCc1cnc(-c2ccc(OCCOC)cc2)c(N2CCC(C(=O)O)CC2)n1]

To a mixture of methyl 1-(3-bromo-6-butyl-pyrazin-2-yl)piperidine-4-carboxylate (Intermediate 4) (100 mg, 280 mol, 1.0 equiv) and [4-(2-methoxyethoxy)phenyl]boronic acid (55 mg, 280 mol, 1.0 equiv) in THF (2.5 mL) and H₂O (0.5 mL) at rt under N₂(g) was added [1,1′-Bis(di-tert-butylphosphino)ferrocene]dichloropalladium(II) (27 mg, 42 mol, 0.15 equiv) and K₂CO₃ (77 mg, 561 mol, 2.0 equiv). The resulting reaction mixture was heated to 80° C. and stirred for 12 hr. The reaction mixture was then cooled to rt and then MeOH (2 mL) was added, followed by aqueous NaOH (4 M, 350 L, 6.0 equiv). The resulting reaction mixture was heated to 50° C. and stirred for 3 hr. The reaction mixture was then cooled to rt and then concentrated to give a residue. The residue was purified by reverse-phase preparative HPLC (column: Waters Xbridge BEH C18 100×30 mm×10 μm; mobile phase: [H₂O (NH₄HCO₃)-ACN]; B %: 25%-55%, 10 min run time) to afford 1-(6-butyl-3-[4-(2-methoxyethoxy)phenyl]pyrazin-2-yl)piperidine-4-carboxylic acid (22 mg, 21% yield, 97% purity by LC/MS) as a yellow solid. ¹H NMR (400 MHz, CD₃OD) δ7.92 (s, 1H), 7.79 (d, J=7.6, 2H), 7.06 (d, J=8.4, 2H), 4.19 (m, 2H), 3.79 (m, 2H), 3.62 (m, 2H), 3.45 (s, 3H), 2.75 (m, 4H), 2.41 (m, 1H), 1.83-1.70 (m, 6H), 1.43 (m, 2H), 0.99 (t, J=7.2, 3H); LCMS calculated for C₂₃H3₁N₃O₄: m/z=413; found: m/z=414 (M+H).

### Example 14: 1-(6-Butyl-3-(4-Methoxyphenyl)Pyrazin-2-yl)-4-Methylpiperidine-4-carboxylic acid (Compound 14)

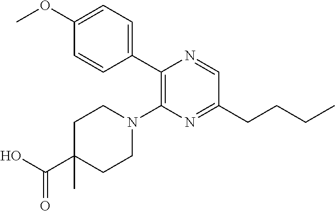 [SMILES: CCCCc1cnc(-c2ccc(OC)cc2)c(N2CCC(C)(C(=O)O)CC2)n1]

### Step 1: Methyl 4-methylpiperidine-4-carboxylate

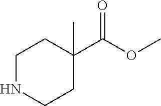

A mixture of 1-tert-butyl 4-methyl 4-methylpiperidine-1,4-dicarboxylate (0.40 g, 1.55 mmol, 1.0 equiv) in HCl/MeOH (4 M, 20 mL) under N₂(g) was stirred at rt for 1 hr. The reaction mixture was concentrated under reduced pressure to afford methyl 4-methylpiperidine-4-carboxylate (0.45 g, crude) as a white solid. ¹H NMR (400 MHz, CD₃OD) δ3.68 (s, 3H), 3.22 (m, 2H), 2.92 (m, 2H), 2.19 (m, 2H), 1.61 (m, 2H), 1.19 (s, 3 H).

### Step 2: Methyl 1-(6-butyl-3-(4-methoxyphenyl)pyrazin-2-yl)-4-methylpiperidine-4-carboxylate

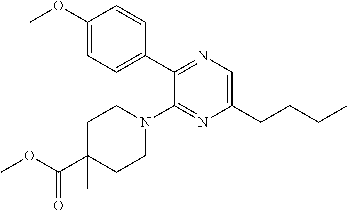 [SMILES: CCCCc1cnc(-c2ccc(OC)cc2)c(N2CCC(C)(C(=O)OC)CC2)n1]

To a mixture of 5-butyl-3-chloro-2-(4-methoxyphenyl)pyrazine (Intermediate 7) (200 mg, 723 mol, 1.0 equiv) and methyl 4-methylpiperidine-4-carboxylate (168 mg, 867 mol, 1.2 equiv) in THF (5 mL) at rt was added t-BuONa (139 mg, 1.45 mmol, 2.0 equiv), RuPhos Pd G3 (60 mg, 72 mol, 0.1 equiv) and RuPhos (34 mg, 72 μmol, 0.1 equiv). The resulting reaction mixture was heated to reflux and stirred for 12 hr. The reaction mixture was then cooled to rt. The reaction mixture was poured into H₂O (20 mL) and extracted with EtOAc (3×20 mL). The combined organic phase was washed with brine (20 mL), dried with anhydrous Na₂SO₄, filtered, and concentrated under vacuum. The residue was purified by silica gel column chromatography (PE:EtOAc=50:1 to 5:1) to afford methyl 1-(6-butyl-3-(4-methoxyphenyl)pyrazin-2-yl)-4-methylpiperidine-4-carboxylate (70 mg, 20% yield) as a yellow solid. ¹H NMR (400 MHz, CD₃OD) δ7.91 (s, 1H), 7.78 (d, J=8.8, 2H), 7.04 (d, J=8.8, 2H), 3.87 (s, 3H), 3.70 (s, 3H), 3.40 (m, 2H), 2.86 (m, 2H), 2.72 (t, J=7.6, 2H), 2.04 (m, 3H), 1.75 (m, 2H), 1.45 (m, 4H), 1.27 (m, 1H), 1.20 (m, 3H), 0.99 (t, J=7.4, 3H).

### Step 3: 1-(6-butyl-3-(4-methoxyphenyl)pyrazin-2-yl)-4-methylpiperidine-4-carboxylic acid (Compound 14)

To a solution of methyl 1-(6-butyl-3-(4-methoxyphenyl)pyrazin-2-yl)-4-methylpiperidine-4-carboxylate (60 mg, 151 μmol, 1.0 equiv) in MeOH (2 mL) at rt was added aqueous NaOH (4 M, 226 L, 6.0 equiv). The resulting reaction mixture was heated to 50° C. and stirred for 12 hr. The reaction mixture was then cooled to rt. The reaction mixture was purified by reverse-phase preparative HPLC (column: Waters Xbridge Prep OBD C18 150×40 mm×10 m; mobile phase: [H₂O (10 mM NH₄HCO₃)-ACN]; B %: 25%-50%, 8 min run time) to afford 1-(6-butyl-3-(4-methoxyphenyl)pyrazin-2-yl)-4-methylpiperidine-4-carboxylic acid (38 mg, 58% yield, 98% purity by LC/MS) as a white solid. ¹H NMR (400 MHz, CD₃OD) δ7.90 (s, 1H), 7.78 (d, J=8.8, 2H), 7.04 (d, J=8.8, 2H), 3.87 (s, 3H), 3.40 (m, 2H), 2.91 (m, 2H), 2.76 (t, J=7.6, 2H), 2.02 (m, 2H), 1.75 (m, 2H), 1.43 (m, 4 H), 1.22 (s, 3H), 0.99 (t, J=7.2, 3H); LCMS calculated for C₂₂H₂₉N₃O₃: m/z=383; found: m/z=384 (M+H).

### Example 15: 2-(1-(6-Butyl-3-(4-Methoxyphenyl)Pyrazin-2-yl)Piperidin-4-yl)Acetic acid (Compound 15)

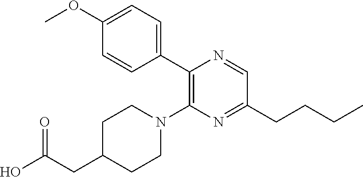 [SMILES: CCCCc1cnc(-c2ccc(OC)cc2)c(N2CCC(CC(=O)O)CC2)n1]

### Step 1: Methyl 2-(1-(6-butyl-3-(4-methoxyphenyl)pyrazin-2-yl)piperidin-4-yl)acetate

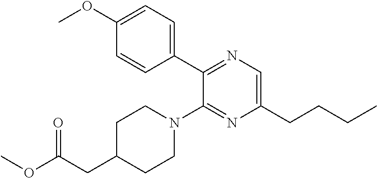 [SMILES: CCCCc1cnc(-c2ccc(OC)cc2)c(N2CCC(CC(=O)OC)CC2)n1]

To a solution of 5-butyl-3-chloro-2-(4-methoxyphenyl)pyrazine (Intermediate 7) (100 mg, 361 mol, 1.0 equiv) in NMP (2 mL) at rt was added methyl 2-(4-piperidyl)acetate (284 mg, 1.81 mmol, 5.0 equiv). The resulting reaction mixture was heated to 160° C. and stirred for 3 hr. The reaction mixture was cooled to rt, diluted with H₂O (100 mL), and then extracted with EtOAc (2×100 mL). The combined organic layers were dried over Na₂SO₄, filtered, and concentrated under reduced pressure to give crude methyl 2-(1-(6-butyl-3-(4-methoxyphenyl)pyrazin-2-yl)piperidin-4-yl)acetate (100 mg) as a yellow solid which was used without further purification. LCMS calculated for C₂₃H₃1N₃O₃: m/z=397; found: m/z=398 (M+H).

### Step 2: 2-(1-(6-Butyl-3-(4-methoxyphenyl)pyrazin-2-yl)piperidin-4-yl)acetic acid (Compound 15)

To a solution of methyl 2-(1-(6-butyl-3-(4-methoxyphenyl)pyrazin-2-yl)piperidin-4-yl)acetate (80 mg, 201 mol, 1.0 equiv) in MeOH (1 mL) at rt was added aqueous NaOH (4 M, 302 L, 6.0 equiv). The resulting reaction mixture was heated to 50° C. and stirred for 3 hr. The reaction mixture was cooled to rt, filtered to remove solids, and the filtrate was concentrated under reduced pressure to give a residue. The residue was purified by reverse-phase preparative HPLC (column: Waters Xbridge BEH C18 100×30 mm×10 m; mobile phase: [H₂O (10 mM NH₄HCO₃)-ACN]; B %: 25%-55%, 10 min run time) to afford 2-(1-(6-butyl-3-(4-methoxyphenyl)pyrazin-2-yl)piperidin-4-yl)acetic acid (49 mg, 60% yield, 98% purity by LC/MS) as a yellow solid. ¹H NMR (400 MHz, CD₃OD) δ 7.87 (s, 1H), 7.77 (d, J=8.8, 2H), 7.02 (d, J=8.8, 2H), 3.86 (s, 3H), 3.64 (m, 2H) 2.69 (m, 4H), 2.11 (d, J=7.6, 2H), 1.87 (m, 1H), 1.79-1.66 (m, 4H), 1.42 (m, 2H), 1.26 (m, 2H) 0.99 (t, J=7.6, 3H); LCMS calculated for C₂₂H₂₉N₃O₃: m/z=383; found: m/z=384 (M+H).

### Example 16: 3-(4-(2H-Tetrazol-5-yl)Piperidin-1-yl)-5-Butyl-2-(4-methoxyphenyl)pyrazine (Compound 16)

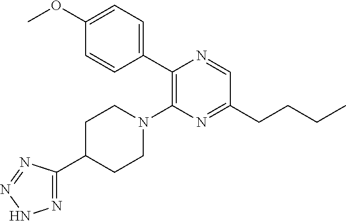 [SMILES: CCCCc1cnc(-c2ccc(OC)cc2)c(N2CCC(c3nn[nH]n3)CC2)n1]

### Step 1: 1-(6-butyl-3-(4-methoxyphenyl)pyrazin-2-yl)piperidine-4-carbonitrile

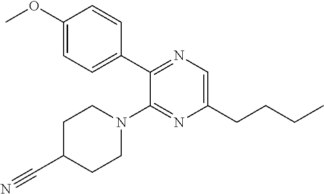 [SMILES: CCCCc1cnc(-c2ccc(OC)cc2)c(N2CCC(C#N)CC2)n1]

To a mixture of 5-butyl-3-chloro-2-(4-methoxyphenyl)pyrazine (Intermediate 7) (300 mg, 1.08 mmol, 1.0 equiv), piperidine-4-carbonitrile hydrochloride (238 mg, 1.63 mmol, 1.5 equiv) and t-BuONa (364 mg, 3.79 mmol, 3.5 equiv) in THF (5 mL) at rt was added Xphos Pd G4 (93 mg, 108 mol, 0.1 equiv) and Xantphos (62 mg, 108 mol, 0.1 equiv). The resulting reaction mixture was heated to reflux and stirred for 12 hr. The reaction was cooled to rt and then poured into H₂O (10 mL) and extracted with EtOAc (2×10 mL). The combined organic phase was washed with brine (20 mL), dried with anhydrous Na₂SO₄, filtered, and concentrated under vacuum. The residue was purified by silica gel column chromatography (PE:EtOAc=50:1 to 1:1) to afford 1-(6-butyl-3-(4-methoxyphenyl)pyrazin-2-yl)piperidine-4-carbonitrile (160 mg, 42% yield) as yellow oil. LCMS calculated for C₂₁H₂₆N₄O: m/z=350; found: m/z=351 (M+H).

### Step 2: 3-(4-(2H-tetrazol-5-yl)piperidin-1-yl)-5-butyl-2-(4-methoxyphenyl)pyrazine (Compound 16)

To a suspension of tributyltin chloride (278 mg, 856 mol, 3.0 equiv) in 1,4-dioxane (3 mL) at rt in a sealed tube under N₂(g) was added NaN₃ (55 mg, 856 mol, 3.0 equiv). The suspension was degassed under vacuum and purged with N₂(g) three times. The resulting reaction mixture was stirred at rt for 30 min. Next, 1-[6-butyl-3-(4-methoxyphenyl)pyrazin-2-yl]piperidine-4-carbonitrile (100 mg, 285 mol, 1.0 equiv) was added at rt as a solid. The resulting mixture was heated to 115° C. and stirred for 48 hr under an N₂(g) atmosphere. The reaction was cooled to rt and then quenched with saturated aqueous potassium fluoride (10 mL) and then extracted with EtOAc (2×10 mL). The combined organic layers were washed with brine (10 mL), dried over Na₂SO₄, filtered, and concentrated under reduced pressure to give a residue. The residue was purified by reverse-phase preparative HPLC (column: Phenomenex C18 80×40 mm×3 μm; mobile phase: [H₂O (NH₄HCO₃)-ACN]; B %: 20%-40%, 8 min run time) to afford 3-(4-(2H-tetrazol-5-yl)piperidin-1-yl)-5-butyl-2-(4-methoxyphenyl)pyrazine (20 mg, 17% yield, 100% purity by LC/MS) as a white solid. ¹H NMR (400 MHz, DMSO-d₆) δ 7.96 (s, 1H), 7.82 (d, J=8.8, 2 H), 7.05 (d, J=8.8, 2H), 3.86 (s, 3H), 3.76 (d, J=13.2, 2H), 3.22 (m, 2H), 2.89 (m, 2H), 2.75 (t, J=7.6, 2H), 2.03 (m, 2H), 1.88 (m, 2H), 1.77 (m, 2H), 1.46 (m, 2H), 0.99 (t, J=7.2, 3H); LCMS calculated for C₂₁H27N₇0: m/z=393; found: m/z=394 (M+H).

### Example 17: 1-(6-Butyl-3-(1H-Indol-6-yl)Pyrazin-2-yl)Piperidine-4-Carboxylic Acid (Compound 17)

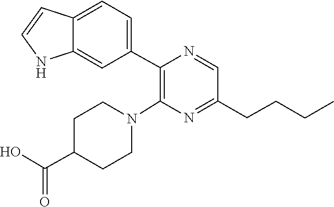 [SMILES: CCCCc1cnc(-c2ccc3cc[nH]c3c2)c(N2CCC(C(=O)O)CC2)n1]

### Step 1: Methyl 1-(6-butyl-3-(1H-indol-6-yl)pyrazin-2-yl)piperidine-4-carboxylate

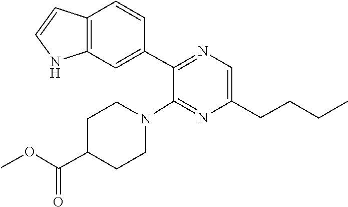 [SMILES: CCCCc1cnc(-c2ccc3cc[nH]c3c2)c(N2CCC(C(=O)OC)CC2)n1]

To a mixture of methyl 1-(3-bromo-6-butylpyrazin-2-yl)piperidine-4-carboxylate (Intermediate 4) (70 mg, 196 mol, 1.0 equiv) and 1H-indol-6-ylboronic acid (38 mg, 236 mol, 1.2 equiv) in THF (2 mL) and H₂O (0.4 mL) at rt was added [1,1′-bis(di-tert-butylphosphino)ferrocene]dichloropalladium(II) (19 mg, 28 mol, 0.15 equiv) and K₂CO₃(54 mg, 393 mol, 2.0 equiv). The resulting reaction mixture was heated to 80° C. and stirred for 12 hr. The reaction mixture was cooled to rt, filtered to remove solids, and the filtrate was concentrated under reduced pressure to give crude methyl 1-(6-butyl-3-(1H-indol-6-yl)pyrazin-2-yl)piperidine-4-carboxylate, which was used directly without further purification. LCMS calculated for C₂₃H₂₈N₄O₂: m/z=392; found: m/z=393 (M+H).

### Step 2: 1-(6-Butyl-3-(1H-indol-6-yl)pyrazin-2-yl)piperidine-4-carboxylic acid (Compound 17)

To a solution of crude methyl 1-(6-butyl-3-(1H-indol-6-yl)pyrazin-2-yl)piperidine-4-carboxylate (77 mg, 196 mol, 1.0 equiv) in MeOH (2 mL) at rt was added aqueous NaOH (4 M, 294 L, 6.0 equiv). The resulting reaction mixture was heated to 50° C. and stirred for 3 hr. The reaction mixture was cooled to rt, filtered to remove solids, and the filtrate was concentrated under reduced pressure to give a residue. The residue was purified by reverse-phase preparative HPLC (column: Waters Xbridge Prep OBD C18 100×30 mm x m; mobile phase: [H₂O (10 mM NH₄HCO₃)-ACN]; B %: 20%-50%, 10 min run time) to afford 1-(6-butyl-3-(1H-indol-6-yl)pyrazin-2-yl)piperidine-4-carboxylic acid (46 mg, 62% yield, 100% purity by LC/MS) as a white solid. ¹H NMR (400 MHz, CD₃OD) δ7.91 (m, 2 H), 7.65 (d, J=8.0, 1H), 7.48 (J=8.0, 1H), 7.33 (d, J=3.2, 1H), 6.50 (d, J=3.2, 1H), 3.68 (m, 2H), 2.74 (m, 4H), 2.37 (m, 1H), 1.81-1.64 (m, 6H), 1.45 (m, 2H) 1.00 (t, J=7.2, 3H); LCMS calculated for C₂₂H₂₆N₄O₂: m/z=378; found: m/z=379 (M+H).

### Example 18: 1-(3-(benzo[d][1,3]dioxol-5-yl)-6-(4,4,4-Trifluorobutyl)Pyrazin-2-yl)piperidine-4-carboxylic acid (Compound 108)

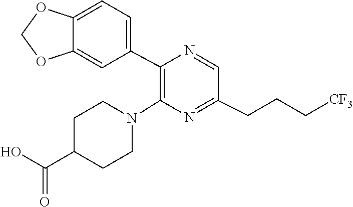 [SMILES: O=C(O)C1CCN(c2nc(CCCC(F)(F)F)cnc2-c2ccc3c(c2)OCO3)CC1]

### Step 1: Ethyl 1-(3-(benzo[d][1,3]dioxol-5-yl)-6-(4,4,4-trifluorobutyl)pyrazin-2-yl)piperidine-4-carboxylate

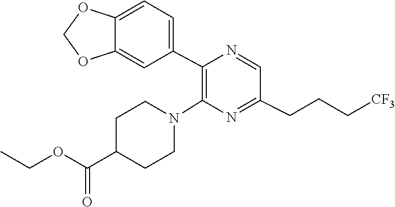 [SMILES: CCOC(=O)C1CCN(c2nc(CCCC(F)(F)F)cnc2-c2ccc3c(c2)OCO3)CC1]

To a mixture of ethyl 1-(3-chloro-6-(4,4,4-trifluorobutyl)pyrazin-2-yl)piperidine-4-carboxylate (Intermediate 12) (130 mg, 342 μmol, 1.0 eq), benzo[d][1,3]dioxol-5-ylboronic acid (57 mg, 342 μmol, 1.0 eq) and K₂CO₃ (95 mg, 684 μmol, 2.0 eq) in THF (2.0 mL) and H₂O (0.4 mL) at rt was added [di-tert-butyl(cyclopentyl)ferrocene]dichloropalladium(II) (22 mg, 34 μmol, 0.1 eq). The resulting reaction mixture was purged with N₂ (g) three times, and then the reaction mixture was heated to 80° C. and stirred for 12 hr under an N₂ (g) atmosphere. The reaction mixture was cooled to rt, diluted with H₂O (30 mL) and extracted with EtOAc (3×20 mL). The combined organic layers were dried over Na₂SO₄, filtered, and concentrated under reduced pressure to give a residue. The residue was purified by silica gel column chromatography (PE:EtOAc=1:0 to 5:1) to afford ethyl 1-(3-(benzo[d][1,3]dioxol-5-yl)-6-(4,4,4-trifluorobutyl)pyrazin-2-yl)piperidine-4-carboxylate (160 mg, 84% yield) as a light yellow oil. ¹H NMR (400 MHz, CD₃OD) δ=7.93 (s, 1H), 7.35 (d, J=8.0, 1H), 7.31 (s, 1H), 6.92 (d, J=8.0, 1H), 6.01 (s, 2H), 4.12 (q, J=7.2, 2H), 3.64 (m, 2H), 2.79 (m, 4 H), 2.46 (m, 1H), 2.25 (m, 2H), 2.02 (m, 2H), 1.85 (m, 2H), 1.66 (m, 2H), 1.24 (t, J=7.2, 3H). LCMS calculated for C₂₃H₂₆F₃N₃O₄: m/z=465; found: m/z=466 (M+H).

### Step 2: 1-(3-(benzo[d][1,3]dioxol-5-yl)-6-(4,4,4-trifluorobutyl)pyrazin-2-yl)piperidine-4-carboxylic acid (Compound 108)

To a solution of ethyl 1-(3-(benzo[d][1,3]dioxol-5-yl)-6-(4,4,4-trifluorobutyl)pyrazin-2-yl)piperidine-4-carboxylate (140 mg, 300 μmol, 1.0 eq) in MeOH (1 mL) was added NaOH (2 M, 0.3 mL, 2.0 eq). The resulting reaction mixture was stirred at 50° C. for 1 hr. The mixture was then cooled to rt, filtered, and concentrated under reduced pressure to give a residue. The residue was purified by reverse-phase preparative HPLC (column: Waters Xbridge BEH C18 100×30 mm×10 μm; mobile phase: [H₂O(10 mM NH₄HCO₃)-ACN];B %: 20%-50%, 8 min run time) to afford 1-(3-(benzo[d][1,3]dioxol-5-yl)-6-(4,4,4-trifluorobutyl)pyrazin-2-yl)piperidine-4-carboxylic acid (62 mg, 45% yield, 100% purity by LC/MS) as a white solid. ¹H NMR (400 MHz, CD₃OD) δ=7.92 (s, 1H), 7.37 (d, J=8.0, 1H), 7.31 (s, 1H), 6.92 (d, J=8.0, 1H), 6.01 (s, 2H), 3.65 (m, 2H), 2.77 (m, 4H), 2.38 (m, 1H), 2.26 (m, 2H), 2.03 (m, 2H), 1.85 (m, 2H), 1.67 (m, 2H); LCMS calculated for C₂₁H₂₂F₃N₃O₄: m/z=437; found: m/z=438 (M+H).

### Example 19: 1-(3-(7-Fluorobenzofuran-5-yl)-6-(3,3,3-Trifluoropropyl)Pyrazin-2-yl)piperidine-4-carboxylic acid (Compound 61)

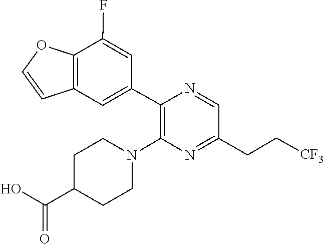 [SMILES: O=C(O)C1CCN(c2nc(CCC(F)(F)F)cnc2-c2cc(F)c3occc3c2)CC1]

### Step 1: Ethyl 1-(3-(7-fluorobenzofuran-5-yl)-6-(3,3,3-trifluoropropyl)pyrazin-2-yl)piperidine-4-carboxylate

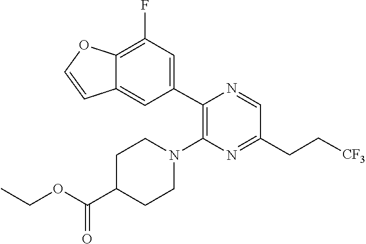 [SMILES: CCOC(=O)C1CCN(c2nc(CCC(F)(F)F)cnc2-c2cc(F)c3occc3c2)CC1]

To a solution of ethyl 1-(3-chloro-6-(3,3,3-trifluoropropyl)pyrazin-2-yl)piperidine-4-carboxylate (Intermediate 35) (60 mg, 164 μmol, 1.0 eq) and 2-(7-fluorobenzofuran-5-yl)-4,4,5,5-tetramethyl-1,3,2-dioxaborolane (Intermediate 41) (56 mg, 213 μmol, 1.3 eq) in THF (1 mL) and H₂O (0.2 mL) at rt under an N2 (g) atmosphere was added [di-tert-butyl(cyclopentyl)ferrocene]dichloropalladium(II) (11 mg, 16 μmol, 0.1 eq) and K₂CO₃ (45 mg, 328 μmol, 2.0 eq). The resulting reaction mixture was heated to 80° C. and stirred for 3 hr. The reaction mixture was cooled to rt, filtered through a pad of celite, and the solvents were removed under reduced pressure to afford crude ethyl 1-(3-(7-fluorobenzofuran-5-yl)-6-(3,3,3-trifluoropropyl)pyrazin-2-yl)piperidine-4-carboxylate, which used directly further purification. LCMS calculated for C₂₃H₂₃F₄N₃O₃: m/z=465; found: m/z=466 (M+H).

### Step 2: 1-(3-(7-fluorobenzofuran-5-yl)-6-(3,3,3-trifluoropropyl)pyrazin-2-yl)piperidine-4-carboxylic acid (Compound 61)

To a solution of ethyl 1-(3-(7-fluorobenzofuran-5-yl)-6-(3,3,3-trifluoropropyl)pyrazin-2-yl)piperidine-4-carboxylate in MeOH (1 mL) was added NaOH (4 M, 193 μL, 6.0 eq). The resulting reaction mixture was heated to 50° C. and stirred for 3 hr. The reaction mixture was cooled to rt, filtered to remove any particulates, and purified by reverse-phase HPLC (column: Phenomenex C18 75×30 mm×3 μm; mobile phase: [H₂O(10 mM NH₄HCO₃)-ACN];B %: 15%-65%, 8 min run time) to afford 1-(3-(7-fluorobenzofuran-5-yl)-6-(3,3,3-trifluoropropyl)pyrazin-2-yl)piperidine-4-carboxylic acid (36 mg, 62% yield, 99% purity by LC/MS) as a white solid. ¹H NMR (400 MHz, CD₃OD) δ8.05 (s, 1H), 7.97 (d, J=1.2, 1H), 7.91 (d, J=2.4, 1H), 7.62 (dd, J=1.2, 11.2, 1H), 7.03 (t, J=2.4, 1H), 3.66 (m,, 2H), 3.02 (m, 2H), 2.84-2.68 (m, 4H), 2.39 (m, 1H), 1.84 (m, 2H), 1.69 (m, 2H); LCMS calculated for C₂₁H₁₉F₄N₃O₃: m/z=437; found: m/z=438 (M+H).

### Example 20: 1-(3-(benzofuran-5-yl)-6-(5,5,5-Trifluoropentyl)Pyrazin-2-yl)Piperidine-4-carboxylic acid (Compound 130)

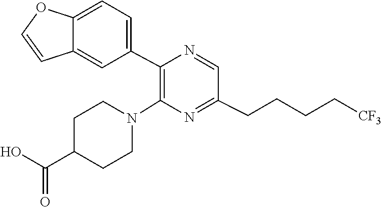 [SMILES: O=C(O)C1CCN(c2nc(CCCCC(F)(F)F)cnc2-c2ccc3occc3c2)CC1]

To a solution of ethyl 1-(3-chloro-6-(5,5,5-trifluoropentyl)pyrazin-2-yl)piperidine-4-carboxylate (Intermediate 52) (150 mg, 380 μmol, 1.0 eq) and benzofuran-5-ylboronic acid (92 mg, 571 μmol, 1.5 eq) in THF (2 mL) and H₂O (0.4 mL) at rt was added [di-tert-butyl(cyclopentyl)ferrocene]dichloropalladium(II) (24 mg, 38 μmol, 0.1 eq) and K₂CO₃ (105 mg, 761 μmol, 2.0 eq). The resulting reaction mixture was heated at 80° C. under an N₂ (g) atmosphere and stirred for 2 hr. The reaction mixture was cooled to rt, poured into H₂O (50 mL), and extracted with EtOAc (2×100 mL). The combined organic phase was washed with brine (20 mL), dried with anhydrous Na₂SO₄, filtered, and concentrated under vacuum. The residue was dissolved in MeOH (2 mL) at rt and then NaOH (4 M, 500 μL, 6.0 eq) was added. The resulting reaction mixture was heated at 50° C. and stirred for 1 hr. The reaction mixture was cooled to rt, filtered, and the filtrate concentrated under reduced pressure. The residue was purified by reverse-phase preparative HPLC (column: Phenomenex C18 80×40 mm×3 μm; mobile phase: [H₂O(10 mM NH₄HCO₃)-ACN];B %: 25%-45%, 8 min run time) to afford 1-(3-(benzofuran-5-yl)-6-(5,5,5-trifluoropentyl)pyrazin-2-yl)piperidine-4-carboxylic acid (100 mg, 68% yield, 99% purity by LC/MS) as a yellow solid. ¹H NMR (400 MHz, CD₃OD) δ8.08 (d, J=1.6, 1H), 7.97 (s, 1H), 7.80 (m, 2H), 7.61 (d, J=8.8, 1H), 6.94 (s, 1 H), 3.63 (m, 2H), 2.77 (m, 4H), 2.41 (m, 1H), 2.24 (m, 2H), 1.84 (m, 4H), 1.67 (m, 4H); LCMS calculated for C₂₃H₂₄F₃N₃O₃: m/z=447; found: m/z=448 (M+H).

### Example 21: 1-(3-(benzofuran-5-yl)-6-(3-Methoxypropyl)Pyrazin-2-yl)Piperidine-4-carboxylic acid (Compound 172)

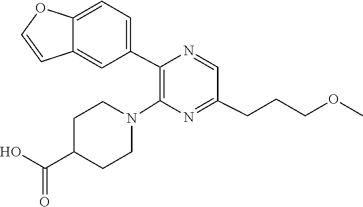 [SMILES: COCCCc1cnc(-c2ccc3occc3c2)c(N2CCC(C(=O)O)CC2)n1]

### Step 1: Ethyl 1-(3-(benzofuran-5-yl)-6-(3-methoxypropyl)pyrazin-2-yl)piperidine-4-carboxylate

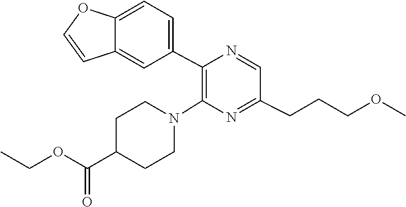 [SMILES: CCOC(=O)C1CCN(c2nc(CCCOC)cnc2-c2ccc3occc3c2)CC1]

To a solution of ethyl 1-(3-chloro-6-(3-methoxypropyl)pyrazin-2-yl)piperidine-4-carboxylate (Intermediate 93) (110 mg, 280 μmol, 1.0 eq) in THF (2.0 mL) and H₂O (0.2 mL) at rt was added benzofuran-5-ylboronic acid (46 mg, 280 μmol, 1.0 eq) and K₂CO₃ (78 mg, 560 μmol, 2.0 eq). The mixture was degassed under vacuum and purged with N₂ (g) three times. Next, [di-tert-butyl(cyclopentyl)ferrocene]dichloropalladium(II) (18 mg, 28 μmol, 0.1 eq) was added and the resulting reaction mixture was heated to 80° C. and stirred for 12 hr under an N₂ (g) atmosphere. The mixture was then cooled to rt, diluted with H₂O (20 mL), and extracted with EtOAc (3×20 mL). The combined organic layers were washed with brine (30 mL), dried over Na₂SO₄, filtered, and concentrated under reduced pressure to give a residue. The residue was purified by silica gel column chromatography (PE:EtOAc=1:0 to 3:1) to afford ethyl 1-(3-(benzofuran-5-yl)-6-(3-methoxypropyl)pyrazin-2-yl)piperidine-4-carboxylate (90 mg, 70% yield) as a brown oil. LCMS calculated for C₂₄H₂₉N₃O₄: m/z=423; found: m/z=424 (M+H).

### Step 2: 1-(3-(benzofuran-5-yl)-6-(3-methoxypropyl)pyrazin-2-yl)piperidine-4-carboxylic acid (Compound 172)

To a solution of ethyl 1-(3-(benzofuran-5-yl)-6-(3-methoxypropyl)pyrazin-2-yl)piperidine-4-carboxylate (85 mg, 200 μmol, 1.0 eq) in MeOH (1.0 mL) was added NaOH (2 M, 0.2 mL, 2.0 eq). The resulting reaction mixture was heated 50° C. and stirred for 1 hr. The mixture was then cooled to rt, filtered, and concentrated under reduced pressure to give a residue. The residue was purified by reverse-phase preparative HPLC (column: Waters Xbridge BEH C18 100×30 mm×10 μm; mobile phase: [H₂O(10 mM NH₄HCO₃)-ACN];B %: 30%-60%, 8 min run time) to afford 1-(3-(benzofuran-5-yl)-6-(3-methoxypropyl)pyrazin-2-yl)piperidine-4-carboxylic acid (49 mg, 62% yield, 99% purity by LC/MS) as a white solid. ¹H NMR (400 MHz, CDCl₃) δ 8.13 (d, J=1.6, 1H), 8.04 (s, 1H), 7.87 (dd, J=2.0, 8.4, 1 H), 7.66 (d, J=2.0, 1H), 7.56 (d, J=8.4, 1H), 6.83 (d, J=1.6, 1H), 3.66 (m, 2H), 3.48 (t, J=6.4, 2H), 3.38 (s, 3H), 2.78 (m, 4H), 2.46 (m, 1H), 2.07 (m, 2H), 1.86 (m, 2H), 1.73 (m, 2H); LCMS calculated for C₂₂H₂₅N₃O₄: m/z=395; found: m/z=396 (M+H).

### Example 22: 1-(3-(7-Fluorobenzo[d][1,3]dioxol-5-yl)-6-(3-Methoxypropyl)Pyrazin-2-yl)piperidine-4-carboxylic acid (Compound 190)

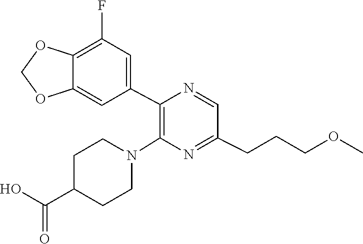 [SMILES: COCCCc1cnc(-c2cc(F)c3c(c2)OCO3)c(N2CCC(C(=O)O)CC2)n1]

### Step 1: Ethyl 1-(3-(7-fluorobenzo[d][1,3]dioxol-5-yl)-6-(3-methoxypropyl)pyrazin-2-yl)piperidine-4-carboxylate

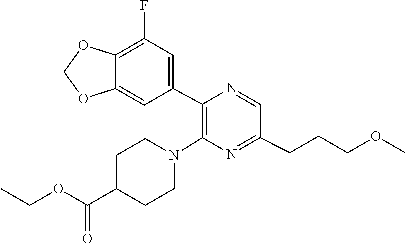 [SMILES: CCOC(=O)C1CCN(c2nc(CCCOC)cnc2-c2cc(F)c3c(c2)OCO3)CC1]

To a solution of ethyl 1-(3-chloro-6-(3-methoxypropyl)pyrazin-2-yl)piperidine-4-carboxylate (Intermediate 93) (270 mg, 789 μmol, 1.0 eq) and 2-(7-fluorobenzo[d][1,3]dioxol-5-yl)-4,4,5,5-tetramethyl-1,3,2-dioxaborolane (Intermediate 88) (420 mg, 1.58 mmol, 2.0 eq) in THF (3 mL) and H₂O (0.6 mL) at rt under an N₂ (g) atmosphere was added K₂CO₃ (327 mg, 2.37 mmol, 3.0 eq). and [di-tert-butyl(cyclopentyl)ferrocene]dichloropalladium(II) (51 mg, 79 μmol, 0.1 eq). The resulting reaction mixture was heated to 80° C. and stirred for 3 hr. The reaction mixture was cooled to rt. poured into H₂O (30 mL) and extracted with EtOAc (3×30 mL). The combined organic phase was washed with brine (30 mL), dried with anhydrous Na₂SO₄, filtered, and concentrated under vacuum. The residue was purified by silica gel column chromatography (PE:EtOAc=10:1 to 5:1) to afford ethyl 1-(3-(7-fluorobenzo[d][1,3]dioxol-5-yl)-6-(3-methoxypropyl)pyrazin-2-yl)piperidine-4-carboxylate (320 mg, 82% yield) as a yellow oil. ¹H NMR (400 MHz, CDCl₃) δ 7.99 (s, 1H), 7.36 (d, J=6.8, 1H), 7.29 (d, J=6.0, 1H), 6.08 (s, 2H), 4.16 (q, J=7.2, 2H), 3.64 (m, 2H), 3.47 (t, J=6.4, 2H), 3.38 (s, 3H), 2.80 (m, 4 H), 2.48 (m, 1H), 2.04 (m, 2H), 1.91 (m, 2H), 1.78 (m, 2H), 1.30 (t, J=7.2, 3H); LCMS calculated for C₂₃H₂₈FN₃O₅: m/z=445; found: m/z=446 (M+H).

### Step 2: 1-(3-(7-fluorobenzo[d][1,3]dioxol-5-yl)-6-(3-methoxypropyl)pyrazin-2-yl)piperidine-4-carboxylic acid (Compound 190)

To a solution of ethyl 1-(3-(7-fluorobenzo[d][1,3]dioxol-5-yl)-6-(3-methoxypropyl)pyrazin-2-yl)piperidine-4-carboxylate (290 mg, 651 μmol, 1.0 eq) in MeOH (4 mL) at rt was added NaOH (4 M, 976 μL, 6.0 eq). The resulting reaction mixture was heated to 50° C. and stirred for 1 hr. The reaction mixture was cooled to rt, filtered, and the filtrate concentrated under reduced pressure. The residue was purified by reverse-phase preparative HPLC (column: Welch Xtimate C18 250×100 mm×10 μm; mobile phase: [H₂O(10 mM NH₄HCO₃)-ACN];B %: 5%-40%, 20 min run time) to afford 1-(3-(7-fluorobenzo[d][1,3]dioxol-5-yl)-6-(3-methoxypropyl)pyrazin-2-yl)piperidine-4-carboxylic acid (64 mg, 21% yield, 98% purity by LC/MS) as a yellow solid. ¹H NMR (400 MHz, CD₃OD) δ7.94 (s, 1H), 7.30 (m, 2H), 6.11 (s, 2H), 3.65 (m, 2H), 3.48 (t, J=6.4, 2H), 3.36 (s, 3H), 2.80 (m, 4H), 2.49 (m, 1H), 2.04 (m, 2H), 1.87 (m, 2H), 1.73 (m, 2H); LCMS calculated for C₂₁H₂₄FN₃O₅: m/z=417; found: m/z=418 (M+H).

### Example 23: 1-(3-(benzofuran-5-yl)-6-(3-Methoxypropyl)Pyrazin-2-yl)-2-methylpiperidine-4-carboxylic acid (Compound 180)

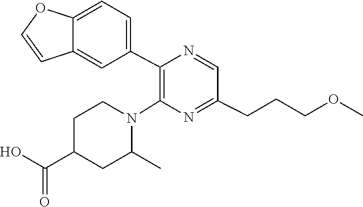 [SMILES: COCCCc1cnc(-c2ccc3occc3c2)c(N2CCC(C(=O)O)CC2C)n1]

### Step 1: 4-(Methoxycarbonyl)-2-methylpiperidinium acetate

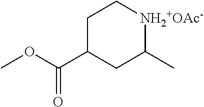

To a solution of methyl 2-methylpyridine-4-carboxylate (2.00 g, 13.23 mmol, 1.0 eq) in AcOH (20 mL) in a high pressure steel bomb reactor at rt under an N₂ (g) atmosphere was added PtO₂ (0.5 g, 2.20 mmol, 0.17 eq). The resulting suspension was degassed under vacuum and purged with H₂ (g) three times. The resulting reaction mixture was stirred at rt under an H₂ (g) atmosphere (290 psi) for 96 hr. The reaction mixture was then degassed under vacuum and purged with N₂ (g), filtered through a pad of celite to remove the catalyst, and the filtrate was concentrated under vacuum to afford 4-(methoxycarbonyl)-2-methylpiperidinium acetate (3.3 g, crude) as a brown liquid. ¹H NMR (400 MHz, CDCl₃) δ 3.70 (s, 3H), 3.42 (m, 1 H), 3.02 (m, 1H), 2.82 (m, 1H), 2.52 (m, 1 H), 2.10 (m, 2H), 1.83 (m, 1H), 1.67 (m, 1H), 1.35 (d, J=6.4, 3H).

### Step 2: Methyl 1-(3,6-dichloropyrazin-2-yl)-2-methylpiperidine-4-carboxylate

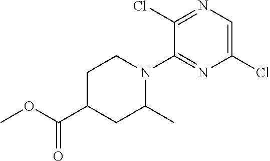 [SMILES: COC(=O)C1CCN(c2nc(Cl)cnc2Cl)C(C)C1]

To a solution of 2,3,5-trichloropyrazine (1.7 g, 9.27 mmol, 1.0 eq) and 4-(methoxycarbonyl)-2-methylpiperidinium acetate (2.91 g, 18.54 mmol, 2.0 eq) in dioxane (40 mL) at rt under an N₂ (g) atmosphere was added Et₃N (9.38 g, 92.68 mmol, 10.0 eq). The resulting reaction mixture was heated to 100° C. and stirred for 12 hr. The reaction mixture was cooled to rt, poured into H₂O (80 mL) and extracted with EtOAc (3×80 mL). The combined organic phase was washed with brine (120 mL), dried with anhydrous Na₂SO₄, filtered, and concentrated under vacuum. The residue was purified by silica gel column chromatography (PE:EtOAc=50:1 to 7:1) to afford methyl 1-(3,6-dichloropyrazin-2-yl)-2-methylpiperidine-4-carboxylate (750 mg) as a yellow oil. LCMS calculated for C₁₂HIsCl₂N₃O₂: m/z=303; found: m/z=304 (M+H).

### Step 3: Methyl 1-(3-(benzofuran-5-yl)-6-chloropyrazin-2-yl)-2-methylpiperidine-4-carboxylate

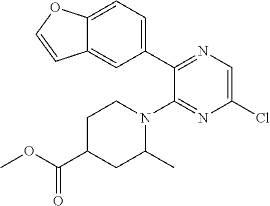 [SMILES: COC(=O)C1CCN(c2nc(Cl)cnc2-c2ccc3occc3c2)C(C)C1]

To a solution of methyl 1-(3,6-dichloropyrazin-2-yl)-2-methylpiperidine-4-carboxylate (350 mg, 1.15 mmol, 1.0 eq) and benzofuran-5-ylboronic acid (186 mg, 1.15 mmol, 1.0 eq) in DME (14 mL) at rt under an N₂(g) atmosphere was added Pd₂(dba)₃ (105 mg, 115 μmol, 0.1 eq), PPh₃ (60 mg, 230 μmol, 0.2 eq) and Na₂CO₃ (1 M, 2.30 mL, 2.0 eq). The resulting reaction mixture was heated to 80° C. and stirred for 12 hr. The reaction mixture was cooled to rt, poured into H₂O (50 mL) and extracted with EtOAc (3×50 mL). The combined organic phase was washed with brine (100 mL), dried with anhydrous Na₂SO₄, filtered, and concentrated under vacuum. The residue was purified by silica gel column chromatography (PE:EtOAc=50:1 to 7:1) to afford methyl 1-(3-(benzofuran-5-yl)-6-chloro-pyrazin-2-yl)-2-methylpiperidine-4-carboxylate (460 mg) as a yellow oil. LCMS calculated for C₂₀H₂₀ClN₃O₃: m/z=385; found: m/z=386 (M+H).

### Step 4: Methyl (E)-1-(3-(benzofuran-5-yl)-6-(3-methoxyprop-1-en-1-yl)pyrazin-2-yl)-2-methylpiperidine-4-carboxylate

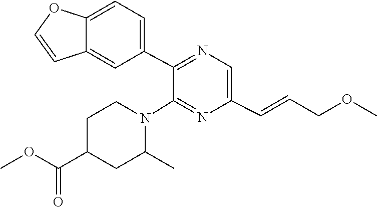

To a solution of methyl 1-(3-(benzofuran-5-yl)-6-chloro-pyrazin-2-yl)-2-methylpiperidine-4-carboxylate (470 mg, 1.22 mmol, 1.0 eq) and 2-[(E)-3-methoxyprop-1-enyl]-4,4,5,5-tetramethyl-1,3,2-dioxaborolane (482 mg, 2.44 mmol, 2.0 eq) in dioxane (10 mL) and H₂O (2 mL) at rt under an N₂ (g) atmosphere was chloro[(di(1-adamantyl)-N-butylphosphine)-2-(2-aminobiphenyl)]palladium(II) (cataCXium A-Pd-G2) (81 mg, 121 μmol, 0.1 eq) and Cs₂CO₃ (1.19 g, 3.65 mmol, 3.0 eq). The resulting reaction mixture was heated to 100° C. and stirred for 1 hr. The reaction mixture was cooled to rt, poured into H₂O (50 mL) and extracted with EtOAc (3×50 mL). The combined organic phase was washed with brine (100 mL), dried with anhydrous Na₂SO₄, filtered, and concentrated under vacuum. The residue was purified by silica gel column chromatography (PE:EtOAc=50:1 to 9:1) to afford methyl (E)-1-(3-(benzofuran-5-yl)-6-(3-methoxyprop-1-en-1-yl)pyrazin-2-yl)-2-methylpiperidine-4-carboxylate (410 mg, 77% yield) as a yellow oil. LCMS calculated for C₂₄H₂₇N₃O₄: m/z=421; found: m/z=422 (M+H).

### Step 5: Methyl 1-(3-(benzofuran-5-yl)-6-(3-methoxypropyl)pyrazin-2-yl)-2-methylpiperidine-4-carboxylate

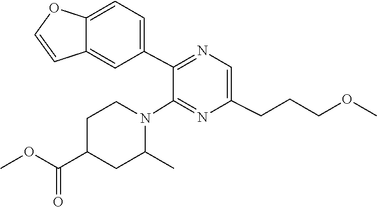

To a solution of methyl (E)-1-(3-(benzofuran-5-yl)-6-(3-methoxyprop-1-en-1-yl)pyrazin-2-yl)-2-methylpiperidine-4-carboxylate (50 mg, 118 μmol, 1.0 eq) in EtOAc (9 mL) at rt was added 10% Pd/C (50 mg). The resulting suspension was degassed under vacuum and purged with H₂ (g) three times. The resulting reaction mixture was stirred at rt under an H₂ (g) atmosphere (15 psi) for 10 min. The reaction mixture was then degassed under vacuum and purged with N₂ (g), filtered through a pad of celite to remove the catalyst, and the filtrate was concentrated under vacuum to afford methyl 1-(3-(benzofuran-5-yl)-6-(3-methoxypropyl)pyrazin-2-yl)-2-methylpiperidine-4-carboxylate (50 mg) as a green oil. LCMS calculated for C₂₄H₂₉N₃O₄: m/z=423; found: m/z=424 (M+H).

### Step 6: 1-(3-(benzofuran-5-yl)-6-(3-methoxypropyl)pyrazin-2-yl)-2-methylpiperidine-4-carboxylic acid (Compound 180)

To a solution of methyl 1-(3-(benzofuran-5-yl)-6-(3-methoxypropyl)pyrazin-2-yl)-2-methylpiperidine-4-carboxylate (80 mg, 188 μmol, 1.0 eq) in MeOH (2 mL) at rt was added NaOH (4 M, 280 μL, 6.0 eq). The resulting reaction mixture was heated at 60° C. and stirred for 1 hr. The reaction mixture was cooled to rt, filtered, and the filtrate concentrated under reduced pressure. The residue was purified by reverse-phase preparative HPLC (column: Waters Xbridge BEH C18 100×30 mm×10 μm; mobile phase: [H₂O(10 mM NH₄HCO₃)-ACN];B %: 15%-55%, 8 min run time) to afford 1-(3-(benzofuran-5-yl)-6-(3-methoxypropyl)pyrazin-2-yl)-2-methylpiperidine-4-carboxylic acid (40 mg, 51% yield, 97% purity by LC/MS) as a yellow solid. The material is a racemic 1:1 mixture of cis and trans diastereomers. ¹H NMR (400 MHz, CD₃OD) δ8.22 (s, 0.5 H), 8.15 (s, 0.5 H), 8.03 (s, 0.5 H), 7.95 (m, 1H), 7.83 (m, 1H), 7.75 (m, 0.5 H), 7.61 (m, 1H), 6.94 (s, 1H), 3.53 (m, 3H), 3.37 (s, 3H) 3.08 (m, 1H), 2.84 (m, 2H), 2.61 (m, 1H), 2.40 (m, 0.5 H), 2.06 (m, 2H), 1.83-1.58 (m, 4H), 1.44 (m, 0.5 H), 1.21 (d, J=6.0, 1.5 H), 1.03 (d, J=6.8, 1.5 H); LCMS calculated for C₂₃H₂₇N₃O₄: m/z=409; found: m/z=410 (M+H).

### Example 24: 1-(2-(benzo[d][1,3]dioxol-5-yl)-5-(3-Methoxypropyl)Pyridin-3-yl)piperidine-4-carboxylic acid (Compound 191)

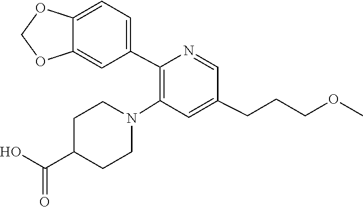 [SMILES: COCCCc1cnc(-c2ccc3c(c2)OCO3)c(N2CCC(C(=O)O)CC2)c1]

### Step 1: 1-(2-(benzo[d][1,3]dioxol-5-yl)-5-(3-methoxypropyl)pyridin-3-yl)piperidine-4-carbonitrile

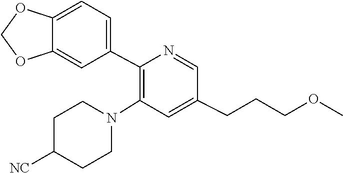 [SMILES: COCCCc1cnc(-c2ccc3c(c2)OCO3)c(N2CCC(C#N)CC2)c1]

To a solution of 3-(4-cyanopiperidin-1-yl)-5-(3-methoxypropyl)pyridin-2-yl trifluoromethanesulfonate (Intermediate 110) (100 mg, 245 μmol, 1.0 eq) and 1,3-benzodioxol-5-ylboronic acid (81 mg, 491 μmol, 2.0 eq) in EtOH (2 mL) and H₂O (0.5 mL) at rt under an N₂ (g) atmosphere was added chloro(2-dicyclohexylphosphino-2′,4′,6′-triisopropyl-1,1′-biphenyl)[2-(2′-amino-1,1′-biphenyl)]palladium(II) (XPhos Pd G2) (19 mg, 26 μmol, 0.1 eq) and K₃PO₄ (156 mg, 736 μmol, 3.0 eq). The resulting reaction mixture was heated to 80° C. and stirred for 2 hr. The reaction mixture was then cooled to rt, poured into H₂O (20 mL), and extracted with EtOAc (3×20 mL). The combined organic phase was washed with brine (20 mL), dried with anhydrous Na₂SO₄, filtered, and concentrated under vacuum. The residue was purified by silica gel column chromatography (PE:EtOAc=5:1 to 0:1) to afford 1-(2-(benzo[d][1,3]dioxol-5-yl)-5-(3-methoxypropyl)pyridin-3-yl)piperidine-4-carbonitrile (80 mg, 72% yield) as a yellow solid. LCMS calculated for C₂₂H₂₅N₃O₃: m/z=379; found: m/z=380 (M+H).

### Step 2: 1-(2-(benzo[d][1,3]dioxol-5-yl)-5-(3-methoxypropyl)pyridin-3-yl)piperidine-4-carboxylic acid (Compound 191)

1-(2-(benzo[d][1,3]dioxol-5-yl)-5-(3-methoxypropyl)pyridin-3-yl)piperidine-4-carbonitrile (60 mg, 158 mmol, 1.0 eq) was dissolved in HCl (6 N, 3.00 mL), and the resulting reaction mixture was heated to 100° C. and stirred for 3 hr. The reaction mixture was cooled to rt and combined with another batch of crude product (20 mg scale). The reaction mixture was filtered and the filtrate was purified by reverse-phase preparative HPLC (column: Phenomenex Luna C18 200×40 mm×10 μm; mobile phase: [H₂O(0.1% FA)-ACN]; B %: 20%-50%, 8 min run time) to afford 1-(2-(benzo[d][1,3]dioxol-5-yl)-5-(3-methoxypropyl)pyridin-3-yl)piperidine-4-carboxylic acid (23 mg, 27% yield, 97% purity by LC/MS) as a white solid. ¹H NMR (400 MHz, CD₃OD) δ8.05 (s, 1H), 7.49 (s, 1H), 7.36 (m, 2H), 6.98 (d, J=8.0, 1H), 6.04 (s, 2H), 3.46 (t, J=6, 2H), 3.37 (s, 3H), 3.33 (m, 2H), 2.78 (m, 2H), 2.67 (m, 2H), 2.41 (m, 1H), 1.94 (m, 4H), 1.72 (m, 2H); LCMS calculated for C₂₂H₂₆N₂O₅: m/z=398; found: m/z=399 (M+H).

### Example 25: 1-(2-(Furo[2,3-b]Pyridin-5-yl)-5-(3-Methoxypropyl)Pyridin-3-yl)piperidine-4-carboxylic acid (Compound 170)

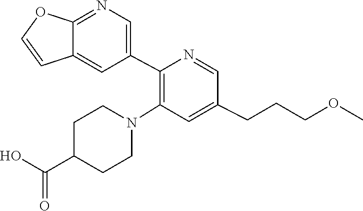 [SMILES: COCCCc1cnc(-c2cnc3occc3c2)c(N2CCC(C(=O)O)CC2)c1]

### Step 1: Ethyl 1-(2-(furo[2,3-b]pyridin-5-yl)-5-(3-methoxypropyl)pyridin-3-yl)piperidine-4-carboxylate

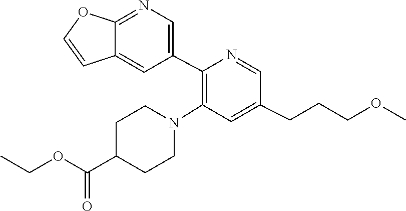 [SMILES: CCOC(=O)C1CCN(c2cc(CCCOC)cnc2-c2cnc3occc3c2)CC1]

To a solution of ethyl 1-(2-bromo-5-(3-methoxypropyl)-3-pyridyl)piperidine-4-carboxylate (Intermediate 123) (50 mg, 129 μmol, 1.0 eq) and 5-(4,4,5,5-tetramethyl-1,3,2-dioxaborolan-2-yl)furo[2,3-b]pyridine (Intermediate 104) (63 mg, 259 μmol, 2.0 eq) in THF (2 mL) and H20 (0.4 mL) at rt under an N₂ (g) atmosphere was added [di-tert-butyl(cyclopentyl)ferrocene]dichloropalladium(II) (16 mg, 25 μmol, 0.2 eq) and K₂CO₃ (35 mg, 259 μmol, 2.0 eq). The resulting reaction mixture was heated to 80° C. and stirred for 2 hr. The reaction mixture was cooled to rt, poured into H₂O (20 mL), and extracted with EtOAc (3×20 mL). The combined organic phase was washed with brine (20 mL), dried with anhydrous Na₂SO₄, filtered, and concentrated under vacuum. The residue was purified by silica gel column chromatography (PE:EtOAc=10:1 to 1:1) to afford ethyl 1-(2-(furo[2,3-b]pyridin-5-yl)-5-(3-methoxypropyl)pyridin-3-yl)piperidine-4-carboxylate (28 mg, 50% yield) as a white solid. LCMS calculated for C₂₄H₂₉N₃O₄: m/z=423; found: m/z=424 (M+H).

### Step 2: 1-(2-(furo[2,3-b]pyridin-5-yl)-5-(3-methoxypropyl)pyridin-3-yl)piperidine-4-carboxylic acid (Compound 170)

To a solution of ethyl 1-(2-(furo[2,3-b]pyridin-5-yl)-5-(3-methoxypropyl)pyridin-3-yl)piperidine-4-carboxylate (30 mg, 70 μmol, 1.0 eq) in MeOH (2 mL) at rt was added NaOH (4 M, 0.1 mL, 6.0 eq). The resulting reaction mixture was heated to 50° C. and stirred for 3 hr. The reaction mixture was cooled to rt, filtered, and the filtrate concentrated under reduced pressure. The residue was purified by reverse-phase preparative HPLC (column: Waters Xbridge BEH C18 100×30 mm×10 μm; mobile phase: [H₂O(10 mM NH₄HCO₃)-ACN]; B %: 10%-50%, 8 min run time) to afford 1-(2-(furo[2,3-b]pyridin-5-yl)-5-(3-methoxypropyl)pyridin-3-yl)piperidine-4-carboxylic acid (10 mg, 36% yield, 100% purity by LC/MS) as a white solid. ¹H NMR (400 MHz, CD₃OD) δ8.71 (s, 1H), 8.65 (s, 1H), 8.13 (s, 1H), 7.95 (d, J=2.4, 1H), 7.49 (s, 1 H), 7.04 (d, J=2.4, 1H), 3.46 (t, J=6.4, 2H), 3.38 (s, 3H), 3.13 (m, 2H), 3.27 (t, J=8.0, 2H), 2.64 (m, 2H), 2.14 (m, 1H), 1.95 (m, 2H), 1.82 (m, 2H), 1.65 (m, 2H); LCMS calculated for C₂₂H₂₅N₃O₄: m/z=395; found: m/z=396 (M+H).

### Example 26: 1-(5-(3-Methoxypropyl)-2-(1-Oxo-1,3-Dihydroisobenzofuran-5-yl)pyridin-3-yl)piperidine-4-carboxylic acid (Compound 231)

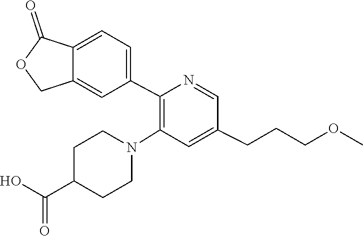 [SMILES: COCCCc1cnc(-c2ccc3c(c2)COC3=O)c(N2CCC(C(=O)O)CC2)c1]

### Step 1: 5-bromo-6-chloro-N-methoxy-N-methylnicotinamide

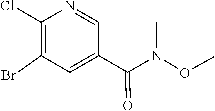

To a stirred solution of 5-bromo-6-chloronicotinic acid (5.0 g, 21.28 mmol, 1.0 eq) in DMF (25 mL) at 0° C. under an N₂ (g) atmosphere was added sequentially O,N-dimethyl hydroxylamine hydrochloride (3.2 g, 31.92 mmol, 1.5 eq), Et₃N (8.6 g, 85.14 mmol, 4 eq), EDC-HCl (8.16 g, 42.57 mmol, 2 eq) and then HOBT (288 mg, 2.13 mmol, 0.1 eq). The resulting reaction mixture was warmed to rt and stirred for 12 hr. The reaction mixture was quenched with cold water (100 mL) and then extracted with ethyl acetate (2×100 mL). The combined organic layer was washed with brine (100 mL), dried over Na₂SO₄, filtered, and concentrated under vacuum. The residue was purified by silica gel column chromatography (PE:EtOAc=1:0 to 5:1) to afford 5-bromo-6-chloro-N-methoxy-N-methylnicotinamide (4.7 g, 79% yield) as a white solid. ¹H NMR (400 MHz, CDCl₃) δ 8.71 (d, J=2.0, 1H), 8.30 (d, J=2.0, 1H), 3. 57 (s, 3H), 3.38 (s, 3H).

### Step 2: 5-bromo-6-chloronicotinaldehyde

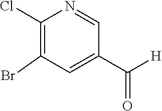

A solution of 5-bromo-6-chloro-N-methoxy-N-methylnicotinamide (Intermediate 133) (4.7 g, 17.27 mmol, 1.0 eq) in THF (50 mL) under an N₂ (g) atmosphere was cooled to −78° C., and then DIBAL-H (1M in toluene, 26 mL, 25.91 mmol, 1.5 eq) was added dropwise over 20 min. The resulting reaction mixture was stirred at −78° C. for 2 hr. The reaction mixture was quenched with saturated aqueous Rochelle's salt (20 mL) at −78° C. and then warmed to rt and stirred for 2 hr. The resulting solution was extracted with ethyl acetate (2×100 mL). The combined organic layer was washed with brine (100 mL), dried over Na₂SO₄, filtered, and concentrated under vacuum. The residue was purified by silica gel column chromatography (PE:EtOAc=1:0 to 8:1) to afford 5-bromo-6-chloronicotinaldehyde (860 mg, 90%) as a white solid. ¹H NMR (400 MHz, CDCl₃) δ 10.05 (s, 1H), 8.79 (d, J=2.0, 1 H), 8.38 (d, J=2.0, 1H).

### Step 3: (E)-3-bromo-2-chloro-5-(3-methoxyprop-1-en-1-yl)pyridine

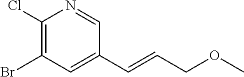

To a stirred solution of 5-bromo-6-chloronicotinaldehyde (Intermediate 134) (3.0 g, 13.75 mmol, 1.0 eq) in THF (30 mL) at rt under an N₂ (g) atmosphere was added (2-methoxyethyl)triphenylphosphonium bromide (8.23 g, 20.55 mmol, 1.5 eq) and K₂CO₃ (4.74 g, 34.26 mmol, 2.5 eq). The resulting reaction mixture was heated to 70° C. and stirred for 16 hr. The reaction mixture was then cooled to rt, poured into H₂O (100 mL), and extracted with EtOAc (2×100 mL). The combined organic phase was washed with brine (100 mL), dried with anhydrous Na₂SO₄, filtered, and concentrated under vacuum. The residue was purified by silica gel column chromatography (PE:EtOAc=10:1 to 8:1) to afford (E)-3-bromo-2-chloro-5-(3-methoxyprop-1-en-1-yl)pyridine (2.8 g, 77% yield) as a white solid. LCMS calculated for C₉H9BrClNO: m/z=261; found: m/z=264 (M+2H).

### Step 4: 3-bromo-2-chloro-5-(3-methoxypropyl)pyridine

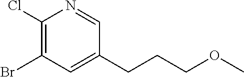

To a stirred solution of (E)-3-bromo-2-chloro-5-(3-methoxyprop-1-en-1-yl)pyridine (2.7 g, 10.35 mmol, 1.0 eq), in 5:1 EtOH:THF (30 mL), was added PtO₂ (380 mg, 1.55 mmol, 0.15 eq). The resulting suspension was degassed under vacuum and purged with H₂ (g) three times. The resulting reaction mixture was stirred at rt under H₂ (g) (15 psi) for 1 hr. The reaction mixture was then degassed under vacuum and purged with N₂ (g) and then filtered through a pad of celite to remove the catalyst. The filtrate was concentrated under vacuum to afford (3-bromo-2-chloro-5-(3-methoxypropyl)pyridine (1.5 g, 56% yield) as a colorless liquid. ¹H NMR (400 MHz, CDCl₃) δ 8.16 (s, 1H), 7.77 (s, 1H), 3.36 (m, 5H), 2.68 (m, 2H), 1.88 (m, 2H).

### Step 5: Benzyl 1-(2-chloro-5-(3-methoxypropyl)pyridin-3-yl)piperidine-4-carboxylate

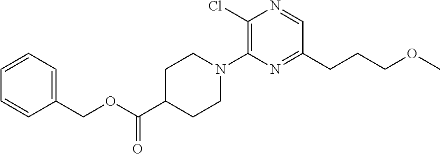 [SMILES: COCCCc1cnc(Cl)c(N2CCC(C(=O)OCc3ccccc3)CC2)n1]

To a stirred solution of 3-bromo-2-chloro-5-(3-methoxypropyl)pyridine (110 mg, 420 μmol, 1.0 eq) in 1,4-dioxane (2 mL) at rt was added benzyl piperidine-4-carboxylate (107 mg, 420 μmol, 1.0 eq) and Cs₂CO₃ (409 mg, 1.25 mmol, 3.0 eq). The resulting reaction mixture was stirred and purged with N₂ (g) for 10 min, and then Pd₂(dba)₃ (16 mg, 17 μmol, 0.04 eq) and 9,9-dimethyl-9H-xanthene-4,5-diyl)bis(diphenylphosphane) (Xantphos) (25 mg, 25 μmol, 0.06 eq) were added respectively. The resulting reaction mixture was again purged with N₂ (g) for 5 min, and then the reaction mixture was placed in a preheated oil bath at 110° C. and stirred for 16 hr. The reaction mixture was then cooled to rt and filtered. The filtrate was concentrated under vacuum. The residue was purified by silica gel column chromatography (PE:EtOAc=10:1 to 5:1) to afford benzyl 1-(2-chloro-5-(3-methoxypropyl)pyridin-3-yl)piperidine-4-carboxylate (80 mg, 48% yield) as a colorless liquid. LCMS calculated for C₂₂H₂₇C₁N₂O₃: m/z=402; found: m/z=403 (M+H).

### Step 6: Benzyl 1-(5-(3-methoxypropyl)-2-(1-oxo-1,3-dihydroisobenzofuran-5-yl)pyridin-3-yl)piperidine-4-carboxylate

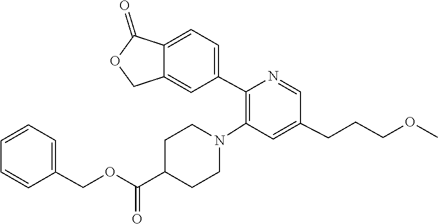 [SMILES: COCCCc1cnc(-c2ccc3c(c2)COC3=O)c(N2CCC(C(=O)OCc3ccccc3)CC2)c1]

To a stirred solution of benzyl 1-(2-chloro-5-(3-methoxypropyl)pyridin-3-yl)piperidine-4-carboxylate (40 mg, 90 μmol, 1.0 eq) in 1,4-dioxane (2 mL) and H₂O (0.2 mL) at rt under an N₂ (g) atmosphere was added 5-(4,4,5,5-tetramethyl-1,3,2-dioxaborolan-2-yl) isobenzofuran-1(3H)-one (34 mg, 130 μmol, 1.3 eq), K₂CO₃ (43 mg, 190 μmol, 2 eq), and [1,1′-bis(di-phenylphosphino)ferrocene]dichloropalladium(II) (5 mg, 6 μmol, 0.06 eq). The resulting reaction mixture was heated to 100° C. and stirred for 12 hr. The reaction mixture was then cooled to rt, poured into H₂O (10 mL), and extracted with EtOAc (2×20 mL). The combined organic phase was washed with brine (20 mL), dried with anhydrous Na₂SO₄, filtered, and concentrated under vacuum. The residue was purified by silica gel column chromatography (PE:EtOAc=1:0 to 1:5) to afford benzyl 1-(5-(3-methoxypropyl)-2-(1-oxo-1,3-dihydroisobenzofuran-5-yl)pyridin-3-yl)piperidine-4-carboxylate (35 mg, 61% yield) as a colorless liquid. LCMS calculated for C₃₀H₃₂N₂O₅: m/z=500; found: m/z=501 (M+H).

### Step 7: 1-(5-(3-methoxypropyl)-2-(1-oxo-1,3-dihydroisobenzofuran-5-yl)pyridin-3-yl)piperidine-4-carboxylic acid (Compound 231)

To a stirred solution of benzyl 1-(5-(3-methoxypropyl)-2-(1-oxo-1,3-dihydroisobenzofuran-5-yl)pyridin-3-yl)piperidine-4-carboxylate (35 mg, 70 μmol, 1.0 eq) in MeOH (2 mL) at rt under an N₂ (g) atmosphere was added 10% Pd(OH)₂ (10 mg, 70 μmol, 1.0 eq). The resulting suspension was degassed and purged with H₂ (g) three times. The resulting reaction mixture was stirred at rt under H₂ (g) at 30 psi for 2 hr. The mixture was then purged with N₂ (g) three times and then filtered through Celite to remove the catalyst. The filtrate was concentrated under reduced pressure to afford 1-(5-(3-methoxypropyl)-2-(1-oxo-1,3-dihydro-2-benzofuran-5-yl)pyridin-3-yl)piperidine-4-carboxylic acid (15 mg, 51% yield, 95% purity by LC/MS) as an off-white sticky solid. ¹H NMR (400 MHz, DMSO-d₆) δ 8.21 (m, 2H), 8.17 (m, 1H), 7.90 (d, J=8.0, 1H), 7.38 (s, 1H), 5.45 (s, 2H), 3.35 (t, J=6.4, 2H), 3.25 (s, 3H), 3.05 (m, 2H), 2.64 (m, 4H), 2.27 (m, 1H), 1.84 (m, 2H), 1.75 (m, 2 H), 1.56 (m, 2H); LCMS calculated for C₂₃H₂₆N₂O₅: m/z=410; found: m/z=411 (M+H).

### Example 27: 1-(2-(7-Fluorobenzofuran-5-yl)-5-(3-Methoxypropyl)Pyridin-3-yl)azepane-4-carboxylic acid (Compound 245)

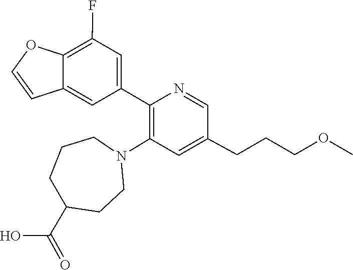 [SMILES: COCCCc1cnc(-c2cc(F)c3occc3c2)c(N2CCCC(C(=O)O)CC2)c1]

### Step 1: Methyl 1-(2-chloro-5-(3-methoxypropyl)pyridin-3-yl)azepane-4-carboxylate

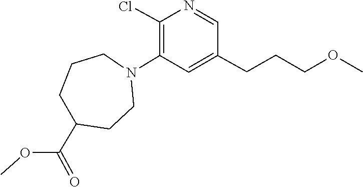

To a stirred solution of 3-bromo-2-chloro-5-(3-methoxypropyl)pyridine (see steps 1-4 in Example 26 for preparation) (200 mg, 756 μmol, 1.0 eq) in toluene (2 mL), was added methyl azepane-4-carboxylate-HCl (190 mg, 983 μmol, 1.3 eq), and Cs₂CO₃ (739 mg, 2.27 mmol, 3 eq). The resulting reaction mixture was purged with N₂ (g) for 10 min. Next, Pd₂(dba)₃ (35 mg, 38 μmol, 0.05 eq) and 9,9-dimethyl-9H-xanthene-4,5-diyl)bis(diphenylphosphane) (Xantphos) (44 mg, 76 μmol, 0.1 eq) were added respectively and the resulting reaction mixture was again purged with N₂ (g) for 5 min. The resulting reaction mixture was heated to 110° C. and stirred for 16 hr. The reaction mixture was then cooled to rt and filtered. The filtrate was concentrated under vacuum. The residue was purified by silica gel column chromatography (PE:EtOAc=10:1 to 5:1) to afford methyl 1-(2-chloro-5-(3-methoxypropyl)pyridin-3-yl)azepane-4-carboxylate (80 mg, 31% yield) as colorless liquid. LCMS calculated for C₁₇H₂₅C₁N₂O₃: m/z=340; found: m/z=341 (M+H).

### Step 2: Methyl 1-(2-(7-fluorobenzofuran-5-yl)-5-(3-methoxypropyl)pyridin-3-yl)azepane-4-carboxylate

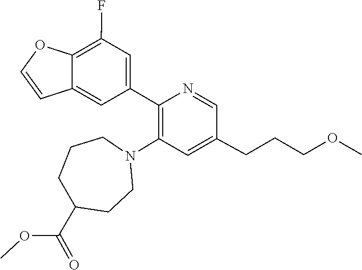

To a solution of methyl 1-(2-chloro-5-(3-methoxypropyl)pyridin-3-yl)azepane-4-carboxylate (55 mg, 161 μmol, 1.0 eq) and 2-(7-fluorobenzofuran-5-yl)-4,4,5,5-tetramethyl-1,3,2-dioxaborolane (Intermediate 41) (94 mg, 356 μmol, 2.2 eq) in 5:1 1,4-dioxane:H₂O at rt was added K₂CO₃ (45 mg, 323 μmol, 2.0 eq). The resulting reaction mixture was degassed with Ar (g) for 10 min at rt. Next, Pd(PPh₃)₄(11 mg, 10 μmol, 0.06 eq) was added to the reaction mixture and it was again degassed with Ar (g) for 10 min. The resulting reaction mixture was heated to 100° C. and stirred under an Ar (g) atmosphere for 12 hr. The reaction mixture was then cooled to rt, poured into H₂O (20 mL), and extracted with EtOAc (2×20 mL). The combined organic phase was washed with brine (20 mL), dried with anhydrous Na₂SO₄, filtered, and concentrated under vacuum. The residue was purified by silica gel column chromatography (PE:EtOAc=10:1 to 5:1) to afford methyl 1-(2-(7-fluoro-1-benzofuran-5-yl)-5-(3-methoxypropyl)pyridin-3-yl)azepane-4-carboxylate (45 mg, 63% yield). LCMS calculated for C₂₅H₂₉FN₂O₄: m/z=440; found: m/z=441 (M+H).

### Step 3: 1-(2-(7-fluorobenzofuran-5-yl)-5-(3-methoxypropyl)pyridin-3-yl)azepane-4-carboxylic acid (Compound 245)

To a solution of methyl 1-(2-(7-fluoro-1-benzofuran-5-yl)-5-(3-methoxypropyl)pyridin-3-yl)azepane-4-carboxylate (45 mg, 102 μmol, 1.0 eq) in MeOH (2 mL) was added NaOH (2N, 0.5 mL). The resulting reaction mixture was stirred at rt for 12 hr. The reaction mixture was then adjusted to pH=5 with 2N HCl and was extracted with EtOAc (3×20 mL). The combined organic layers were washed with brine (20 mL), dried over Na₂SO₄, filtered, and concentrated under vacuum to afford 1-(2-(7-fluoro-1-benzofuran-5-yl)-5-(3-methoxypropyl) pyridin-3-yl)azepane-4-carboxylic acid (39 mg, 91% yield, 97% purity by LC/MS) as a white sticky solid. ¹H NMR (400 MHz, DMSO-d₆) δ 12.01 (s, br, 1 H), 8.10 (d, J=13.5, 2H), 7.85 (s, 1H), 7.58 (d, J=12.8, 1H), 7.38 (s, 1H), 7.10 (s, 1H), 3.36 (t, J=6.4, 2H), 3.25 (s, 3H), 3.16 (m, 1H), 2.99 (m, 3H), 2.63 (t, J=7.6, 2H), 2.41 (m, 1H), 1.84 (m, 3H), 1.78 (m, 1H), 1.58 (m, 2H), 1.23 (m, 2H); LCMS calculated for C₂₄H27FN204: m/z=426; found: m/z=427 (M+H).

### Example 28: 1-(3-(7-Fluorobenzofuran-5-yl)-6-(3-Methoxy-2-(methoxymethyl)propyl)pyrazin-2-yl)piperidine-4-carboxylic acid (Compound 53)

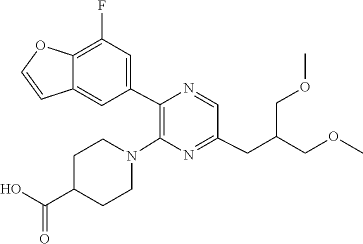 [SMILES: COCC(COC)Cc1cnc(-c2cc(F)c3occc3c2)c(N2CCC(C(=O)O)CC2)n1]

### Step 1: 3-methoxy-2-(methoxymethyl)prop-1-ene

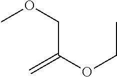

To a suspension of NaH (22.70 g, 567.51 mmol, 60% in oil, 5.0 eq) in THF (300 mL) at 0° C. was added dropwise 2-methylenepropane-1,3-diol (10.00 g, 113.50 mmol, 1.0 eq). The resulting reaction mixture was stirred at 0° C. under an N₂ (g) atmosphere for 30 min, then CH₃I (161.10 g, 1.14 mol, 10.0 eq) was added dropwise. The resulting reaction mixture was then warmed to rt and stirred for 12 hr. The reaction mixture was poured into H₂O (500 mL) and extracted with MTBE (1000 mL). The combined organic phase was washed with brine (100 mL), dried with anhydrous Na₂SO₄, filtered, and concentrated under vacuum. The residue was purified by silica gel column chromatography (PE:EtOAc=20:1 to 15:1) to afford 3-methoxy-2-(methoxymethyl)prop-1-ene (28 g) as a yellow oil. ¹H NMR (400 MHz, CDCl₃) δ 5.16 (s, 2H), 3.90 (s, 4H), 3.31 (s, 6H).

### Step 2: Ethyl 1-(6-(3-methoxy-2-(methoxymethyl)prop-1-en-1-yl)pyrazin-2-yl)piperidine-4-carboxylate

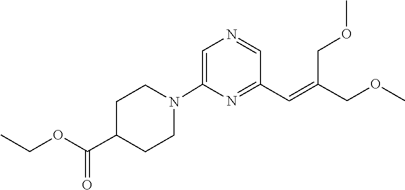 [SMILES: CCOC(=O)C1CCN(c2cncc(C=C(COC)COC)n2)CC1]

A solution of ethyl 1-(6-chloropyrazin-2-yl)piperidine-4-carboxylate (Intermediate 8) (300 mg, 1.11 mmol, 1.0 eq), 3-methoxy-2-(methoxymethyl)prop-1-ene (1.94 g, 16.68 mmol, 15.0 eq), chloro[(tri-tert-butylphosphine)-2-(2-aminobiphenyl)]palladium (II) (P(t-Bu)₃ Pd G2) (56 mg, 111 μmol, 0.1 eq) and N-cyclohexyl-N-methyl-cyclohexanamine (1.09 g, 5.56 mmol, 1.2 mL, 5.0 eq) in DMF (2 mL) was transferred into a microwave tube. The sealed tube was heated at 100° C. for 2.5 hr under microwave irradiation. The reaction mixture was cooled to rt, poured into H₂O (50 mL) and extracted with EtOAc (100 mL). The combined organic phase was washed with brine (20 mL), dried with anhydrous Na₂SO₄, filtered, and concentrated under vacuum. The residue was purified by silica gel column chromatography (PE:EtOAc=8:1 to 2:1) to afford ethyl 1-(6-(3-methoxy-2-(methoxymethyl)prop-1-en-1-yl)pyrazin-2-yl)piperidine-4-carboxylate (285 mg, 73% yield) as a yellow oil. ¹H NMR (400 MHz, CD₃OD) δ8.05 (s, 1H), 7.74 (s, 1H), 6.57 (s, 1H), 4.64 (s, 2H), 4.32 (m, 2H), 4.17 (m, 4H), 3.42 (s, 3H), 3.36 (s, 3H), 3.14 (m, 2H), 2.71-2.58 (m, 2H), 1.78-1.64 (m, 4H); 1.29 (t, 3H, J=7.2); LCMS calculated for C₁₈H₂₇N₃O₄: m/z=349; found: m/z=350 (M+H).

### Step 3: Ethyl 1-(6-(3-methoxy-2-(methoxymethyl)propyl)pyrazin-2-yl)piperidine-4-carboxylate

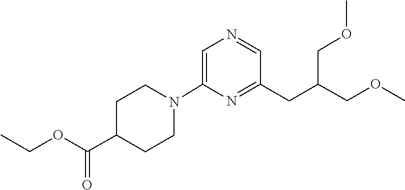 [SMILES: CCOC(=O)C1CCN(c2cncc(CC(COC)COC)n2)CC1]

To a solution of ethyl 1-(6-(3-methoxy-2-(methoxymethyl)prop-1-enyl)pyrazin-2-yl)piperidine-4-carboxylate (500 mg, 1.43 mmol, 1.0 eq) in MeOH (20 mL) at rt under an N₂ (g) atmosphere was added 10% Pd/C (500 mg). The resulting suspension was degassed under vacuum and purged with H₂ (g) three times. The resulting reaction mixture was stirred at rt under H₂(g) (15 psi) for 24 hr. The reaction mixture was then purged with N₂(g), filtered through a pad of celite to remove the catalyst, and the filtrate was concentrated under reduce pressure. The residue was purified by silica gel column chromatography (PE:EtOAc=10:1 to 4:1) to afford ethyl 1-(6-(3-methoxy-2-(methoxymethyl)propyl)pyrazin-2-yl)piperidine-4-carboxylate (256 mg, 25% yield) as a yellow oil. ¹H NMR (400 MHz, CDCl₃) δ 7.95 (s, 1H), 7.69 (s, 1H), 4.26 (m, 2H), 4.16 (q, J=7.2, 2H), 3.36 (d, J=5.6, 4H), 3.32 (s, 6H), 3.00 (m, 2H), 2.67 (d, J=7.2, 2H), 2.55 (m, 1H), 2.41 (m, 1H), 2.01 (m, 2H), 1.76 (m, 2H), 1.28 (t, J=7.2, 3H); LCMS calculated for C₁₈H₂₉N₃O₄: m/z=351; found: m/z=352 (M+H).

### Step 4: Ethyl 1-(5-iodo-6-(3-methoxy-2-(methoxymethyl)propyl)pyrazin-2-yl)piperidine-4-carboxylate

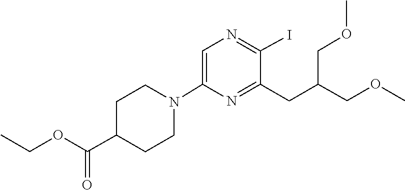 [SMILES: CCOC(=O)C1CCN(c2cnc(I)c(CC(COC)COC)n2)CC1]

To a solution of ethyl 1-(6-(3-methoxy-2-(methoxymethyl)propyl)pyrazin-2-yl)piperidine-4-carboxylate (670 mg, 1.91 mmol, 1.0 eq) in DMF (7 mL) at rt was added TFA (21 mg, 190 μmol, 0.1 eq) and NIS (471 mg, 2.10 mmol, 1.1 eq). The resulting reaction mixture was stirred at rt for 1 hr. The reaction mixture was quenched by saturated aqueous NaHCO₃ (15 mL), poured into H₂O (70 mL) and extracted with EtOAc (2×140 mL). The combined organic phase was washed with brine (50 mL), dried with anhydrous Na₂SO₄, filtered, and concentrated under vacuum. The residue was purified by silica gel column chromatography (PE:EtOAc=15:1 to 10:1) to afford ethyl 1-(5-iodo-6-(3-methoxy-2-(methoxymethyl)propyl)pyrazin-2-yl)piperidine-4-carboxylate (650 mg, 71% yield) as a yellow solid. ¹H NMR (400 MHz, CDCl₃) δ 7.75 (s, 1H), 4.17 (m, 4H), 3.42 (d, J=5.6, 4 H), 3.39 (s, 6H), 3.02 (m, 2H), 2.84 (d, J=7.2, 2H), 2.55 (m, 2H), 2.01 (m, 2H), 1.74 (m, 2H), 1.27 (t, J=7.2, 3H); LCMS calculated for C₁₈H₂₈IN₃O₄: m/z=477; found: m/z=478 (M+H).

### Step 5: Ethyl 1-(3-chloro-5-iodo-6-(3-methoxy-2-(methoxymethyl)propyl)pyrazin-2-yl)piperidine-4-carboxylate

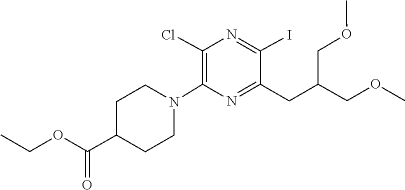 [SMILES: CCOC(=O)C1CCN(c2nc(CC(COC)COC)c(I)nc2Cl)CC1]

To a solution of ethyl 1-(5-iodo-6-(3-methoxy-2-(methoxymethyl)propyl)pyrazin-2-yl)piperidine-4-carboxylate (900 mg, 1.89 mmol, 1.0 eq) in DMF (10 mL) at rt was added NCS (276 mg, 2.07 mmol, 1.1 eq) and TFA (21 mg, 188 μmol, 0.1 eq). The resulting reaction mixture was heated at 100° C. and stirred for 2 hr. The reaction mixture was cooled to rt and quenched by addition of saturated aqueous NaHCO₃ (20 mL), poured into H₂O (100 mL) and extracted with EtOAc (2×200 mL). The combined organic phase was washed with brine (50 mL), dried with anhydrous Na₂SO₄, filtered, and concentrated under vacuum. The residue was purified by silica gel column chromatography (PE:EtOAc=15:1 to 10:1) to afford ethyl 1-(3-chloro-5-iodo-6-(3-methoxy-2-(methoxymethyl)propyl)pyrazin-2-yl)piperidine-4-carboxylate (810 mg, 84% yield) as a yellow solid. ¹H NMR (400 MHz, CDCl₃) δ 4.16 (q, J=7.2, 2H), 3.94 (m, 2H), 3.42 (d, J=5.6, 4H), 3.39 (s, 6H), 3.02 (m, 2H), 2.84 (d, J=7.2, 2H), 2.52 (m, 2H), 2.02 (m, 2H), 1.87 (m, 2H), 1.28 (t, J=7.2, H); LCMS calculated for C₁₈H₂₇ClIN₃O₄: m/z=511; found: m/z=512 (M+H).

### Step 6: Ethyl 1-(3-chloro-6-(3-methoxy-2-(methoxymethyl)propyl)pyrazin-2-yl)piperidine-4-carboxylate

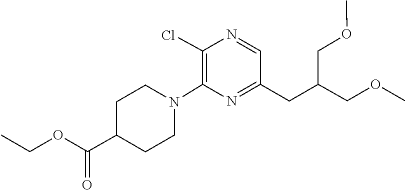 [SMILES: CCOC(=O)C1CCN(c2nc(CC(COC)COC)cnc2Cl)CC1]

To a solution of ethyl 1-(3-chloro-5-iodo-6-(3-methoxy-2-(methoxymethyl)propyl)pyrazin-2-yl)piperidine-4-carboxylate (800 mg, 1.56 mmol, 1.0 eq) in MeOH (50 mL) at rt under an N₂ (g) atmosphere was added 10% Pd/C (600 mg). The resulting suspension was degassed under vacuum and purged with H₂ (g) three times. The resulting reaction mixture was stirred at rt under H₂ (g) (30 psi) for 12 hr. The reaction mixture was then purged with N₂(g), filtered through a pad of celite to remove the catalyst, and the filtrate was concentrated under reduced pressure. The residue was purified by silica gel column chromatography (PE:EtOAc=15:1 to 10:1) to afford ethyl 1-(3-chloro-6-(3-methoxy-2-(methoxymethyl)propyl)pyrazin-2-yl)piperidine-4-carboxylate (500 mg, 82% yield) as a yellow oil. ¹H NMR (400 MHz, CDCl₃) δ 7.72 (s, 1H), 4.15 (q, J=7.2, 2H), 3.95 (m, 2H), 3.42 (d, J=5.6, 4H), 3.39 (s, 6H), 3.02 (m, 2H), 2.84 (d, J=7.2, 2H), 2.52 (m, 1 H), 2.25 (m, 1H), 2.03 (m, 2H), 1.87 (m, 2H), 1.27 (t, J=7.2, H); LCMS calculated for C₁₈H₂sClN₃O₄: m/z=385; found: m/z=386 (M+H).

### Step 7: 1-(3-(7-fluorobenzofuran-5-yl)-6-(3-methoxy-2-(methoxymethyl)propyl)pyrazin-2-yl)piperidine-4-carboxylic acid (Compound 53)

To a solution of ethyl 1-(3-chloro-6-(3-methoxy-2-(methoxymethyl)propyl)pyrazin-2-yl)piperidine-4-carboxylate (60 mg, 155 μmol, 1.0 eq) and 2-(7-fluorobenzofuran-5-yl)-4,4,5,5-tetramethyl-1,3,2-dioxaborolane (Intermediate 41) (44 mg, 171 μmol, 1.1 eq) in THF (1 mL) and H₂O (0.2 mL) at rt under an N₂ (g) atmosphere was added [di-tert-butyl(cyclopentyl)ferrocene]dichloropalladium(II) (10 mg, 15 μmol, 0.1 eq) and K₂CO₃ (42 mg, 310 μmol, 2.0 eq). The resulting reaction mixture was heated at 80° C. and stirred for 12 hr. The reaction mixture was cooled to rt and 1 mL of MeOH was added. Next, NaOH (4 M, 200 μL, 6.0 eq) was added and the resulting reaction mixture was heated at 50° C. and stirred for 3 hr. The reaction mixture was cooled to rt, filtered, and the filtrate concentrated under reduced pressure. The residue was purified by reverse-phase preparative HPLC (column: Phenomenex C18 80×40 mm×3 μm; mobile phase: [H₂O(10 mM NH₄HCO₃)-ACN]; B %: 15%-45%, 8 min run time) to afford 1-(3-(7-fluorobenzofuran-5-yl)-6-(3-methoxy-2-(methoxymethyl)propyl)pyrazin-2-yl)piperidine-4-carboxylic acid (45 mg, 42% yield, 98% purity by LC/MS) as a yellow solid. ¹H NMR (400 MHz, CDCl₃) δ 8.03 (s, 1 H), 7.95 (s, 1H), 7.68 (m, 2H), 6.86 (s, 1H), 3.63 (m, 2H), 3.42 (d, J=5.6, 4H) 3.35 (s, 6 H), 2.77 (m, 4H), 2.46 (m, 2H), 1.88 (m, 2H), 1.69 (m, 2H); LCMS calculated for C₂₄H₂₈FN₃O₅: m/z=457; found: m/z=458 (M+H).

### Example 29: 1-(2-(7-Fluorobenzofuran-5-yl)-5-(3-Methoxypropyl)Pyridin-3-yl)piperidine-4-carboxylic acid (Compound 168)

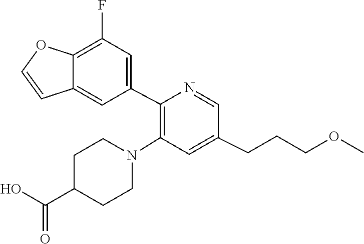 [SMILES: COCCCc1cnc(-c2cc(F)c3occc3c2)c(N2CCC(C(=O)O)CC2)c1]

### Step 1: 2-(7-fluorobenzofuran-5-yl)-3-(methoxymethoxy)-5-(3-methoxypropyl)pyridine

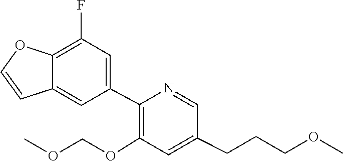 [SMILES: COCCCc1cnc(-c2cc(F)c3occc3c2)c(OCOC)c1]

To a solution of 2-(7-fluorobenzofuran-5-yl)-4,4,5,5-tetramethyl-1,3,2-dioxaborolane (Intermediate 41) (800 mg, 3.05 mmol, 1.5 eq) and 2-chloro-3-(methoxymethoxy)-5-(3-methoxypropyl)pyridine (Intermediate 86) (500 mg, 2.03 mmol, 1.0 eq) in THF (10 mL) and H₂O (2 mL) at rt under an N₂ (g) atmosphere was added K₂CO₃ (843 mg, 6.10 mmol, 3.0 eq) and Pd(dppf)Cl₂-CH₂Cl₂ (166 mg, 203 μmol, 0.1 eq). The resulting reaction mixture was heated to 80° C. and stirred for 16 hr. The reaction mixture was cooled to rt, diluted with H₂O (30 mL), and extracted with EtOAc (3×20 mL). The combined organic layers were washed with brine (2×20 mL), dried over Na₂SO₄, filtered, and concentrated under reduced pressure to give a residue. The residue was purified by silica gel column chromatography (PE:EtOAc=10:1 to 3:1) to afford 2-(7-fluorobenzofuran-5-yl)-3-(methoxymethoxy)-5-(3-methoxypropyl)pyridine (500 mg, 66% yield) as a yellow oil. LCMS calculated for C₁₉H₂₀FNO₄: m/z=345; found: m/z=346 (M+H).

### Step 2: 2-(7-fluorobenzofuran-5-yl)-5-(3-methoxypropyl)pyridin-3-ol

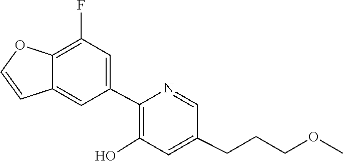 [SMILES: COCCCc1cnc(-c2cc(F)c3occc3c2)c(O)c1]

To a solution of 2-(7-fluorobenzofuran-5-yl)-3-(methoxymethoxy)-5-(3-methoxypropyl)pyridine (0.46 g, 1.33 mmol, 1.0 eq) in DCM (3 mL) at rt was added TFA (4.25 g, 37.28 mmol, 2.76 mL, 28 eq). The resulting reaction mixture was stirred at rt for 4 hr. The reaction mixture was adjusted pH=8 with saturated aqueous NaHCO₃ (20 mL) and extracted with EtOAc (3×20 mL). The combined organic layers were dried over Na₂SO₄, filtered, and concentrated under reduced pressure to give a residue. The residue was purified by silica gel column chromatography (PE:EtOAc=5:1 to 1:3) to afford 2-(7-fluorobenzofuran-5-yl)-5-(3-methoxypropyl)pyridin-3-ol (750 mg, 54% yield) as a yellow solid. ¹H NMR (400 MHz, CDCl₃) δ 8.02 (s, 1H), 7.79 (s, 1H), 7.67 (d, J=2.0, 1H), 7.51 (d, J=12.0, 1H), 6.95 (d, J=1.2, 1H), 6.81 (t, J=2.4, 1H), 3.38 (t, J=6.4, 2H), 3.33 (s, 3 H), 2.59 (t, J=7.6, 2H), 1.82 (m, 2H); LCMS calculated for C₁₇H₁₆FNO₃: m/z=301; found: m/z=302 (M+H).

### Step 3: 2-(7-fluorobenzofuran-5-yl)-5-(3-methoxypropyl)pyridin-3-yl trifluoromethanesulfonate

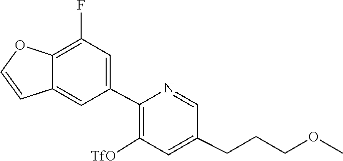 [SMILES: COCCCc1cnc(-c2cc(F)c3occc3c2)c(OS(=O)(=O)C(F)(F)F)c1]

To a solution of 2-(7-fluorobenzofuran-5-yl)-5-(3-methoxypropyl)pyridin-3-ol (250 mg, 829 umol, 1.0 eq) in DCM (5 mL) at 0° C. under an N₂ (g) atmosphere was added Et₃N (335 mg, 3.32 mmol, 461 μL, 4.0 eq) followed by trifluoromethanesulfonic anhydride (468 mg, 1.66 mmol, 273 μL, 2.0 eq). The resulting reaction mixture was warmed to rt and stirred for 1 hr. The reaction mixture was diluted with saturated aqueous Na₂CO₃ (20 mL) and then extracted with EtOAc (3×20 mL). The combined organic layers were washed with brine (2×20 mL), dried over Na₂SO₄, filtered, and concentrated under reduced pressure to give a residue. The residue was purified by silica gel column chromatography (PE:EtOAc=30:1 to 10:1) to afford 2-(7-fluorobenzofuran-5-yl)-5-(3-methoxypropyl)pyridin-3-yl trifluoromethanesulfonate (300 mg, 83% yield) as a yellow oil. LCMS calculated for C₁₈H₁₅F₄NO₅S; m/z=433; found: m/z=434 (M+H).

### Step 4: Ethyl 1-(2-(7-fluorobenzofuran-5-yl)-5-(3-methoxypropyl)pyridin-3-yl)piperidine-4-carboxylate

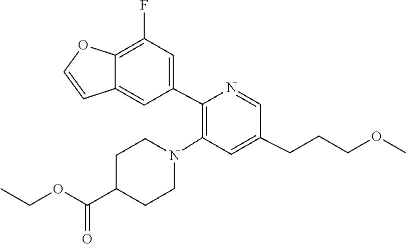 [SMILES: CCOC(=O)C1CCN(c2cc(CCCOC)cnc2-c2cc(F)c3occc3c2)CC1]

A mixture of 2-(7-fluorobenzofuran-5-yl)-5-(3-methoxypropyl)-3-pyridyl trifluoromethane sulfonate (100 mg, 230 μmol, 1.0 eq), ethyl piperidine-4-carboxylate (181 mg, 1.15 mmol, 177 μL, 5.0 eq) in NMP (2.5 mL) was heated to 220° C. and stirred for 3 hr under microwave irradiation. The reaction mixture was cooled to rt, diluted with H20 (10 mL) and extracted with EtOAc (3×10 mL). The combined organic layers were washed with brine (2×10 mL), dried over Na₂SO₄, filtered, and concentrated under reduced pressure to give a residue. This identical procedure was conducted twice more, and the crude products were combined and purified by preparative TLC (PE:EtOAc=1:1) to afford ethyl 1-(2-(7-fluorobenzofuran-5-yl)-5-(3-methoxypropyl)pyridin-3-yl)piperidine-4-carboxylate (25 mg, 7% yield) as a yellow oil. LCMS calculated for C₂₅H₂₉FN₂O₄: m/z=440; found: m/z=441 (M+H).

### Step 5: 1-(2-(7-fluorobenzofuran-5-yl)-5-(3-methoxypropyl)pyridin-3-yl)piperidine-4-carboxylic acid (Compound 168)

A solution of ethyl 1-(2-(7-fluorobenzofuran-5-yl)-5-(3-methoxypropyl)-3-pyridyl)piperidine-4-carboxylate (20 mg, 45 μmol, 1.0 eq) in MeOH (0.5 mL) at rt was treated with NaOH (4 M, 191 μL, 16.9 eq). The resulting reaction mixture was heated to 50° C. and stirred for 1 hr. The reaction mixture was cooled to rt, filtered, and the filtrate concentrated under reduced pressure. The residue was purified by reverse-phase preparative HPLC (column: Waters Xbridge Prep OBD C18 150×40 mm×10 μm; mobile phase: [H₂O(10 mM NH₄HCO₃)-ACN]; B %: 20%-50%, 8 min run time) to afford 1-(2-(7-fluorobenzofuran-5-yl)-5-(3-methoxypropyl)pyridin-3-yl)piperidine-4-carboxylic acid (7 mg, 36% yield, 97% purity by LC/MS) as a white solid. ¹H NMR (400 MHz, CDCl₃) δ 8.21 (d, J=1.6, 1H), 7.96 (d, J=1.2, 1H), 7.75 (d, J=11.2, 1H), 7.68 (d, J=2.0, 1H), 7.20 (d, J=1.6, 1H), 6.85 (t, J=2.4, 1H), 3.44 (t, J=6.4, 2H), 3.38 (s, 3H), 3.16 (m, 2H), 2.74 (m, 2H), 2.61 (m, 2H), 2.40 (m, 1H), 1.91 (m, 4H), 1.76 (m, 2H); LCMS calculated for C₂₃H₂₅FN₂O₄: m/z=412; found: m/z=413 (M+H).

### Example 30: 1-(3-(7-Fluorobenzofuran-5-yl)-6-(3-Methoxypropyl)Pyrazin-2-yl)piperidine-4-carboxylic acid (Compound 173)

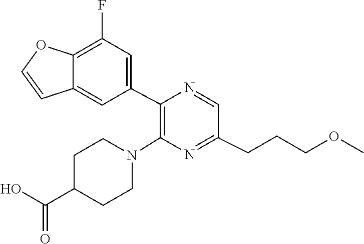 [SMILES: COCCCc1cnc(-c2cc(F)c3occc3c2)c(N2CCC(C(=O)O)CC2)n1]

### Step 1: Ethyl 1-(3-(7-fluorobenzofuran-5-yl)-6-(3-methoxypropyl)pyrazin-2-yl)piperidine-4-carboxylate

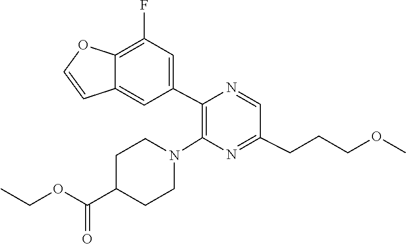 [SMILES: CCOC(=O)C1CCN(c2nc(CCCOC)cnc2-c2cc(F)c3occc3c2)CC1]

To a solution of ethyl 1-(3-chloro-6-(3-methoxypropyl)pyrazin-2-yl)piperidine-4-carboxylate (Intermediate 93) (140 mg, 400 μmol, 1.0 eq) in THF (3.0 mL) and H₂O (0.3 mL) at rt was added 2-(7-fluorobenzofuran-5-yl)-4,4,5,5-tetramethyl-1,3,2-dioxaborolane (Intermediate 41) (130 mg, 440 μmol, 1.2 eq) and K₂CO₃ (115 mg, 720 μmol, 2.0 eq). The mixture was degassed under vacuum and purged with N₂ (g) three times, then [di-tert-butyl(cyclopentyl)ferrocene]dichloropalladium(II) (24 mg, 38 μmol, 0.1 eq) was added. The resulting reaction mixture was heated to 80° C. and stirred for 12 hr under an N₂ (g) atmosphere. The mixture was cooled to rt, diluted with H₂O (20 mL), and extracted with EtOAc (3×20 mL). The combined organic layers were washed with brine (30 mL), dried over Na₂SO₄, filtered, and concentrated under reduced pressure to give a residue. The residue was purified by silica gel column chromatography (PE:EtOAc=1:0 to 0:1) to afford ethyl 1-(3-(7-fluorobenzofuran-5-yl)-6-(3-methoxypropyl)pyrazin-2-yl)piperidine-4-carboxylate (170 mg, 89% yield) as a brown oil. ¹H NMR (400 MHz, CDCl₃) δ 8.03 (s, 1H), 7.96 (d, J=1.2, 1 H), 7.72-7.66 (m, 2H), 6.87 (d, J=2.4, 1H), 4.17-4.11 (q, J=7.2, 2H), 3.64 (m, 2H), 3.48 (t, J=6.4, 2H), 3.38 (s, 3H), 2.77 (m, 4H), 2.43 (m, 1H), 2.05 (m, 2H), 1.87 (m, 2H), 1.72 (m, 2H), 1.28 (t, J=7.2, 3H); LCMS calculated for C₂₄H₂₈FN₃O₄: m/z=441; found: m/z=442 (M+H).

### Step 2: 1-(3-(7-fluorobenzofuran-5-yl)-6-(3-methoxypropyl)pyrazin-2-yl)piperidine-4-carboxylic acid (Compound 173)

To a solution of ethyl 1-(3-(7-fluorobenzofuran-5-yl)-6-(3-methoxypropyl)pyrazin-2-yl)piperidine-4-carboxylate (150 mg, 340 μmol, 1.0 eq) in MeOH (2.0 mL) was added NaOH (2 M, 0.3 mL, 1.8 eq). The resulting reaction mixture was heated to 50° C. and stirred for 1 hr. The reaction mixture was cooled to rt, filtered, and the filtrate concentrated under reduced pressure. The residue was purified by reverse-phase preparative HPLC (column: Phenomenex C18 75×30 mm×3 μm; mobile phase: [H₂O(10 mM NH₄HCO₃)-ACN]; B %: 15%-45%, 8 min run time) to afford 1-(3-(7-fluorobenzofuran-5-yl)-6-(3-methoxypropyl)pyrazin-2-yl)piperidine-4-carboxylic acid (78 mg, 55% yield, 99% purity by LC/MS) as a white solid. ¹H NMR (400 MHz, CD₃OD) δ7.95 (m, 2H), 7.88 (d, J=2.0, 1H), 7.59 (dd, J=1.6, 12.0, 1H), 7.01 (t, J=2.4, 1H), 3.64 (m, 2H), 3.48 (t, J=6.4, 2H), 3.35 (s, 3H), 2.83-2.71 (m, 4H), 2.32 (m, 1H), 2.06 (m, 2H), 1.81 (m, 2H), 1.69 (m, 2H); LCMS calculated for C₂₂H₂₄FN₃O₄: m/z=413; found: m/z=414 (M+H).

### Example 31: 1-(3-(benzo[d][1,3]dioxol-5-yl)-6-(3-Methoxypropyl)Pyrazin-2-yl)piperidine-4-carboxylic acid (Compound 189)

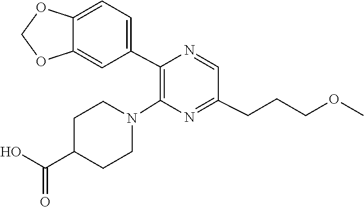 [SMILES: COCCCc1cnc(-c2ccc3c(c2)OCO3)c(N2CCC(C(=O)O)CC2)n1]

### Step 1: Ethyl 1-(3-(benzo[d][1,3]dioxol-5-yl)-6-(3-methoxypropyl)pyrazin-2-yl)piperidine-4-carboxylate

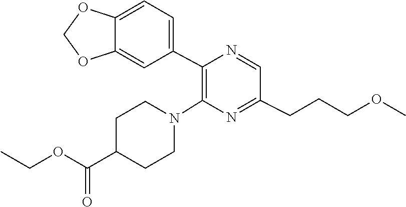 [SMILES: CCOC(=O)C1CCN(c2nc(CCCOC)cnc2-c2ccc3c(c2)OCO3)CC1]

To a solution of ethyl 1-(3-chloro-6-(3-methoxypropyl)pyrazin-2-yl)piperidine-4-carboxylate (Intermediate 93) (500 mg, 1.46 mmol, 1.0 eq) and benzo[d][1,3]dioxol-5-ylboronic acid (485 mg, 2.93 mmol, 2.0 eq) in THF (10 mL) and H₂O (2 mL) at rt under an N₂ (g) atmosphere was added [di-tert-butyl(cyclopentyl)ferrocene]dichloropalladium(II) (95 mg, 146 μmol, 0.1 eq) and K₂CO₃ (606 mg, 4.39 mmol, 3.0 eq). The resulting reaction mixture was heated to 80° C. and stirred for 3 hr. The reaction mixture was then cooled to rt and combined with another batch (100 mg). The reaction mixture was poured into H₂O (50 mL) and extracted with EtOAc (3×50 mL). The combined organic phase was washed with brine (30 mL), dried with anhydrous Na₂SO₄, filtered, and concentrated under vacuum. The residue was purified by silica gel column chromatography (PE:EtOAc=10:1 to 5:1) to afford ethyl 1-(3-(benzo[d][1,3]dioxol-5-yl)-6-(3-methoxypropyl)pyrazin-2-yl)piperidine-4-carboxylate (500 mg, 66% yield) as a yellow solid. ¹H NMR (400 MHz, CDCl₃) δ 7.90 (s, 1 H), 7.37 (d, J=8.0, 1H), 7.33 (d, J=1.6, 1H), 6.80 (d, J=8.0, 1H), 5.93 (s, 2H), 4.08 (q, J=7.2, 2H), 3.60 (m, 2H), 3.38 (t, J=6.0, 2H), 3.29 (s, 3H), 2.70 (m, 4H), 2.31 (m, 1H), 1.96 (m, 2H), 1.81 (m, 2H), 1.67 (m, 2H), 1.19 (t, J=7.2, 3H).

### Step 2: 1-(3-(benzo[d][1,3]dioxol-5-yl)-6-(3-methoxypropyl)pyrazin-2-yl)piperidine-4-carboxylic acid (Compound 189)

To a solution of ethyl 1-(3-(benzo[d][1,3]dioxol-5-yl)-6-(3-methoxypropyl)pyrazin-2-yl)piperidine-4-carboxylate (250 mg, 585 μmol, 1.0 eq) in MeOH (3 mL) at rt was added NaOH (4 M, 880 μL, 6.0 eq). The resulting reaction mixture was heated at 60° C. and stirred for 1 hr. The reaction mixture was then cooled to rt, and the pH was adjusted to 5-6 by addition of aqueous citric acid. The reaction mixture was diluted with H₂O (50 mL) and extracted with EtOAc (2×50 mL). The combined organic phase was washed with brine (30 mL), dried with anhydrous Na₂SO₄, filtered, and concentrated under vacuum. The residue was purified by reverse-phase preparative HPLC (column: Phenomenex C18 80×40 mm×3 μm; mobile phase: [H₂O(10 mM NH₄HCO₃)-ACN]; B %: 10%-80%, 8 min run time) to afford 1-(3-(benzo[d][1,3]dioxol-5-yl)-6-(3-methoxypropyl)pyrazin-2-yl)piperidine-4-carboxylic acid (66.2 mg, 27% yield, 100% purity by LC/MS) as a yellow solid. ¹H NMR (400 MHz, CD₃OD) δ7.90 (s, 1H), 7.37 (dd, J=2.0, 8.0. 1 H), 7.31 (d, J=2.0, 1H), 6.93 (d, J=8.0. 1 H), 6.02 (s, 2H), 3.67 (m, 2H), 3.48 (t, J=6.4, 2H), 3.36 (s, 3 H), 2.77 (m, 4H), 2.36 (m, 1H), 2.04 (m, 2H), 1.86 (m, 2H), 1.69 (m, 2H); LCMS calculated for C₂₁H₂₅N₃O₅: m/z=399; found: m/z=400 (M+H).

The following compounds were prepared similarly to Examples 1-31 with the appropriate reagents and intermediates. Some examples may require additional functional group transformations or deprotection to arrive at the final compounds.

| Cmpd # | Characterization Data |
|---|---|
| 18 | LCMS: 363 (M + H) |
| 19 | LCMS: 368 (M + H) |
| 20 | LCMS: 357 (M + H) |
| 21 | LCMS: 351 (M + H) |
| 22 | LCMS: 359 (M + H) |
| 23 | LCMS: 364 (M + H) |
| 24 | LCMS: 361 (M + H) |
| 25 | LCMS: 405 (M + H) |
| 26 | LCMS: 324 (M + H) |
| 27 | LCMS: 373 (M + H) |
| 28 | LCMS: 359 (M + H) |
| 29 | LCMS: 367 (M + H) |
| 30 | LCMS: 460 (M + H) |
| 31 | LCMS: 424 (M + H) |
| 32 | LCMS: 426 (M + H) |
| 33 | LCMS: 470 (M + H) |
| 34 | LCMS: 468 (M + H) |
| 35 | LCMS: 375 (M + H) |
| 36 | LCMS: 382 (M + H) |
| 37 | LCMS: 433 (M + H) |
| 38 | LCMS: 450 (M + H) |
| 39 | LCMS: 401 (M + H) |
| 40 | LCMS: 402 (M + H) |
| 41 | LCMS: 425 (M + H) |
| 42 | LCMS: 468 (M + H) |
| 43 | LCMS: 376 (M + H) |
| 44 | LCMS: 377 (M + H) |
| 45 | LCMS: 384 (M + H) |
| 46 | LCMS: 402 (M + H) |
| 47 | LCMS: 396 (M + H) |
| 48 | LCMS: 399 (M + H) |
| 49 | LCMS: 386 (M + H) |
| 50 | LCMS: 443 (M + H) |
| 51 | LCMS: 399 (M + H) |
| 52 | LCMS: 399 (M + H) |
| 54 | LCMS: 461 (M + H) |
| 55 | LCMS: 388 (M + H) |
| 56 | LCMS: 434 (M + H) |
| 57 | LCMS: 454 (M + H) |
| 58 | LCMS: 515 (M + H) |
| 59 | LCMS: 471 (M + H) |
| 60 | LCMS: 438 (M + H) |
| 62 | LCMS: 445 (M + H) |
| 63 | LCMS: 469 (M + H) |
| 64 | LCMS: 437 (M + H) |
| 65 | LCMS: 478 (M + H) |
| 66 | LCMS: 532 (M + H) |
| 67 | LCMS: 548 (M + H) |
| 68 | LCMS: 494 (M + H) |
| 69 | LCMS: 462 (M + H) |
| 70 | LCMS: 532 (M + H) |
| 71 | LCMS: 478 (M + H) |
| 72 | LCMS: 439 (M + H) |
| 73 | LCMS: 410 (M + H) |
| 74 | LCMS: 427 (M + H) |
| 75 | LCMS: 452 (M + H) |
| 76 | LCMS: 470 (M + H) |
| 77 | LCMS: 466 (M + H) |
| 78 | LCMS: 484 (M + H) |
| 79 | LCMS: 411 (M + H) |
| 80 | LCMS: 561 (M + H) |
| 81 | LCMS: 510 (M + H) |
| 82 | LCMS: 494 (M + H) |
| 83 | LCMS: 514 (M + H) |
| 84 | LCMS: 448 (M + H) |
| 85 | LCMS: 465 (M + H) |
| 86 | LCMS: 454 (M + H) |
| 87 | LCMS: 423 (M + H) |
| 88 | LCMS: 438 (M + H) |
| 89 | LCMS: 438 (M + H) |
| 90 | LCMS: 424 (M + H) |
| 91 | LCMS: 454 (M + H) |
| 92 | LCMS: 468 (M + H) |
| 93 | LCMS: 463 (M + H) |
| 94 | LCMS: 450 (M + H) |
| 95 | LCMS: 478 (M + H) |
| 96 | LCMS: 468 (M + H) |
| 97 | LCMS: 455 (M + H) |
| 98 | LCMS: 473 (M + H) |
| 99 | LCMS: 440 (M + H) |
| 100 | LCMS: 440 (M + H) |
| 101 | LCMS: 446 (M + H) |
| 102 | LCMS: 446 (M + H); |
| | ¹H NMR (400 MHz, CD₃OD) δ 7.92 (s, 1 H), 7.90 (s, 1H), 7.83 (d, J = 2.4, 1 |
| | H), 7.59 (m, 2 H), 6.94 (d, J = 1.6, 1 H), 3.19 (m, 2 H), 3.10 (m, 2 H), 3.01 (m, 2 |
| | H), 2.72 (m, 3 H), 2.61 (m, 2 H), 2.13 (m, 2 H), 1.62 (m, 2 H) |
| 103 | LCMS: 453 (M + H) |
| 104 | LCMS: 468 (M + H) |
| 105 | LCMS: 381 (M + H) |
| 106 | LCMS: 485 (M + H) |
| 107 | LCMS: 436 (M + H) |
| 109 | LCMS: 435 (M + H) |
| 110 | LCMS: 452 (M + H) |
| 111 | LCMS: 465 (M + H) |
| 112 | LCMS: 450 (M + H) |
| 113 | LCMS: 450 (M + H) |
| 114 | LCMS: 451 (M + H) |
| 115 | LCMS: 465 (M + H) |
| 116 | LCMS: 435 (M + H) |
| 117 | LCMS: 513 (M + H) |
| 118 | LCMS: 459 (M + H) |
| 119 | LCMS: 422 (M + H) |
| 120 | LCMS: 424 (M + H) |
| 121 | LCMS: 420 (M + H) |
| 122 | LCMS: 438 (M + H) |
| 123 | LCMS: 434 (M + H) |
| 124 | LCMS: 470 (M + H) |
| 125 | LCMS: 504 (M + H) |
| 126 | LCMS: 474 (M + H) |
| 127 | LCMS: 492 (M + H) |
| 128 | LCMS: 429 (M + H) |
| 129 | LCMS: 452 (M + H) |
| 131 | LCMS: 450 (M + H) |
| 132 | LCMS: 452 (M + H) |
| 133 | LCMS: 483 (M + H) |
| 134 | LCMS: 416 (M + H) |
| 135 | LCMS: 418 (M + H) |
| 136 | LCMS: 420 (M + H) |
| 137 | LCMS: 450 (M + H) |
| 138 | LCMS: 410 (M + H) |
| 139 | LCMS: 413 (M + H) |
| 140 | LCMS: 414 (M + H) |
| 141 | LCMS: 445 (M + H) |
| 142 | LCMS: 416 (M + H) |
| 143 | LCMS: 418 (M + H) |
| 144 | LCMS: 420 (M + H) |
| 145 | LCMS: 450 (M + H) |
| 146 | LCMS: 478 (M + H) |
| 147 | LCMS: 412 (M + H) |
| 148 | LCMS: 450 (M + H) |
| 149 | LCMS: 416 (M + H) |
| 150 | LCMS: 447 (M + H) |
| 151 | LCMS: 450 (M + H) |
| 152 | LCMS: 450 (M + H) |
| 153 | LCMS: 479 (M + H) |
| 154 | LCMS: 500 (M + H) |
| 155 | LCMS: 475 (M + H) |
| 156 | LCMS: 471 (M + H) |
| 157 | LCMS: 478 (M + H) |
| 158 | LCMS: 462 (M + H) |
| 159 | LCMS: 460 (M + H) |
| 160 | LCMS: 474 (M + H) |
| 161 | LCMS: 464 (M + H) |
| 162 | LCMS: 448 (M + H) |
| 163 | LCMS: 446 (M + H) |
| 164 | LCMS: 460 (M + H) |
| 165 | LCMS: 468 (M + H) |
| 166 | LCMS: 454 (M + H) |
| 167 | LCMS: 484 (M + H) |
| 169 | LCMS: 417 (M + H) |
| 171 | LCMS: 416 (M + H) |
| 174 | LCMS: 450 (M + H) |
| 175 | LCMS: 368 (M + H) |
| 176 | LCMS: 372 (M + H) |
| 177 | LCMS: 464 (M + H) |
| 178 | LCMS: 468 (M + H) |
| 179 | LCMS: 441 (M + H) |
| 181 | LCMS: 408 (M + H) |
| 182 | LCMS: 394 (M + H) |
| 183 | LCMS: 428 (M + H); |
| | ¹H NMR (400 MHz, CD₃OD) δ 7.93 (s, 1 H), 7.90 (t, J = 2.4, 2 H), 7.55 (dd, J = |
| | 1.2, 12.0, 1 H), 7.03 (t, J = 2.4, 1 H), 4.07 (m, 1 H), 3.52 (m, 1 H), 3.48 (t, J = |
| | 6.4, 2 H), 3.36 (s, 3 H), 3.11 (m, 1 H), 2.81 (t, J = 7.6, 2 H), 2.56 (m, 1 H) 2.06 |
| | (m, 2 H), 1.84-1.60 (m, 4 H), 1.04 (d, J = 6.8, 3 H) |
| 184 | LCMS: 428 (M + H) |
| 185 | LCMS: 430 (M + H) |
| 186 | LCMS: 426 (M + H) |
| 187 | LCMS: 450 (M + H) |
| 188 | LCMS: 434 (M + H) |
| 192 | LCMS: 430 (M + H) |
| 193 | LCMS: 396 (M + H) |
| 194 | LCMS: 396 (M + H) |
| 195 | LCMS: 397 (M + H) |
| 196 | LCMS: 397 (M + H) |
| 197 | LCMS: 410 (M + H) |
| 198 | LCMS: 410 (M + H) |
| 199 | LCMS: 407 (M + H) |
| 200 | LCMS: 407 (M + H) |
| 201 | LCMS: 428 (M + H) |
| 202 | LCMS: 417 (M + H) |
| 203 | LCMS: 419 (M + H) |
| 204 | LCMS: 415 (M + H) |
| 205 | LCMS: 396 (M + H) |
| 206 | LCMS: 400 (M + H) |
| 207 | LCMS: 396 (M + H) |
| 208 | LCMS: 396 (M + H) |
| 209 | LCMS: 402 (M + H) |
| 210 | LCMS: 414 (M + H) |
| 211 | LCMS: 397 (M + H) |
| 212 | LCMS: 397 (M + H) |
| 213 | LCMS: 410 (M + H) |
| 214 | LCMS: 410 (M + H) |
| 215 | LCMS: 410 (M + H) |
| 216 | LCMS: 414 (M + H) |
| 217 | LCMS: 411 (M + H) |
| 218 | LCMS: 411 (M + H) |
| 219 | LCMS: 411 (M + H) |
| 220 | LCMS: 426 (M + H) |
| 221 | LCMS: 438 (M + H) |
| 222 | LCMS: 456 (M + H) |
| 223 | LCMS: 432 (M + H) |
| 224 | LCMS: 431 (M + H) |
| 225 | LCMS: 431 (M + H) |
| 226 | LCMS: 413 (M + H) |
| 227 | LCMS: 428 (M + H) |
| 228 | LCMS: 443 (M + H) |
| 229 | LCMS: 428 (M + H); |
| | ¹H NMR (400 MHz, CD₃OD) δ 8.19 (s, 1 H), 8.14 (s, 1 H), 7.90 (s, 1 H), 7.82 |
| | (dd, J = 2.4, 12.3, 1 H), 7.03 (t, J = 2.4, 1 H), 3.57-3.48 (m, 3 H), 3.36 (s, 3 H), |
| | 3.13 (m, 1 H), 2.87 (t, J = 7.2, 2 H), 2.62 (m, 1 H), 2.44 (m, 1 H), 2.08 (m, 3 H), |
| | 1.70 (m, 2 H), 1.51 (m, 1 H), 1.20 (d, J = 6.0, 3 H) |
| 230 | LCMS: 411 (M + H) |
| 232 | LCMS: 422 (M + H) |
| 233 | LCMS: 447 (M + H) |
| 234 | LCMS: 461 (M + H) |
| 235 | LCMS: 396 (M + H); |
| | ¹H NMR (400 MHz, DMSO-d₆) δ 12.21 (s, br, 1 H), 8.14 (d, J = 1.4, 1 H), 8.07 |
| | (s, 1 H), 8.03 (d, J = 4.0, 1 H), 7.82 (dd, J = 1.6, 7.0, 1 H), 7.64 (d, J = 8.6, 1 H), |
| | 7.02 (d, J = 1.9, 1 H), 3.67 (m, 1 H), 3.41 (t, J = 6.4, 2 H), 3.25 (s, 3 H), 3.20 |
| | (m, 1 H), 2.96 (m, 1 H), 2.71 (m, 2 H), 2.66 (m, 1 H), 2.54 (m, 1 H), 1.95 (m, 2 |
| | H), 1.84 (m, 1 H), 1.52 (m, 2 H) |
| 236 | LCMS: 396 (M + H); |
| | ¹H NMR (400 MHz, DMSO-d₆) δ 12.21 (s, br, 1 H), 8.14 (d, J = 1.4, 1 H), 8.07 |
| | (s, 1 H), 8.03 (d, J = 4.0, 1 H), 7.82 (dd, J = 1.6, 7.0, 1 H), 7.64 (d, J = 8.6, 1 H), |
| | 7.02 (d, J = 1.9, 1 H), 3.67 (m, 1 H), 3.41 (t, J = 6.4, 2 H), 3.25 (s, 3 H), 3.20 |
| | (m, 1 H), 2.96 (m, 1 H), 2.71 (m, 2 H), 2.66 (m, 1 H), 2.54 (m, 1 H), 1.95 (m, 2 |
| | H), 1.84 (m, 1 H), 1.52 (m, 2 H) |
| 237 | LCMS: 464 (M + H) |
| 238 | LCMS: 480 (M + H) |
| 239 | LCMS: 410 (M + H) |
| 240 | LCMS: 446 (M + H) |
| 241 | LCMS: 397 (M + H) |
| 242 | LCMS: 410 (M + H) |
| 243 | LCMS: 428 (M + H) |
| 244 | LCMS: 432 (M + H) |
| 246 | LCMS: 431 (M + H) |

The following compounds can also generally be made using the methods described above. It is expected that these compounds when made will have activity similar to those that have been made in the examples disclosed herein.

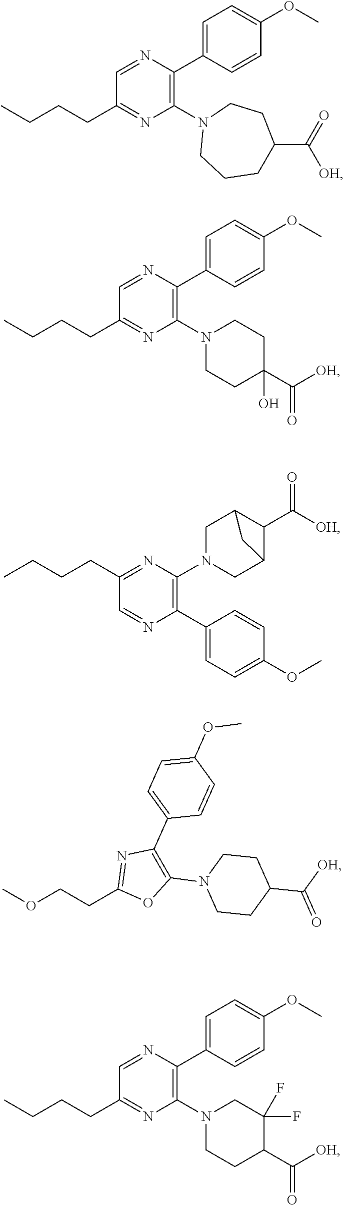

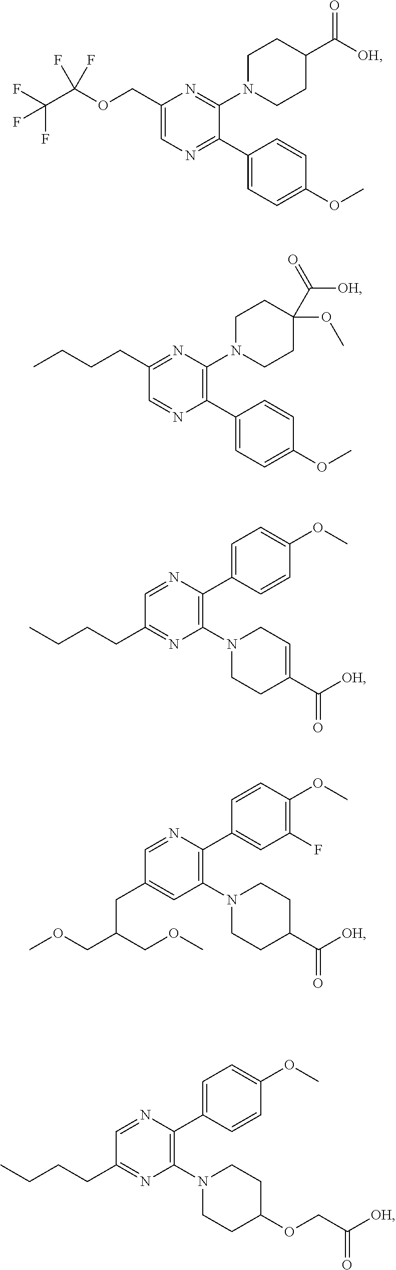

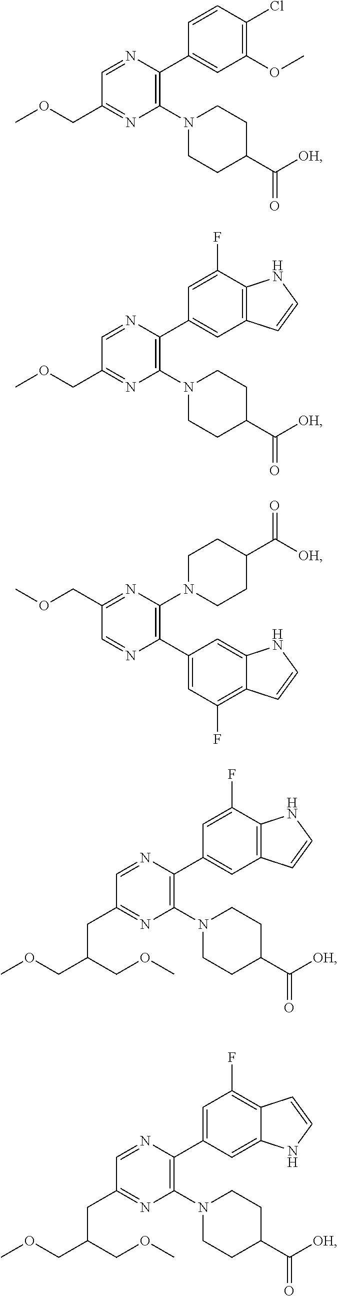

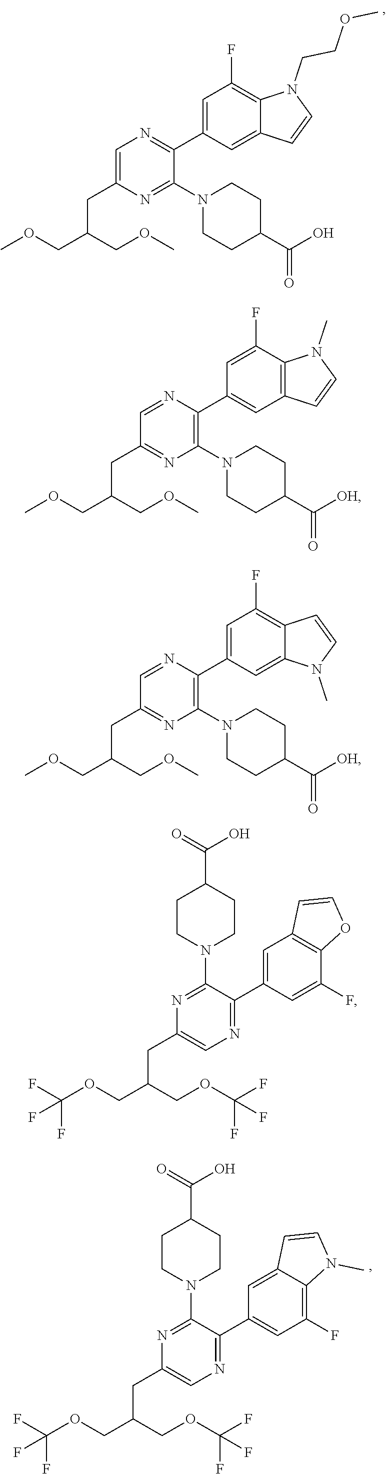

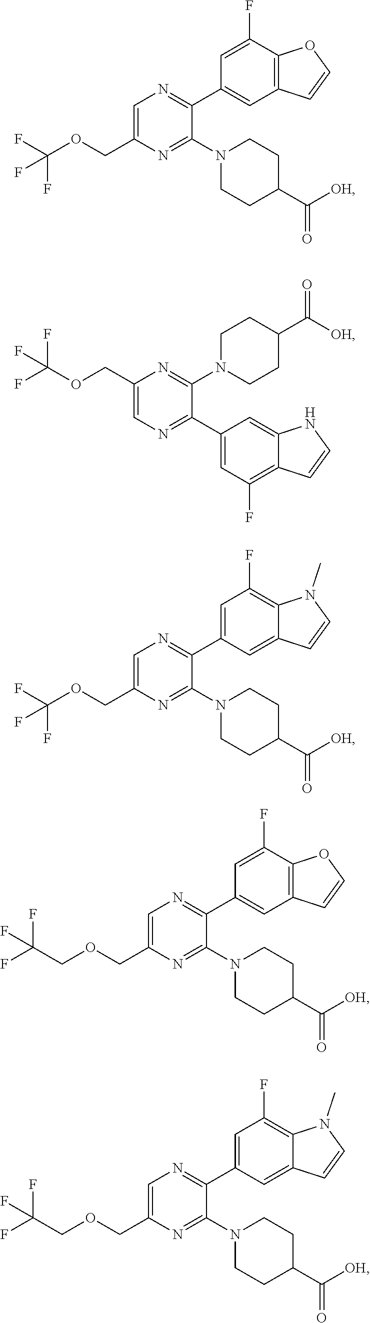

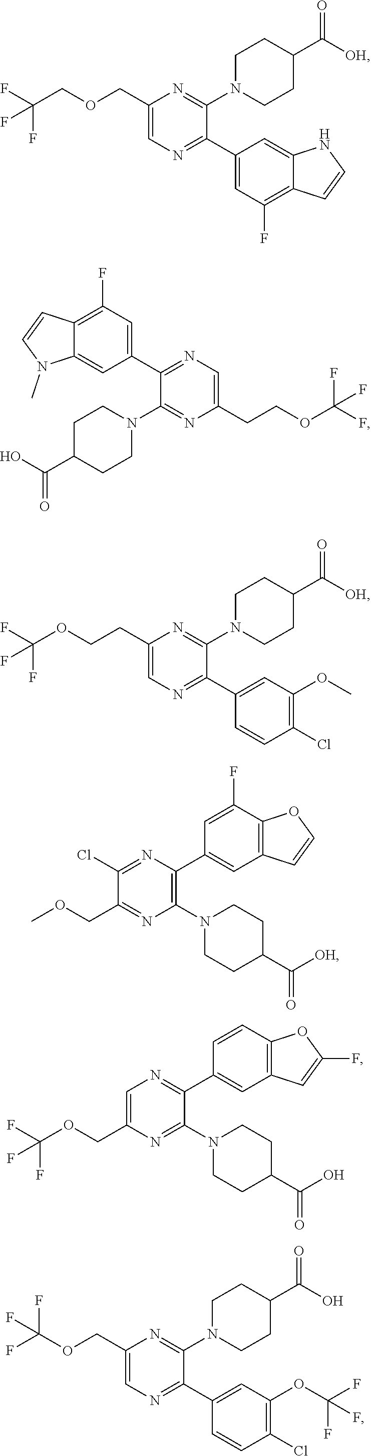

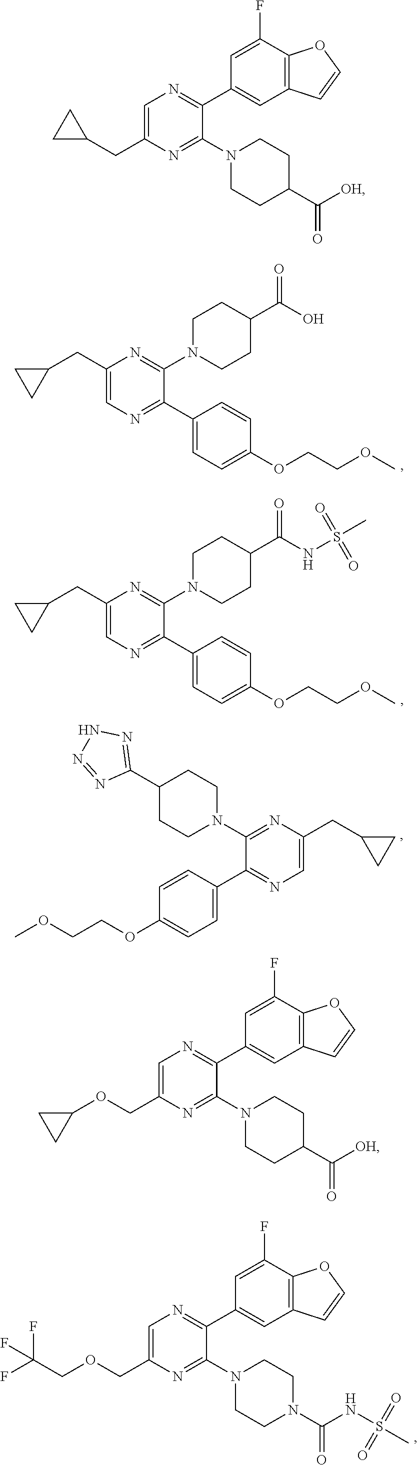

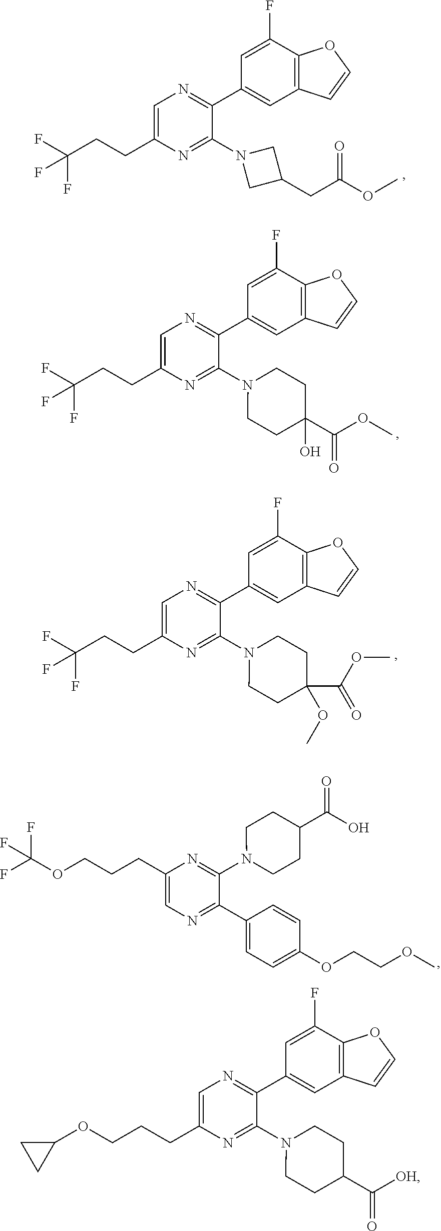

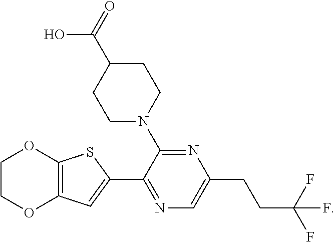 [SMILES: O=C(O)C1CCN(c2nc(CCC(F)(F)F)cnc2-c2cc3c(s2)OCCO3)CC1]

### II. Biologic and Pharmacologic Assays and Methods

The activity of the compounds as GLUT9 inhibitors is illustrated in the following assay.

### Example A-1: GLUT9 Assay

Uric acid stocks (9 mM, Millipore-Sigma, U0881) were generated in 125 mM choline chloride (Millipore-Sigma, C7017) and the concentration was determined at 293 nm using a molar extinction coefficient of 9600 M- ¹ cm- ¹) after collecting the absorbance spectra from 220-350 nm. Allantoin stocks (12 mM Toronto Research Chemicals, A540500) were generated in LCMS grade water (Thermo Scientific, 51140). The concentration of allantoin was determined by qNMR (Michigan State University). HEK293T cells were purchased from ATCC (HEK 293T/17 SF). Hanks buffered saline salts (HBSS) was purchased from Millipore-Sigma (H6648-1L).

SLC2A9 variant 2 was assayed in sodium gluconate buffer was composed of 125 mM sodium gluconate (TCI Chemicals, G0041-500G), 4.8 mM potassium gluconate (TCI Chemicals, G0040-500G), 1.2 mM calcium gluconate (TCI Chemicals, G0037-500G), 1.2 mM potassium phosphate (pH 7.4, Millipore-Sigma, 17835), 1.2 mM MgSO4, Millipore-Sigma, M2773-500G), 25 mM HEPES pH 7.3, (Millipore-Sigma, 54457). The final pH of the buffer was 7.4. Urate 1 was assayed in the same buffer save the pH was made to be 5.5 using 25 mM MES (pH 5.5) buffer vs 25 mM HEPES (pH 7.3) buffer.

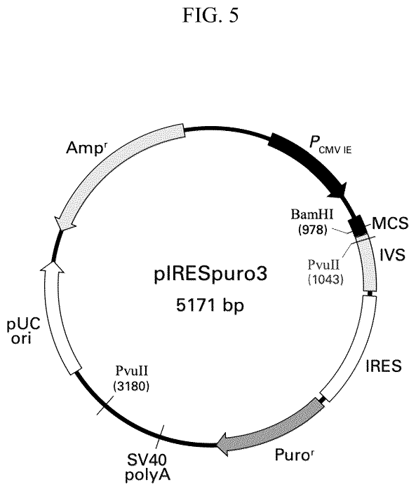

The pIRESpuro3 vector (FIG. 5) was purchased from Takara Bio (Cat #3P 631619). The vector was transformed into E. coli (DH5a Invitrogen). Fermentation of the said stocks were used to generate midi-prep DNA (Qiagen) for the generation of the pIRESpuro-SLC2A9 variant 2 vector.

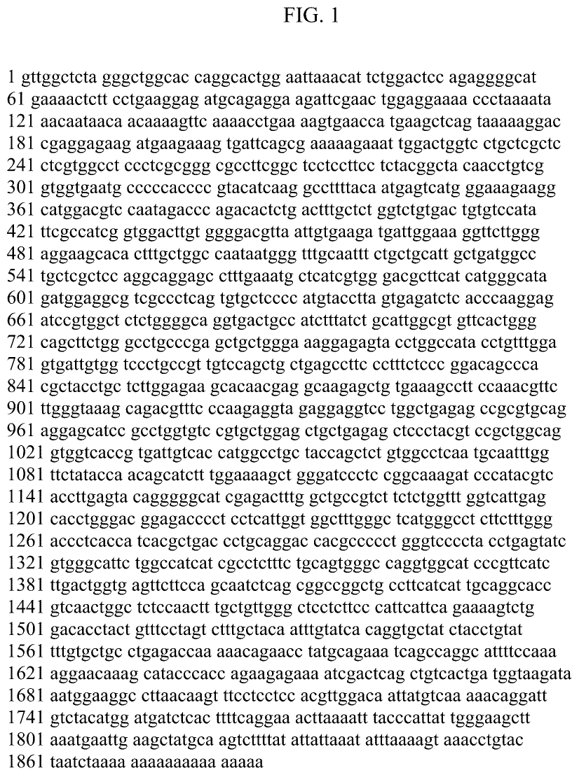

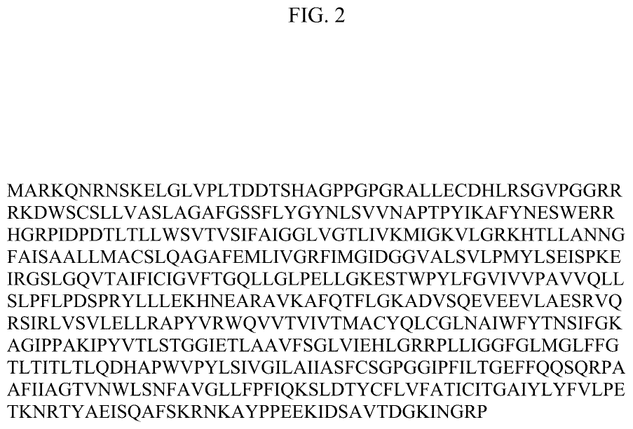

Human untagged clone for (untagged)-human solute carrier family 2 member 9 (SLC2A9), transcript variant (SLC2A9/NM_001001290, FIG. 1 and FIG. 2; SEQ ID NOS: 3 and 4) was purchased from Origene (Cat #SC319239). SLC2A9 variant 2 (GLUT9B) gene was amplified by PCR using the following primer to allow for the introduction of EcoR1 and Not1 sites using the primers below.

5′EcoRI.Kozack.Glut9 Variant 2 (SEQ ID NO: 1):
5′-ACGGAATTCGCCACCATGAAGCTCAGTAAAAAGGACCGAGGAG-3′
3′-Glut9 Variant 2.Not1 (SEQ ID NO: 2):
5′-CGTTGCGGCCGCATGAGCACTTTTAAAGTTCTGCTATGTGGC-3′.

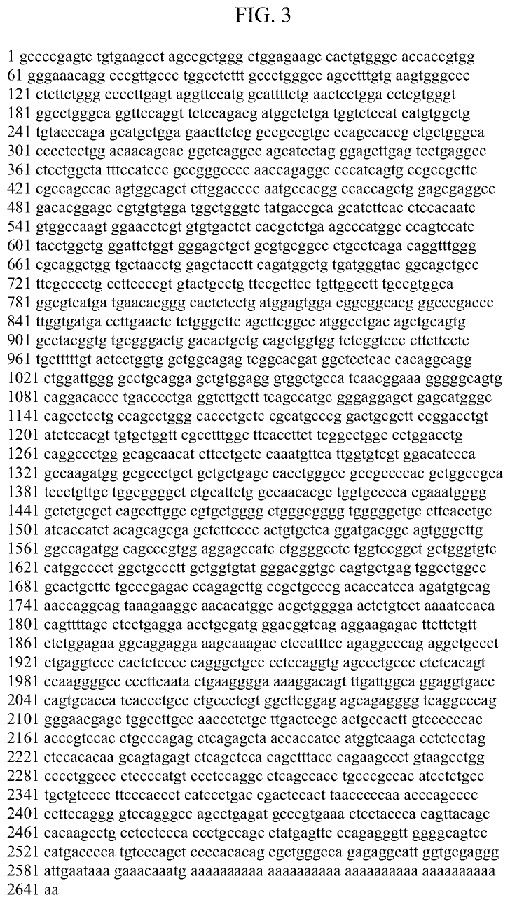

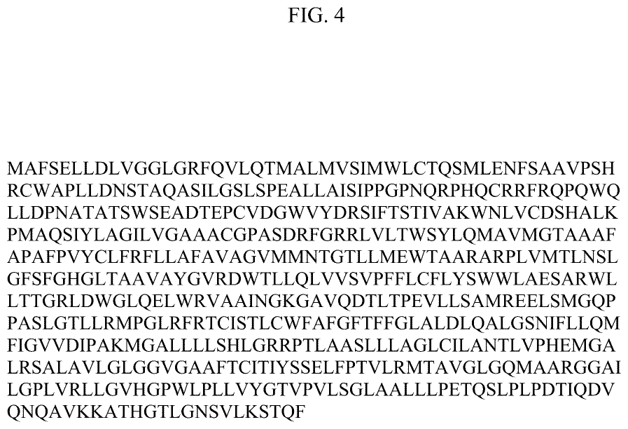

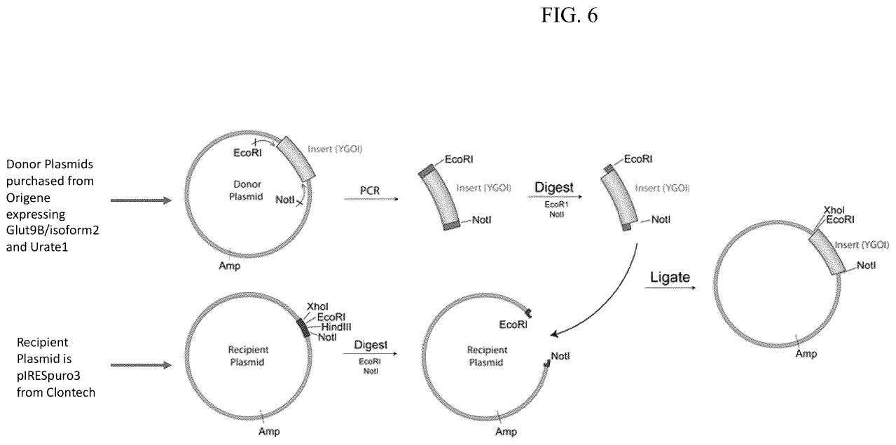

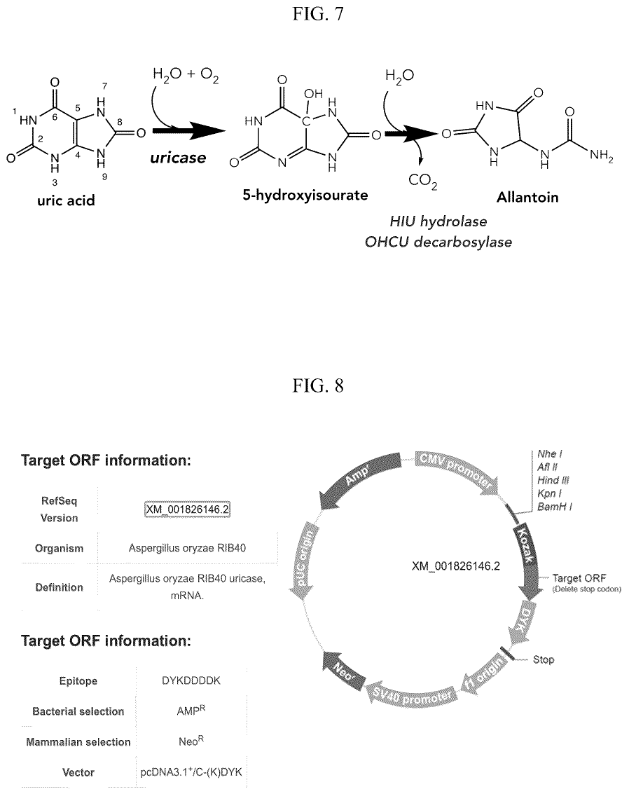

The vector and Glut9 variant 2 DNA fragments were digested with EcoR1 and Not1, purified and ligated (FIG. 6). The vectors were transformed into E. coli strain DH5a. Subsequent midi-prep DNA samples (Qiagen) were generated. Positive clones were tested for expression by qPCR. Positive clones were sequenced. A similar procedure was used to create a vector for expression of the Aspergillus oryzae uricase open reading frame (URAT1; OAg09316/XM_001826146.3, FIG. 3 and FIG. 4; SEQ ID NOS: 5 and 6; and FIG. 8) from GenScript.

HEK293T clonal stable cell lines containing the SLC2A9 variant 2 construct in the bicistronic pIRESpuro vector were generated using Viafect™ (Promega E4981) as per the manufactures instructions and puromycin selection (5 μg×mL⁻¹).

### Functional Assay

Uric acid uptake was measured in HEK293T cells stably expressing H. sapiens SLC2A9 variant 2 (GLUT9B) and transiently expressing A. oryzae uricase. Thus, decreased allantoin formation was used as a proxy of GLUT9B inhibition. Briefly, clonal cells were plated (53,000 cells/cm²) in DMEM with 10% FBS. Twenty-four hours later, A. oryzae uricase was transfected, and assay conducted 3 days later across a 12-point curve in buffer (in mM: 125 sodium gluconate, 4.8 potassium gluconate, 1.2 calcium gluconate, 1.2 potassium phosphate, 1.2 magnesium sulfate4, 25 HEPES; pH 7.4) with uric acid at the GLUT9B Km (0.3 mM) for 15 min or for 60 min, at 37° C. Following wash (Hanks, 37° C.), plates were sealed, incubated (5 min, RT) and cells freeze-thawed prior to lysing in water and shaking (30 min RT). Lysates were filtered through PVDF and subjected to MSMS using a 10 mM ammonium acetate-based mobile phase on a porous graphitic carbon column. Allantoin was detected in positive ion mode. Desired analyte concentrations were quantitated by integrating the area under the curve followed by a log-logistic regression model and data compared to a standard curve prepared in a matching HEK293T biological matrix.

Mass spectrometry measurement of allantoin was completed using an Agilent RapidFire 300 coupled to a Sciex API 4000 mass spectrometer. The stationary phase was an Agilent cartridge D (hypercarb). Mobile phase A was 10 mM ammonium acetate. Mobile phase B was 5 mM ammonium acetate, 25% acetonitrile, 25% acetone. Allantoin was detected in positive ion mode with the following compound ID settings: Q1 159, Q3 115.8, DP 57, CE 12.5 Dwell 100 ms. The source settings were CAD 10, CUR 20, GS1 50, GS250 IS/NC: 5000, TEM 650, EP 10, CXP 10 where Q1 and Q3 resolution were set to low.

Data was quantitated using Area under the curve integration was performed with the RapidFire™ Integrator (Agilent Technologies) for the desired analytes. Quantitation was completed using in a using a log-logistic regression model using allantoin in matching HEK293T biological matrix.

The activity for representative examples of the invention is shown in the following table, wherein A: IC₅₀<500 nM; B: 500 nM<IC₅₀<2 lM; D: IC₅₀>2 M. For compounds tested in both the 15 minute and 60 minute assays, both values are reported.

TABLE 2
Biological Activity against GLUT9B

| Cmpd # | 15 min IC50^{a} | 60 min IC50^{b} |
|---|---|---|
| 1 | C | |
| 2 | C | |
| 3 | A | A |
| 4 | A | |
| 5 | C | |
| 6 | C | |
| 7 | B | |
| 8 | C | |
| 9 | A | |
| 10 | A | |
| 11 | A | |
| 12 | C | |
| 13 | A | |
| 14 | A | |
| 15 | A | |
| 16 | A | |
| 17 | B | |
| 18 | C | |
| 19 | B | |
| 20 | C | |
| 21 | C | |
| 22 | C | |
| 23 | C | |
| 24 | C | |
| 25 | C | |
| 26 | C | |
| 27 | B | |
| 28 | B | |
| 29 | A | |
| 30 | A | |
| 31 | A | |
| 32 | C | |
| 33 | B | |
| 34 | B | |
| 35 | B | |
| 36 | B | |
| 37 | A | |
| 38 | B | |
| 39 | A | |
| 40 | A | |
| 41 | B | |
| 42 | C | |
| 43 | C | |
| 44 | C | |
| 45 | A | |
| 46 | A | |
| 47 | B | |
| 48 | A | |
| 49 | B | |
| 50 | C | |
| 51 | C | |
| 52 | A | |
| 53 | A | |
| 54 | C | |
| 55 | B | |
| 56 | C | |
| 57 | C | |
| 58 | C | |
| 59 | A | |
| 60 | B | |
| 61 | A | |
| 62 | C | |
| 63 | B | |
| 64 | C | |
| 65 | A | |
| 66 | B | |
| 67 | C | |
| 68 | C | |
| 69 | A | |
| 70 | B | |
| 71 | A | |
| 72 | C | |
| 73 | A | |
| 74 | B | |
| 75 | C | |
| 76 | B | |
| 77 | C | |
| 78 | C | |
| 79 | C | |
| 80 | B | |
| 81 | B | |
| 82 | A | |
| 83 | B | |
| 84 | C | |
| 85 | C | |
| 86 | B | |
| 87 | C | |
| 88 | B | |
| 89 | B | |
| 90 | B | |
| 91 | A | |
| 92 | B | |
| 93 | B | |
| 94 | B | |
| 95 | A | |
| 96 | | A |
| 97 | C | |
| 98 | C | |
| 99 | C | |
| 100 | A | |
| 101 | C | |
| 102 | B | |
| 103 | B | |
| 104 | B | |
| 105 | C | |
| 106 | C | |
| 107 | A | |
| 108 | A | A |
| 109 | B | |
| 110 | B | |
| 111 | A | |
| 112 | B | |
| 113 | B | |
| 114 | B | |
| 115 | C | |
| 116 | A | |
| 117 | B | |
| 118 | B | |
| 119 | A | |
| 120 | A | |
| 121 | A | |
| 122 | B | |
| 123 | A | |
| 124 | A | |
| 125 | B | |
| 126 | A | |
| 127 | B | |
| 128 | A | |
| 129 | A | |
| 130 | A | |
| 131 | A | |
| 132 | A | |
| 133 | A | |
| 134 | A | |
| 135 | B | |
| 136 | A | |
| 137 | B | |
| 138 | A | A |
| 139 | A | |
| 140 | A | |
| 141 | A | |
| 142 | A | A |
| 143 | A | |
| 144 | A | |
| 145 | A | |
| 146 | A | |
| 147 | A | |
| 148 | C | |
| 149 | A | |
| 150 | B | C |
| 151 | A | |
| 152 | A | |
| 153 | A | |
| 154 | B | |
| 155 | C | |
| 156 | C | |
| 157 | A | |
| 158 | B | |
| 159 | B | |
| 160 | B | |
| 161 | B | |
| 162 | B | |
| 163 | B | |
| 164 | B | |
| 165 | A | |
| 166 | B | |
| 167 | C | |
| 168 | A | A |
| 169 | A | |
| 170 | B | C |
| 171 | C | |
| 172 | A | A |
| 173 | A | A |
| 174 | B | |
| 175 | C | C |
| 176 | C | |
| 177 | A | |
| 178 | | B |
| 179 | | B |
| 180 | | A |
| 181 | | A |
| 182 | | C |
| 183 | | A |
| 184 | | A |
| 185 | | B |
| 186 | | A |
| 187 | | A |
| 188 | | A |
| 189 | | A |
| 190 | | A |
| 191 | | A |
| 192 | | A |
| 193 | | C |
| 194 | | C |
| 195 | | C |
| 196 | | C |
| 197 | | C |
| 198 | | C |
| 199 | | C |
| 200 | | C |
| 201 | | C |
| 202 | | C |
| 203 | | C |
| 204 | | C |
| 205 | | C |
| 206 | | C |
| 207 | | C |
| 208 | | C |
| 209 | | C |
| 210 | | B |
| 211 | | C |
| 212 | | C |
| 213 | | C |
| 214 | | C |
| 215 | | C |
| 216 | | C |
| 217 | | C |
| 218 | | C |
| 219 | | C |
| 220 | | C |
| 221 | | C |
| 222 | | C |
| 223 | | A |
| 224 | | A |
| 225 | | A |
| 226 | | A |
| 227 | | A |
| 228 | | A |
| 229 | | A |
| 230 | | A |
| 231 | | C |
| 232 | | A |
| 233 | | C |
| 234 | | C |
| 235 | | C |
| 236 | | B |
| 237 | C | |
| 238 | | B |
| 239 | | A |
| 240 | | A |
| 241 | | C |
| 242 | | B |
| 243 | | A |
| 244 | | A |
| 245 | | A |
| 246 | | A |
| 247 | | B |
| 248 | | C |
| 249 | | C |
| 250 | | C |
| 251 | | C |
| 252 | | C |
| 253 | | A |
| 254 | | A |
| 255 | | B |

^{a}15 minute assay;
^{b}60 minute assay

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of Formula (I′): 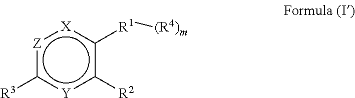
or a pharmaceutically acceptable salt thereof, wherein:
m is an integer from 0 to 4;
X is selected from CH and N;
Y is selected from CH and N;
Z is CR⁷;
R¹ is selected from aryl and heteroaryl;
R² is a mono- or bi-cyclic heterocycloalkyl or a mono- or bi-cyclic heteroaryl, either of which may be optionally substituted with one or more R;
R³ is selected from:
alkyl optionally substituted with SO₂-alkyl,
alkoxyalkyl,
alkoxyalkoxyalkyl,
haloalkyl,
haloalkoxyalkyl,
cycloalkylalkyl, and
cycloalkoxyalkyl;
each R⁴ is independently selected from:
alkoxy optionally substituted with R,
alkylamino optionally substituted with R⁵,
dialkylamino optionally substituted with R⁵,
SO₂R^{8a},
alkylthio,
haloalkoxy,
cycloalkoxy,
cycloalkylalkoxy,
halo,
alkyl, and
haloalkyl;
or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl;
each R⁵ is independently selected from hydroxyl, alkoxy, amino, alkylamino, dialkylamino, haloalkoxy, and alkoxyalkoxy;
each R⁶ is independently selected from hydroxyl, hydroxyalkoxy, carboxyl, methylcarboxyl, carboxylalkyl, carboxylalkoxy, halo, alkyl, alkoxy, SO₂R^{8b}, C(═O)NHSO₂R^{8b}, heterocycloalkyl, cyano, and tetrazolyl;
R⁷ is selected from hydrogen and halo; and
R^{8a} and R^{8b} are each independently selected from alkyl, amino, alkylamino, dialkylamino;
wherein X and Y are not both CH.

2. (canceled)

3. (canceled)

4. The compound of claim 1, or a pharmaceutically acceptable salt thereof, wherein:
X is N; Y is N; and Z is CH.

5. (canceled)

6. (canceled)

7. The compound of claim 1, or a pharmaceutically acceptable salt thereof, wherein:
R¹ is bicyclic heteroaryl.

8. The compound of claim 1, or a pharmaceutically acceptable salt thereof, wherein:
each R⁴ is independently selected from alkoxy optionally substituted with R, SO₂R^{8a}, haloalkoxy, cycloalkoxy, cycloalkylalkoxy, halo, alkyl, and haloalkyl;
or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl;
each R⁵ is independently selected from hydroxyl, alkoxy, amino, alkylamino, dialkylamino, and haloalkoxy; and
m is an integer from 0 to 3.

9. The compound of claim 1, or a pharmaceutically acceptable salt thereof, wherein:
each R⁴ is independently selected from alkoxy, haloalkoxy, and halo;
or two R⁴ substituents on adjacent carbon atoms of R¹ are taken together to form a fused heterocycloalkyl or a fused heteroaryl; and
m is an integer from 0 to 3.

10. (canceled)

11. (canceled)

12. (canceled)

13. The compound of claim 1, or a pharmaceutically acceptable salt thereof, wherein:
R³ is alkoxyalkyl.

14. The compound of claim 1, or a pharmaceutically acceptable salt thereof, wherein:
R² is a mono- or bi-cyclic heterocycloalkyl, which is substituted with one or more R;
each R⁶ is independently selected from hydroxyl, carboxyl, carboxylalkyl, carboxylalkoxy, SO₂R^{8b}, C(═O)NHSO₂R^{8b}, heterocycloalkyl, and tetrazolyl; and
R^{8b} is selected from alkyl, amino, alkylamino, dialkylamino.

15. (canceled)

16. The compound of claim 1, or a pharmaceutically acceptable salt thereof, wherein:
R² is a piperidinyl, which is substituted with one R; and
R⁶ is selected from carboxyl, carboxylalkyl, and heterocycloalkyl.

17. The compound of claim 1, wherein the compound is a compound of Formula (IV): 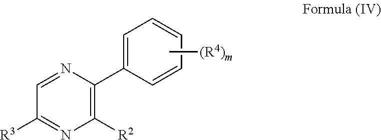
or a pharmaceutically acceptable salt thereof, wherein:
m is an integer from 0 to 3;
R² is a mono- or bi-cyclic heterocycloalkyl, which is substituted with one or more R⁶;
R³ is selected from alkyl optionally substituted with SO₂-alkyl, alkoxyalkyl, alkoxyalkoxyalkyl, haloalkyl, haloalkoxyalkyl, cycloalkylalkyl, and cycloalkoxyalkyl;
each R⁴ is independently selected from alkoxy optionally substituted with R, SO₂Ra, haloalkoxy, cycloalkoxy, cycloalkylalkoxy, halo, alkyl, and haloalkyl;
or two R⁴ substituents on adjacent carbon atoms of R¹ are taken together to form a fused heterocycloalkyl or a fused heteroaryl, either of which may be optionally substituted with one or more alkyl, halo, oxo, alkoxy, or alkoxyalkyl;
each R⁵ is independently selected from hydroxyl, alkoxy, amino, alkylamino, dialkylamino, and haloalkoxy;
each R⁶ is independently selected from hydroxyl, carboxyl, carboxylalkyl, carboxylalkoxy, SO₂R^{8b}, C(═O)NHSO₂R^{8b}, heterocycloalkyl, and tetrazolyl; and
R^{8b} is selected from alkyl, amino, alkylamino, dialkylamino.

18. (canceled)

19. (canceled)

20. The compound of claim 17, or a pharmaceutically acceptable salt thereof, wherein:
R³ is selected from alkyl, alkoxyalkyl, haloalkyl, and haloalkoxyalkyl;
each R⁴ is independently selected from alkoxy, haloalkoxy, and halo;
or two R⁴ substituents on adjacent carbon atoms of R¹ are taken together to form a fused heterocycloalkyl or a fused heteroaryl;
R⁶ is selected from carboxyl, carboxylalkyl, C(═O)NHSO₂R^{8b}, heterocycloalkyl, and tetrazolyl; and
R^{8b} is selected from alkyl, amino, alkylamino, dialkylamino.

21. (canceled)

22. (canceled)

23. (canceled)

24. (canceled)

25. The compound of claim 1, wherein the compound is a compound of Formula (VI): 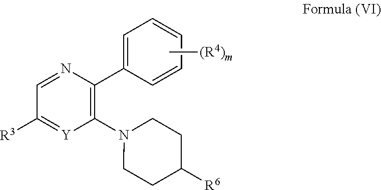
or a pharmaceutically acceptable salt thereof, wherein:
m is an integer from 0 to 4;
Y is N or CH;
R³ is selected from:
C₁-C₆alkyl optionally substituted with SO₂-(C₁-C₆alkyl),
(C₁-C₆alkoxy)-C₁-C₆alkyl,
(C₁-C₆alkoxy)-(C₁-C₆alkoxy)-C₁-C₆alkyl,
C₁-C₆haloalkyl,
(C₁-C₆haloalkoxy)-C₁-C₆alkyl,
(C₃-C₆cycloalkyl)-C₁-C₆alkyl, and
(C₃-C₆cycloalkoxy)-C₁-C₆alkyl;
each R⁴ is independently selected from:
C₁-C₆alkoxy optionally substituted with R⁵,
C₁-C₆alkylamino optionally substituted with R⁵,
di-(C₁-C₆alkyl)-amino optionally substituted with R,
SO₂R^{8a},
C₁-C₆alkylthio,
C₁-C₆haloalkoxy,
C₃-C₆cycloalkoxy,
(C₃-C₆cycloalkyl)-C₁-C₆alkoxy,
halo,
C₁-C₆alkyl, and
C₁-C₆haloalkyl;
or two R⁴ substituents on adjacent carbon atoms of R¹ are taken together to form a fused heterocycloalkyl or a fused heteroaryl, either of which may be optionally substituted with one or more C₁-C₆alkyl, halo, oxo, C₁-C₆alkoxy, or (C₁-C₆alkoxy)-C₁-C₆alkyl;
each R⁵ is independently selected from hydroxyl, C₁-C₆alkoxy, amino, C₁-C₆alkylamino, di-C₁-C₆alkylamino, C₁-C₆haloalkoxy, and (C₁-C₆alkoxy)-C₁-C₆alkoxy;
R⁶ is selected from —C(═O)OH, —CH₂C(═O)OH, —C(═O)O—C₁-C₆alkyl, —C(═O)NHSO₂R^{8b}, heterocycloalkyl, and tetrazolyl;
R^{8a} and R^{8b} are each independently selected from C₁-C₆alkyl, amino, C₁-C₆alkylamino, di-(C₁-C₆alkyl)-amino.

26. (canceled)

27. (canceled)

28. (canceled)

29. The compound of claim 25, wherein the compound is a compound of Formula (VI-3): 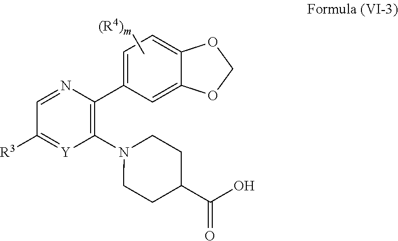
or a pharmaceutically acceptable salt thereof, wherein:
m is an integer from 0 to 2;
Y is N or CH;
R³ is selected from: C₁-C₆alkyl, (C₁-C₄alkoxy)-C₁-C₆alkyl, C₁-C₆haloalkyl, and (C₁-C₄haloalkoxy)-C₁-C₆alkyl; and
each R⁴ is independently selected from: C₁-C₆alkoxy, C₁-C₄haloalkoxy, halo, C₁-C₄alkyl, and C₁-C₄haloalkyl.

30. The compound of claim 25, wherein the compound is a compound of Formula (VI-4): 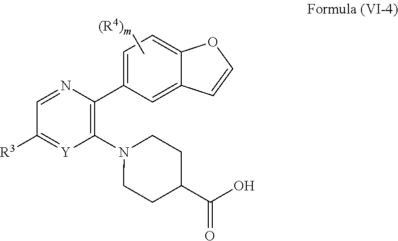
or a pharmaceutically acceptable salt thereof, wherein:
m is an integer from 0 to 2;
Y is N or CH;
R³ is selected from: C₁-C₆alkyl, (C₁-C₄alkoxy)-C₁-C₆alkyl, C₁-C₆haloalkyl, and (C₁-C₄haloalkoxy)-C₁-C₆alkyl; and
each R⁴ is independently selected from: C₁-C₆alkoxy, C₁-C₄haloalkoxy, halo, C₁-C₄alkyl, and C₁-C₄haloalkyl.

31. (canceled)

32. The compound of claim 25, or a pharmaceutically acceptable salt thereof, wherein:
R³ is selected from: C₃-C₆alkyl, (C₁-C₄alkoxy)-C₃-C₆alkyl, C₃-C₆haloalkyl, and (C₁-C₄haloalkoxy)-C₃-C₆alkyl.

33. (canceled)

34. The compound of claim 25, or a pharmaceutically acceptable salt thereof, wherein:
Y is N.

35. A compound selected from: 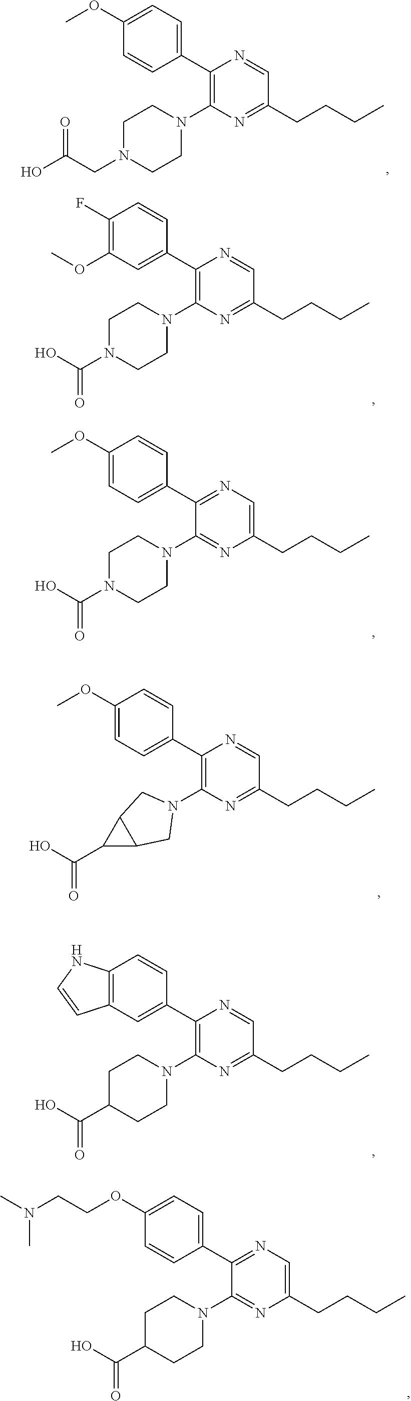 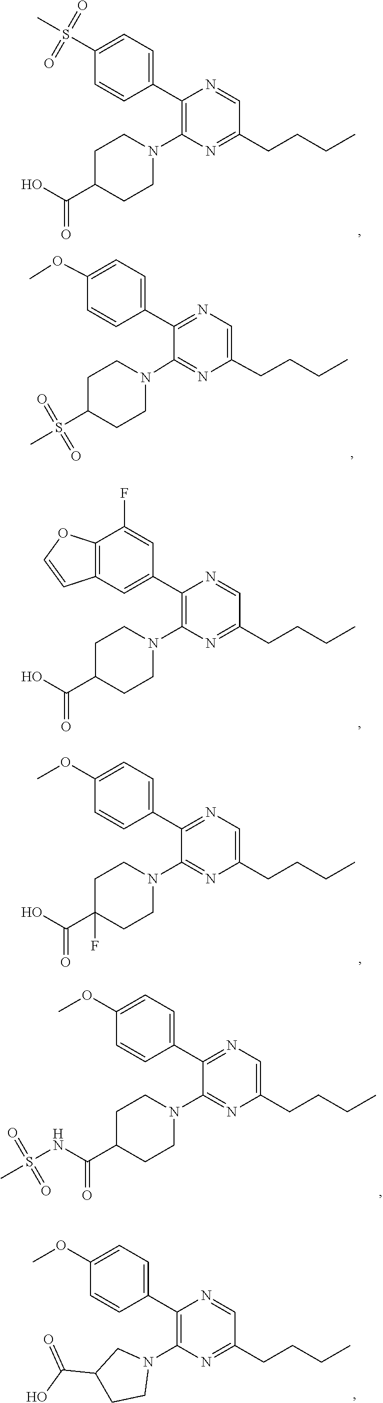 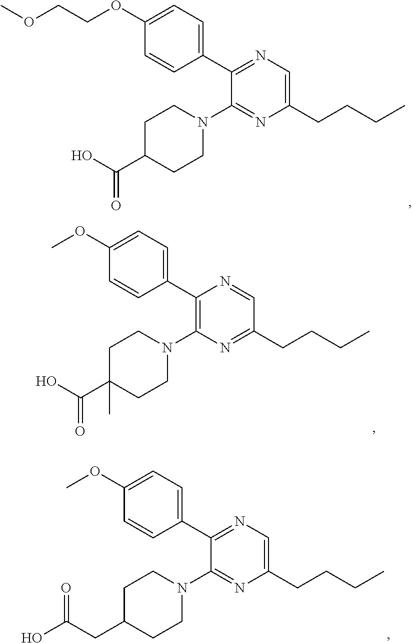 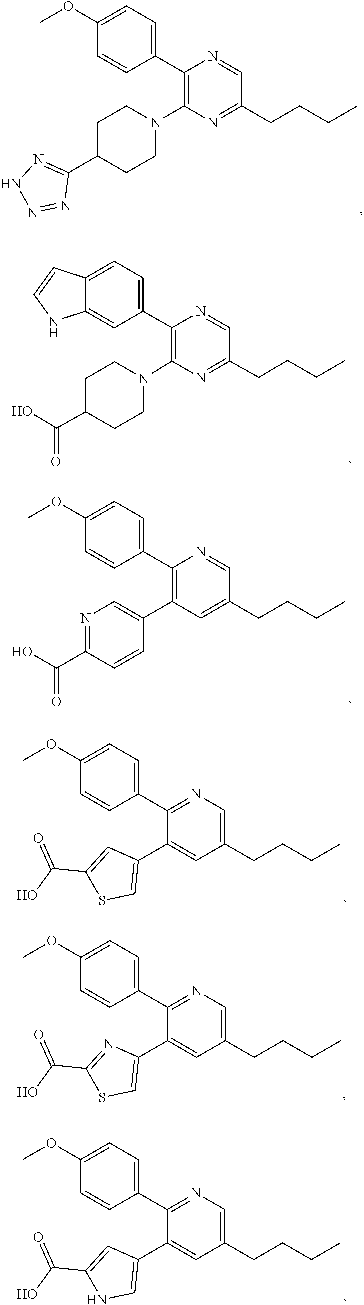 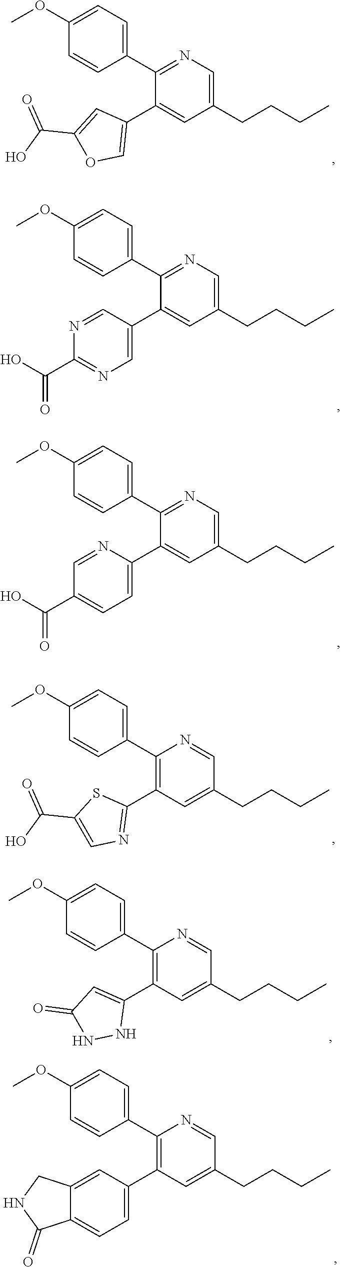 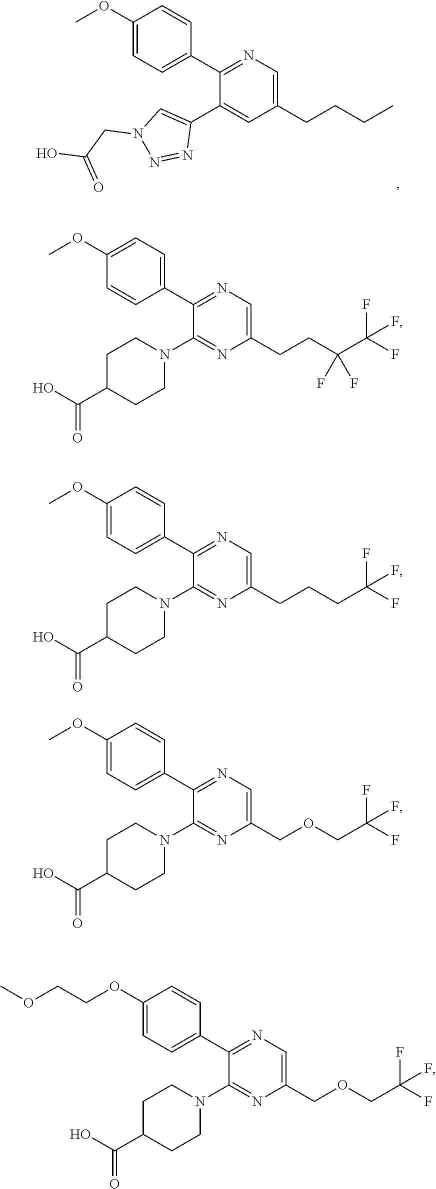 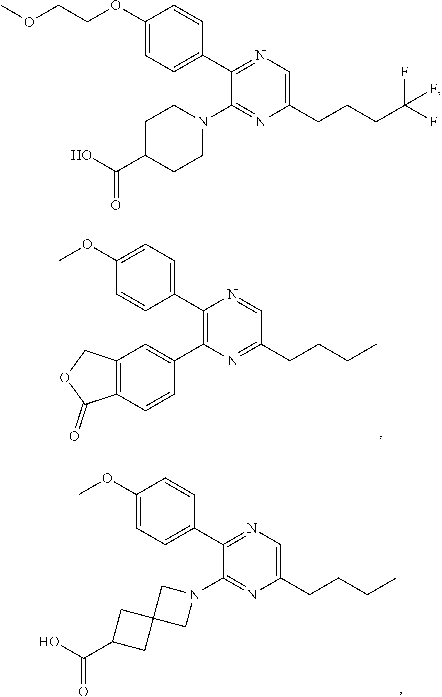 [SMILES: COCCOc1ccc(-c2ncc(CCCC(F)(F)F)nc2N2CCC(C(=O)O)CC2)cc1 ; CCCCc1cnc(-c2ccc(OC)cc2)c(-c2ccc3c(c2)COC3=O)n1 ; CCCCc1cnc(-c2ccc(OC)cc2)c(N2CC3(CC(C(=O)O)C3)C2)n1] 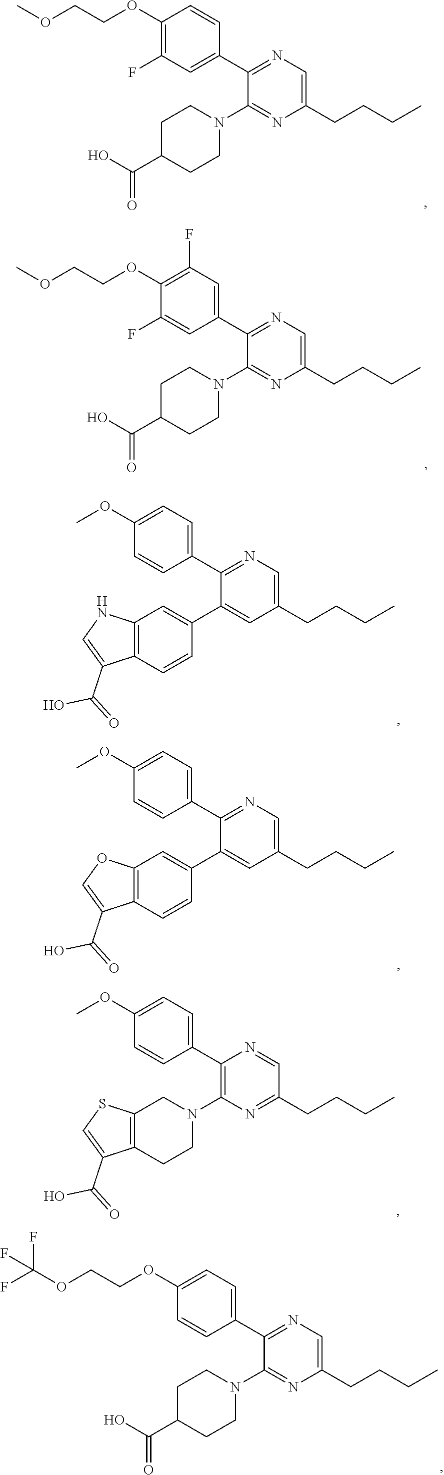 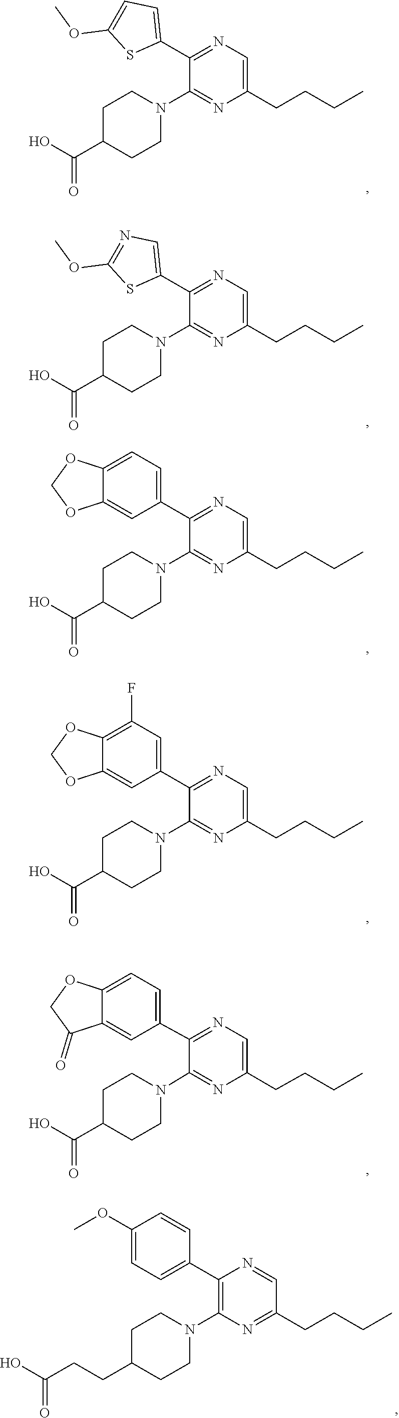 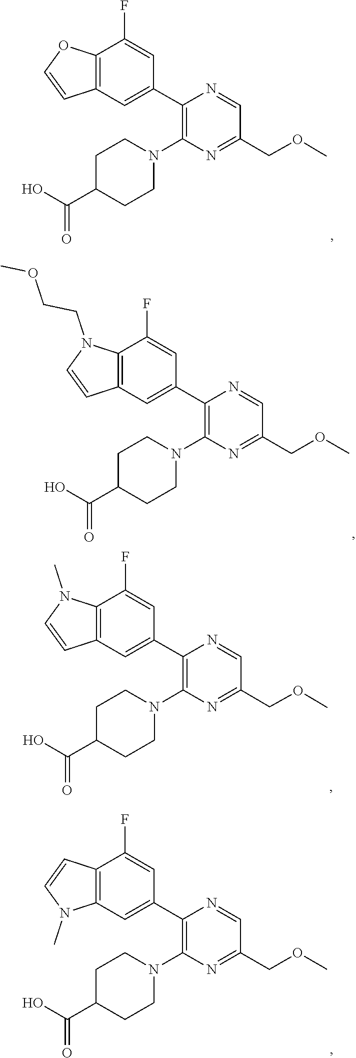 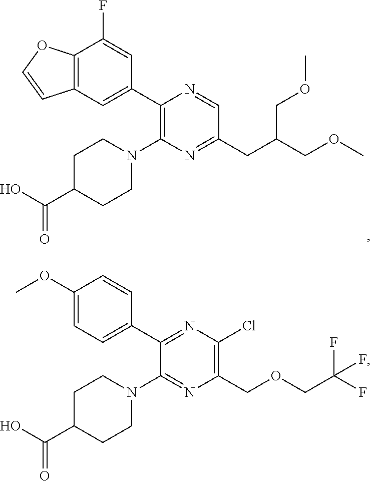 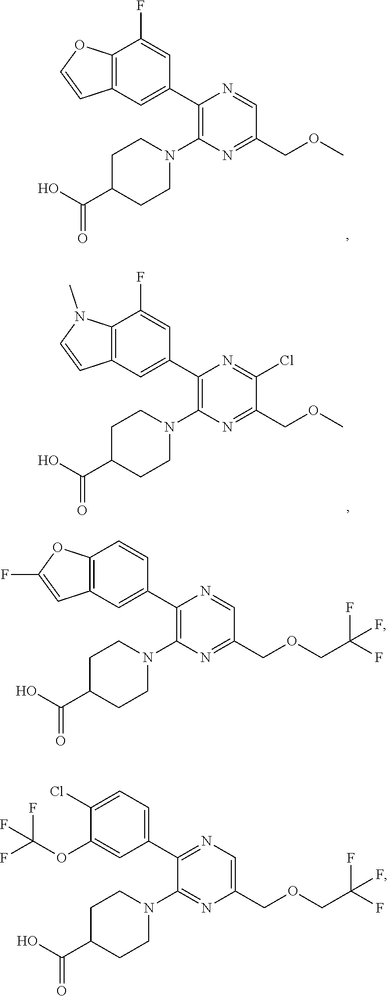 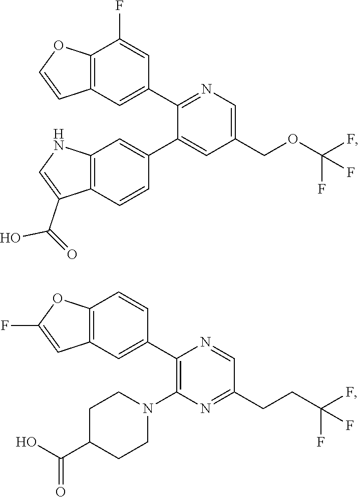 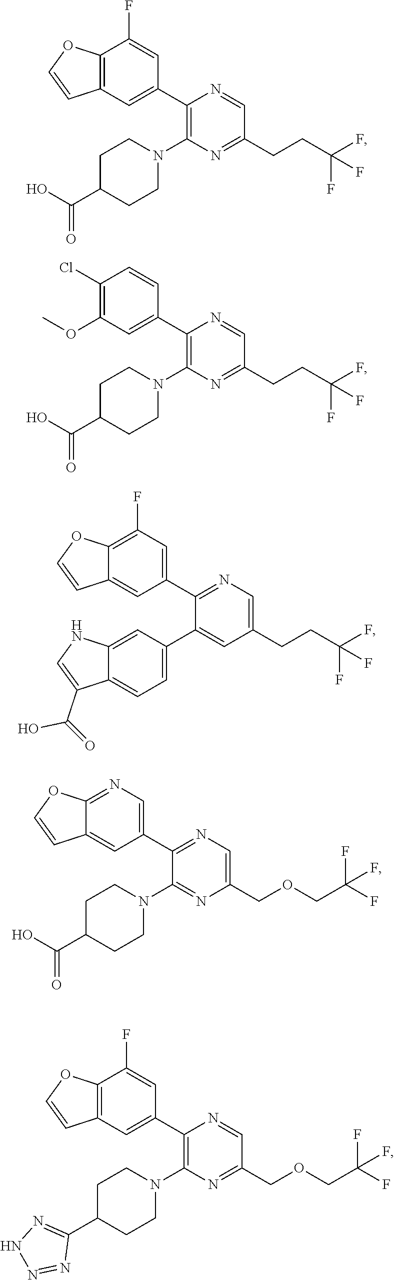 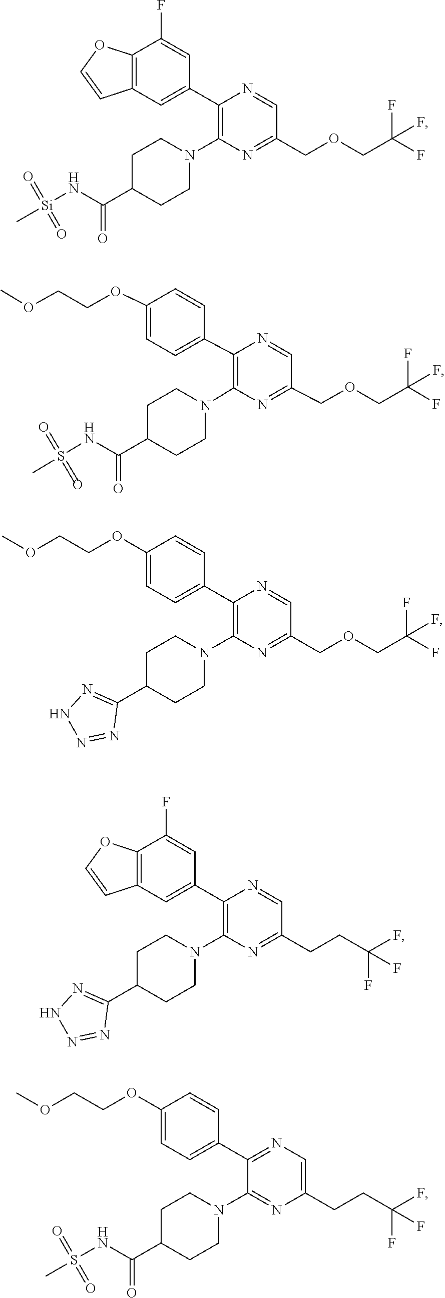 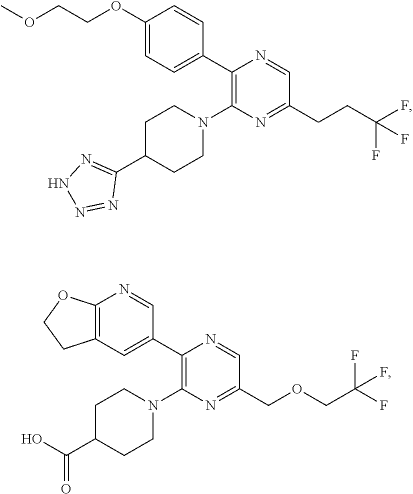 [SMILES: COCCOc1ccc(-c2ncc(CCC(F)(F)F)nc2N2CCC(c3nn[nH]n3)CC2)cc1 ; O=C(O)C1CCN(c2nc(COCC(F)(F)F)cnc2-c2cnc3c(c2)CCO3)CC1] 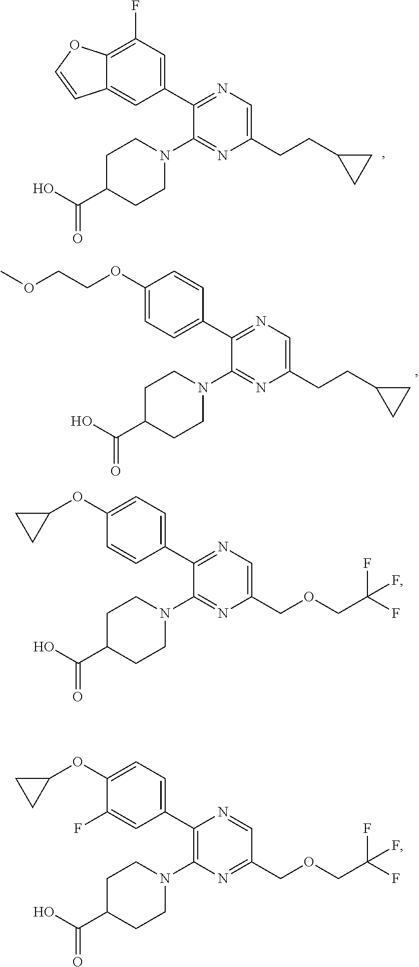 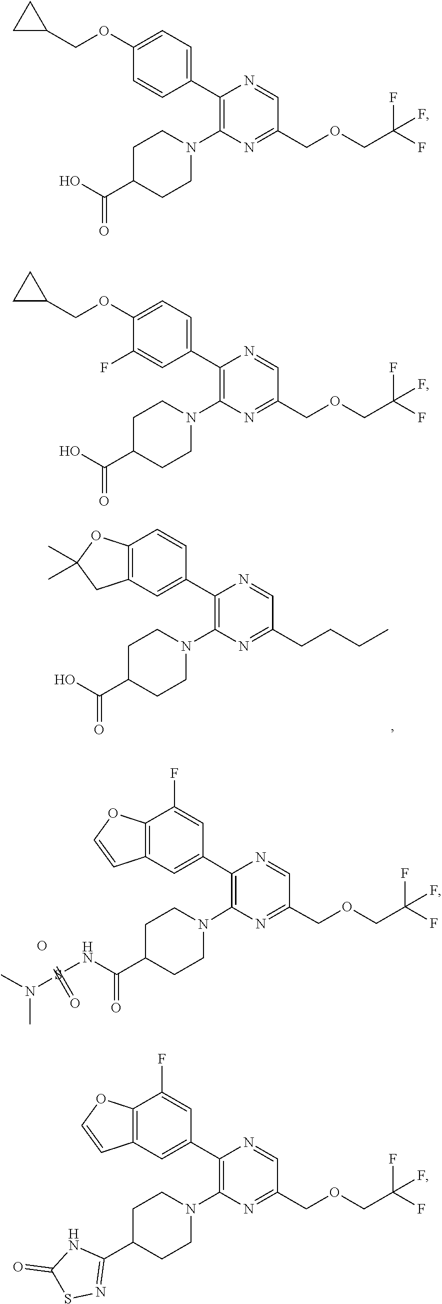 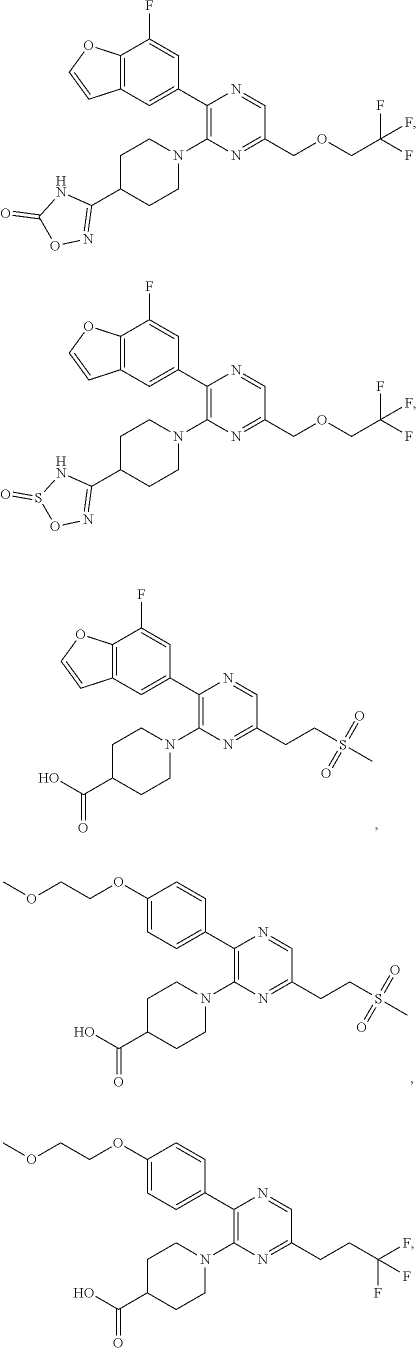 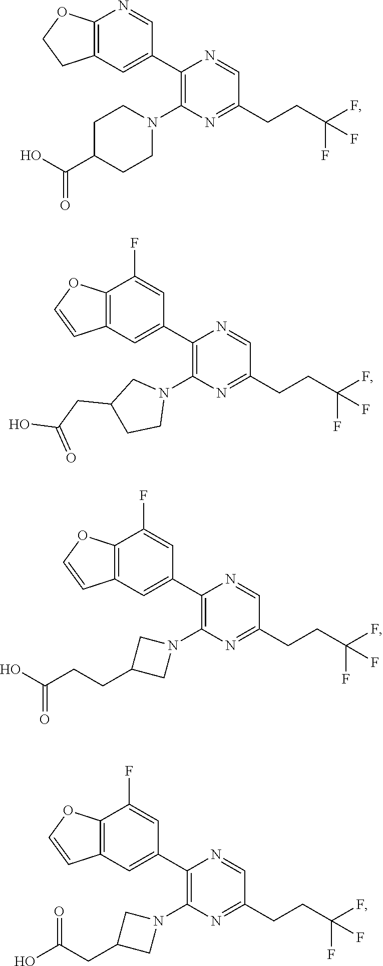 [SMILES: O=C(O)C1CCN(c2nc(CCC(F)(F)F)cnc2-c2cnc3c(c2)CCO3)CC1 ; O=C(O)CC1CCN(c2nc(CCC(F)(F)F)cnc2-c2cc(F)c3occc3c2)C1 ; O=C(O)CCC1CN(c2nc(CCC(F)(F)F)cnc2-c2cc(F)c3occc3c2)C1 ; O=C(O)CC1CN(c2nc(CCC(F)(F)F)cnc2-c2cc(F)c3occc3c2)C1] 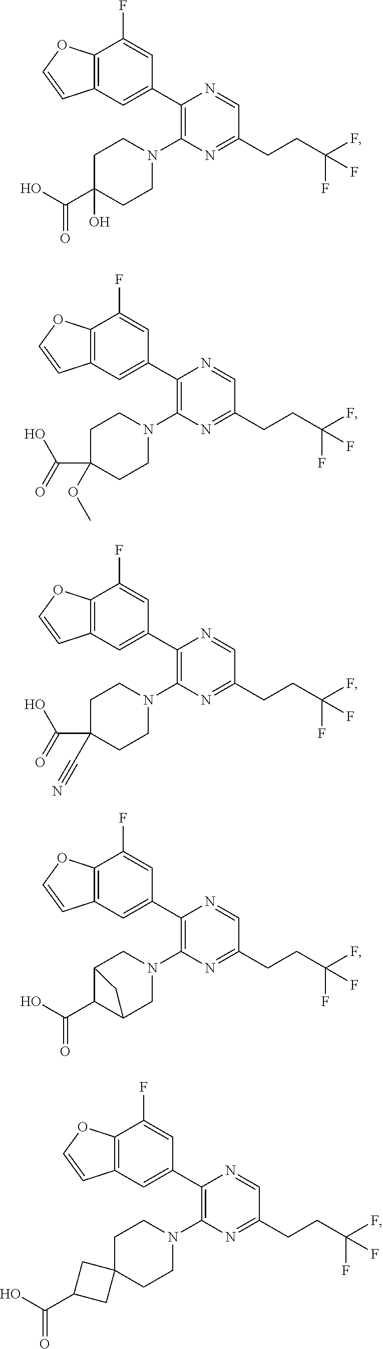 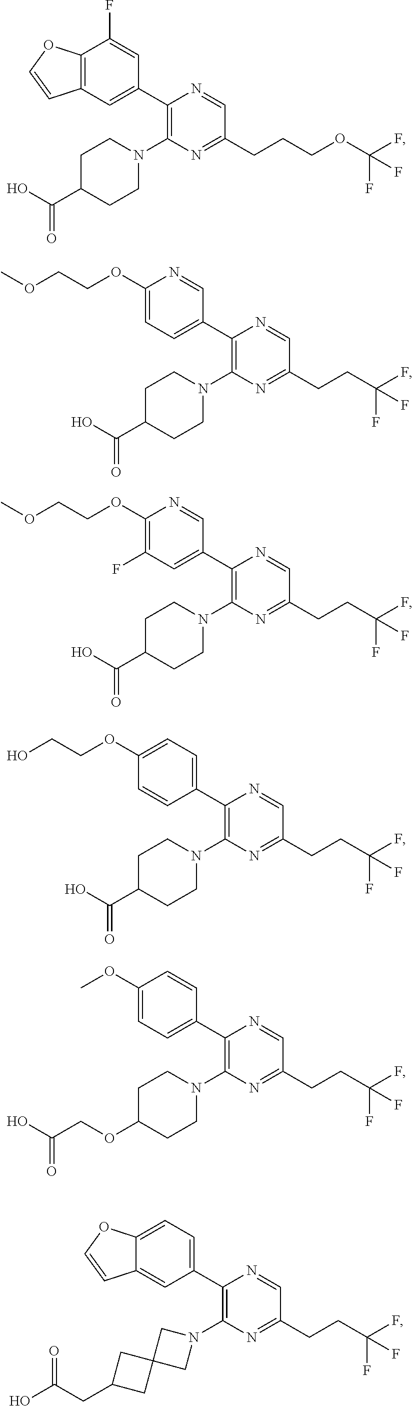 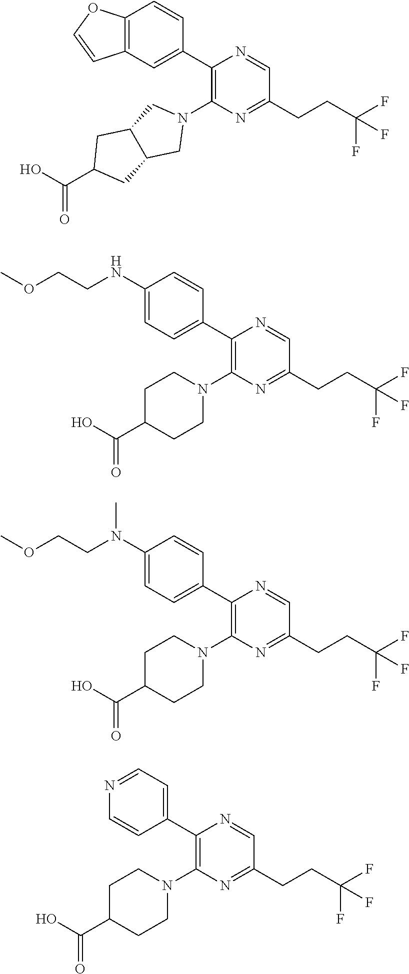 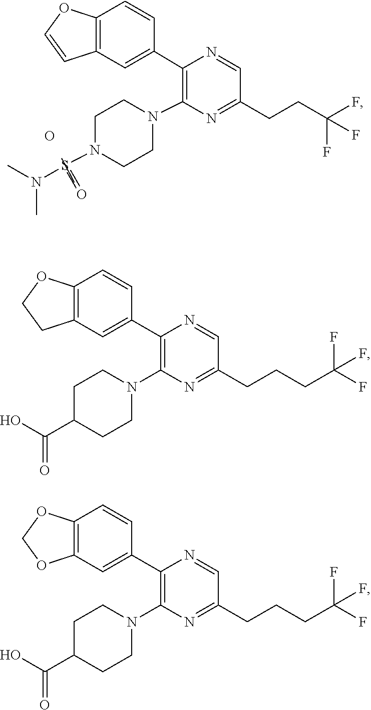 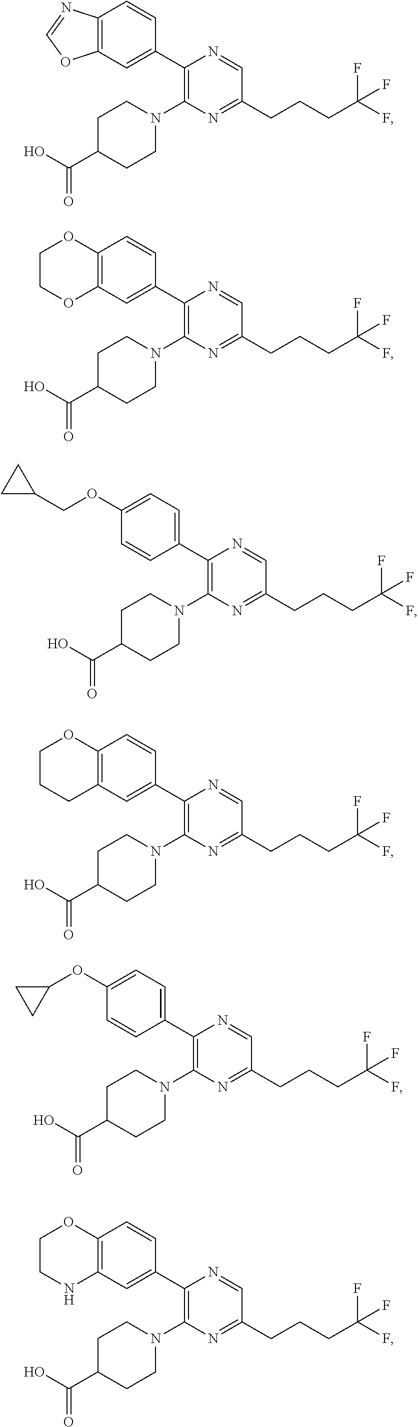 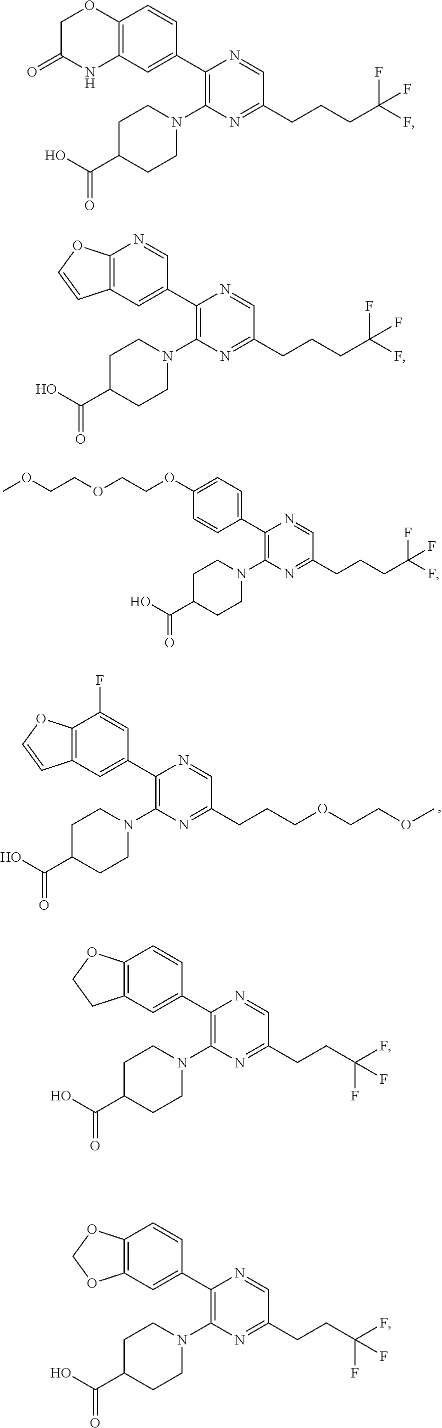 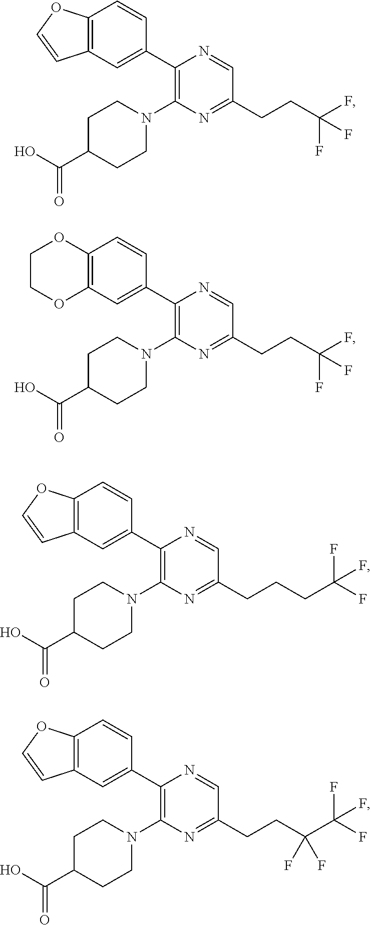 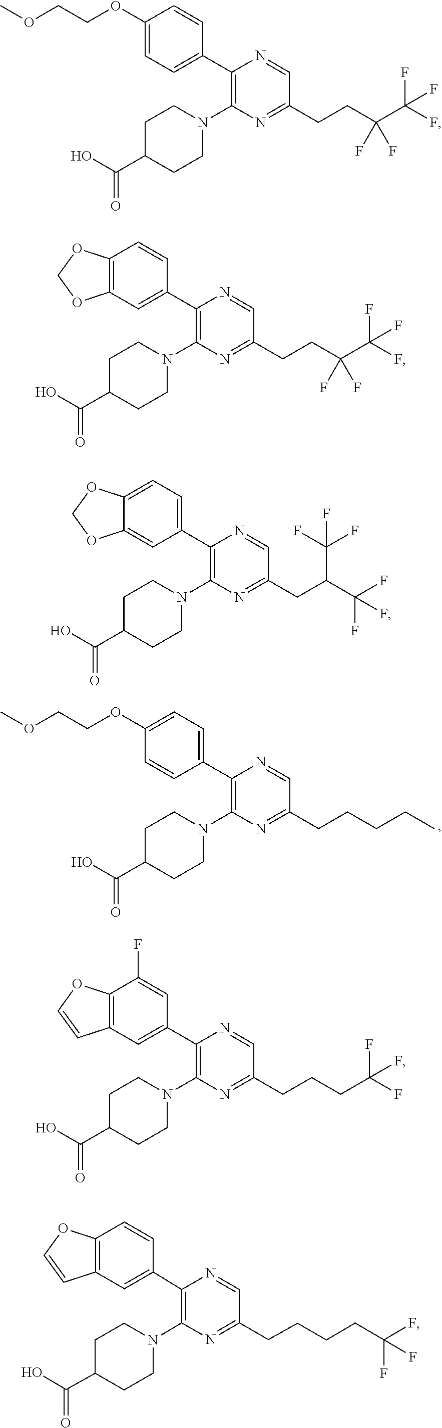 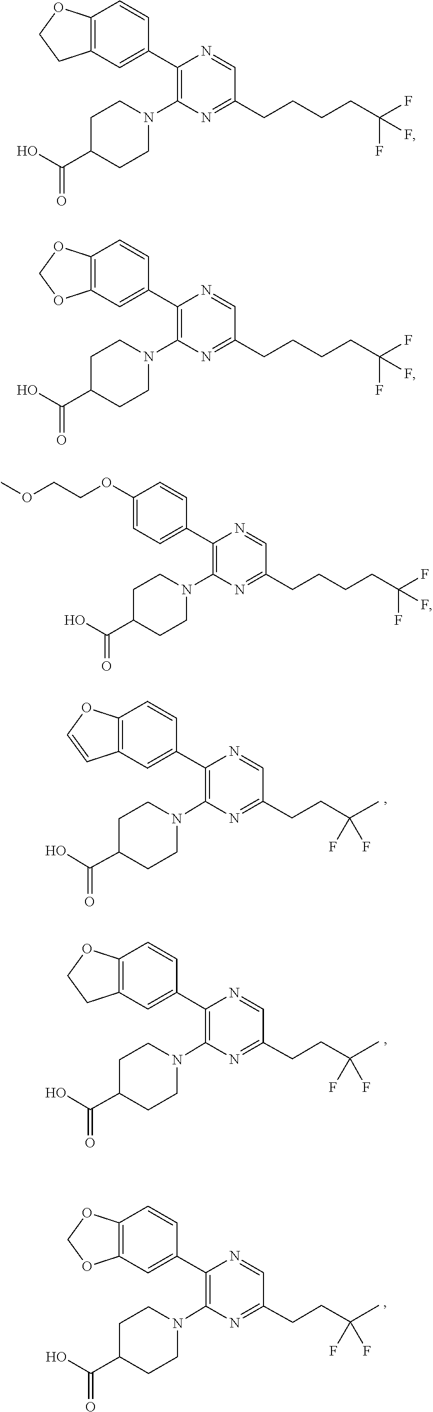 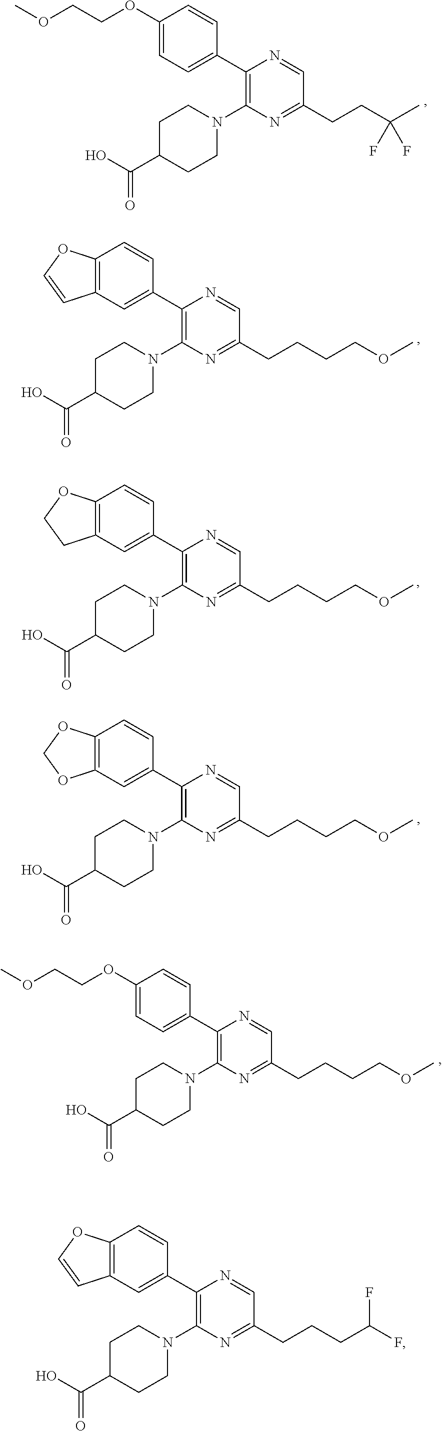 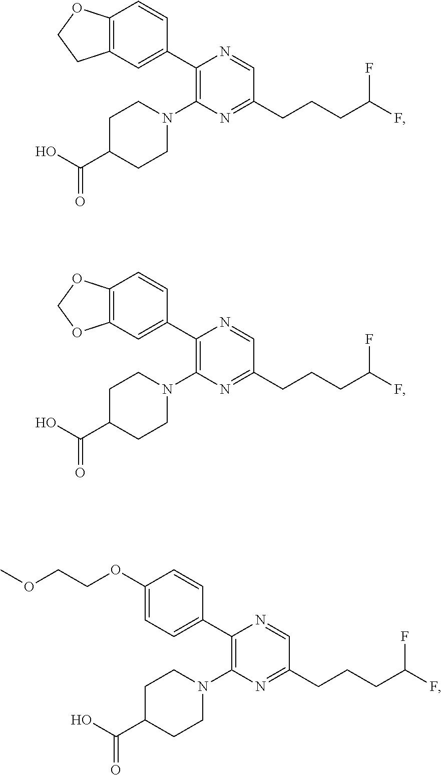 [SMILES: O=C(O)C1CCN(c2nc(CCCC(F)F)cnc2-c2ccc3c(c2)CCO3)CC1 ; O=C(O)C1CCN(c2nc(CCCC(F)F)cnc2-c2ccc3c(c2)OCO3)CC1 ; COCCOc1ccc(-c2ncc(CCCC(F)F)nc2N2CCC(C(=O)O)CC2)cc1] 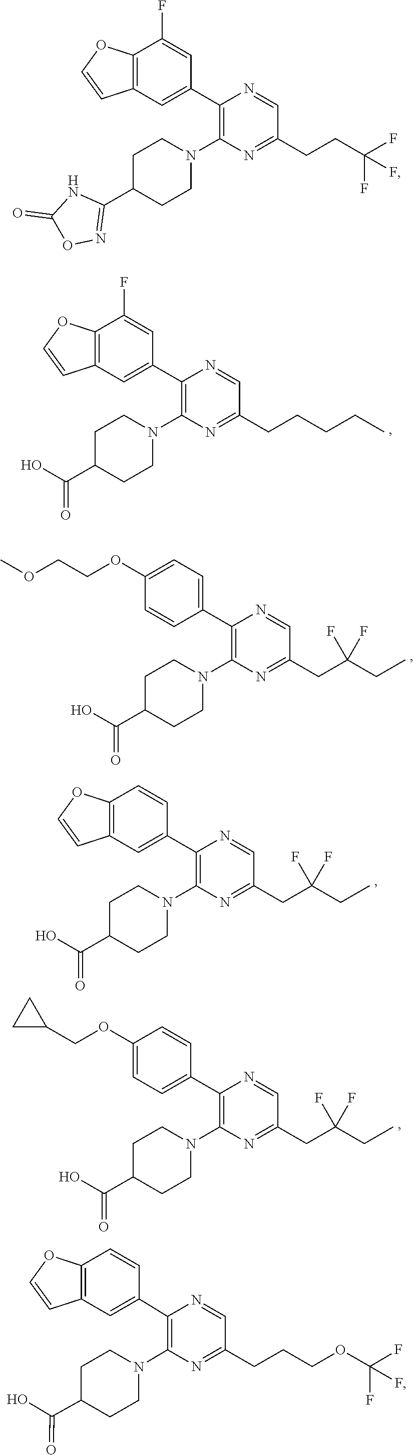 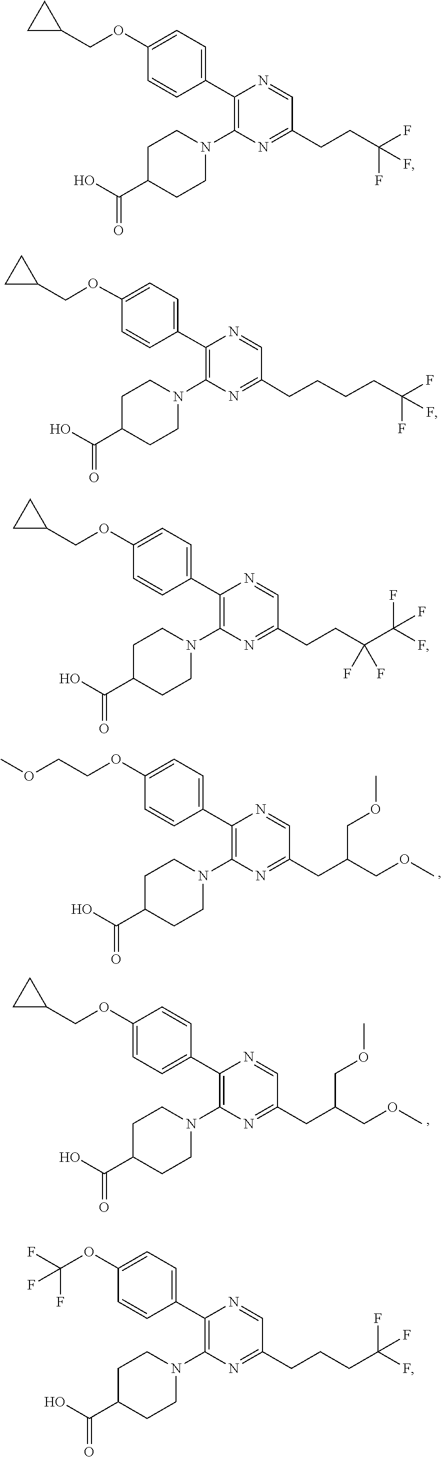 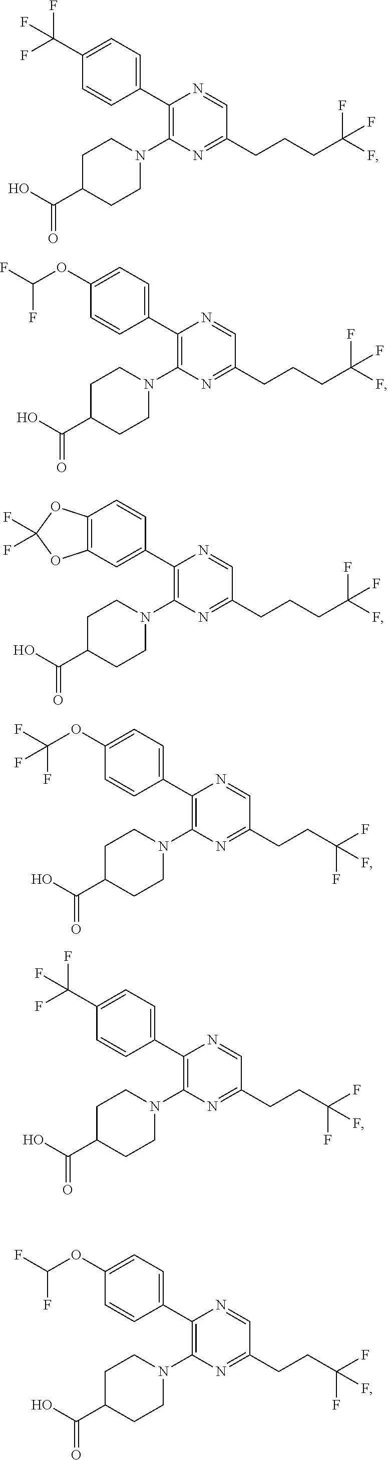 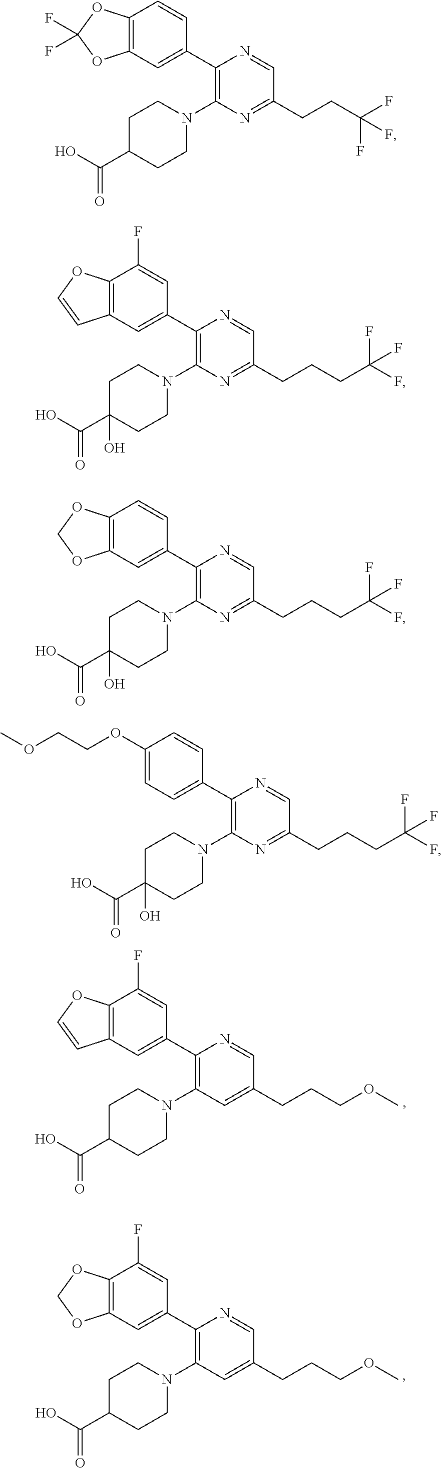 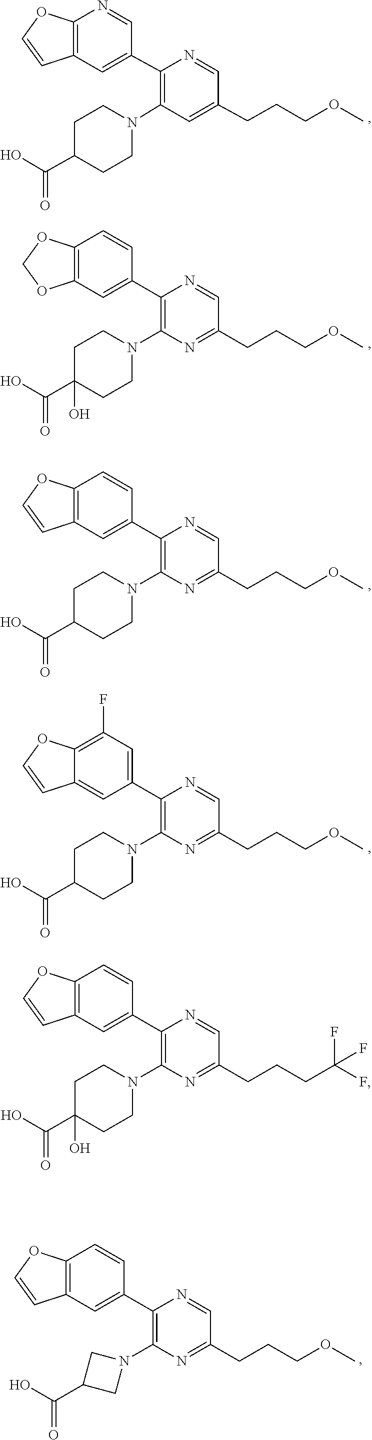 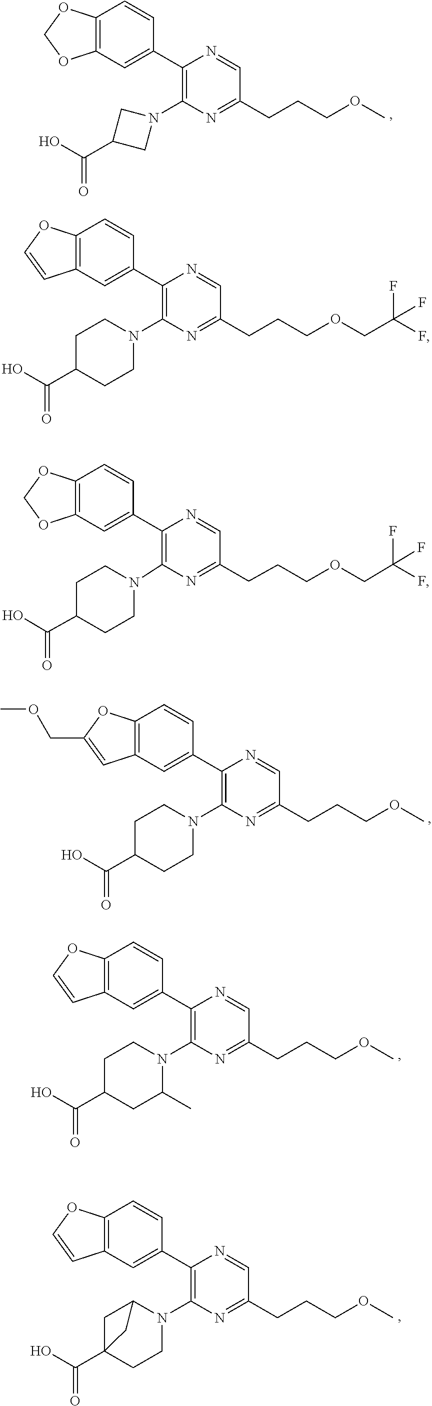 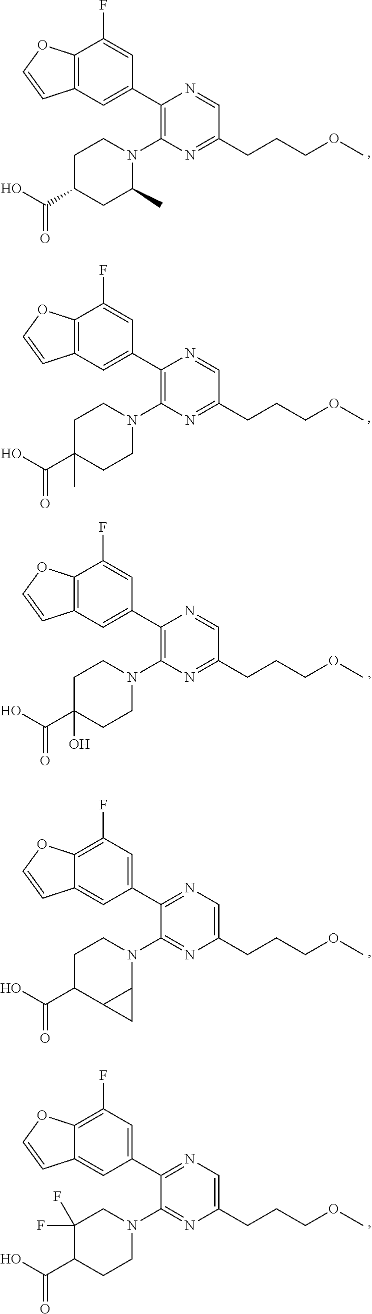 [SMILES: COCCCc1cnc(-c2cc(F)c3occc3c2)c(N2CC[C@@H](C(=O)O)C[C@@H]2C)n1 ; COCCCc1cnc(-c2cc(F)c3occc3c2)c(N2CCC(C)(C(=O)O)CC2)n1 ; COCCCc1cnc(-c2cc(F)c3occc3c2)c(N2CCC(O)(C(=O)O)CC2)n1 ; COCCCc1cnc(-c2cc(F)c3occc3c2)c(N2CCC(C(=O)O)C3CC32)n1 ; COCCCc1cnc(-c2cc(F)c3occc3c2)c(N2CCC(C(=O)O)C(F)(F)C2)n1] 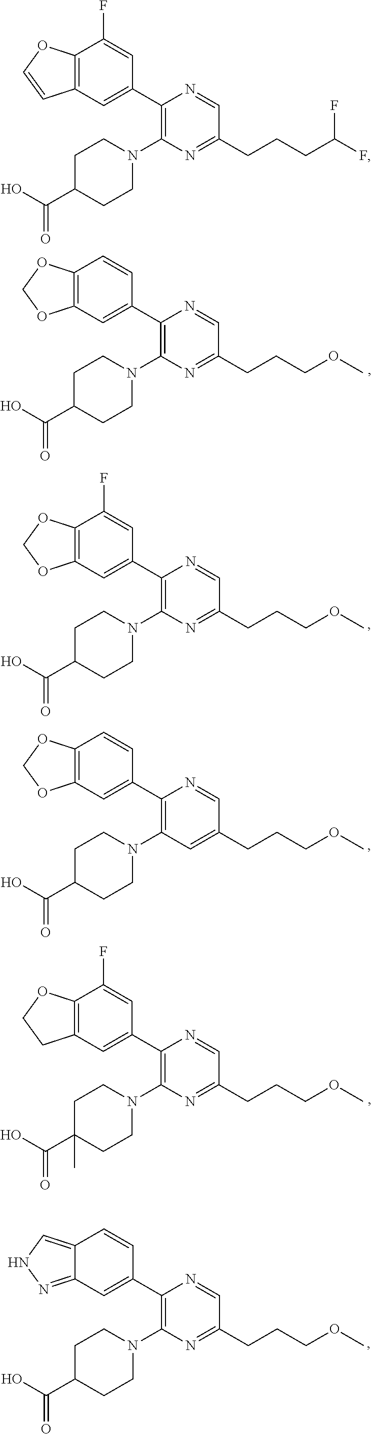 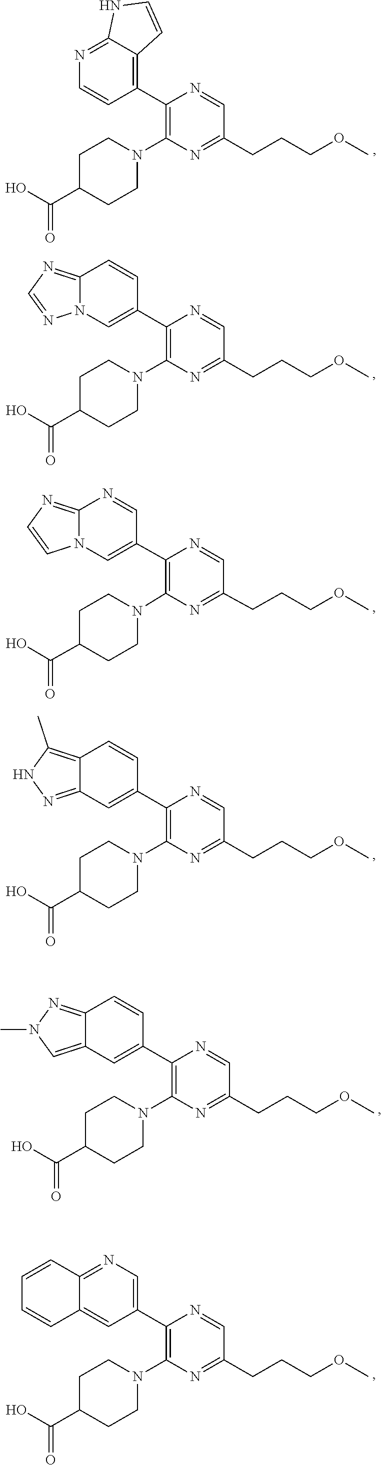 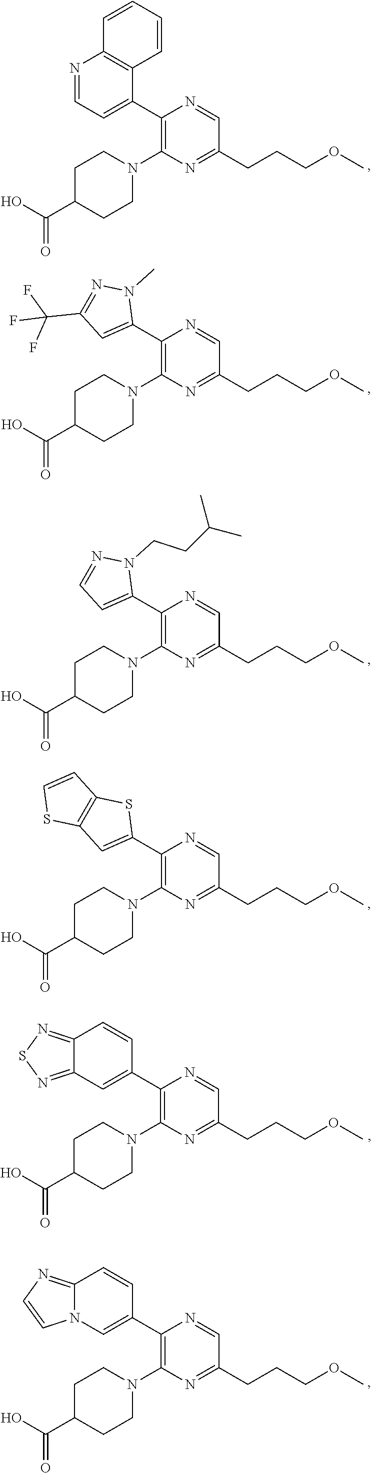 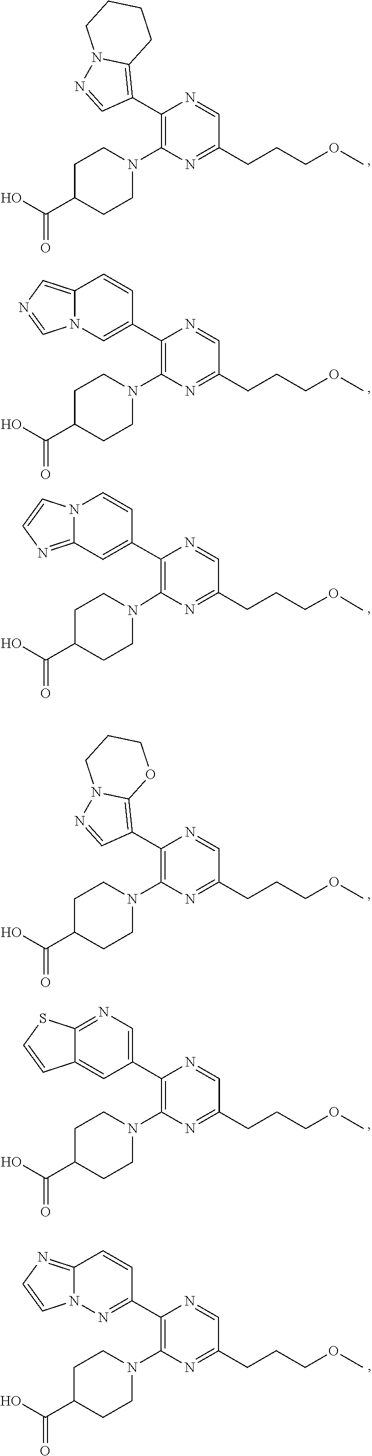 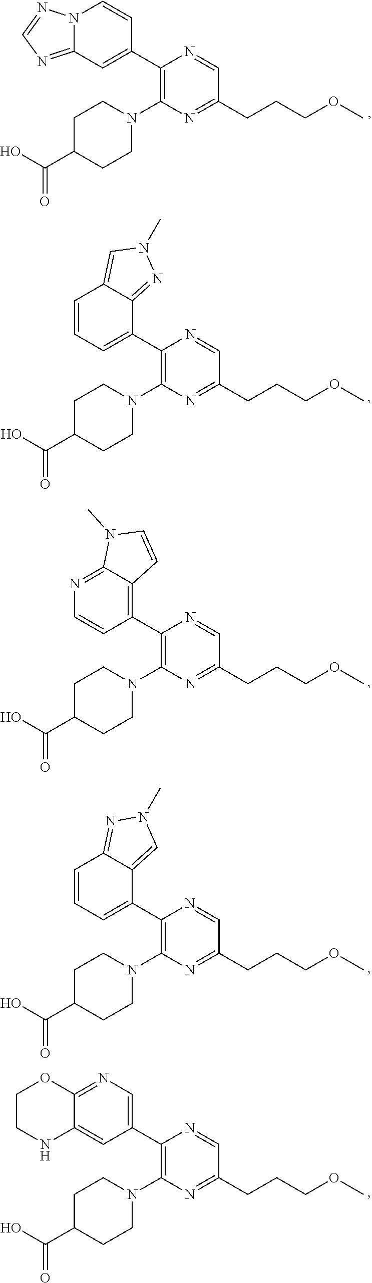 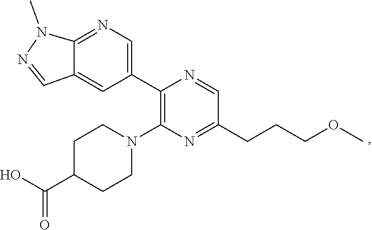 [SMILES: COCCCc1cnc(-c2cnc3c(cnn3C)c2)c(N2CCC(C(=O)O)CC2)n1] 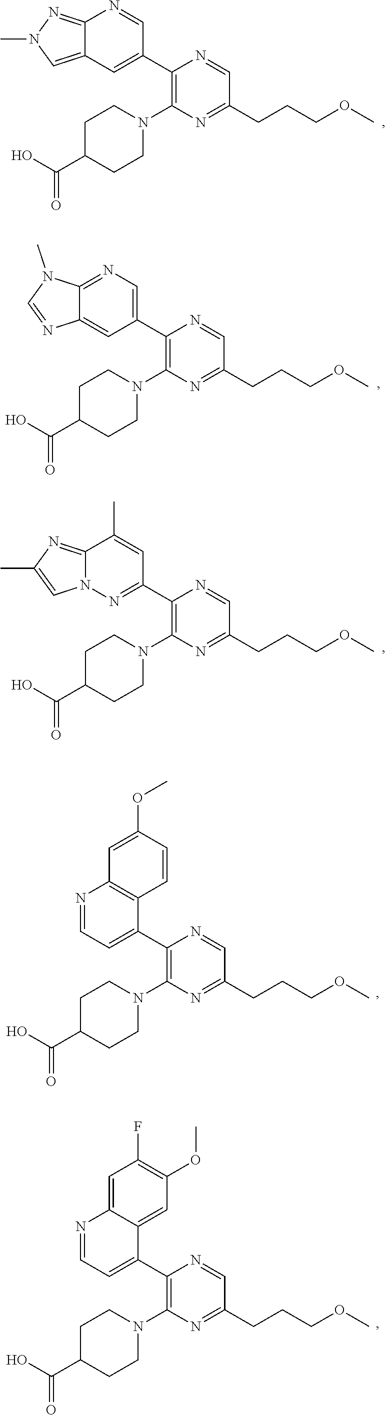 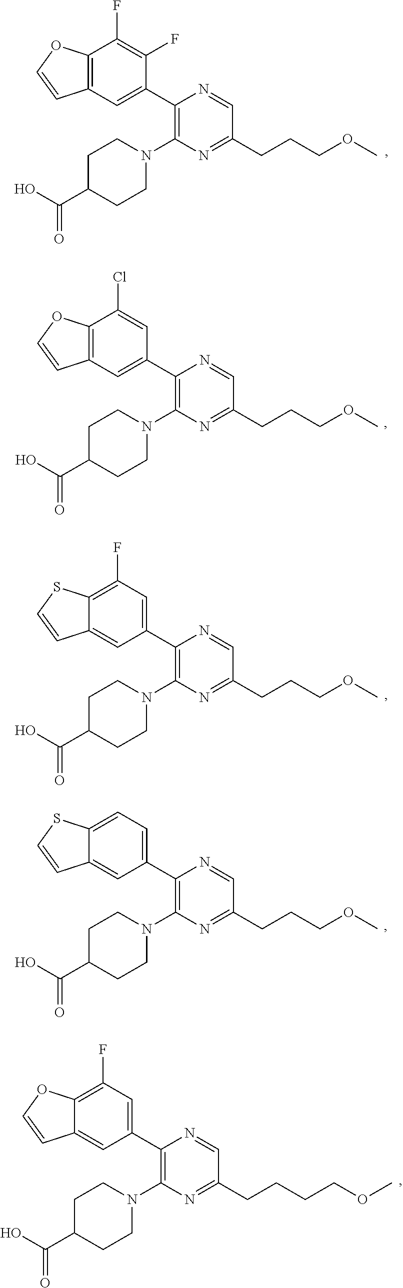 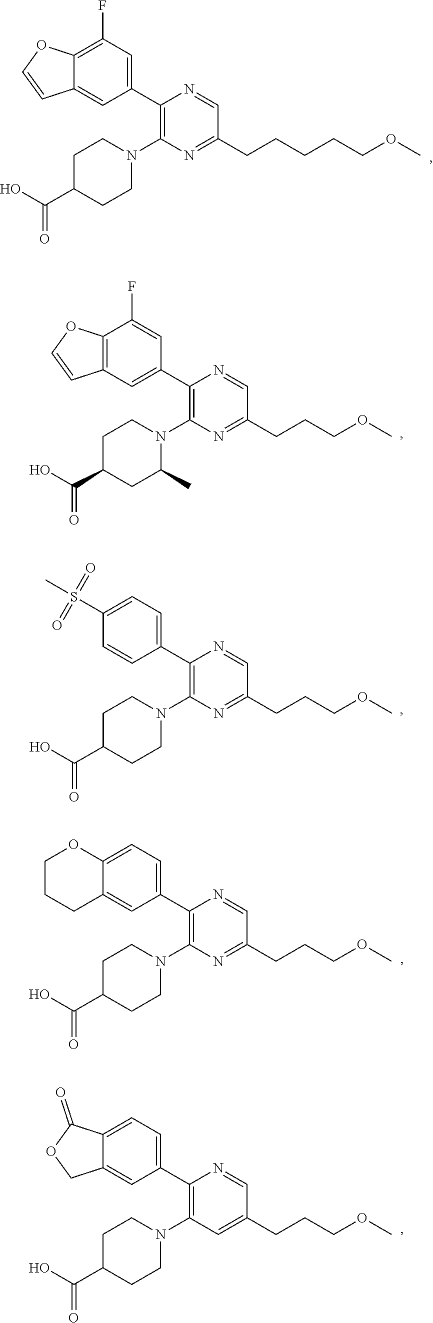 [SMILES: COCCCCCc1cnc(-c2cc(F)c3occc3c2)c(N2CCC(C(=O)O)CC2)n1 ; COCCCc1cnc(-c2cc(F)c3occc3c2)c(N2CC[C@H](C(=O)O)C[C@@H]2C)n1 ; COCCCc1cnc(-c2ccc(S(C)(=O)=O)cc2)c(N2CCC(C(=O)O)CC2)n1 ; COCCCc1cnc(-c2ccc3c(c2)CCCO3)c(N2CCC(C(=O)O)CC2)n1 ; COCCCc1cnc(-c2ccc3c(c2)COC3=O)c(N2CCC(C(=O)O)CC2)c1] 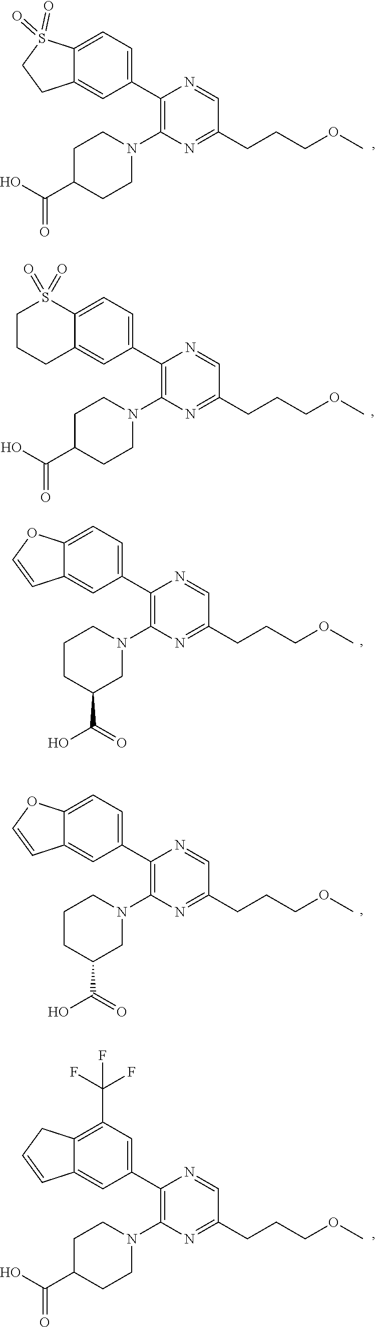 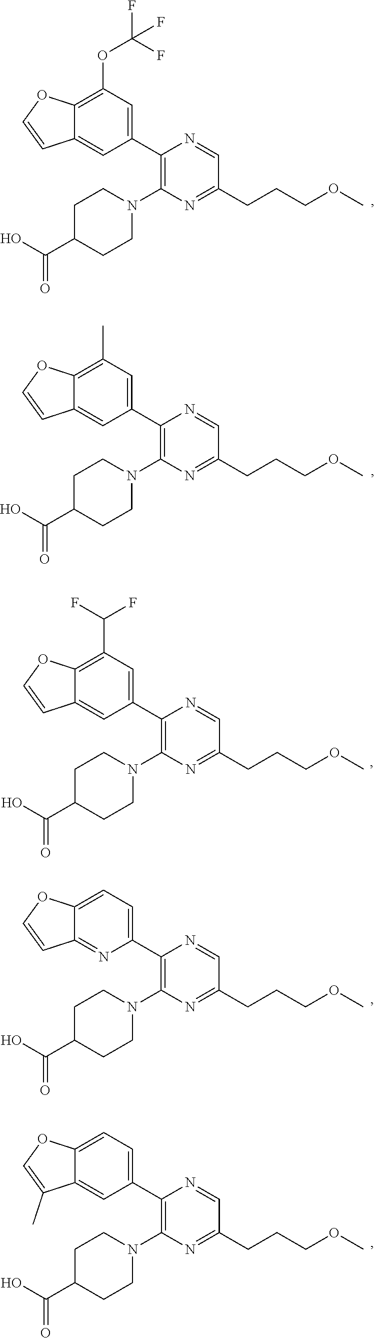 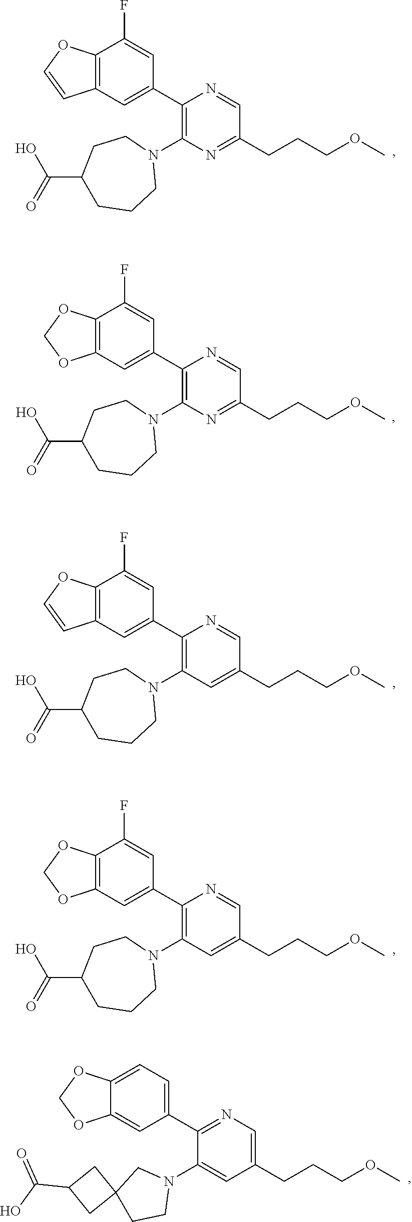 [SMILES: COCCCc1cnc(-c2cc(F)c3occc3c2)c(N2CCCC(C(=O)O)CC2)n1 ; COCCCc1cnc(-c2cc(F)c3c(c2)OCO3)c(N2CCCC(C(=O)O)CC2)n1 ; COCCCc1cnc(-c2cc(F)c3occc3c2)c(N2CCCC(C(=O)O)CC2)c1 ; COCCCc1cnc(-c2cc(F)c3c(c2)OCO3)c(N2CCCC(C(=O)O)CC2)c1 ; COCCCc1cnc(-c2ccc3c(c2)OCO3)c(N2CCC3(CC(C(=O)O)C3)C2)c1] 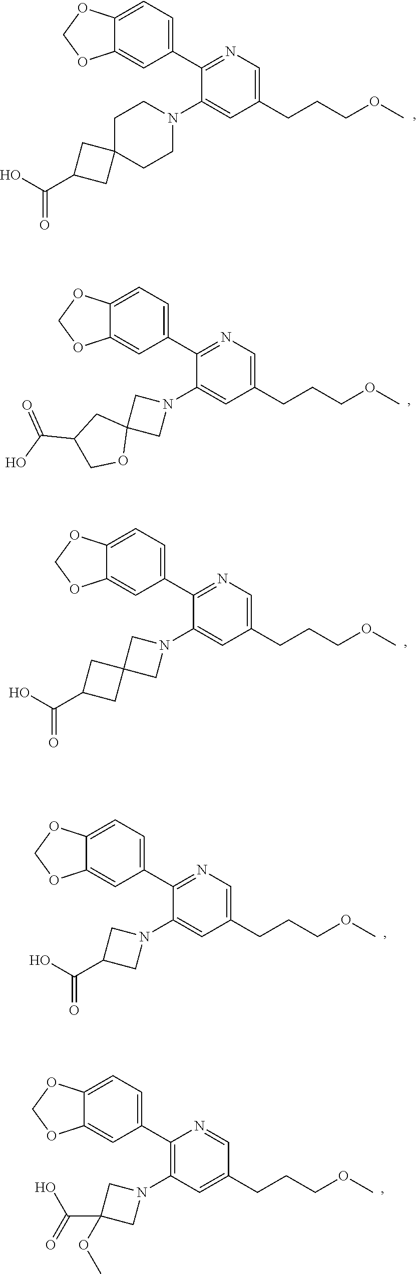 [SMILES: COCCCc1cnc(-c2ccc3c(c2)OCO3)c(N2CCC3(CC2)CC(C(=O)O)C3)c1 ; COCCCc1cnc(-c2ccc3c(c2)OCO3)c(N2CC3(CC(C(=O)O)CO3)C2)c1 ; COCCCc1cnc(-c2ccc3c(c2)OCO3)c(N2CC3(CC(C(=O)O)C3)C2)c1 ; COCCCc1cnc(-c2ccc3c(c2)OCO3)c(N2CC(C(=O)O)C2)c1 ; COCCCc1cnc(-c2ccc3c(c2)OCO3)c(N2CC(OC)(C(=O)O)C2)c1] 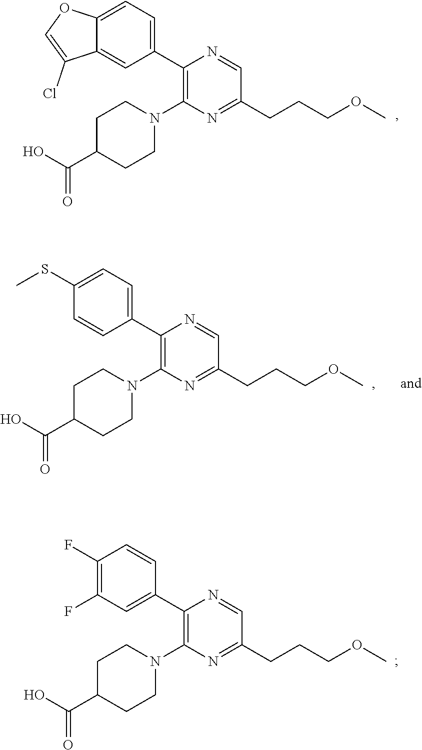 [SMILES: COCCCc1cnc(-c2ccc3occ(Cl)c3c2)c(N2CCC(C(=O)O)CC2)n1 ; COCCCc1cnc(-c2ccc(SC)cc2)c(N2CCC(C(=O)O)CC2)n1 ; COCCCc1cnc(-c2ccc(F)c(F)c2)c(N2CCC(C(=O)O)CC2)n1]
or a pharmaceutically acceptable salt thereof.

36. A pharmaceutical composition comprising the compound of claim 1, or a pharmaceutically acceptable salt thereof, and at least one pharmaceutically acceptable excipient.

37. (canceled)

38. (canceled)

39. (canceled)

40. (canceled)

41. A method of treatment of a GLUT9-mediated disease in a mammal comprising the administration of a therapeutically effective amount of a compound of claim 1, or a pharmaceutically acceptable salt thereof, to a mammal in need thereof.

42. The method of claim 41, wherein said disease is selected from hyperuricemia, gout, and uncontrolled gout including comorbidities and associated diseases.

43. A method of treatment of a GLUT9-mediated disease in a mammal comprising the administration of:
a. a therapeutically effective amount of a compound of claim 1, or a pharmaceutically acceptable salt thereof; and
b. another therapeutic agent;
to a mammal in need thereof.

44. (canceled)

45. (canceled)

46. (canceled)

47. (canceled)

48. (canceled)

49. (canceled)
